SEQUENCE LISTING (566 sequences)

SEQ ID NO: 1 - DNA, 64 bp, Mus musculus
  source 1..64: mol_type = genomic DNA
gataatcttgctccaacttggatggggtggagcggtggttcctcccctcagccctttattatgg

SEQ ID NO: 2 - DNA, 64 bp, Homo sapiens
  source 1..64: mol_type = genomic DNA
gataatcttgctccaacttggatggggtggagcgctggttcctcccctgagccctttattatgg

SEQ ID NO: 3 - RNA, 45 nt, Homo sapiens
  source 1..45: mol_type = unassigned RNA
annaatttttggagcaggttttctgacttcggtcggaaaacccct

SEQ ID NO: 4 - RNA, 31 nt, Homo sapiens
  source 1..31: mol_type = unassigned RNA
caggttttctgacttcggtcggaaaacccct

SEQ ID NO: 5 - DNA, 255 bp, Mus musculus
  source 1..255: mol_type = genomic DNA
aacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 6 - DNA, 411 bp, Mus musculus
  source 1..411: mol_type = genomic DNA
taaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 7 - DNA, 297 bp, Homo sapiens
  source 1..297: mol_type = genomic DNA
aacaacataggagctgtgattggctgttttcagccaatcagcactgactcagggcgacttctatgtagatgaggcagcgcagaggctgctgcctttacaagcggtcacaaactcaagaaacgagcggttttaatagtcttttagaatattgtttatcgaaccgaataaggaactgtgctttgtgattcacatatcagtggaggggtgtggaaatggcaccttgatgggagtgcgcgaggcaagtgaccgtgtgtggagttgatgtccttccctggctcgctacagacgcacttccgc

SEQ ID NO: 8 - DNA, 286 bp, Mus musculus
  source 1..286: mol_type = other DNA
aacaacataggagctgtgattggctgttttcagccaatcagcactgactcagggcgacttctatgtagatgaggcagcgcagaggctgctgcctttacaagcggtcacaaactcaagaaacgagcggttttaatagtcttttagaatattgtttatcgaaccgaataaggaactgtgctttgtgattcacatatcagtggaggggtgtggaaatggcaccttgatttaccgtaactatgaaatggagttgatgtccttccctggctcgctacagacgcacttccgc

SEQ ID NO: 9 - DNA, 104 bp, Mus musculus
  source 1..104: mol_type = unassigned DNA
cccaatttcactggtctacaatgaaagcaaaacagttctcttccccgctccccggtgtgtgagaggggctttgatccttctctggtttcctaggaaacgcgtat

SEQ ID NO: 10 - DNA, 35 bp, Homo sapiens
  source 1..35: mol_type = unassigned DNA
actttctggagtttcaaaagtagactgtacgctaa

SEQ ID NO: 11 - DNA, 20 bp, Homo sapiens
  source 1..20: mol_type = genomic DNA
atgatagggacttagggtga

SEQ ID NO: 12 - DNA, 20 bp, Homo sapiens
  source 1..20: mol_type = genomic DNA
tttgttccgtgggtggttta

SEQ ID NO: 13 - DNA, 20 bp, Homo sapiens
  source 1..20: mol_type = genomic DNA
tggggggaggtaggtaggta

SEQ ID NO: 14 - RNA, 20 nt, Mus musculus
  source 1..20: mol_type = unassigned RNA
gcaccccatcccccactaca

SEQ ID NO: 15 - RNA, 20 nt, Mus musculus
  source 1..20: mol_type = unassigned RNA
ggagaatcaccttataggat

SEQ ID NO: 16 - RNA, 20 nt, Mus musculus
  source 1..20: mol_type = unassigned RNA
gaataataacataataatat

SEQ ID NO: 17 - RNA, 20 nt, Mus musculus
  source 1..20: mol_type = unassigned RNA
gaaatttatgtattgattat

SEQ ID NO: 18 - RNA, 20 nt, Mus musculus
  source 1..20: mol_type = unassigned RNA
aaccaccgctccaccccatc

SEQ ID NO: 19 - RNA, 20 nt, Mus musculus
  source 1..20: mol_type = unassigned RNA
caagttggagcaagattatc

SEQ ID NO: 20 - RNA, 20 nt, Mus musculus
  source 1..20: mol_type = unassigned RNA
ctatataaaatagaaatata

SEQ ID NO: 21 - RNA, 20 nt, Mus musculus
  source 1..20: mol_type = unassigned RNA
tagtttattattagttagaa

SEQ ID NO: 22 - RNA, 24 nt, Mus musculus
  source 1..24: mol_type = unassigned RNA
gaggaaccaccgctccaccccatc

SEQ ID NO: 23 - RNA, 24 nt, Mus musculus
  source 1..24: mol_type = unassigned RNA
catccaagttggagcaagattatc

SEQ ID NO: 24 - RNA, 24 nt, Mus musculus
  source 1..24: mol_type = unassigned RNA
tatcctatataaaatagaaatata

SEQ ID NO: 25 - RNA, 24 nt, Mus musculus
  source 1..24: mol_type = unassigned RNA
tatatagtttattattagttagaa

SEQ ID NO: 26 - RNA, 26 nt, Mus musculus
  source 1..26: mol_type = unassigned RNA
gggaggaaccaccgctccaccccatc

SEQ ID NO: 27 - RNA, 26 nt, Mus musculus
  source 1..26: mol_type = unassigned RNA
cccatccaagttggagcaagattatc

SEQ ID NO: 28 - RNA, 26 nt, Mus musculus
  source 1..26: mol_type = unassigned RNA
attatcctatataaaatagaaatata

SEQ ID NO: 29 - RNA, 26 nt, Mus musculus
  source 1..26: mol_type = unassigned RNA
aatatatagtttattattagttagaa

SEQ ID NO: 30 - RNA, 30 nt, Mus musculus
  source 1..30: mol_type = unassigned RNA
tgaggggaggaaccaccgctccaccccatc

SEQ ID NO: 31 - RNA, 30 nt, Mus musculus
  source 1..30: mol_type = unassigned RNA
ccaccccatccaagttggagcaagattatc

SEQ ID NO: 32 - RNA, 30 nt, Mus musculus
  source 1..30: mol_type = unassigned RNA
caagattatcctatataaaatagaaatata

SEQ ID NO: 33 - RNA, 30 nt, Mus musculus
  source 1..30: mol_type = unassigned RNA
tagaaatatatagtttattattagttagaa

SEQ ID NO: 34 - RNA, 20 nt, Mus musculus
  source 1..20: mol_type = unassigned RNA
agtgcaaggattaaaggtag

SEQ ID NO: 35 - RNA, 20 nt, Mus musculus
  source 1..20: mol_type = unassigned RNA
caaaaggggtaatacagtac

SEQ ID NO: 36 - RNA, 20 nt, Mus musculus
  source 1..20: mol_type = unassigned RNA
ccataataaagggctgaggg

SEQ ID NO: 37 - RNA, 20 nt, Mus musculus
  source 1..20: mol_type = unassigned RNA
gaggaaccaccgctccaccc

SEQ ID NO: 38 - RNA, 24 nt, Mus musculus
  source 1..24: mol_type = unassigned RNA
tcacagtgcaaggattaaaggtag

SEQ ID NO: 39 - RNA, 24 nt, Mus musculus
  source 1..24: mol_type = unassigned RNA
gtagcaaaaggggtaatacagtac

SEQ ID NO: 40 - RNA, 24 nt, Mus musculus
  source 1..24: mol_type = unassigned RNA
gtacccataataaagggctgaggg

SEQ ID NO: 41 - RNA, 24 nt, Mus musculus
  source 1..24: mol_type = unassigned RNA
aggggaggaaccaccgctccaccc

SEQ ID NO: 42 - RNA, 26 nt, Mus musculus
  source 1..26: mol_type = unassigned RNA
agtcacagtgcaaggattaaaggtag

SEQ ID NO: 43 - RNA, 26 nt, Mus musculus
  source 1..26: mol_type = unassigned RNA
aggtagcaaaaggggtaatacagtac

SEQ ID NO: 44 - RNA, 26 nt, Mus musculus
  source 1..26: mol_type = unassigned RNA
cagtacccataataaagggctgaggg

SEQ ID NO: 45 - RNA, 26 nt, Mus musculus
  source 1..26: mol_type = unassigned RNA
tgaggggaggaaccaccgctccaccc

SEQ ID NO: 46 - RNA, 30 nt, Mus musculus
  source 1..30: mol_type = unassigned RNA
cataagtcacagtgcaaggattaaaggtag

SEQ ID NO: 47 - RNA, 30 nt, Mus musculus
  source 1..30: mol_type = unassigned RNA
ttaaaggtagcaaaaggggtaatacagtac

SEQ ID NO: 48 - RNA, 30 nt, Mus musculus
  source 1..30: mol_type = unassigned RNA
aatacagtacccataataaagggctgaggg

SEQ ID NO: 49 - RNA, 30 nt, Mus musculus
  source 1..30: mol_type = unassigned RNA
gggctgaggggaggaaccaccgctccaccc

SEQ ID NO: 50 - RNA, 20 nt, Homo sapiens
  source 1..20: mol_type = unassigned RNA
ctatacaaaatagaaatata

SEQ ID NO: 51 - RNA, 20 nt, Homo sapiens
  source 1..20: mol_type = unassigned RNA
tagtttgttattagttagaa

SEQ ID NO: 52 - RNA, 24 nt, Homo sapiens
  source 1..24: mol_type = unassigned RNA
gaggaaccagcgctccaccccatc

SEQ ID NO: 53 - RNA, 24 nt, Homo sapiens
  source 1..24: mol_type = unassigned RNA
tatcctatacaaaatagaaatata

SEQ ID NO: 54 - RNA, 24 nt, Homo sapiens
  source 1..24: mol_type = unassigned RNA
tatatagtttgttattagttagaa

SEQ ID NO: 55 - RNA, 26 nt, Homo sapiens
  source 1..26: mol_type = unassigned RNA
gggaggaaccagcgctccaccccatc

SEQ ID NO: 56 - RNA, 26 nt, Homo sapiens
  source 1..26: mol_type = unassigned RNA
attatcctatacaaaatagaaatata

SEQ ID NO: 57 - RNA, 26 nt, Homo sapiens
  source 1..26: mol_type = unassigned RNA
aatatatagtttgttattagttagaa

SEQ ID NO: 58 - RNA, 30 nt, Homo sapiens
  source 1..30: mol_type = unassigned RNA
tcaggggaggaaccagcgctccaccccatc

SEQ ID NO: 59 - RNA, 30 nt, Homo sapiens
  source 1..30: mol_type = unassigned RNA
caagattatcctatacaaaatagaaatata

SEQ ID NO: 60 - RNA, 30 nt, Homo sapiens
  source 1..30: mol_type = unassigned RNA
tagaaatatatagtttgttattagttagaa

SEQ ID NO: 61 - RNA, 20 nt, Homo sapiens
  source 1..20: mol_type = unassigned RNA
ccataataaagggctcaggg

SEQ ID NO: 62 - RNA, 20 nt, Homo sapiens
  source 1..20: mol_type = unassigned RNA
gaggaaccagcgctccaccc

SEQ ID NO: 63 - RNA, 24 nt, Homo sapiens
  source 1..24: mol_type = unassigned RNA
gtacccataataaagggctcaggg

SEQ ID NO: 64 - RNA, 24 nt, Homo sapiens
  source 1..24: mol_type = unassigned RNA
aggggaggaaccagcgctccaccc

SEQ ID NO: 65 - RNA, 26 nt, Homo sapiens
  source 1..26: mol_type = unassigned RNA
cagtacccataataaagggctcaggg

SEQ ID NO: 66 - RNA, 26 nt, Homo sapiens
  source 1..26: mol_type = unassigned RNA
tcaggggaggaaccagcgctccaccc

SEQ ID NO: 67 - RNA, 30 nt, Homo sapiens
  source 1..30: mol_type = unassigned RNA
aatacagtacccataataaagggctcaggg

SEQ ID NO: 68 - RNA, 30 nt, Homo sapiens
  source 1..30: mol_type = unassigned RNA
gggctcaggggaggaaccagcgctccaccc

SEQ ID NO: 69 - RNA, 20 nt, Homo sapiens
  source 1..20: mol_type = unassigned RNA
aaccagcgctccaccccatc

SEQ ID NO: 70 - RNA, 20 nt, Homo sapiens
  source 1..20: mol_type = unassigned RNA
ctatacaaaatagaaatata

SEQ ID NO: 71 - RNA, 20 nt, Homo sapiens
  source 1..20: mol_type = unassigned RNA
tagtttgttattagttagaa

SEQ ID NO: 72 - RNA, 24 nt, Homo sapiens
  source 1..24: mol_type = unassigned RNA
gaggaaccagcgctccaccccatc

SEQ ID NO: 73 - RNA, 24 nt, Homo sapiens
  source 1..24: mol_type = unassigned RNA
tatcctatacaaaatagaaatata

SEQ ID NO: 74 - RNA, 24 nt, Homo sapiens
  source 1..24: mol_type = unassigned RNA
tatatagtttgttattagttagaa

SEQ ID NO: 75 - RNA, 26 nt, Homo sapiens
  source 1..26: mol_type = unassigned RNA
gggaggaaccagcgctccaccccatc

SEQ ID NO: 76 - RNA, 26 nt, Homo sapiens
  source 1..26: mol_type = unassigned RNA
attatcctatacaaaatagaaatata

SEQ ID NO: 77 - RNA, 26 nt, Homo sapiens
  source 1..26: mol_type = unassigned RNA
aatatatagtttgttattagttagaa

SEQ ID NO: 78 - RNA, 30 nt, Homo sapiens; identical to SEQ ID NO: 58
  source 1..30: mol_type = unassigned RNA

SEQ ID NO: 79 - RNA, 30 nt, Homo sapiens; identical to SEQ ID NO: 59
  source 1..30: mol_type = unassigned RNA

SEQ ID NO: 80 - RNA, 30 nt, Homo sapiens; identical to SEQ ID NO: 60
  source 1..30: mol_type = unassigned RNA

SEQ ID NO: 81 - RNA, 20 nt, Homo sapiens
  source 1..20: mol_type = unassigned RNA
ccataataaagggctcaggg

SEQ ID NO: 82 - RNA, 20 nt, Homo sapiens
  source 1..20: mol_type = unassigned RNA
gaggaaccagcgctccaccc

SEQ ID NO: 83 - RNA, 24 nt, Homo sapiens
  source 1..24: mol_type = unassigned RNA
gtacccataataaagggctcaggg

SEQ ID NO: 84 - RNA, 24 nt, Homo sapiens
  source 1..24: mol_type = unassigned RNA
aggggaggaaccagcgctccaccc

SEQ ID NO: 85 - RNA, 26 nt, Homo sapiens
  source 1..26: mol_type = unassigned RNA
cagtacccataataaagggctcaggg

SEQ ID NO: 86 - RNA, 26 nt, Homo sapiens
  source 1..26: mol_type = unassigned RNA
tcaggggaggaaccagcgctccaccc

SEQ ID NO: 87 - RNA, 30 nt, Homo sapiens; identical to SEQ ID NO: 67
  source 1..30: mol_type = unassigned RNA

SEQ ID NO: 88 - RNA, 30 nt, Homo sapiens; identical to SEQ ID NO: 68
  source 1..30: mol_type = unassigned RNA

SEQ ID NO: 89 - RNA, 20 nt, Homo sapiens
  source 1..20: mol_type = unassigned RNA
aaccagcgctccaccccatc

SEQ ID NO: 90 - RNA, 20 nt, Homo sapiens
  source 1..20: mol_type = unassigned RNA
ctatacaaaatagaaatata

SEQ ID NO: 91 - RNA, 20 nt, Homo sapiens
  source 1..20: mol_type = unassigned RNA
tagtttgttattagttagaa

SEQ ID NO: 92 - RNA, 24 nt, Homo sapiens
  source 1..24: mol_type = unassigned RNA
gaggaaccagcgctccaccccatc

SEQ ID NO: 93 - RNA, 24 nt, Homo sapiens
  source 1..24: mol_type = unassigned RNA
tatcctatacaaaatagaaatata

SEQ ID NO: 94 - RNA, 24 nt, Homo sapiens
  source 1..24: mol_type = unassigned RNA
tatatagtttgttattagttagaa

SEQ ID NO: 95 - RNA, 26 nt, Homo sapiens
  source 1..26: mol_type = unassigned RNA
gggaggaaccagcgctccaccccatc

SEQ ID NO: 96 - RNA, 26 nt, Homo sapiens
  source 1..26: mol_type = unassigned RNA
attatcctatacaaaatagaaatata

SEQ ID NO: 97 - RNA, 30 nt, Homo sapiens; identical to SEQ ID NO: 58
  source 1..30: mol_type = unassigned RNA

SEQ ID NO: 98 - RNA, 30 nt, Homo sapiens; identical to SEQ ID NO: 59
  source 1..30: mol_type = unassigned RNA

SEQ ID NO: 99 - RNA, 20 nt, Homo sapiens
  source 1..20: mol_type = unassigned RNA
ccataataaagggctcaggg

SEQ ID NO: 100 - RNA, 20 nt, Homo sapiens
  source 1..20: mol_type = unassigned RNA
gaggaaccagcgctccaccc

SEQ ID NO: 101 - RNA, 24 nt, Homo sapiens
  source 1..24: mol_type = unassigned RNA
gtacccataataaagggctcaggg

SEQ ID NO: 102 - RNA, 24 nt, Homo sapiens
  source 1..24: mol_type = unassigned RNA
aggggaggaaccagcgctccaccc

SEQ ID NO: 103 - RNA, 26 nt, Homo sapiens
  source 1..26: mol_type = unassigned RNA
cagtacccataataaagggctcaggg

SEQ ID NO: 104 - RNA, 30 nt, Homo sapiens; identical to SEQ ID NO: 67
  source 1..30: mol_type = unassigned RNA

SEQ ID NO: 105 - RNA, 24 nt, Homo sapiens
  source 1..24: mol_type = unassigned RNA
gtaatacagtacccataataaagg

SEQ ID NO: 106 - RNA, 28 nt, Homo sapiens
  source 1..28: mol_type = unassigned RNA
gggtaatacagtacccataataaagggc

SEQ ID NO: 107 - RNA, 30 nt, Homo sapiens
  source 1..30: mol_type = unassigned RNA
ggggtaatacagtacccataataaagggct

SEQ ID NO: 108 - RNA, 24 nt, Homo sapiens
  source 1..24: mol_type = unassigned RNA
aggggtaatacagtacccataata

SEQ ID NO: 109 - RNA, 28 nt, Homo sapiens
  source 1..28: mol_type = unassigned RNA
aaaaggggtaatacagtacccataataa

SEQ ID NO: 110 - RNA, 30 nt, Homo sapiens
  source 1..30: mol_type = unassigned RNA
caaaaggggtaatacagtacccataataaa

SEQ ID NO: 111 - RNA, 24 nt, Homo sapiens
  source 1..24: mol_type = unassigned RNA
tacagtacccataataaagggctg

SEQ ID NO: 112 - RNA, 28 nt, Homo sapiens
  source 1..28: mol_type = unassigned RNA
atacagtacccataataaagggctgagg

SEQ ID NO: 113 - RNA, 30 nt, Homo sapiens; identical to SEQ ID NO: 48
  source 1..30: mol_type = unassigned RNA

SEQ ID NO: 114 - RNA, 24 nt, Homo sapiens
  source 1..24: mol_type = unassigned RNA
aggggtaatacagtacccataata

SEQ ID NO: 115 - RNA, 28 nt, Homo sapiens
  source 1..28: mol_type = unassigned RNA
aaaaggggtaatacagtacccataataa

SEQ ID NO: 116 - RNA, 30 nt, Homo sapiens; identical to SEQ ID NO: 110
  source 1..30: mol_type = unassigned RNA

SEQ ID NO: 117 - RNA, 24 nt, Homo sapiens
  source 1..24: mol_type = unassigned RNA
tacagtacccataataaagggctg

SEQ ID NO: 118 - RNA, 28 nt, Homo sapiens
  source 1..28: mol_type = unassigned RNA
atacagtacccataataaagggctgagg

SEQ ID NO: 119 - RNA, 30 nt, Homo sapiens; identical to SEQ ID NO: 48
  source 1..30: mol_type = unassigned RNA

SEQ ID NO: 120 - RNA, 24 nt, Homo sapiens
  source 1..24: mol_type = unassigned RNA
agcaagattatcctatataaaata

SEQ ID NO: 121 - RNA, 28 nt, Homo sapiens
  source 1..28: mol_type = unassigned RNA
ggagcaagattatcctatataaaataga

SEQ ID NO: 122 - RNA, 30 nt, Homo sapiens
  source 1..30: mol_type = unassigned RNA
tggagcaagattatcctatataaaatagaa

SEQ ID NO: 123 - RNA, 24 nt, Homo sapiens
  source 1..24: mol_type = unassigned RNA
agattatcctatataaaatagaaa

SEQ ID NO: 124 - RNA, 28 nt, Homo sapiens
  source 1..28: mol_type = unassigned RNA
aagattatcctatataaaatagaaatat

SEQ ID NO: 125 - RNA, 30 nt, Homo sapiens; identical to SEQ ID NO: 32
  source 1..30: mol_type = unassigned RNA

SEQ ID NO: 126 - RNA, 24 nt, Homo sapiens
  source 1..24: mol_type = unassigned RNA
ttggagcaagattatcctatataa

SEQ ID NO: 127 - RNA, 28 nt, Homo sapiens
  source 1..28: mol_type = unassigned RNA
aagttggagcaagattatcctatataaa

SEQ ID NO: 128 - RNA, 30 nt, Homo sapiens
  source 1..30: mol_type = unassigned RNA
caagttggagcaagattatcctatataaaa

SEQ ID NO: 129 - RNA, 24 nt, Homo sapiens
  source 1..24: mol_type = unassigned RNA
ttggagcaagattatcctatataa

SEQ ID NO: 130 - RNA, 28 nt, Homo sapiens
  source 1..28: mol_type = unassigned RNA
aagttggagcaagattatcctatataaa

SEQ ID NO: 131 - RNA, 30 nt, Homo sapiens; identical to SEQ ID NO: 128
  source 1..30: mol_type = unassigned RNA

SEQ ID NO: 132 - RNA, 24 nt, Homo sapiens
  source 1..24: mol_type = unassigned RNA
agattatcctatataaaatagaaa

SEQ ID NO: 133 - RNA, 28 nt, Homo sapiens
  source 1..28: mol_type = unassigned RNA
aagattatcctatataaaatagaaatat

SEQ ID NO: 134 - RNA, 30 nt, Homo sapiens; identical to SEQ ID NO: 32
  source 1..30: mol_type = unassigned RNA

SEQ ID NO: 135 - RNA, 48 nt, Homo sapiens
  source 1..48: mol_type = unassigned RNA
gtaatacagtacccataataaaggagcaagattatcctatataaaata

SEQ ID NO: 136 - RNA, 56 nt, Homo sapiens
  source 1..56: mol_type = unassigned RNA
gggtaatacagtacccataataaagggcggagcaagattatcctatataaaataga

SEQ ID NO: 137 - RNA, 60 nt, Homo sapiens
  source 1..60: mol_type = unassigned RNA
ggggtaatacagtacccataataaagggcttggagcaagattatcctatataaaatagaa

SEQ ID NO: 138 - RNA, 48 nt, Homo sapiens
  source 1..48: mol_type = unassigned RNA
aggggtaatacagtacccataataagattatcctatataaaatagaaa

SEQ ID NO: 139 - RNA, 56 nt, Homo sapiens
  source 1..56: mol_type = unassigned RNA
aaaaggggtaatacagtacccataataaaagattatcctatataaaatagaaatat

SEQ ID NO: 140 - RNA, 60 nt, Homo sapiens
  source 1..60: mol_type = unassigned RNA
caaaaggggtaatacagtacccataataaacaagattatcctatataaaatagaaatata

SEQ ID NO: 141 - RNA, 48 nt, Homo sapiens
  source 1..48: mol_type = unassigned RNA
tacagtacccataataaagggctgttggagcaagattatcctatataa

SEQ ID NO: 142 - RNA, 56 nt, Homo sapiens
  source 1..56: mol_type = unassigned RNA
atacagtacccataataaagggctgaggaagttggagcaagattatcctatataaa

SEQ ID NO: 143 - RNA, 60 nt, Homo sapiens
  source 1..60: mol_type = unassigned RNA
aatacagtacccataataaagggctgagggcaagttggagcaagattatcctatataaaa

SEQ ID NO: 144 - RNA, 48 nt, Homo sapiens
  source 1..48: mol_type = unassigned RNA
aggggtaatacagtacccataatattggagcaagattatcctatataa

SEQ ID NO: 145 - RNA, 56 nt, Homo sapiens
  source 1..56: mol_type = unassigned RNA
aaaaggggtaatacagtacccataataaaagttggagcaagattatcctatataaa

SEQ ID NO: 146 - RNA, 60 nt, Homo sapiens
  source 1..60: mol_type = unassigned RNA
caaaaggggtaatacagtacccataataaacaagttggagcaagattatcctatataaaa

SEQ ID NO: 147 - RNA, 48 nt, Homo sapiens
  source 1..48: mol_type = unassigned RNA
tacagtacccataataaagggctgagattatcctatataaaatagaaa

SEQ ID NO: 148 - RNA, 56 nt, Homo sapiens
  source 1..56: mol_type = unassigned RNA
atacagtacccataataaagggctgaggaagattatcctatataaaatagaaatat

SEQ ID NO: 149 - RNA, 60 nt, Homo sapiens
  source 1..60: mol_type = unassigned RNA
aatacagtacccataataaagggctgagggcaagattatcctatataaaatagaaatata

SEQ ID NO: 150 - RNA, 11 nt, Homo sapiens
  source 1..11: mol_type = unassigned RNA
aatttgtctag

SEQ ID NO: 151 - RNA, 11 nt, Homo sapiens
  source 1..11: mol_type = unassigned RNA
aatttttggag

SEQ ID NO: 152 - RNA, 20 nt, Homo sapiens
  source 1..20: mol_type = unassigned RNA
aaccagcgctccaccccatc

SEQ ID NO: 153 - DNA, 164 bp, Mus musculus
  source 1..164: mol_type = genomic DNA
catatcagctttctaactaataataaactatatatttctattttatataggataatcttgctccaacttggatggggtggagcggtggttcctcccctcagccctttattatgggtactgtattaccccttttgctacctttaatccttgcactgtgacttatg

SEQ ID NO: 154 - DNA, 164 bp, Homo sapiens
  source 1..164: mol_type = genomic DNA
catatcagatttctaactaataacaaactatatatttctattttgtataggataatcttgctccaacttggatggggtggagcgctggttcctcccctgagccctttattatgggtactgtattaccccttttgctacctttaatccttgcactgtgacttatg

SEQ ID NO: 155 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 156-317 - skipped (162 entries)

SEQ ID NO: 318 - DNA, 500 bp, Homo sapiens
  source 1..500: mol_type = genomic DNA
tttcttttgtaatccgaaacattctagtctgcgaattaaaagccattatttgaagaaggatgccccggctccatctggccaccgaaagattgctccttaacacaggctaaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 319 - DNA, 500 bp, Homo sapiens
  source 1..500: mol_type = genomic DNA
gtttcttctgtaatccgaaacattctagtctgcgaattaaaagccattatttgaagaaggatgccccggctccatctggccaccgaaaggttgctccttaacacaggctaaggaccagcttttttgggagagaacagacgcaggggcgggagggaaaaagggagaggcagacgtcacttcctcttggcggctctggcagcagattggtcagttgagtggcagaaaagcagacggggactgggcaaggcactgtcggtgacatcacggacagggcgacttctatgtagatgaggcagcgcagaggctgctgcttcgccacttgctgcttcgccacgaaggagttcccctgccctgggagcgggttcaggaccgcggatcggaagagagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 320 - DNA, 500 bp, Homo sapiens
  source 1..500: mol_type = genomic DNA
gtttcttttgtaatccgaaacattctagtctgcgaattaaaagccattatttgaagaaggatgccccggctccatctggccaccgaaaggttgctccttaacacaggctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaaggagttcccgtgccctgggagcgggttcaggaccgcggatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 321 - DNA, 500 bp, Homo sapiens
  source 1..500: mol_type = genomic DNA
tctccccgcccgcgggcggcgaagtaaaggcccagcgcagcccgcgctcctgccctggagcctcgtctttctccaggaaaacgtggaccgctctccgccgacaggtctcttccacagacccctgtcgccttcgcccccggtctcttccggttctgtcttttcgctggctcgatacgaacaaggaagtcgcccccagcggagccccggctcccccaggcagaggcggccccgggggcggagtcaacggcggaggccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 322 - DNA, 500 bp, Homo sapiens
  source 1..500: mol_type = genomic DNA
ccaacaagagagaccctcatctctacaaaatattttttaaaataactgggcctcatggtgcattcctgtaatctgagctagttgggaggatagctagagcccaggagatcgaggctgcagagaaccgtgatcacacgactgcactccagcctgggtgacagagcaagaccctgtctcaatagatagatagatagatagatagatagatagatagatagatagatagataaaaacagagaggtgaaaaatatggttttaagggttctgtttaagaatactctgttccaacatggagtttgtcaactctaaagtggtaaaaaacaagacagattgtagacattttcataaaaggcaggattatttggtgtcaccaaattaaatatgttaatggtcacagcggaggccgactcaatcagccttttattctgccaggctatttgatactgctgagggcttacctatttcctgaggagaaaaatagtcattaaaatgaccttata

SEQ ID NO: 323 - DNA, 500 bp, Homo sapiens
  source 1..500: mol_type = genomic DNA
gggcatgaaagataaagttctggaggtaagtagtgatgatggttacacaacaatgtgaacatattctatgccagtgaatttacacttaaggttaaaatggtaaattttatgtatactttggcataatttaaaaaaaagtttgaaaaagaaaaaagacaaaacaataatcgtctttggagaagtctgttacactgaatctggagccaagtgaacagaaccctgattttgatcccttttctctcaaaagcccttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 324 - DNA, 494 bp, Homo sapiens
  source 1..494: mol_type = genomic DNA
gagacagggcttaaaagtcctgggcaattagctgggcacagtgtcgtgcgcctgtaatcccacctagttgggaggctgaggcagaagaatctcttgaacccgaggtagaggttgcagtgagccgagatcgcgccactgcactccagcctggatgacagagtgagaccctgtctcaaaaaaaaaaaaagtcctgggcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 325 - DNA, 500 bp, Homo sapiens
  source 1..500: mol_type = genomic DNA
cgactcgcaaccttttcggggtcccgagtccaacacccgtgggaatcccatgggcaccatggcccctcgctccaaaaatgctttcgcgtcgcgcagacactgctcggtagtttcggggatcagcgtttgagtaagagcccgcgtctgaaccctccgcgccgccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 326 - DNA, 500 bp, Homo sapiens
  source 1..500: mol_type = genomic DNA
caaagccggttatgaagcgggggtggggtgggctagttttaataggtccaggcgattagtacctggcatccttaaccacctacagtttgagaagggagtgggtgataaaagcctggaagggaagggaaatcgggccgggcattaggctccatcgctcatcaatagacaaggcctttaggaaactgcgacaacggcttttgctctgggcctttactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcag

SEQ ID NO: 327 - DNA, 500 bp, Homo sapiens
  source 1..500: mol_type = genomic DNA
cacgcctgcctgggctgctccaaggtggtggctaggggtggcacaagttacacattttcaatttggataggttgtgattcttgtcttccccagtcagatacctacagccgctgagagccggtattccaacgccatgagacgccttctgtcgtgagcagttgacaagaaagacccagcacttactgcaaccggaaggagcggttcctccctcgctcgcgcagccgctcttccccgccactccctcggtgcccgccagcacattcccagcaagccctgagtatatttgcatatcaactcactacatttttttcttctaactaaaaaatcgaaaggacaaattccagattctccttgtgaagtcttcctttcagttcagaagaaatggaattcgctcttcaacttcaggaagttgaaataaagagttgcttggattttgtgttcacctttaccaaaaaatggatttggtaacactgccaccctgctttggtgacagagaaagc

SEQ ID NO: 328 - DNA, 500 bp, Homo sapiens
  source 1..500: mol_type = genomic DNA
tcgagctccactaggccgaggaccctaaaccccgaagaaaggaaaggcctctcaccgtcctttcgccgccgccccgcgcttcctgatgagttcgtccagggagatgtccgccatcttgctccgccgagcaagccgaaagcagtcgagaccccgcgagcccgccccttcggccgctagggagcgacgaactacttccggcgtccgcggaccaactctcgcgacagccagctcaaagcaggcaagaaccggaaggggcggggacgttccccgtgagccttcgcggtgctggctgctcatctgcatacggaagttcggcacattatgaattatttattttcctcgagggaaaaaattaaatgaaaagcaacaaaatacattattaacaagtgagacaaacttcaatggaactggatcatgacctcaacagtcaactacgatagtcatcatacgcctaatgagaatagaattcattacctaggaaataaactaaaaacgtcctt

SEQ ID NO: 329 - DNA, 500 bp, Homo sapiens
  source 1..500: mol_type = genomic DNA
gacaagagaaggactgaactgggggagtgagggaagaatgcgagagtgagactcagagagtaaaggggagggaaaaaaaagaagagagaaagggcgaggaagggggcgaccgcaggagggagacagacctggagtccactcggaggcctggagccgcacaaagcgccaggtcagcggtcccggctgggtgagaccagcaggcggctctagcgcgcgggagctgggcgaggctccgggacgacctcaccaatggagactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcg

SEQ ID NO: 330 - DNA, 500 bp, Mus musculus
  source 1..500: mol_type = genomic DNA
cacaagtaatgtacttttaaaattaacctttctataccggttttcacagtgcggctgtttttgttgttcctcttagtgttaattcacactaaagactgtgcatccgactcctacatttatgaaagtaaatgcctattgttagaacaaaaaaggctacagaacaaaaaacaaagcgaaataccatctgctttaggttcagtggtattttcccgctgacagggaggcgggtttttgggtacaggaaacgagtcactatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 331 - DNA, 500 bp, Mus musculus
  source 1..500: mol_type = genomic DNA
agtccttgctcttaactactgaaccaactctcccgtcctataagtaaattttcagagcactaatctattctacttcaagtttctcctgcatcagatccaggccagctggagcgagctacacggttaccccccctcctcccaaaggggaaaataaggatcaggtgaaaatgtttggaaattacaatagtagaatataaagaaagaatatgaaggcggccatgaaggggtagaaaggtagatgaccaggcgtcatccctgatgtcacgggtatgggcgtggtgtatgcagataagcgcgcagggtctgccgggtccgcgctccgtgcagaaatgtgaggggtttctcccgcggcgagggagggagcgcggatacgcgggtgtcagcagagcgcaagaatgtcgtgtgcgacgagggagacccgagatcggccagggctaagtgaccgtgtgttaagagtgtagaggcgacgggtgtgagccggggccgggcagtgggaaagc

SEQ ID NO: 332 - DNA, 500 bp, Mus musculus
  source 1..500: mol_type = genomic DNA
cggtggggctggctcctgtgctgcccgactcctgtgctccgacttggagaagacgggcgtccgttcgtccatacccaaaagagtcggccacaaggatgttggcacctgggggtgggccacgagcggccagttaggggtaagagacggtgcaaggtgacttgtccctggtccttcaccgctccagcactgcgccagtgagaaagtccaacaggccccgggatcgggcggcgaaaggaaggggcgtgatgtcactcctgggtggctgacggggcggggtgtatgcagatgagggtcgcgcaggggctgcggcgtccgcagcggccggggaaacgcgagagatttctcgcgcggcgtgggagagaacgcgggtcgccagatgtcagcggagcgccgagacggcgtgtgcgacgagggagacccgagatcgggcggggctaagtgaccgtgtgttaagagtgcaggggcggcgggtgtgagccggggccgggcgctgggagagc

SEQ ID NO: 333 - DNA, 500 bp, Mus musculus
  source 1..500: mol_type = genomic DNA
gctcctcctaggagctaacactggagccagggggatcaaagctgtgtagtgagtgtcacaaaacatcctagcactaaggagactgagacgggaacattgtagtaagttctagcctgggctacagtaagagacgtagtctcaaacaaggaaatgcgctataaataaataagatggagcttcaatggtctgtgcataactggggggttgaaggagccttccaaaggctgggttccggctccgttcagtcgttgacttgagctgggcggggcatgcaaataaggacgccgcctggtctccgcggaactctgggaacgaaaagcgcaacaaaatcctctcggcccccttggattcctcgggctttccctggcgtaaaggtggtggacaggatagaagaggagtttcggcgtcgggcgcgtgtttgcggtcgcgcgagtgttggcgaccgtgagttgaaggtgtgtgtggtgttgagtgtatggggcgtgtgggcgcagttcggt

SEQ ID NO: 334 - DNA, 501 bp, Mus musculus
  source 1..501: mol_type = genomic DNA
acagaatttactaaatagagagtgcagtcatggaattgccattttccccacgtggtttttcgtttgttcctcaccggaggcctccggtttgagcccgaagtctaaaccggtgcgcgtctgcgagctcgcgtgcgcctccgcggccggagcacagtcgcgggcgggcatcagggcggtgcgtggccgtcagtgacgcatgacccttcgcaggcagaagtgggacgggtcaagagaggactgtgatccgattggctgttcggcgaccgtatgctaatcgcggtgacagtctgcgtggtgtagggacacggtagcgactcaaaatgccccgtggacggggtctggagaggtgtcggtctgtcagagaaggtgtccccgcgagggcagacgcggtgtgagatagaggcgcggacagggatgtcacgggtcgtctgtgcgtcagtcgccgtgactaagatgtgggggcggccggtgcagtgcggcgcggtgtggagcgtgaggttt

SEQ ID NO: 335 - DNA, 500 bp, Mus musculus
  source 1..500: mol_type = genomic DNA
ttatctacgttggttttactacagaaactactgttctcagtaaaagcagagagagtacaatgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 336 - DNA, 500 bp, Mus musculus
  source 1..500: mol_type = genomic DNA
ggcatgtgattttgttttgtttaggtttttttttaactttatttgatcaacatgaaataatacaataagctatgtcttctaaccagcaggcagccagccaggagccatgtgtagcagtgttagaagctgcagcaggcatgtgggtaattgtggggagcttttaggaacagctagggagctgtgtggagctgcagagatgcagaggagcctggaggaacagaccagtgctgtgggctgggaggagcccccctcacaggtacttgctcaacggctccacgaccataagccttgaataagttacatggacattttacaaatggcacaggattgattatgtctgctgccagaagcctcatgtcgataaggagagagctggccaaaaggtaaagtatgctagctccatacagtgcaatagccacgtagtctcacagagaaagaggcaggcctgcatcatatccaaatatggccaagaattttattaaattaaaatgcaaacagca

SEQ ID NO: 337 - DNA, 500 bp, Mus musculus
  source 1..500: mol_type = genomic DNA
tatttttaatgaagcagggccatggagcccaagctggccttgaacttctcagcttttgtgctggtatcacagaaatgtggcgctatgcccggtcgggcctgatgttccagagtctttgcagtgtggggaacttgtaaggatgagactacaagacatcgggccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 338 - DNA, 500 bp, Mus musculus
  source 1..500: mol_type = genomic DNA
gttctttgcttttacagaccgtctcctgtgtgtccctgtcagagacctgaattcggttcctagcacccatatgttagaacataacgtccaagttggaggctacaagtggcactaaattacacattatcagttttgacaggttggggcaccgtgtaaccctagtaacttagcgaaaggctcctcgaacccaacatcccacctctagctgggagcagctgttttgcaccagcgccgcctacctctccctccgcgtcggccagagcattcccagcaagcactgctctgatttgcatattgactcacggactttcctttactgctatacaaaaaacgacacaacagttctctttgtgaagagctttccctcagcccgagaagaatcgatagtttgccttttagcttcgacaatatacagttgaaagttggttagactttattttcacctttaccgtaagatgggtggaccgacaatgccactgtgcttccgaacgcgctcgagc

SEQ ID NO: 339 - DNA, 500 bp, Mus musculus
  source 1..500: mol_type = genomic DNA
gaagcagcgaggcccctacgagcctcgaggaaaagggaaagggccgctcaccgtcccttggccgccgtcccgcgcttccggatgagctcgtccagcgagagatcggccatcttgcggcgcgcagctagcggagaacggaggagaagccgactgcggcgagcccgccccgcggccggagtcaagagcggcttccggcaacccggacggccaactctcgcgacagctcaaacgaaaagctcgagacggaaggggtggagcctctctccccgtgagcctttgcggtgctcgcttctaatttgcatggaggagtccggagccttatgggctctctctcccccctcctccttttgctaatgaggaattagaattatatcgatcataaaccattaaaaagcattatcaagaggacaagtcttttagtcatacagccgatcggacactcaccatacttgtaatgagaatataatttcttacccctgaggaacgtagacacgttcctc

SEQ ID NO: 340 - DNA, 500 bp, Mus musculus
  source 1..500: mol_type = genomic DNA
cgcttcggtgtgtcacttcggccacagagggagacagacgattacggcccacggaaggccgcgtgagtctcgtgaagctggagatgcgctgggtctacctttcgccgtccgctccgccttggcagaggcgccgacacgcaccgaaggcagagaacccatctcgtatgggaccgcaccggacacgggcgcctgggccaggagcagagccgggccgtagaatagacatggccgtcgggggcggggcttcggaaggtttaaccaatccaaactgttgtattttgcatagccccaaagcattttggttaacagtaaaaacatcctaaatttaagtattttaatttaaacttagaacgaagcgagtataaaaaggattatttaaccctaaaacggattcaggatttgttataatatcaagtacagtcggctacataaggtcaccacatgtgtaaagttacaaaattctatggccttatatacctaccaagagcctgcttactctc

SEQ ID NO: 341 - DNA, 500 bp, Homo sapiens
  source 1..500: mol_type = genomic DNA
actttctggagtttcaaaaacagactgtacgccaagggtcatatctttttttgtattggtttgtgtcttggttggtgtcttaggtgttaatcctacagtggagggtttatgggaataggaagtaacatgtcgcctgcatgtcataggagaaaaagcgagcatcagccgtatcggctttgtaacacaaattagctatcgtgaagtccgctcagctcttccctttctaccctggctgctttttgcagggattggtccgtggtctccagtctcttgggttctcaccctgtgtgaaaatcttcgtgtttttccctaccccccagtcacctcttacacagcctctgcttccaagcgcagcccccacaggagtttgtaggatttctgtgctagcggggaatgtgttctcacctcatagagccaggtagaaattatgcagatgggcagttctctgggaagaaagcagggcctttggggctctcagtgtccccgttgggttgtagaca

SEQ ID NO: 342 - DNA, 500 bp, Homo sapiens
  source 1..500: mol_type = genomic DNA
actttctggagtttctaaaagtagactgtacgctaagggtcatatctttttttgttttggtttgtgtcttggttggcgtcttaaatgttaatcctacagtggagggctggcgaataggaagtaacatgtcgcctgcacgccataggagaaaaagcgagcatcagccgtatcggctttgtaacacaaattagctatcgtgaagtccgctcagctcttccctttctaccctggctgctttttgcagggattggtccgtggtctccagtctcttgggttctcaccctgtgtgaaaatcttcctgtttttccctaccccccaagtcacctcttacacagcctctgcttccaagcgcagcccccacaggagtttgtaggatttctgtgctagcggggagtgtgttctcacctcatagagccaggtagaaactacgcagatgggtgctgttctccaggaagaaagcagggcctttggggctctcagtgtccccgttgggttgtaga

SEQ ID NO: 343 - DNA, 500 bp, Homo sapiens
  source 1..500: mol_type = genomic DNA
actttctggagtttcaaaaacagaccgtacgccaagggtcatgtcttttttcgtattggtttgtgtcttagttgttaatcctacagtggaggcctggggaataagaagtaacatgtggcctgcacgccataggagaaaaagcgagcatcagccgtatcggctttgtaacacaaattagctatcgtgaagtccgctcagctcttccatttctaccctggctgctttttgcagggattggtccgtggtctccagtctcttgggttctcaccctgtgtgaaaatcttcgtgtttttccctaccccccaagtcacctcttacacagcctctgcttccaagcgcagcccccacaggagtttgtaggatttctgtgctagcggggagtgtgttctcacctcatagagccaggtagaaattatgcagatgggcgctgttctctgggaagaaagcagggcctttggggctctcagtgtccccgttgggttgtagacataacactctta

SEQ ID NO: 344 - DNA, 304 bp, Homo sapiens
  source 1..304: mol_type = genomic DNA
cctccggggatacaacgtgtttcctaaaagtagagggaggtaagagacggtagcacctgcggggcggcttgcacgccgagtgcctgtgacgcgccggcttaacttaactgcttccctgaagtaccttgaggttcctgatgtgcgggcggtagacggtaggcttatgcggcacgctgtcgtttccaccgtggctactgcgctttgggaaggccacgacctcctcctttggggaggtccttaggatctcagcttggcagtcgagtgggtggcgaccttttaaaggaatgggacccacccggagttc

SEQ ID NO: 345 - DNA, 492 bp, Homo sapiens
  source 1..492: mol_type = genomic DNA
ttcccttcttgtaatacaaaataataacaaccacagaggacatcgcaagtaaaatgtgtataacaaccccatctaaagaaccctatcatctttctactatctgtctacctaaatatgtatagtataaataaccctatatagcactctaagcaacttaagttaggtaacttagaaaactcttttttttttttgagactgagtctcactctgtcgcacccaggctggagtgcagtggcgtgatctcggctcactgcaacctccaccttctgggttcaagtgattctcccatctcagtctcccaagtagctgggattacaggcacgccaccatgccctgctacatttttttttttttagtagagatgaggtttcaccacattggccaggctgttctcgaactcctgacctcaggtgatccgcccaccttggcctcatgaagtgctgggattacaggtgtgagccactgtacccagccagaaaactcttaaaaaac

SEQ ID NO: 346 - DNA, 382 bp, Homo sapiens
  source 1..382: mol_type = genomic DNA
ctgaattttcttgcagttgaacaacagaggctttttttgtgtgtgtgggggtgcttggttttgggaggttgaagagtacttgttcgcaaactctctaaatgagagatggggaagtgtcttaccagctgattcgcgagagcacctgcaagtttacctgttttagtgatttgcgtttatgaaagttggagttgctgattttgtgatctctacttttaaggtaaaagcagggcggtgttgacttttcttgccagttggctgtcctgttttctttctcgcactgttttctttttgagacgaagtctgtcgctcaggctggagtgcactggcgtgatctcggctcgctgcaaactttgccgaacccctgtttagcgatttccgtttg

SEQ ID NO: 347 - DNA, 500 bp, Homo sapiens
  source 1..500: mol_type = genomic DNA
atatgtggtaatccaacaatagaaattatttttaagtttgtgtgttcctttttctgttcaatggtgcttttgatattgttgtaaagcagtgactagcagattctgtgtggtaaaagcactgaaccctgtttagttcttttctccctaccttattggtaattatttctgtgtatatatgttccaaagaaatgcggcttgtatattcatgcttttcaactttttgtgatagacagttgatacacaagtagtacctcctccgggatcttcttagtcaagactgtttggtagatgcatgttttttgttacttgtaatgtacagtattcagttcattttctttttgcagcactgtccggttgaaacacaacgcgaggatcgggcgtggtggctcacgcctgtaatcccagcactttgggaggccgaggcaggacaatcaagaccaatcctggtcatcaaggcaggagaatcaagaccatcgaggtcaggagatcaagaccatcct

SEQ ID NO: 348 - DNA, 498 bp, Homo sapiens
  source 1..498: mol_type = genomic DNA
acatcaggttgtttttctgtttttacatcaggttgtttttctgtttggttttttttttacaccacgtttatacgccggtgcacggtttaccactgaaaacacctttcatctacaggtgatatcttttaacacaaataaaatgtagtagtcctaggagacggaatagaaggaggtggggcctaggcagattcatctctgcggtgcattttgcctctggccctcgggccctagtttgcgatctttcccatcttgcctctgcttccctctgcacaatcctgagtattgcagtaccttacacttaaggaaatttcgtcttgccaaagttgatccatttcagctccgaaacataacaggaactagtgcaacgataaacgtatgaggaacttgcaataagagtgacagaatcctttgaatcaaaaaaaaaaaaagccaccacttaatgagtatctaccatatgtcaagggcttcaccaatatttcattgaaatattccagacca

SEQ ID NO: 349 - DNA, 500 bp, Homo sapiens
  source 1..500: mol_type = genomic DNA
cttatgatgtttgttgccaatgatagattgttttcactgtgcaaaaattatgggtagttttggtggtcttgatgcagttgtaagcttggggtatgaaggtttgggccacgcctgggcgcttccggctgcgccggatgctgtttcctttccgctcccaggggcgttgggaacggttgtaggacgtggctctttattcgtgagttttccatttacctccgctgaacctagagcttcagacgccctatggcgtccgcctcgacccaaccggcggccttgagcgctgagcaagcaaagggtgagaatcgtcctagtcaaggcataggctgctggcctggggtagtcaaggcatgggctgctggtctggggaggatgcgggcggaggatgtggggcgacaaacctggctacgtccgccgggaaaatggggtaggggacgccgggtaccgtccttctaagtggggcgcttgcccccaagacttgggatcttattgggttacctaca

SEQ ID NO: 350 - DNA, 498 bp, Homo sapiens
  source 1..498: mol_type = genomic DNA
tctttactgttatatgttaggcgaaatattacgcgtttggagtaagtggtgctttttgtaactgaaaagagattctgtgtgtgtttttttttttttagaggctgcatagttatttgtatgaaccgcaggtgacctaattagcaaggtaattcagacctgtatcctagcacttaagaagtggaagtgggataaccctgcgtgttctcgtgtgggaggctagcttaagcaacaaagcaggaaggccccaccaacaaaagaatttaaaaattatacgtgcgtggtggcgcgcgtatgaggaaattatttggcaactgtgttacagtatagcagcaaatggaatagacctagtccctgactttaggagacctgcctcagccccccgaatggctgggactacaggcgtgcgcctccgcgcccagctaatttttgcatttttggtagagacgggttcaccatgttggccaggatggtctccatctcttcatctggtgatccgcc

SEQ ID NO: 351 - DNA, 500 bp, Homo sapiens
  source 1..500: mol_type = genomic DNA
accttattcacgcctaaaaagtagactgactgtggggtggtcgtgttttttgtttcttgttggtaggtggtgaatgcgtttttttcgttgttttctccgttactcaggctgccagttgcttggcagtcttgtcgctggctgtggacgctctgcacttcatcaccgtccccagctgcgctttgagccggtctaaggttgcccacagtattgacgttgggacccacgaaactggcaatttccatgtgtgcggccttacccagttctcccaaacttctgcgtataaccgcttctctgtgcttcagaaagataagtccggccccggcctcacacagcagctcagttcgtgacagcgtttaatatgtaaaatggagcaaataatttatagggcgttgggagagttaaagagactaattcatttacgtgttaaatacagggaatggcttttgcattgatataaccccagcctccagcatgcgccagacctgacgtaggtgttcggc

SEQ ID NO: 352 - DNA, 500 bp, Homo sapiens
  source 1..500: mol_type = genomic DNA
tttgacccattacccatctaagttagatgcttttttaaatgttttttaatttttaaatttttaatttttttcattatttattttttatttttgagacggatctcggctcactgtaacctccacctcccgggttcaagcgattctcctgactcagcctcccgagtagctgggattacaggcgcgcgccaccatgcccggctaatttttctatttttagtaaagacggggtttcgccatgttgaccagggtggtcttgaactcctgacgtcaggtgatcctcctgcctcgtgctgggattgcaggcgtgagccaccgctcccggcccttagatgctttttaatcagcaagtacgcaatgctttcctgacaggaatgtcaagtccgtcaggaaatccacataagggtgttaagcaggcaaagtaccacatacagattaaaatcaaaagcactgtcaacgcttatttaatttatacagtagaccctaacttttctaattttgtg

SEQ ID NO: 353 - DNA, 500 bp, Mus musculus
  source 1..500: mol_type = genomic DNA
gtttacttggttttaaaaatagcttgcactagcgataccgcgaatatggttattaggtttgttaggcacagtcgtgtcttactatagaaaaataacgtagtgttcattttgctgcctgtatgtgttaatttgtccttattgcgccattgttcttgttaagtcttctgtaaggagttgcgggtttcaaactgtcagtctgagagcataagctcacttttcagtcagttgaattgaatgttacattcgattggttctggatattgatattacccaaggcttgtcctgtttgtaagtcttttcctacacctgacaatatgattatgtttacaatgtccctagaactctgggtgtatcgttcccactgaataaaatttgatctctaagttgacctataaatttaagtaccaaaaaactggtttgtaagattttggaaggcagaggcagggaagagaccaacgtggtctacatataaagtaccaggtacatataaagtaccaggt

SEQ ID NO: 354 - DNA, 496 bp, Mus musculus
  source 1..496: mol_type = genomic DNA
atttttgtggtgctaaaagttagattgcattccgcccttctcacgtctctttacgtgttgtttgcgaggcgtggcctcggaggtaaatggtctccttttacctcagataagcttacctttgctctgtgagtaagaatgtagctacacttccaaatttagctattgtcctttgcggtccaaatgaaaatattaccttccgatctaacaatgttaaactagtcccaggcccttctatcgattgccagaatagactagactactacacagacacaccctctcaaatcaggccagctcctgcaacctgcacctacagtttgtcccctgggacaaatgaaaggacaggccctctttcccccccccccccaaatcagagcacacacagatcttggggtctggggcagaaatttcgttcatggaaaaaacaaacaaacaaacaaacacgatcaaaggaagctcgttccctttggcctcagggtttcttagacgccgtgttccc

SEQ ID NO: 355 - DNA, 497 bp, Mus musculus
  source 1..497: mol_type = genomic DNA
ataactggtaaccaaaagcagattactttcccccttgtggtcttcgcgtttgcggacgggagagaactttcggccattttccttttagaaatttaacttcttgaaggccaccaggtgacccttacttccctctcacctttccttctacctgtactttctctattaaaaagcctgcagaagtacccttgacaaattgccaaaaatgacacctaaaaaaaaaaaaaggaaaaacgacaccttaggtaattataggttgtaagaaaaagatttgacctcttggagtttttatttcttaataccaaaggacacccaaggaacgtatctctcaaaattgggtttcaaacctcagccgtttccttcttagttccagtgcttaacacgcaacagacctgcacaccactacactgcatacgagttttaaagttaataaatcagtcacccctccatagtcttctacctacttgaagacagaaacggctgctaggaagcaattcttc

SEQ ID NO: 356 - DNA, 500 bp, Mus musculus
  source 1..500: mol_type = genomic DNA
ctggggaataagagtggtttaaagaaagaacaatttgagagatctgcttagatctaataaaacccaagtatagtttctttagacgtcttatatctcacgtaggacctcttatttcagactcactgcaagaacaagctaaagaactatatacagtcaggaacaaggtgagccatcgcggttctcagtcccatgatcttaagagtttttcctcttcccctcttccctggctgtcctggagctcactctgtagaccaggctggtctcaaactcagaaatccacttacctctgcctcccaagggctgggattaaaggcatgcaccaccattgcccggcagggtatgtggactatgctgtccttcaactcagagatcgtggtactttaaatctcattttccgaaagaaaagaaaagaaaggaaggggggaaataaagccagtatattttgcacccacaatctgaagttctacaaatattgttagaaaatgcaaaggacccaagtc

SEQ ID NO: 357 - DNA, 500 bp, Mus musculus
  source 1..500: mol_type = genomic DNA
ctcggtttatcttaaagggttttaaatatagaaagcttccttagtcttatgggtgagagtgctgtttaagccttacctaggtatgtctttgaacatttattacagattgttatttgtgactatgaccactcaggtgttctgttcttgtgaatttagacacactcagaaaggctacacactcaaagaccacaccaaagtgaagtactatctgggccgtgaaactggtcacatatcagttattttgcagtaaaacaaggaagtcaatcatactgcttcagtgggtcagggagtcaagaatgcacagtacaagagctggtgagattcctcagcaggtaagagcactgaatgctcttccaaagattctgagttcaaatcccagcaaccacgtggtggatttgaacaaaggatgaaaacaaccatccataaccagatctgatgccctcttctggagtgtcaaaagacagctatggtgtacttacataaataaataagtaaaaaca

SEQ ID NO: 358 - DNA, 500 bp, Mus musculus
  source 1..500: mol_type = genomic DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttgagccaccatgtggttgctgggatttgaactcaggacctttagaaaagtagttgggtgctcttacccgctgagccatctcaccagccctttttttttcttttttccagttcgggtttctctgtgtgtagccctggctgtcctggaactcattctgtagaccgggctggcctcgaactcagaaatctgcctgcctctgcctcccaagtgctgggattaaaggaatgtgccttcattgcccggctctagggctttttgttgttgtttgtttgtttgtttgtttgtttgcttttgtggatggcct

SEQ ID NO: 359 - DNA, 500 bp, Mus musculus
  source 1..500: mol_type = genomic DNA
aaaaccagtaagggagaaaggtcaagtaacatataaaggaaggcctatcagaattacaccagacttttcaccagagactatgaaagccagaagagcctggacagatgttatacagacactaagagaacacaaatgccagcccaggctactatacctggccaaactctcaattaccatagatggagaaaccaaagtattccacgacaaaaacaaattcacacaatatctttccacgaatccagcccttcaaaggataatgacagaaaagaagcaatacaaggacggaaatcacgccctagaacaaccaagaaactaatcattcaacaaaccaaaaagaagacagccacaagaacagaatgccaactctaacaacaaaaataaaaggaagcaacaattacttttccttaatatctcttaatatcaatggactcaattccccaataaaaagacatagactaacagactggctacacaaacaggacccaacattctgctgctta

SEQ ID NO: 360 - DNA, 500 bp, Mus musculus
  source 1..500: mol_type = genomic DNA
cccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcctgcatccgtgggggaggtggctggctgcagggttctccggatctcttctgtgttcccactactcaagcaccgagtggcgttctatggcgtccgcctcggctcagcccgcggccctgagcgcggagcaggccaagggtgaggaaagagcctagtctgggtgtgggccgtcttctggggggagggcgcgatactattctttgcctctccaagtggggcccttgcccattagggcgaagggttgggaggacaagcttatcgggttgcttgcaggcctcggcccactaattccctgcctcttccctcctctgggctcgcccgccgtcctccgcg

SEQ ID NO: 361 - DNA, 500 bp, Mus musculus
  source 1..500: mol_type = genomic DNA
gaatttgttaaattacaggaggaagcttttttagttcattttacaaatactttcttgttaggtagataataccttctaaggttattatctggatcttctaagtctttttatcgctagcaagtctaagtttctggactttggaattagaagttaatgtcgtaggcgggcatggtggtccacgcctttaatcccagagtttgggaggcagacgcaggtggttttctgagttcgaggccatcctgctcttcaatgttagttccaggacagccagggttatacaaagaaaccctgtctcgagaaacaaaacaaacaaaaaaagagtagctgagtgctctgagttgtctctccagccccttagctatttattttcatttaaattatttttgttttattcgcttgtgtgcctgagtttgaatttcccgggattaacagttgaagactagggctggtgagatggctcagcggttaagattgccgactgctcttccaaaggtcctgag

SEQ ID NO: 362 - DNA, 500 bp, Mus musculus
  source 1..500: mol_type = genomic DNA
accccctaattctacaagaatagaaagcttagcagctcctctgaatggttgctctcaggtgctagtggttgcaagtcgtatgttgatgcctgcctttgccctacctggtgtgtgagcgtgagcgcgccccacctctccagcctctctcttactgtttttcagacaagatgggactgttgttctcctactgctatgtacctcaatttcaattgatcatgcctggacataactgcccatgttgtaaggtcgtctacgttgtaaggtcgtgctcagtttctgatgcctagactagtcacctttccgtttgcctcctgactgtgtaaatgaggcaaatgaaggtggtctcttaactgacatgttagtgtttgacctatgggatagtttctcattgttgagacagaatttctagaagctggtccgctgagcggctaagagcacttgctgatattgcaaaggatctacctgggtttggttctcagcactcacatggaagttcacat

SEQ ID NO: 363 - DNA, 67 bp, Mus musculus
  source 1..67: mol_type = genomic DNA
tgtagttcctgcaatttaattttcgtttaaaactttattttattctttaacttaaaaaaaatatata

SEQ ID NO: 364 - RNA, 82 nt, Mus musculus
  source 1..82: mol_type = unassigned RNA
atgatagggacttagggtgagcaccccatcccccactacaaatttttggagcaggttttctgacttcggtcggaaaacccct

SEQ ID NO: 365 - RNA, 82 nt, Mus musculus
  source 1..82: mol_type = unassigned RNA
atgatagggacttagggtgaaaccaccgctccaccccatcaatttttggagcaggttttctgacttcggtcggaaaacccct

SEQ ID NO: 366 - RNA, 82 nt, Mus musculus
  source 1..82: mol_type = unassigned RNA
atgatagggacttagggtgacaagttggagcaagattatcaatttttggagcaggttttctgacttcggtcggaaaacccct

SEQ ID NO: 367 - RNA, 82 nt, Mus musculus
  source 1..82: mol_type = unassigned RNA
atgatagggacttagggtgactatataaaatagaaatataaatttttggagcaggttttctgacttcggtcggaaaacccct

SEQ ID NO: 368 - RNA, 82 nt, Mus musculus
  source 1..82: mol_type = unassigned RNA
atgatagggacttagggtgatagtttattattagttagaaaatttttggagcaggttttctgacttcggtcggaaaacccct

SEQ ID NO: 369 - RNA, 86 nt, Mus musculus
  source 1..86: mol_type = unassigned RNA
atgatagggacttagggtgagaggaaccaccgctccaccccatcaatttttggagcaggttttctgacttcggtcggaaaacccct

SEQ ID NO: 370 - RNA, 86 nt, Mus musculus
  source 1..86: mol_type = unassigned RNA
atgatagggacttagggtgacatccaagttggagcaagattatcaatttttggagcaggttttctgacttcggtcggaaaacccct

SEQ ID NO: 371 - RNA, 86 nt, Mus musculus
  source 1..86: mol_type = unassigned RNA
atgatagggacttagggtgatatcctatataaaatagaaatataaatttttggagcaggttttctgacttcggtcggaaaacccct

SEQ ID NO: 372 - RNA, 86 nt, Mus musculus
  source 1..86: mol_type = unassigned RNA
atgatagggacttagggtgatatatagtttattattagttagaaaatttttggagcaggttttctgacttcggtcggaaaacccct

SEQ ID NO: 373 - RNA, 88 nt, Mus musculus
  source 1..88: mol_type = unassigned RNA
atgatagggacttagggtgagggaggaaccaccgctccaccccatcaatttttggagcaggttttctgacttcggtcggaaaacccct

SEQ ID NO: 374 - RNA, 88 nt, Mus musculus
  source 1..88: mol_type = unassigned RNA
atgatagggacttagggtgacccatccaagttggagcaagattatcaatttttggagcaggttttctgacttcggtcggaaaacccct

SEQ ID NO: 375 - RNA, 88 nt, Mus musculus
  source 1..88: mol_type = unassigned RNA
atgatagggacttagggtgaattatcctatataaaatagaaatataaatttttggagcaggttttctgacttcggtcggaaaacccct

SEQ ID NO: 376 - RNA, 88 nt, Mus musculus
  source 1..88: mol_type = unassigned RNA
atgatagggacttagggtgaaatatatagtttattattagttagaaaatttttggagcaggttttctgacttcggtcggaaaacccct

SEQ ID NO: 377 - RNA, 92 nt, Mus musculus
  source 1..92: mol_type = unassigned RNA
atgatagggacttagggtgatgaggggaggaaccaccgctccaccccatcaatttttggagcaggttttctgacttcggtcggaaaacccct

SEQ ID NO: 378 - RNA, 92 nt, Mus musculus
  source 1..92: mol_type = unassigned RNA
atgatagggacttagggtgaccaccccatccaagttggagcaagattatcaatttttggagcaggttttctgacttcggtcggaaaacccct

SEQ ID NO: 379 - RNA, 92 nt, Mus musculus
  source 1..92: mol_type = unassigned RNA
atgatagggacttagggtgacaagattatcctatataaaatagaaatataaatttttggagcaggttttctgacttcggtcggaaaacccct

SEQ ID NO: 380 - RNA, 92 nt, Mus musculus
  source 1..92: mol_type = unassigned RNA
atgatagggacttagggtgatagaaatatatagtttattattagttagaaaatttttggagcaggttttctgacttcggtcggaaaacccct

SEQ ID NO: 381 - RNA, 82 nt, Mus musculus
  source 1..82: mol_type = unassigned RNA
atgatagggacttagggtgaagtgcaaggattaaaggtagaatttttggagcaggttttctgacttcggtcggaaaacccct

SEQ ID NO: 382 - RNA, 82 nt, Mus musculus
  source 1..82: mol_type = unassigned RNA
atgatagggacttagggtgacaaaaggggtaatacagtacaatttttggagcaggttttctgacttcggtcggaaaacccct

SEQ ID NO: 383 - RNA, 82 nt, Mus musculus
  source 1..82: mol_type = unassigned RNA
atgatagggacttagggtgaccataataaagggctgagggaatttttggagcaggttttctgacttcggtcggaaaacccct

SEQ ID NO: 384 - RNA, 82 nt, Mus musculus
  source 1..82: mol_type = unassigned RNA
atgatagggacttagggtgagaggaaccaccgctccacccaatttttggagcaggttttctgacttcggtcggaaaacccct

SEQ ID NO: 385 - RNA, 86 nt, Mus musculus
  source 1..86: mol_type = unassigned RNA
atgatagggacttagggtgatcacagtgcaaggattaaaggtagaatttttggagcaggttttctgacttcggtcggaaaacccct

SEQ ID NO: 386 - RNA, 86 nt, Mus musculus
  source 1..86: mol_type = unassigned RNA
atgatagggacttagggtgagtagcaaaaggggtaatacagtacaatttttggagcaggttttctgacttcggtcggaaaacccct

SEQ ID NO: 387 - RNA, 86 nt, Mus musculus
  source 1..86: mol_type = unassigned RNA
atgatagggacttagggtgagtacccataataaagggctgagggaatttttggagcaggttttctgacttcggtcggaaaacccct

SEQ ID NO: 388 - RNA, 86 nt, Mus musculus
  source 1..86: mol_type = unassigned RNA
atgatagggacttagggtgaaggggaggaaccaccgctccacccaatttttggagcaggttttctgacttcggtcggaaaacccct

SEQ ID NO: 389 - RNA, 88 nt, Mus musculus
  source 1..88: mol_type = unassigned RNA
atgatagggacttagggtgaagtcacagtgcaaggattaaaggtagaatttttggagcaggttttctgacttcggtcggaaaacccct

SEQ ID NO: 390 - RNA, 88 nt, Mus musculus
  source 1..88: mol_type = unassigned RNA
atgatagggacttagggtgaaggtagcaaaaggggtaatacagtacaatttttggagcaggttttctgacttcggtcggaaaacccct

SEQ ID NO: 391 - RNA, 88 nt, Mus musculus
  source 1..88: mol_type = unassigned RNA
atgatagggacttagggtgacagtacccataataaagggctgagggaatttttggagcaggttttctgacttcggtcggaaaacccct

SEQ ID NO: 392 - RNA, 88 nt, Mus musculus
  source 1..88: mol_type = unassigned RNA
atgatagggacttagggtgatgaggggaggaaccaccgctccacccaatttttggagcaggttttctgacttcggtcggaaaacccct

SEQ ID NO: 393 - RNA, 92 nt, Mus musculus
  source 1..92: mol_type = unassigned RNA
atgatagggacttagggtgacataagtcacagtgcaaggattaaaggtagaatttttggagcaggttttctgacttcggtcggaaaacccct

SEQ ID NO: 394 - RNA, 92 nt, Mus musculus
  source 1..92: mol_type = unassigned RNA
atgatagggacttagggtgattaaaggtagcaaaaggggtaatacagtacaatttttggagcaggttttctgacttcggtcggaaaacccct

SEQ ID NO: 395 - RNA, 92 nt, Mus musculus
  source 1..92: mol_type = unassigned RNA
atgatagggacttagggtgaaatacagtacccataataaagggctgagggaatttttggagcaggttttctgacttcggtcggaaaacccct

SEQ ID NO: 396 - RNA, 92 nt, Mus musculus
  source 1..92: mol_type = unassigned RNA
atgatagggacttagggtgagggctgaggggaggaaccaccgctccacccaatttttggagcaggttttctgacttcggtcggaaaacccct

SEQ ID NO: 397 - RNA, 82 nt, Mus musculus
  source 1..82: mol_type = unassigned RNA
tttgttccgtgggtggtttaaaccaccgctccaccccatcaatttttggagcaggttttctgacttcggtcggaaaacccct

SEQ ID NO: 398 - RNA, 82 nt, Mus musculus
  source 1..82: mol_type = unassigned RNA
tttgttccgtgggtggtttacaagttggagcaagattatcaatttttggagcaggttttctgacttcggtcggaaaacccct

SEQ ID NO: 399 - RNA, 82 nt, Mus musculus
  source 1..82: mol_type = unassigned RNA
tttgttccgtgggtggtttactatataaaatagaaatataaatttttggagcaggttttctgacttcggtcggaaaacccct

SEQ ID NO: 400 - RNA, 82 nt, Mus musculus
  source 1..82: mol_type = unassigned RNA
tttgttccgtgggtggtttatagtttattattagttagaaaatttttggagcaggttttctgacttcggtcggaaaacccct

SEQ ID NO: 401 - RNA, 86 nt, Mus musculus
  source 1..86: mol_type = unassigned RNA
tttgttccgtgggtggtttagaggaaccaccgctccaccccatcaatttttggagcaggttttctgacttcggtcggaaaacccct

SEQ ID NO: 402 - RNA, 86 nt, Mus musculus
  source 1..86: mol_type = unassigned RNA
tttgttccgtgggtggtttacatccaagttggagcaagattatcaatttttggagcaggttttctgacttcggtcggaaaacccct

SEQ ID NO: 403 - RNA, 86 nt, Mus musculus
  source 1..86: mol_type = unassigned RNA
tttgttccgtgggtggtttatatcctatataaaatagaaatataaatttttggagcaggttttctgacttcggtcggaaaacccct

SEQ ID NO: 404 - RNA, 86 nt, Mus musculus
  source 1..86: mol_type = unassigned RNA
tttgttccgtgggtggtttatatatagtttattattagttagaaaatttttggagcaggttttctgacttcggtcggaaaacccct

SEQ ID NO: 405 - RNA, 88 nt, Mus musculus
  source 1..88: mol_type = unassigned RNA
tttgttccgtgggtggtttagggaggaaccaccgctccaccccatcaatttttggagcaggttttctgacttcggtcggaaaacccct

SEQ ID NO: 406 - RNA, 82 nt, Mus musculus
  source 1..82: mol_type = unassigned RNA
tttgttccgtgggtggtttaggagaatcaccttataggataatttttggagcaggttttctgacttcggtcggaaaacccct

SEQ ID NO: 407 - RNA, 88 nt, Mus musculus
  source 1..88: mol_type = unassigned RNA
tttgttccgtgggtggtttacccatccaagttggagcaagattatcaatttttggagcaggttttctgacttcggtcggaaaacccct

SEQ ID NO: 408 - RNA, 88 nt, Mus musculus
  source 1..88: mol_type = unassigned RNA
tttgttccgtgggtggtttaattatcctatataaaatagaaatataaatttttggagcaggttttctgacttcggtcggaaaacccct

SEQ ID NO: 409 - RNA, 88 nt, Mus musculus
  source 1..88: mol_type = unassigned RNA
tttgttccgtgggtggtttaaatatatagtttattattagttagaaaatttttggagcaggttttctgacttcggtcggaaaacccct

SEQ ID NO: 410 - RNA, 92 nt, Mus musculus
  source 1..92: mol_type = unassigned RNA
tttgttccgtgggtggtttatgaggggaggaaccaccgctccaccccatcaatttttggagcaggttttctgacttcggtcggaaaacccct

SEQ ID NO: 411 - RNA, 92 nt, Mus musculus
  source 1..92: mol_type = unassigned RNA
tttgttccgtgggtggtttaccaccccatccaagttggagcaagattatcaatttttggagcaggttttctgacttcggtcggaaaacccct

SEQ ID NO: 412 - RNA, 92 nt, Mus musculus
  source 1..92: mol_type = unassigned RNA
tttgttccgtgggtggtttacaagattatcctatataaaatagaaatataaatttttggagcaggttttctgacttcggtcggaaaacccct

SEQ ID NO: 413 - RNA, 92 nt, Mus musculus
  source 1..92: mol_type = unassigned RNA
tttgttccgtgggtggtttatagaaatatatagtttattattagttagaaaatttttggagcaggttttctgacttcggtcggaaaacccct

SEQ ID NO: 414 - RNA, 82 nt, Mus musculus
  source 1..82: mol_type = unassigned RNA
tttgttccgtgggtggtttaagtgcaaggattaaaggtagaatttttggagcaggttttctgacttcggtcggaaaacccct

SEQ ID NO: 415 - RNA, 82 nt, Mus musculus
  source 1..82: mol_type = unassigned RNA
tttgttccgtgggtggtttacaaaaggggtaatacagtacaatttttggagcaggttttctgacttcggtcggaaaacccct

SEQ ID NO: 416 - RNA, 82 nt, Mus musculus
  source 1..82: mol_type = unassigned RNA
tttgttccgtgggtggtttaccataataaagggctgagggaatttttggagcaggttttctgacttcggtcggaaaacccct

SEQ ID NO: 417 - RNA, 82 nt, Mus musculus
  source 1..82: mol_type = unassigned RNA
tttgttccgtgggtggtttagaggaaccaccgctccacccaatttttggagcaggttttctgacttcggtcggaaaacccct

SEQ ID NO: 418 - RNA, 82 nt, Mus musculus
  source 1..82: mol_type = unassigned RNA
tttgttccgtgggtggtttagaataataacataataatataatttttggagcaggttttctgacttcggtcggaaaacccct

SEQ ID NO: 419 - RNA, 86 nt, Mus musculus
  source 1..86: mol_type = unassigned RNA
tttgttccgtgggtggtttatcacagtgcaaggattaaaggtagaatttttggagcaggttttctgacttcggtcggaaaacccct

SEQ ID NO: 420 - RNA, 86 nt, Mus musculus
  source 1..86: mol_type = unassigned RNA
tttgttccgtgggtggtttagtagcaaaaggggtaatacagtacaatttttggagcaggttttctgacttcggtcggaaaacccct

SEQ ID NO: 421 - RNA, 86 nt, Mus musculus
  source 1..86: mol_type = unassigned RNA
tttgttccgtgggtggtttagtacccataataaagggctgagggaatttttggagcaggttttctgacttcggtcggaaaacccct

SEQ ID NO: 422 - RNA, 86 nt, Mus musculus
  source 1..86: mol_type = unassigned RNA
tttgttccgtgggtggtttaaggggaggaaccaccgctccacccaatttttggagcaggttttctgacttcggtcggaaaacccct

SEQ ID NO: 423 - RNA, 88 nt, Mus musculus
  source 1..88: mol_type = unassigned RNA
tttgttccgtgggtggtttaagtcacagtgcaaggattaaaggtagaatttttggagcaggttttctgacttcggtcggaaaacccct

SEQ ID NO: 424 - RNA, 88 nt, Mus musculus
  source 1..88: mol_type = unassigned RNA
tttgttccgtgggtggtttaaggtagcaaaaggggtaatacagtacaatttttggagcaggttttctgacttcggtcggaaaacccct

SEQ ID NO: 425 - RNA, 88 nt, Mus musculus
  source 1..88: mol_type = unassigned RNA
tttgttccgtgggtggtttacagtacccataataaagggctgagggaatttttggagcaggttttctgacttcggtcggaaaacccct

SEQ ID NO: 426 - RNA, 88 nt, Mus musculus
  source 1..88: mol_type = unassigned RNA
tttgttccgtgggtggtttatgaggggaggaaccaccgctccacccaatttttggagcaggttttctgacttcggtcggaaaacccct

SEQ ID NO: 427 - RNA, 92 nt, Mus musculus
  source 1..92: mol_type = unassigned RNA
tttgttccgtgggtggtttacataagtcacagtgcaaggattaaaggtagaatttttggagcaggttttctgacttcggtcggaaaacccct

SEQ ID NO: 428 - RNA, 92 nt, Mus musculus
  source 1..92: mol_type = unassigned RNA
tttgttccgtgggtggtttattaaaggtagcaaaaggggtaatacagtacaatttttggagcaggttttctgacttcggtcggaaaacccct

SEQ ID NO: 429 - RNA, 92 nt, Mus musculus
  source 1..92: mol_type = unassigned RNA
tttgttccgtgggtggtttaaatacagtacccataataaagggctgagggaatttttggagcaggttttctgacttcggtcggaaaacccct

SEQ ID NO: 430 - RNA, 92 nt, Mus musculus
  source 1..92: mol_type = unassigned RNA
tttgttccgtgggtggtttagggctgaggggaggaaccaccgctccacccaatttttggagcaggttttctgacttcggtcggaaaacccct

SEQ ID NO: 431 - RNA, 82 nt, Mus musculus
  source 1..82: mol_type = unassigned RNA
tggggggaggtaggtaggtaaaccaccgctccaccccatcaatttttggagcaggttttctgacttcggtcggaaaacccct

SEQ ID NO: 432 - RNA, 82 nt, Mus musculus
  source 1..82: mol_type = unassigned RNA
tggggggaggtaggtaggtacaagttggagcaagattatcaatttttggagcaggttttctgacttcggtcggaaaacccct

SEQ ID NO: 433 - RNA, 82 nt, Mus musculus
  source 1..82: mol_type = unassigned RNA
tggggggaggtaggtaggtactatataaaatagaaatataaatttttggagcaggttttctgacttcggtcggaaaacccct

SEQ ID NO: 434 - RNA, 82 nt, Mus musculus
  source 1..82: mol_type = unassigned RNA
tggggggaggtaggtaggtatagtttattattagttagaaaatttttggagcaggttttctgacttcggtcggaaaacccct

SEQ ID NO: 435 - RNA, 86 nt, Mus musculus
  source 1..86: mol_type = unassigned RNA
tggggggaggtaggtaggtagaggaaccaccgctccaccccatcaatttttggagcaggttttctgacttcggtcggaaaacccct

SEQ ID NO: 436 - RNA, 86 nt, Mus musculus
  source 1..86: mol_type = unassigned RNA
tggggggaggtaggtaggtacatccaagttggagcaagattatcaatttttggagcaggttttctgacttcggtcggaaaacccct

SEQ ID NO: 437 - RNA, 86 nt, Mus musculus
  source 1..86: mol_type = unassigned RNA
tggggggaggtaggtaggtatatcctatataaaatagaaatataaatttttggagcaggttttctgacttcggtcggaaaacccct

SEQ ID NO: 438 - RNA, 86 nt, Mus musculus
  source 1..86: mol_type = unassigned RNA
tggggggaggtaggtaggtatatatagtttattattagttagaaaatttttggagcaggttttctgacttcggtcggaaaacccct

SEQ ID NO: 439 - RNA, 88 nt, Mus musculus
  source 1..88: mol_type = unassigned RNA
tggggggaggtaggtaggtagggaggaaccaccgctccaccccatcaatttttggagcaggttttctgacttcggtcggaaaacccct

SEQ ID NO: 440 - RNA, 88 nt, Mus musculus
  source 1..88: mol_type = unassigned RNA
tggggggaggtaggtaggtacccatccaagttggagcaagattatcaatttttggagcaggttttctgacttcggtcggaaaacccct

SEQ ID NO: 441 - RNA, 88 nt, Mus musculus
  source 1..88: mol_type = unassigned RNA
tggggggaggtaggtaggtaattatcctatataaaatagaaatataaatttttggagcaggttttctgacttcggtcggaaaacccct

SEQ ID NO: 442 - RNA, 92 nt, Mus musculus
  source 1..92: mol_type = unassigned RNA
tggggggaggtaggtaggtatgaggggaggaaccaccgctccaccccatcaatttttggagcaggttttctgacttcggtcggaaaacccct

SEQ ID NO: 443 - RNA, 92 nt, Mus musculus
  source 1..92: mol_type = unassigned RNA
tggggggaggtaggtaggtaccaccccatccaagttggagcaagattatcaatttttggagcaggttttctgacttcggtcggaaaacccct

SEQ ID NO: 444 - RNA, 92 nt, Mus musculus
  source 1..92: mol_type = unassigned RNA
tggggggaggtaggtaggtacaagattatcctatataaaatagaaatataaatttttggagcaggttttctgacttcggtcggaaaacccct

SEQ ID NO: 445 - RNA, 82 nt, Mus musculus
  source 1..82: mol_type = unassigned RNA
tggggggaggtaggtaggtaagtgcaaggattaaaggtagaatttttggagcaggttttctgacttcggtcggaaaacccct

SEQ ID NO: 446 - RNA, 82 nt, Mus musculus
  source 1..82: mol_type = unassigned RNA
tggggggaggtaggtaggtacaaaaggggtaatacagtacaatttttggagcaggttttctgacttcggtcggaaaacccct

SEQ ID NO: 447 - RNA, 82 nt, Mus musculus
  source 1..82: mol_type = unassigned RNA
tggggggaggtaggtaggtaccataataaagggctgagggaatttttggagcaggttttctgacttcggtcggaaaacccct

SEQ ID NO: 448 - RNA, 82 nt, Mus musculus
  source 1..82: mol_type = unassigned RNA
tggggggaggtaggtaggtagaggaaccaccgctccacccaatttttggagcaggttttctgacttcggtcggaaaacccct

SEQ ID NO: 449 - RNA, 86 nt, Mus musculus
  source 1..86: mol_type = unassigned RNA
tggggggaggtaggtaggtatcacagtgcaaggattaaaggtagaatttttggagcaggttttctgacttcggtcggaaaacccct

SEQ ID NO: 450 - RNA, 86 nt, Mus musculus
  source 1..86: mol_type = unassigned RNA
tggggggaggtaggtaggtagtagcaaaaggggtaatacagtacaatttttggagcaggttttctgacttcggtcggaaaacccct

SEQ ID NO: 451 - RNA, 86 nt, Mus musculus
  source 1..86: mol_type = unassigned RNA
tggggggaggtaggtaggtagtacccataataaagggctgagggaatttttggagcaggttttctgacttcggtcggaaaacccct

SEQ ID NO: 452 - RNA, 86 nt, Mus musculus
  source 1..86: mol_type = unassigned RNA
tggggggaggtaggtaggtaaggggaggaaccaccgctccacccaatttttggagcaggttttctgacttcggtcggaaaacccct

SEQ ID NO: 453 - RNA, 88 nt, Mus musculus
  source 1..88: mol_type = unassigned RNA
tggggggaggtaggtaggtaagtcacagtgcaaggattaaaggtagaatttttggagcaggttttctgacttcggtcggaaaacccct

SEQ ID NO: 454 - RNA, 88 nt, Mus musculus
  source 1..88: mol_type = unassigned RNA
tggggggaggtaggtaggtaaggtagcaaaaggggtaatacagtacaatttttggagcaggttttctgacttcggtcggaaaacccct

SEQ ID NO: 455 - RNA, 88 nt, Mus musculus
  source 1..88: mol_type = unassigned RNA
tggggggaggtaggtaggtacagtacccataataaagggctgagggaatttttggagcaggttttctgacttcggtcggaaaacccct

SEQ ID NO: 456 - RNA, 92 nt, Mus musculus
  source 1..92: mol_type = unassigned RNA
tggggggaggtaggtaggtacataagtcacagtgcaaggattaaaggtagaatttttggagcaggttttctgacttcggtcggaaaacccct

SEQ ID NO: 457 - RNA, 92 nt, Mus musculus
  source 1..92: mol_type = unassigned RNA
tggggggaggtaggtaggtattaaaggtagcaaaaggggtaatacagtacaatttttggagcaggttttctgacttcggtcggaaaacccct

SEQ ID NO: 458 - RNA, 92 nt, Mus musculus
  source 1..92: mol_type = unassigned RNA
tggggggaggtaggtaggtaaatacagtacccataataaagggctgagggaatttttggagcaggttttctgacttcggtcggaaaacccct

SEQ ID NO: 459 - RNA, 82 nt, Mus musculus
  source 1..82: mol_type = unassigned RNA
tggggggaggtaggtaggtagaaatttatgtattgattataatttttggagcaggttttctgacttcggtcggaaaacccct

SEQ ID NO: 460 - RNA, 82 nt, Homo sapiens
  source 1..82: mol_type = unassigned RNA
atgatagggacttagggtgaaaccagcgctccaccccatcaatttttggagcaggttttctgacttcggtcggaaaacccct

SEQ ID NO: 461 - RNA, 82 nt, Homo sapiens; identical to SEQ ID NO: 366
  source 1..82: mol_type = unassigned RNA

SEQ ID NO: 462 - RNA, 82 nt, Homo sapiens
  source 1..82: mol_type = unassigned RNA
atgatagggacttagggtgactatacaaaatagaaatataaatttttggagcaggttttctgacttcggtcggaaaacccct

SEQ ID NO: 463 - RNA, 82 nt, Homo sapiens
  source 1..82: mol_type = unassigned RNA
atgatagggacttagggtgatagtttgttattagttagaaaatttttggagcaggttttctgacttcggtcggaaaacccct

SEQ ID NO: 464 - RNA, 86 nt, Homo sapiens
  source 1..86: mol_type = unassigned RNA
atgatagggacttagggtgagaggaaccagcgctccaccccatcaatttttggagcaggttttctgacttcggtcggaaaacccct

SEQ ID NO: 465 - RNA, 86 nt, Homo sapiens; identical to SEQ ID NO: 370
  source 1..86: mol_type = unassigned RNA

SEQ ID NO: 466 - RNA, 86 nt, Homo sapiens
  source 1..86: mol_type = unassigned RNA
atgatagggacttagggtgatatcctatacaaaatagaaatataaatttttggagcaggttttctgacttcggtcggaaaacccct

SEQ ID NO: 467 - RNA, 86 nt, Homo sapiens
  source 1..86: mol_type = unassigned RNA
atgatagggacttagggtgatatatagtttgttattagttagaaaatttttggagcaggttttctgacttcggtcggaaaacccct

SEQ ID NO: 468 - RNA, 88 nt, Homo sapiens
  source 1..88: mol_type = unassigned RNA
atgatagggacttagggtgagggaggaaccagcgctccaccccatcaatttttggagcaggttttctgacttcggtcggaaaacccct

SEQ ID NO: 469 - RNA, 88 nt, Homo sapiens; identical to SEQ ID NO: 374
  source 1..88: mol_type = unassigned RNA

SEQ ID NO: 470 - RNA, 88 nt, Homo sapiens
  source 1..88: mol_type = unassigned RNA
atgatagggacttagggtgaattatcctatacaaaatagaaatataaatttttggagcaggttttctgacttcggtcggaaaacccct

SEQ ID NO: 471 - RNA, 88 nt, Homo sapiens
  source 1..88: mol_type = unassigned RNA
atgatagggacttagggtgaaatatatagtttgttattagttagaaaatttttggagcaggttttctgacttcggtcggaaaacccct

SEQ ID NO: 472 - RNA, 92 nt, Homo sapiens
  source 1..92: mol_type = unassigned RNA
atgatagggacttagggtgatcaggggaggaaccagcgctccaccccatcaatttttggagcaggttttctgacttcggtcggaaaacccct

SEQ ID NO: 473 - RNA, 92 nt, Homo sapiens; identical to SEQ ID NO: 378
  source 1..92: mol_type = unassigned RNA

SEQ ID NO: 474 - RNA, 92 nt, Homo sapiens
  source 1..92: mol_type = unassigned RNA
atgatagggacttagggtgacaagattatcctatacaaaatagaaatataaatttttggagcaggttttctgacttcggtcggaaaacccct

SEQ ID NO: 475 - RNA, 92 nt, Homo sapiens
  source 1..92: mol_type = unassigned RNA
atgatagggacttagggtgatagaaatatatagtttgttattagttagaaaatttttggagcaggttttctgacttcggtcggaaaacccct

SEQ ID NO: 476 - RNA, 82 nt, Homo sapiens; identical to SEQ ID NO: 381
  source 1..82: mol_type = unassigned RNA

SEQ ID NO: 477 - RNA, 82 nt, Homo sapiens; identical to SEQ ID NO: 382
  source 1..82: mol_type = unassigned RNA

SEQ ID NO: 478 - RNA, 82 nt, Homo sapiens
  source 1..82: mol_type = unassigned RNA
atgatagggacttagggtgaccataataaagggctcagggaatttttggagcaggttttctgacttcggtcggaaaacccct

SEQ ID NO: 479 - RNA, 82 nt, Homo sapiens
  source 1..82: mol_type = unassigned RNA
atgatagggacttagggtgagaggaaccagcgctccacccaatttttggagcaggttttctgacttcggtcggaaaacccct

SEQ ID NO: 480 - RNA, 86 nt, Homo sapiens; identical to SEQ ID NO: 385
  source 1..86: mol_type = unassigned RNA

SEQ ID NO: 481 - RNA, 86 nt, Homo sapiens; identical to SEQ ID NO: 386
  source 1..86: mol_type = unassigned RNA

SEQ ID NO: 482 - RNA, 86 nt, Homo sapiens
  source 1..86: mol_type = unassigned RNA
atgatagggacttagggtgagtacccataataaagggctcagggaatttttggagcaggttttctgacttcggtcggaaaacccct

SEQ ID NO: 483 - RNA, 86 nt, Homo sapiens
  source 1..86: mol_type = unassigned RNA
atgatagggacttagggtgaaggggaggaaccagcgctccacccaatttttggagcaggttttctgacttcggtcggaaaacccct

SEQ ID NO: 484 - RNA, 88 nt, Homo sapiens; identical to SEQ ID NO: 389
  source 1..88: mol_type = unassigned RNA

SEQ ID NO: 485 - RNA, 88 nt, Homo sapiens; identical to SEQ ID NO: 390
  source 1..88: mol_type = unassigned RNA

SEQ ID NO: 486 - RNA, 88 nt, Homo sapiens
  source 1..88: mol_type = unassigned RNA
atgatagggacttagggtgacagtacccataataaagggctcagggaatttttggagcaggttttctgacttcggtcggaaaacccct

SEQ ID NO: 487 - RNA, 88 nt, Homo sapiens
  source 1..88: mol_type = unassigned RNA
atgatagggacttagggtgatcaggggaggaaccagcgctccacccaatttttggagcaggttttctgacttcggtcggaaaacccct

SEQ ID NO: 488 - RNA, 92 nt, Homo sapiens; identical to SEQ ID NO: 393
  source 1..92: mol_type = unassigned RNA

SEQ ID NO: 489 - RNA, 92 nt, Homo sapiens; identical to SEQ ID NO: 394
  source 1..92: mol_type = unassigned RNA

SEQ ID NO: 490 - RNA, 92 nt, Homo sapiens
  source 1..92: mol_type = unassigned RNA
atgatagggacttagggtgaaatacagtacccataataaagggctcagggaatttttggagcaggttttctgacttcggtcggaaaacccct

SEQ ID NO: 491 - RNA, 92 nt, Homo sapiens
  source 1..92: mol_type = unassigned RNA
atgatagggacttagggtgagggctcaggggaggaaccagcgctccacccaatttttggagcaggttttctgacttcggtcggaaaacccct

SEQ ID NO: 492 - RNA, 82 nt, Homo sapiens; identical to SEQ ID NO: 460
  source 1..82: mol_type = unassigned RNA

SEQ ID NO: 493 - RNA, 82 nt, Homo sapiens; identical to SEQ ID NO: 398
  source 1..82: mol_type = unassigned RNA

SEQ ID NO: 494 - RNA, 82 nt, Homo sapiens
  source 1..82: mol_type = unassigned RNA
tttgttccgtgggtggtttactatacaaaatagaaatataaatttttggagcaggttttctgacttcggtcggaaaacccct

SEQ ID NO: 495 - RNA, 82 nt, Homo sapiens
  source 1..82: mol_type = unassigned RNA
tttgttccgtgggtggtttatagtttgttattagttagaaaatttttggagcaggttttctgacttcggtcggaaaacccct

SEQ ID NO: 496 - RNA, 86 nt, Homo sapiens
  source 1..86: mol_type = unassigned RNA
tttgttccgtgggtggtttagaggaaccagcgctccaccccatcaatttttggagcaggttttctgacttcggtcggaaaacccct

SEQ ID NO: 497 - RNA, 86 nt, Homo sapiens; identical to SEQ ID NO: 402
  source 1..86: mol_type = unassigned RNA

SEQ ID NO: 498 - RNA, 86 nt, Homo sapiens
  source 1..86: mol_type = unassigned RNA
tttgttccgtgggtggtttatatcctatacaaaatagaaatataaatttttggagcaggttttctgacttcggtcggaaaacccct

SEQ ID NO: 499 - RNA, 86 nt, Homo sapiens
  source 1..86: mol_type = unassigned RNA
tttgttccgtgggtggtttatatatagtttgttattagttagaaaatttttggagcaggttttctgacttcggtcggaaaacccct

SEQ ID NO: 500 - RNA, 88 nt, Homo sapiens
  source 1..88: mol_type = unassigned RNA
tttgttccgtgggtggtttagggaggaaccagcgctccaccccatcaatttttggagcaggttttctgacttcggtcggaaaacccct

SEQ ID NO: 501 - RNA, 88 nt, Homo sapiens; identical to SEQ ID NO: 407
  source 1..88: mol_type = unassigned RNA

SEQ ID NO: 502 - RNA, 88 nt, Homo sapiens
  source 1..88: mol_type = unassigned RNA
tttgttccgtgggtggtttaattatcctatacaaaatagaaatataaatttttggagcaggttttctgacttcggtcggaaaacccct

SEQ ID NO: 503 - RNA, 88 nt, Homo sapiens
  source 1..88: mol_type = unassigned RNA
tttgttccgtgggtggtttaaatatatagtttgttattagttagaaaatttttggagcaggttttctgacttcggtcggaaaacccct

SEQ ID NO: 504 - RNA, 92 nt, Homo sapiens
  source 1..92: mol_type = unassigned RNA
tttgttccgtgggtggtttatcaggggaggaaccagcgctccaccccatcaatttttggagcaggttttctgacttcggtcggaaaacccct

SEQ ID NO: 505 - RNA, 92 nt, Homo sapiens; identical to SEQ ID NO: 411
  source 1..92: mol_type = unassigned RNA

SEQ ID NO: 506 - RNA, 92 nt, Homo sapiens
  source 1..92: mol_type = unassigned RNA
tttgttccgtgggtggtttacaagattatcctatacaaaatagaaatataaatttttggagcaggttttctgacttcggtcggaaaacccct

SEQ ID NO: 507 - RNA, 92 nt, Homo sapiens
  source 1..92: mol_type = unassigned RNA
tttgttccgtgggtggtttatagaaatatatagtttgttattagttagaaaatttttggagcaggttttctgacttcggtcggaaaacccct

SEQ ID NO: 508 - RNA, 82 nt, Homo sapiens; identical to SEQ ID NO: 414
  source 1..82: mol_type = unassigned RNA

SEQ ID NO: 509 - RNA, 82 nt, Homo sapiens; identical to SEQ ID NO: 415
  source 1..82: mol_type = unassigned RNA

SEQ ID NO: 510 - RNA, 82 nt, Homo sapiens
  source 1..82: mol_type = unassigned RNA
tttgttccgtgggtggtttaccataataaagggctcagggaatttttggagcaggttttctgacttcggtcggaaaacccct

SEQ ID NO: 511 - RNA, 82 nt, Homo sapiens
  source 1..82: mol_type = unassigned RNA
tttgttccgtgggtggtttagaggaaccagcgctccacccaatttttggagcaggttttctgacttcggtcggaaaacccct

SEQ ID NO: 512 - RNA, 86 nt, Homo sapiens; identical to SEQ ID NO: 419
  source 1..86: mol_type = unassigned RNA

SEQ ID NO: 513 - RNA, 86 nt, Homo sapiens; identical to SEQ ID NO: 420
  source 1..86: mol_type = unassigned RNA

SEQ ID NO: 514 - RNA, 86 nt, Homo sapiens
  source 1..86: mol_type = unassigned RNA
tttgttccgtgggtggtttagtacccataataaagggctcagggaatttttggagcaggttttctgacttcggtcggaaaacccct

SEQ ID NO: 515 - RNA, 86 nt, Homo sapiens
  source 1..86: mol_type = unassigned RNA
tttgttccgtgggtggtttaaggggaggaaccagcgctccacccaatttttggagcaggttttctgacttcggtcggaaaacccct

SEQ ID NO: 516 - RNA, 88 nt, Homo sapiens; identical to SEQ ID NO: 423
  source 1..88: mol_type = unassigned RNA

SEQ ID NO: 517 - RNA, 88 nt, Homo sapiens; identical to SEQ ID NO: 424
  source 1..88: mol_type = unassigned RNA

SEQ ID NO: 518 - RNA, 88 nt, Homo sapiens
  source 1..88: mol_type = unassigned RNA
tttgttccgtgggtggtttacagtacccataataaagggctcagggaatttttggagcaggttttctgacttcggtcggaaaacccct

SEQ ID NO: 519 - RNA, 88 nt, Homo sapiens
  source 1..88: mol_type = unassigned RNA
tttgttccgtgggtggtttatcaggggaggaaccagcgctccacccaatttttggagcaggttttctgacttcggtcggaaaacccct

SEQ ID NO: 520 - RNA, 92 nt, Homo sapiens; identical to SEQ ID NO: 427
  source 1..92: mol_type = unassigned RNA

SEQ ID NO: 521 - RNA, 92 nt, Homo sapiens; identical to SEQ ID NO: 428
  source 1..92: mol_type = unassigned RNA

SEQ ID NO: 522 - RNA, 92 nt, Homo sapiens
  source 1..92: mol_type = unassigned RNA
tttgttccgtgggtggtttaaatacagtacccataataaagggctcagggaatttttggagcaggttttctgacttcggtcggaaaacccct

SEQ ID NO: 523 - RNA, 92 nt, Homo sapiens
  source 1..92: mol_type = unassigned RNA
tttgttccgtgggtggtttagggctcaggggaggaaccagcgctccacccaatttttggagcaggttttctgacttcggtcggaaaacccct

SEQ ID NO: 524 - RNA, 82 nt, Homo sapiens
  source 1..82: mol_type = unassigned RNA
tttgttccgtgggtggtttaaaccagcgctccaccccatcaatttttggagcaggttttctgacttcggtcggaaaacccct

SEQ ID NO: 525 - RNA, 82 nt, Homo sapiens; identical to SEQ ID NO: 398
  source 1..82: mol_type = unassigned RNA

SEQ ID NO: 526 - RNA, 82 nt, Homo sapiens; identical to SEQ ID NO: 494
  source 1..82: mol_type = unassigned RNA

SEQ ID NO: 527 - RNA, 82 nt, Homo sapiens
  source 1..82: mol_type = unassigned RNA
tggggggaggtaggtaggtatagtttgttattagttagaaaatttttggagcaggttttctgacttcggtcggaaaacccct

SEQ ID NO: 528 - RNA, 86 nt, Homo sapiens
  source 1..86: mol_type = unassigned RNA
tggggggaggtaggtaggtagaggaaccagcgctccaccccatcaatttttggagcaggttttctgacttcggtcggaaaacccct

SEQ ID NO: 529 - RNA, 86 nt, Homo sapiens; identical to SEQ ID NO: 436
  source 1..86: mol_type = unassigned RNA

SEQ ID NO: 530 - RNA, 86 nt, Homo sapiens
  source 1..86: mol_type = unassigned RNA
tggggggaggtaggtaggtatatcctatacaaaatagaaatataaatttttggagcaggttttctgacttcggtcggaaaacccct

SEQ ID NO: 531 - RNA, 86 nt, Homo sapiens
  source 1..86: mol_type = unassigned RNA
tggggggaggtaggtaggtatatatagtttgttattagttagaaaatttttggagcaggttttctgacttcggtcggaaaacccct

SEQ ID NO: 532 - RNA, 88 nt, Homo sapiens
  source 1..88: mol_type = unassigned RNA
tggggggaggtaggtaggtagggaggaaccagcgctccaccccatcaatttttggagcaggttttctgacttcggtcggaaaacccct

SEQ ID NO: 533 - RNA, 88 nt, Homo sapiens; identical to SEQ ID NO: 440
  source 1..88: mol_type = unassigned RNA

SEQ ID NO: 534 - RNA, 88 nt, Homo sapiens
  source 1..88: mol_type = unassigned RNA
tggggggaggtaggtaggtaattatcctatacaaaatagaaatataaatttttggagcaggttttctgacttcggtcggaaaacccct

SEQ ID NO: 535 - RNA, 92 nt, Homo sapiens
  source 1..92: mol_type = unassigned RNA
tggggggaggtaggtaggtatcaggggaggaaccagcgctccaccccatcaatttttggagcaggttttctgacttcggtcggaaaacccct

SEQ ID NO: 536 - RNA, 92 nt, Homo sapiens; identical to SEQ ID NO: 443
  source 1..92: mol_type = unassigned RNA

SEQ ID NO: 537 - RNA, 92 nt, Homo sapiens
  source 1..92: mol_type = unassigned RNA
tggggggaggtaggtaggtacaagattatcctatacaaaatagaaatataaatttttggagcaggttttctgacttcggtcggaaaacccct

SEQ ID NO: 538 - RNA, 82 nt, Homo sapiens; identical to SEQ ID NO: 445
  source 1..82: mol_type = unassigned RNA

SEQ ID NO: 539 - RNA, 82 nt, Homo sapiens; identical to SEQ ID NO: 446
  source 1..82: mol_type = unassigned RNA

SEQ ID NO: 540 - RNA, 82 nt, Homo sapiens
  source 1..82: mol_type = unassigned RNA
tggggggaggtaggtaggtaccataataaagggctcagggaatttttggagcaggttttctgacttcggtcggaaaacccct

SEQ ID NO: 541 - RNA, 82 nt, Homo sapiens
  source 1..82: mol_type = unassigned RNA
tggggggaggtaggtaggtagaggaaccagcgctccacccaatttttggagcaggttttctgacttcggtcggaaaacccct

SEQ ID NO: 542 - RNA, 86 nt, Homo sapiens; identical to SEQ ID NO: 449
  source 1..86: mol_type = unassigned RNA

SEQ ID NO: 543 - RNA, 86 nt, Homo sapiens; identical to SEQ ID NO: 450
  source 1..86: mol_type = unassigned RNA

SEQ ID NO: 544 - RNA, 86 nt, Homo sapiens
  source 1..86: mol_type = unassigned RNA
tggggggaggtaggtaggtagtacccataataaagggctcagggaatttttggagcaggttttctgacttcggtcggaaaacccct

SEQ ID NO: 545 - RNA, 86 nt, Homo sapiens
  source 1..86: mol_type = unassigned RNA
tggggggaggtaggtaggtaaggggaggaaccagcgctccacccaatttttggagcaggttttctgacttcggtcggaaaacccct

SEQ ID NO: 546 - RNA, 88 nt, Homo sapiens; identical to SEQ ID NO: 453
  source 1..88: mol_type = unassigned RNA

SEQ ID NO: 547 - RNA, 88 nt, Homo sapiens; identical to SEQ ID NO: 454
  source 1..88: mol_type = unassigned RNA

SEQ ID NO: 548 - RNA, 88 nt, Homo sapiens
  source 1..88: mol_type = unassigned RNA
tggggggaggtaggtaggtacagtacccataataaagggctcagggaatttttggagcaggttttctgacttcggtcggaaaacccct

SEQ ID NO: 549 - RNA, 92 nt, Homo sapiens; identical to SEQ ID NO: 456
  source 1..92: mol_type = unassigned RNA

SEQ ID NO: 550 - RNA, 92 nt, Homo sapiens; identical to SEQ ID NO: 457
  source 1..92: mol_type = unassigned RNA

SEQ ID NO: 551 - RNA, 92 nt, Homo sapiens
  source 1..92: mol_type = unassigned RNA
tggggggaggtaggtaggtaaatacagtacccataataaagggctcagggaatttttggagcaggttttctgacttcggtcggaaaacccct

SEQ ID NO: 552 - RNA, 82 nt, Homo sapiens; identical to SEQ ID NO: 364
  source 1..82: mol_type = unassigned RNA

SEQ ID NO: 553 - RNA, 110 nt, Homo sapiens
  source 1..110: mol_type = unassigned RNA
atgatagggacttagggtgagtaatacagtacccataataaaggagcaagattatcctatataaaataaatttttggagcaggttttctgacttcggtcggaaaacccct

SEQ ID NO: 554 - RNA, 118 nt, Homo sapiens
  source 1..118: mol_type = unassigned RNA
atgatagggacttagggtgagggtaatacagtacccataataaagggcggagcaagattatcctatataaaatagaaatttttggagcaggttttctgacttcggtcggaaaacccct

SEQ ID NO: 555 - RNA, 122 nt, Homo sapiens
  source 1..122: mol_type = unassigned RNA
atgatagggacttagggtgaggggtaatacagtacccataataaagggcttggagcaagattatcctatataaaatagaaaatttttggagcaggttttctgacttcggtcggaaaacccct

SEQ ID NO: 556 - RNA, 110 nt, Homo sapiens
  source 1..110: mol_type = unassigned RNA
atgatagggacttagggtgaaggggtaatacagtacccataataagattatcctatataaaatagaaaaatttttggagcaggttttctgacttcggtcggaaaacccct

SEQ ID NO: 557 - RNA, 118 nt, Homo sapiens
  source 1..118: mol_type = unassigned RNA
atgatagggacttagggtgaaaaaggggtaatacagtacccataataaaagattatcctatataaaatagaaatataatttttggagcaggttttctgacttcggtcggaaaacccct

SEQ ID NO: 558 - RNA, 122 nt, Homo sapiens
  source 1..122: mol_type = unassigned RNA
atgatagggacttagggtgacaaaaggggtaatacagtacccataataaacaagattatcctatataaaatagaaatataaatttttggagcaggttttctgacttcggtcggaaaacccct

SEQ ID NO: 559 - RNA, 110 nt, Homo sapiens
  source 1..110: mol_type = unassigned RNA
atgatagggacttagggtgatacagtacccataataaagggctgttggagcaagattatcctatataaaatttttggagcaggttttctgacttcggtcggaaaacccct

SEQ ID NO: 560 - RNA, 118 nt, Homo sapiens
  source 1..118: mol_type = unassigned RNA
atgatagggacttagggtgaatacagtacccataataaagggctgaggaagttggagcaagattatcctatataaaaatttttggagcaggttttctgacttcggtcggaaaacccct

SEQ ID NO: 561 - RNA, 122 nt, Homo sapiens
  source 1..122: mol_type = unassigned RNA
atgatagggacttagggtgaaatacagtacccataataaagggctgagggcaagttggagcaagattatcctatataaaaaatttttggagcaggttttctgacttcggtcggaaaacccct

SEQ ID NO: 562 - RNA, 110 nt, Homo sapiens
  source 1..110: mol_type = unassigned RNA
atgatagggacttagggtgaaggggtaatacagtacccataatattggagcaagattatcctatataaaatttttggagcaggttttctgacttcggtcggaaaacccct

SEQ ID NO: 563 - RNA, 118 nt, Homo sapiens
  source 1..118: mol_type = unassigned RNA
atgatagggacttagggtgaaaaaggggtaatacagtacccataataaaagttggagcaagattatcctatataaaaatttttggagcaggttttctgacttcggtcggaaaacccct

SEQ ID NO: 564 - RNA, 122 nt, Homo sapiens
  source 1..122: mol_type = unassigned RNA
atgatagggacttagggtgacaaaaggggtaatacagtacccataataaacaagttggagcaagattatcctatataaaaaatttttggagcaggttttctgacttcggtcggaaaacccct

SEQ ID NO: 565 - RNA, 110 nt, Homo sapiens
  source 1..110: mol_type = unassigned RNA
atgatagggacttagggtgatacagtacccataataaagggctgagattatcctatataaaatagaaaaatttttggagcaggttttctgacttcggtcggaaaacccct

SEQ ID NO: 566 - RNA, 118 nt, Homo sapiens
  source 1..118: mol_type = unassigned RNA
atgatagggacttagggtgaatacagtacccataataaagggctgaggaagattatcctatataaaatagaaatataatttttggagcaggttttctgacttcggtcggaaaacccct

(19) United States
(12) Patent Application Publication
(10) Pub. No.: US 2026/0209779 A1
(43) Pub. Date: Jul. 23, 2026

(54) THERAPEUTIC REGULATION OF SCN1A SPLICING

(71) Applicant: Emugen Therapeutics LLC, Woburn, MA (US)

(72) Inventors: Jonathan J. Wilde, Waltham, MA (US); Tobias Kaiser, Boston, MA (US); Xian Gao, Belmont, MA (US); Olga Brevnova, Somerville, MA (US); Tomomi Aida, Arlington, MA (US); Yuanyuan Hou, Cambridge, MA (US); Tyler Brown, Somerville, MA (US)

(21) Appl. No.: 19/636,483

(22) Filed: Apr. 1, 2026

Related U.S. Application Data
(63) Continuation of application No. PCT/US24/50793, filed on Oct. 10, 2024.
(60) Provisional application No. 63/589,499, filed on Oct. 11, 2023.

(51) Int. Cl. C12N 15/113; A61K 38/17; A61K 48/00; A61P 25/08; C12N 15/86

(52) U.S. Cl. CPC C12N 15/1138; A61K 38/177; A61K 48/005; A61P 25/08; C12N 15/86; C12N 2310/11; C12N 2320/33; C12N 2750/14143

(57) ABSTRACT

Disclosed herein are systems for modifying nucleic acid splicing of a target RNA such as an SCN1A mRNA. The system may be useful in a method such as a method for treating a genetic disorder such as epilepsy. The system may include an expression system, or a modified small nuclear RNA (snRNA) that includes an exonic splicing silencer (ESS) sequence, and an antisense nucleic acid sequence that targets the target RNA.

### CROSS-REFERENCE

This application is a continuation application of International Patent Application No. PCT/US2024/050793 filed Oct. 10, 2024, which claims the benefit of U.S. Provisional Application No. 63/589,499, filed Oct. 11, 2023, which are incorporated herein by reference.

### INCORPORATION BY REFERENCE OF SEQUENCE LISTING

The present application is being filed along with a Sequence Listing in electronic format. The Sequence Listing is provided as a file entitled 062692-504001WO.xml, created Oct. 9, 2024, which is 428,506 bytes in size. The information in the electronic format of the Sequence Listing is incorporated by reference in its entirety.

### BACKGROUND

Epileptic disorders such as Dravet syndrome are a concern for many individuals, and improved treatments are needed.

### SUMMARY

Disclosed herein, in some embodiments, are polynucleotides, comprising: an exonic splicing silencer (ESS) nucleic acid sequence; and an antisense nucleic acid sequence that binds to an alternatively spliced region of a ribonucleic acid (RNA) encoding sodium channel protein type 1 subunit alpha (SCN1A). In some embodiments, the ESS nucleic acid sequence comprises a sequence having at least 80%, at least 85%, at least 90%, at least 95%, at least 98%, at least 99%, or 100% identity to any one of SEQ ID NOs: 11-13, optionally wherein the ESS nucleic acid sequence comprises the nucleic acid sequence of any one of SEQ ID NOs: 11-13. In some embodiments, the antisense nucleic acid sequence comprises a sequence having at least 80%, at least 85%, at least 90%, at least 95%, at least 98%, at least 99%, or 100% identity to any one of SEQ ID NOs: 14 and 18-104, optionally wherein the antisense nucleic acid sequence comprises the nucleic acid sequence of any one of SEQ ID NOs: 14 and 18-104. In some embodiments, the polynucleotide further comprises a Sm binding site. In some embodiments, the Sm binding site comprises a nucleic acid sequence having at least 80%, at least 85%, at least 90%, at least 95%, at least 98%, at least 99%, or 100% identity to the nucleic acid sequence of any one of SEQ ID NOs: 150-151, optionally wherein the Sm binding site comprises the nucleic acid sequence of any one of SEQ ID NOs: 150-151. In some embodiments, the polynucleotide further comprises a hairpin sequence. In some embodiments, the hairpin sequence comprises a nucleic acid sequence having at least 80%, at least 85%, at least 90%, at least 95%, at least 98%, at least 99%, or 100% identity to the nucleic acid sequence of SEQ ID NO: 4, optionally wherein the hairpin sequence comprises the nucleic acid sequence of SEQ ID NO: 4. In some embodiments, the polynucleotide comprising a nucleic acid sequence having at least 80%, at least 85%, at least 90%, at least 95%, at least 98%, at least 99%, or 100% identity to the nucleic acid sequence of any one of SEQ ID NOs: 364-566.

Disclosed herein, in some embodiments, are polynucleotides, comprising: a first sequence comprising a nucleic acid sequence having at least 80%, at least 85%, at least 90%, at least 95%, at least 98%, at least 99%, or 100% identity to any one of SEQ ID NOs: 11-13; and a second sequence comprising a nucleic acid sequence having at least 80%, at least 85%, at least 90%, at least 95%, at least 98%, at least 99%, or 100% identity to any one of SEQ ID NOs: 14 and 18-104. In some embodiments, the first sequence comprises the nucleic acid sequence of any one of SEQ ID NOs: 11-13. In some embodiments, the second sequence comprises the nucleic acid sequence of any one of SEQ ID NOs: 14 and 18-104. In some embodiments, the polynucleotide further a third sequence, wherein the third sequence comprises a nucleic acid sequence having at least 80%, at least 85%, at least 90%, at least 95%, at least 98%, at least 99%, or 100% identity to the nucleic acid sequence of any one of SEQ ID NOs: 150-151, optionally wherein the third sequence comprises the nucleic acid sequence of any one of SEQ ID NOs: 150-151. In some embodiments, the polynucleotide further comprises a fourth sequence, wherein the fourth sequence comprises a nucleic acid sequence having at least 80%, at least 85%, at least 90%, at least 95%, at least 98%, at least 99%, or 100% identity to the nucleic acid sequence of SEQ ID NO: 4, optionally wherein the fourth sequence comprises the nucleic acid sequence of SEQ ID NO: 4. In some embodiments, the polynucleotide comprising a nucleic acid sequence having at least 90% identity to the nucleic acid sequence of any one of SEQ ID NOs: 364-566.

Disclosed herein, in some embodiments, are RNA polynucleotides, comprising a nucleic acid sequence having at least 80%, at least 85%, at least 90%, at least 95%, at least 98%, at least 99%, or 100% identity to the nucleic acid sequence of any one of SEQ ID NOs: 364-566.

Disclosed herein, in some embodiments, are polynucleotides, comprising a promoter, a coding sequence, and a terminator sequence, wherein the coding sequence comprises a nucleic acid sequence having at least 80%, at least 85%, at least 90%, at least 95%, at least 98%, at least 99%, or 100% identity to the nucleic acid sequence of any one of SEQ ID NOs: 364-566.

Disclosed herein, in some embodiments, are viral particles, comprising a polynucleotide comprising one or more copies of a cassette comprising a promoter, a coding sequence, and a terminator sequence, wherein the coding sequence comprises a nucleic acid sequence having at least 80%, at least 85%, at least 90%, at least 95%, at least 98%, at least 99%, or 100% identity to the nucleic acid sequence of any one of SEQ ID NOs: 364-566. In some embodiments, the viral particle is an AAV viral particle. In some embodiments, the polynucleotide comprises 2, 3, or 4 copies of the cassette.

Disclosed herein, in some embodiments, are systems for modifying nucleic acid splicing. The system may include an engineered U7 small nuclear RNA (snRNA). The engineered U7 snRNA may include an antisense nucleic acid sequence that binds or targets an alternatively spliced region of a RNA encoding a sodium channel protein type 1 subunit alpha (SCN1A) protein. The system may include an expression vector encoding the engineered U7 snRNA. The system may be useful in a method such as a method for treating of an epileptic disorder. The disclosure herein helps address unmet needs for improved, safe and effective treatments for epileptic disorders such as Dravet syndrome.

Disclosed herein, in some embodiments, are systems for modifying nucleic acid splicing, comprising: an engineered U7 snRNA comprising an antisense nucleic acid sequence that binds an alternatively spliced region of a RNA encoding an SCN1A protein. In some embodiments, the engineered U7 snRNA further comprises an exonic splicing silencer (ESS) nucleic acid sequence. In some embodiments, the engineered U7 snRNA does not comprise an ESS nucleic acid sequence.

Disclosed herein, in some embodiments, are systems for modifying nucleic acid splicing, comprising: an ESS nucleic acid sequence; and an antisense nucleic acid sequence that targets an alternatively spliced region of an RNA encoding an SCN1A protein. In some embodiments, the system comprises or encodes one or more RNA molecules comprising: the ESS nucleic acid sequence and the antisense nucleic acid sequence. In some embodiments, the system comprises the one or more RNA molecules. In some embodiments, the one or more RNA molecules comprise an engineered U7 snRNA. In some embodiments, the system comprises an engineered U7 snRNA comprising the ESS nucleic acid sequence and the antisense nucleic acid sequence.

In some embodiments, the alternatively spliced region is out of frame with an exon of a productive transcript. In some embodiments, the alternatively spliced region comprises a stop codon, or wherein inclusion of the alternatively spliced region results in a premature stop codon of a mature SCN1A mRNA. In some embodiments, the SCN1A comprises a human SCN1A. In some embodiments, the alternatively spliced region is an alternatively spliced exon. In some embodiments, the alternatively spliced region comprises an alternative exon of the RNA encoding SCN1A, wherein inclusion of the alternative exon in a mature SCN1A mRNA results in nonsense mediated decay (NMD) of a transcription product of the mature SCN1A mRNA. In some embodiments, the alternatively spliced region comprises exon 20N of the RNA encoding SCN1A. In some embodiments, the exon 20N comprises a nucleic acid sequence at least 80%, at least 85%, at least 90%, at least 95%, at least 98%, at least 99%, or 100% identical to SEQ ID NO: 2. In some embodiments, the ESS recruits a protein factor or group of factors that reduce or silence splicing of the RNA encoding SCN1A. In some embodiments, the ESS nucleic acid sequence comprises a nucleic acid sequence at least 80%, at least 85%, at least 90%, at least 95%, at least 98%, at least 99%, or 100% identical to an ESS nucleic acid sequence in Tables 2A-2B. In some embodiments, the antisense nucleic acid sequence is 10-60 nucleotides in length. In some embodiments, the antisense nucleic acid sequence binds to at least a portion of the alternatively spliced region. In some embodiments, the antisense nucleic acid sequence is partially reverse complementary to a portion of the alternatively spliced region. In some embodiments, the antisense nucleic acid sequence is fully reverse complementary to a portion of the alternatively spliced region. In some embodiments, the portion of the alternatively spliced region is within a 5′ half or 5′ end of the alternatively spliced region. In some embodiments, the portion of the alternatively spliced region comprises nucleotide positions within 50 bp of a 5′ or 3′ end of the alternatively spliced region. In some embodiments, the antisense nucleic acid sequence comprises a nucleic acid sequence at least 80%, at least 85%, at least 90%, at least 95%, at least 98%, at least 99%, or 100% identical to a target nucleic acid sequence in Tables 2A-2B. In some embodiments, the antisense nucleic acid sequence is 3′ or downstream relative to the ESS nucleic acid sequence. In some embodiments, the engineered U7 snRNA further comprises a Sm binding site. In some embodiments, the Sm binding site comprises AAUUUGUCUAG (SEQ ID NO: 150) or AAUUUUUGGAG (SEQ ID NO: 150; smOPT). In some embodiments, the Sm binding site is 3′ or downstream relative to the ESS nucleic acid sequence or antisense nucleic acid sequence. In some embodiments, the system further comprises a hairpin sequence. Some embodiments include a U7 snRNA hairpin sequence. In some embodiments, the U7 snRNA hairpin sequence comprises the nucleic acid sequence of SEQ ID NO: 4. In some embodiments, the hairpin sequence comprises a 3′ hairpin sequence. In some embodiments, the engineered U7 snRNA further comprises a 3′ U7 snRNA hairpin sequence. In some embodiments, the hairpin sequence is 3′ or downstream relative to the ESS nucleic acid sequence, the antisense nucleic acid sequence, or the Sm binding site. In some embodiments, the system encodes the one or more RNA molecules. In some embodiments, the system comprises a deoxyribonucleic acid (DNA) encoding the engineered U7 snRNA. In some embodiments, the system comprises an expression cassette. In some embodiments, the system comprises an expression cassette encoding an engineered U7 snRNA, the engineered U7 snRNA comprising the ESS nucleic acid sequence and the antisense nucleic acid sequence. In some embodiments, the expression cassette comprises a promoter operably linked to a sequence encoding the engineered U7 snRNA. In some embodiments, the expression cassette comprises a second sequence encoding a second copy of the engineered U7 snRNA, and the promoter is further operably linked to the second sequence. In some embodiments, the expression cassette comprises a third sequence encoding a third copy of the engineered U7 snRNA, and the promoter is further operably linked to the third sequence. In some embodiments, the promoter comprises a mouse U1 snRNA promoter sequence, a human U1 snRNA promoter sequence, a mouse U7 snRNA promoter sequence, a human U7 snRNA promoter sequence, or a combination thereof. In some embodiments, the system comprises a promoter sequence. In some embodiments, the promoter sequence comprises a mouse or human promoter sequence. In some embodiments, the promoter sequence comprises a U7 snRNA promoter sequence. In some embodiments, the promoter sequence comprises a U1 promoter sequence. In some embodiments, the promoter sequence comprises a mouse U7 snRNA (“Mm U7”) promoter sequence, a human U7 snRNA (“Hs U7”) promoter sequence, a mouse U1a1 (“mu1a1” or “Mm U1a1”) promoter sequence, or a human U1-1 (“HU1” or “Hs U1-1”) promoter sequence, or a fragment or combination of fragments thereof. In some embodiments, the promoter sequence comprises a U7 snRNA promoter sequence having a distal sequence element (DSE) replaced with a DSE of a U1-1 or U1a1 promoter sequence. In some embodiments, the promoter sequence comprises a mouse U7 promoter sequence having a proximal sequence element (PSE) replaced with a PSE of a U1-1 or U1a1 promoter sequence. In some embodiments, the promoter sequence comprises a nucleic acid sequence at least 80%, at least 85%, at least 90%, at least 95%, at least 98%, at least 99%, or 100% identical to a promoter sequence in Table 1. In some embodiments, the promoter sequence comprises a nucleic acid sequence at least 80%, at least 85%, at least 90%, at least 95%, at least 98%, at least 99%, or 100% identical to a promoter sequence in Table 5. In some embodiments, the promoter sequence is 5′ or upstream relative to the ESS nucleic acid sequence, the antisense nucleic acid sequence, the Sm binding site, or the hairpin sequence. In some embodiments, the system further comprises a terminator sequence. In some embodiments, the expression cassette comprises a 3′ terminator sequence operably linked to a sequence encoding the engineered U7 snRNA. In some embodiments, the 3′ terminator sequence comprises a mouse U1 snRNA terminator sequence, a human U1 snRNA terminator sequence, a mouse U7 snRNA terminator sequence, a human U7 snRNA terminator sequence, or a combination thereof. In some embodiments, the terminator sequence comprises mouse or human terminator sequence. In some embodiments, the terminator sequence comprises a U7 snRNA terminator sequence. In some embodiments, the terminator sequence comprises a U1 terminator sequence. In some embodiments, the terminator sequence comprises a Mm U7 terminator sequence, a Hs U7 terminator sequence, a mu1a1 terminator sequence, or a HU1 terminator sequence, or a fragment or combination of fragments thereof. In some embodiments, the terminator sequence comprises a U7 snRNA terminator sequence having a distal sequence element (DSE) replaced with a DSE of a U1-1 or U1a1 terminator sequence. In some embodiments, the terminator sequence comprises a mouse U7 snRNA terminator sequence having a proximal sequence element (PSE) replaced with a PSE of a U1-1 or U1a1 terminator sequence. In some embodiments, the terminator sequence comprises a nucleic acid sequence at least 80%, at least 85%, at least 90%, at least 95%, at least 98%, at least 99%, or 100% identical to a terminator sequence in Table 1. In some embodiments, the terminator sequence comprises a nucleic acid sequence at least 80%, at least 85%, at least 90%, at least 95%, at least 98%, at least 99%, or 100% identical to a terminator sequence in Table 6. In some embodiments, the terminator sequence comprises a 3′ terminator sequence. In some embodiments, the terminator sequence is 3′ or downstream relative to the promoter sequence, the ESS nucleic acid sequence, the antisense nucleic acid sequence, the Sm binding site, or the hairpin sequence. In some embodiments, the components are combined together within a single nucleic acid. In some embodiments, the components are separated among multiple nucleic acids. In some embodiments, expression of the system in a cell or a population of cells reduces an exon 20N measurement in a cell or population of cells by at least 10%, at least 20%, at least 30%, at least 40%, at least 50%, at least 60%, at least 70%, at least 80%, or at least 90%, relative to a baseline exon 20N measurement. In some embodiments, expression of the system in a cell or a population of cells reduces an SCN1A NMD transcript measurement in a cell or population of cells by at least 10%, relative to a baseline SCN1A NMD transcript measurement. In some embodiments, expression of the system in a cell or a population of cells increases a productive form of an SCN1A transcript measurement in a cell or population of cells by at least 10%, at least 20%, at least 30%, at least 40%, at least 50%, at least 60%, at least 70%, at least 80%, or at least 90%, relative to a baseline SCN1A transcript measurement. In some embodiments, expression of the system in a cell or a population of cells increases a productive form of an SCN1A transcript measurement in a cell or population of cells by more than 70%, relative to a baseline SCN1A transcript measurement. In some embodiments, expression of the system in a cell or a population of cells an SCN1A protein measurement in a cell or population of cells by at least 10%, at least 20%, at least 30%, at least 40%, at least 50%, at least 60%, at least 70%, at least 80%, or at least 90%, relative to a baseline SCN1A protein measurement. In some embodiments, expression of the system in a cell or a population of cells an SCN1A protein measurement in a cell or population of cells by at least 70%, relative to a baseline SCN1A protein measurement.

Disclosed herein, in some embodiments, are pharmaceutical compositions comprising a system described herein and a pharmaceutically acceptable carrier. Disclosed herein, in some embodiments, are engineered viruses comprising a system described herein. Disclosed herein, in some embodiments, are methods, comprising: administering a pharmaceutical composition or virus disclosed herein to a subject. In some embodiments, the virus comprises a parvovirus. In some embodiments, the virus is an adeno-associated virus (AAV). Disclosed herein, in some embodiments, are methods, comprising: administering the engineered virus to a subject.

Disclosed herein, in some embodiments, are cells comprising a system described herein. In some embodiments, the cell is a neural cell

Disclosed herein, in some embodiments, are methods of modifying splicing, comprising: contacting a pre-mRNA encoding SCN1A with an engineered U7 snRNA or with a vector encoding the engineered U7 snRNA, wherein the engineered U7 snRNA induces exclusion of exon 20N from a mature mRNA generated by the pre-mRNA. In some embodiments, pre-mRNA is in a cell. In some embodiments, the cell is a cell of a subject. In some embodiments, the subject has or is at risk of having epilepsy. In some embodiments, the subject has or is at risk of having Dravet syndrome. In some embodiments, the method comprises administering to the subject a composition that silences or reduces said splicing, the composition comprising a system, virus, or composition herein. In some embodiments, the method increases an amount of a productive isoform of SCN1A, relative to a control or baseline amount of said productive isoform. In some embodiments, the contact or administration decreases an amount of a non-productive isoform of SCN1A in the subject, relative to a control or baseline amount of said non-productive isoform. In some embodiments, the method increases an amount of a NaV1.1, relative to a control or baseline amount of the NaV1.1. In some embodiments, the method improves sodium transport, relative to a control or baseline amount.

Disclosed herein, in some embodiments, are methods of treating or preventing epilepsy in a subject in need thereof, comprising: administering a therapeutically effective amount of a recombinant U7 snRNA composition that silences or reduces splicing of an alternatively spliced region of an RNA encoding SCN1A. Some embodiments include identifying the subject as having epilepsy or as being at risk of having epilepsy, and selecting the treatment based on said identifying. In some embodiments, the alternatively spliced region comprises exon 20N of the RNA. In some embodiments, the subject is in need of treatment for Dravet syndrome. In some embodiments, the composition comprises a system, virus, or composition herein. In some embodiments, the administration increases an amount of a productive isoform of SCN1A in the subject, relative to a control or baseline amount of said productive isoform. In some embodiments, the administration increases an amount of a NaV1.1 channel or protein (e.g. SCN1A protein) in the subject, relative to a control or baseline amount of the NaV1.1 channel or protein. In some embodiments, the administration improves sodium transport in the subject, relative to a control or baseline amount. In some embodiments, the administration prevents the subject from having seizures, or reduces an amount or severity of the seizures of the subject relative to a baseline amount or severity. In some embodiments, the administration reduce a seizure rate in the subject, relative to a baseline seizure rate before the administration. In some embodiments, the seizure rate is reduced in the subject by at least 10%, at least 20%, at least 30%, at least 40%, at least 50%, at least 60%, at least 70%, at least 80%, or at least 90%, relative to the baseline seizure rate. In some embodiments, the administration reduce a seizure duration in the subject, relative to a baseline seizure duration before the administration. In some embodiments, the seizure duration is reduced in the subject by at least 10%, at least 20%, at least 30%, at least 40%, at least 50%, at least 60%, at least 70%, at least 80%, or at least 90%, relative to the baseline seizure duration.

Disclosed herein, in some embodiments, are methods of treating or Dravet Syndrome in a subject in need thereof, comprising: administering a therapeutically effective amount of a synthetic composition comprising a recombinant U7 snRNA that silences or reduces splicing of an alternatively spliced region of a RNA encoding SCN1A. Some embodiments include identifying the subject as having Dravet syndrome, and selecting the treatment based on said identifying. In some embodiments, the alternatively spliced region comprises exon 20N of the RNA. In some embodiments, the composition comprises a system, virus, or composition described herein. In some embodiments, the administration increases an amount of a productive isoform of SCN1A in the subject, relative to a control or baseline amount of said productive isoform. In some embodiments, the administration increases an amount of a NaV1.1 channel or protein (e.g. SCN1A protein) in the subject, relative to a control or baseline amount of the NaV1.1 channel or protein. In some embodiments, the administration improves sodium transport in the subject, relative to a control or baseline amount. In some embodiments, the administration reduces or improves a symptom of the Drave syndrome in the subject. In some embodiments, the administration prevents the subject from having seizures, or reduces an amount or severity of the seizures of the subject relative to a baseline amount or severity.

### BRIEF DESCRIPTION OF THE DRAWINGS

FIG. 1 shows a pairwise alignment of mouse Scn1A and human SCN1A genomic regions including an NMD exon referred to as exon 20N.

FIG. 2 includes the results of an initial U7 screen showing the rank of 96 U7 constructs based on the percent of productive Scn1A isoform expressed.

FIG. 3 includes an analysis of relative NMD transcript levels from Neuro-2a cells transfected with constructs expressing U7 cassettes containing single targeting sequences complementary to regions at both the 5′ and 3′ ends of mouse Scn1A exon 21N.

FIG. 4 includes an analysis of relative NMD transcript levels from Neuro-2a cells transfected with constructs expressing either single U7 cassettes or two U7 cassettes, with one targeting the 5′ region of exon 21N and one targeting the 3′ region of exon 21N (N=3 per construct).

FIG. 5 shows results of end-point RT-PCR for NMD (upper band) and productive (lower band) Scn1A isoform expression in brain tissue samples collected from animals transduced with AAV expressing either Scramble (Ctrl), Candidate 1, or Candidate 2 with 1× U7 cassettes. Each lane represents an individual animal.

FIG. 6 shows results of end-point RT-PCR for NMD (upper band) and productive (lower band) Scn1A isoform expression in samples collected from primary mouse cortical neurons transduced with AAVs expressing either Scramble (Ctrl), Candidate 1, or Candidate 2 with 4× U7 cassettes. Each pair of lanes shows independent biological replicates.

FIG. 7 shows results of end-point RT-PCR for NMD and productive Scn1A isoform expression in wildtype mice injected with increasing doses of AAVs expressing U7 candidates targeting mouse exon 21N.

FIG. 8 shows a dose-dependent increase in NaV1.1 protein expression in wildtype mice injected with AAVs expressing candidate U7 constructs targeting the 5′ end of mouse exon 21N. NaV1.1 levels are normalized to total protein levels in each sample.

FIG. 9 shows an analysis of relative NMD transcript levels in Neuro-2a cells transfected with constructs expressing the core U7 structural sequence with Candidate 1 targeting sequence under the control of different promoters and 3′ elements.

FIG. 10 shows a summary of end-point RT-PCR for relative amounts of productive Scn1A splice isoform in primary mouse cortical neurons transduced with increasing doses of AAVs expressing U7 candidate-1 driven by different combinations of mouse and human U1- and U7-related promoter and 3′ terminator elements.

FIG. 11 shows NaV1.1 levels in whole-brain lysates from mice injected with AAVs expressing U7 candidate 1 driven by different combinations of mouse and human U1-related promoter and 3′ terminator elements. NaV1.1 levels are normalized to total protein levels in each sample.

FIG. 12 shows survival curves of Scn1A control and heterozygous mutant mice injected with either AAV-mu1a1-Scramble-HU1 or AAV-mu1a1-Candidate 1-HU1.

FIG. 13 shows relative NMD transcript levels in Scn1A control and heterozygous mutant mice injected with either AAV-mu1a1-Scramble-HU1 or AAV-mu1a1-Candidate 1-HU1.

FIG. 14 shows total SCN1A mRNA levels in Scn1A control and heterozygous mutant mice injected with either AAV-mu1a1-Scramble-HU1 or AAV-mu1a1-Candidate 1-HU1.

FIG. 15 shows relative NaV1.1 levels in Scn1A control and heterozygous mutant mice injected with either AAV-mu1a1-Scramble-HU1 or AAV-mu1a1-Candidate 1-HU1.

FIG. 16 shows spontaneous seizure frequencies (seizures/24 hr) measured by EEG in Scn1A control and heterozygous mutant mice injected with either saline or U7 AAV (Candidate 3; AAV9) at P2 via ICV. EEG recordings were made from P24-P45 and total seizure counts were averaged over the entire 21-day recording period.

FIG. 17 shows mean seizure durations in heterozygous Scn1A^{+/-} animals exhibiting spontaneous seizures after injection with either saline or U7 AAV (Candidate 3; AAV9) injected via intracerebroventricular (ICV) injection at P2. EEG recording was performed from P24-P45.

FIG. 18 shows survival curves of Scn1A control and heterozygous mutant mice injected with either saline or U7 AAV (Candidate 3; AAV9) at P2 via ICV injection.

FIG. 19 shows survival curves of Scn1A control and heterozygous mutant mice injected with either saline or U7 AAV (Candidate 3; AAV9) at P2 via ICV injection.

FIG. 20 shows relative NaV1.1 protein levels at P90 in Scn1A control and heterozygous mutant mice injected with either saline or U7 AAV (Candidate 3; AAV9) at P2 via ICV.

FIG. 21 shows relative NaV1.1 protein levels at P90 in Scn1A control and heterozygous mutant mice injected with either saline or U7 AAV (Candidate 3; PHP.eB) at P14 via retroorbital injection.

FIG. 22 shows quantification of SCN1A NMD isoform levels by end-point RT-PCR from differentiated human neuronal cultures (ReNcell CX) treated with formulation buffer (vehicle) or three different doses of AAV encoding Candidate 3.

FIG. 23 shows quantification of SCN1A productive isoform levels by end-point RT-PCR from differentiated human neuronal cultures (ReNcell CX) treated with formulation buffer (vehicle) or three different doses of AAV encoding Candidate 3.

### DETAILED DESCRIPTION

Mutation of SCN1A, which encodes sodium channel NaV1.1, is a leading genetic cause of neurodevelopmental disorders such as Dravet syndrome. Analysis of ribonucleic acid (RNA) sequence data has identified an alternate SCN1A mRNA isoform that includes an extra exon, referred to as exon 20N. This alternate isoform is out of frame and results in nonsense-mediated decay (NMD).

Precise pre-mRNA splicing may be essential for appropriate protein translation and may depend on the presence of consensus ‘cis’ sequences that define exon-intron boundaries and regulatory sequences recognized by splicing machinery. Point mutations at these consensus sequences can cause improper exon and intron recognition and may result in the formation of an aberrant transcript of the mutated gene. The splicing mutation may occur in both introns and exons and disrupt existing splice sites or splicing regulatory sequences (intronic and exonic silencers and enhancers), create new ones, or activate crypticones. Usually, such mutations result in errors during the splicing process and may lead to improper intron removal and thus cause alterations of an open reading frame (ORF). Alterations in the open reading frame may lead to truncated or extended mRNA products resulting in the translated protein unable to perform its normal function due to misfolding or degradation. Loss or reduced expression of proteins due to mRNA spicing errors lead to a number of genetic disorders, including Dravet syndrome.

Described herein are recombinant regulatory elements for expression systems. Also described are nucleic acid expression systems with recombinant regulatory elements. The regulatory elements and systems described herein address needs for improved expression systems, such as for gene editing or for transgenic expression.

Dravet syndrome is a devastating epileptic encephalopathy that may arise in otherwise normal babies in the first year of life, later accompanied by developmental delay, intellectual disability and mood disorders. The majority of cases (80%) may be caused by haploinsufficiency of SCN1A, a gene that encodes alpha subunit of the voltage-gated sodium channel protein, NaV1.1. NaV1.1 may also be referred to as sodium channel protein type 1 subunit alpha (SCN1A) protein. Current pharmacological treatments for Dravet syndrome are unable to completely control the epileptic seizures associated with the syndrome. Additionally, the pharmacological treatments are ineffective at inhibiting the subsequent neurological symptoms associated with Dravet syndrome. As a result, there are a number of gene-based therapies being developed for Dravet syndrome. One major obstacle for gene therapies for Dravet syndrome is that the SCN1A-coding sequence is too large to package in adeno-associated vectors (AAV) that are commonly employed for therapeutic gene delivery in the central nervous system. In recent years, alternative genetic approaches aiming to restore physiological levels of NaV1.1 to treat Dravet syndrome have been developed. These strategies rely on boosting the expression of the healthy copy of an SCN1A gene at the transcriptional or post-transcriptional level. One strategy to restore physiological levels of NaV1.1 is through targeted augmentation of nuclear gene output (TANGO). TANGO utilizes antisense oligonucleotides that are specifically designed to target the non-productive splicing site associated with Dravet syndrome and results in expression of the functional NaV1.1 protein. The current application includes a method of treating Dravet syndrome through the delivery of a modified U7 small nuclear RNA (snRNA) targeting exon 20N in humans (21N in mice) of neural cells via AAV so that a functional NaV1.1 protein is expressed and results in increased function of the NaV1.1 protein.

The compositions and methods provided herein may improve upon pervious methods and systems. Some previous systems include described at WO2017106377, WO2019040923, Han et al., Antisense oligonucleotides increase Scn1a expression and reduce seizures and SUDEP incidence in a mouse model of Dravet syndrome (Sci Transl Med. 2020 Aug. 26; 12(558):eaaz6100), and Tanenhaus et al., Cell-Selective Adeno-Associated Virus-Mediated SCN1A Gene Regulation Therapy Rescues Mortality and Seizure Phenotypes in a Dravet Syndrome Mouse Model and Is Well Tolerated in Nonhuman Primates (Hum Gene Ther. 2022 June; 33(11-12):579-597), which references are incorporated by reference in their entirety.

Described herein are compositions and methods for treating a neurodevelopmental disorder in a subject in need thereof. The compositions and methods described herein address an unmet need for safe and effective treatment of neurodevelopmental disorders. The neurodevelopmental disorder may be genetically caused, such as by a mutation in SCN1A (e.g. resulting in SCN1A haploinsufficiency). Described herein are compositions and methods for treating epileptic disorders such as Dravet syndrome in a subject in need thereof. The compositions and methods described herein address an unmet need for safe and effective treatment of epileptic disorders such as Dravet syndrome. The mutation may lead to aberrant SCN1A splicing, or result in inclusion of exon 20N in a mature SCN1A mRNA transcript. Examples of neurodevelopmental disorders may include an intellectual disability or epilepsy (e.g. including seizures).

Some embodiments include a system for modifying nucleic acid splicing. The system may include an exonic splicing silencer (ESS) nucleic acid sequence. An ESS nucleic acid sequence is a sequence capable of enhancing splicing suppression. An ESS may be or include a 10-20 nt sequence at a 5′ terminus of an snRNA (e.g. engineered snRNA) capable of enhancing splicing suppression. The system may include an antisense nucleic acid sequence that targets an alternatively spliced region of an RNA. The antisense nucleic acid sequence may encode an SCN1A protein. Described herein, in some embodiments, is a system for modifying nucleic acid splicing comprising ESS nucleic acid sequence and an antisense nucleic acid sequence that targets an alternatively spliced region of an RNA encoding an SCN1A protein.

Some embodiments relate to a system for modifying nucleic acid splicing, comprising: an engineered U7 snRNA comprising an antisense nucleic acid sequence that targets an alternatively spliced region of an RNA encoding an SCN1A protein. In some embodiments, the engineered U7 snRNA further comprises an ESS nucleic acid sequence. In some embodiments, the engineered U7 snRNA does not comprise an ESS nucleic acid sequence.

Disclosed herein, in some embodiments, are systems for modifying nucleic acid splicing. In some embodiments, the system may include an ESS nucleic acid sequence. In some embodiments, the system may include an antisense nucleic acid sequence that targets an alternatively spliced region of an RNA. In some embodiments, the targeted RNA encodes an SCN1A protein.

Described herein, in some embodiments, is a nucleic acid system that may include deoxyribonucleic acid (DNA). The DNA may include an expression cassette. In some embodiments, the nucleic acid system that may include ribonucleic acid (RNA). The RNA may be encoded by the expression cassette.

### Targeted RNAs and Alternatively Spliced Regions of Targeted RNAs

Described herein, in some embodiments, are methods or systems that affect splicing of a target RNA such as an SCN1A RNA. A target RNA may be referred to as a targeted RNA. A target RNA may include a targeted region. A targeted region may bind with or be bound by an antisense nucleic acid sequence. A targeted region may include an alternatively spliced region such as an alternative exon (e.g. exon 20N). A targeted region may include a splice junction of an alternative exon (e.g. an intron/exon junction comprising at least part of exon 20N). A targeted region may include a region near an alternative exon such as an intron sequence. A targeted region may include an intron sequence. A targeted region may include an alternative exon. A targeted region may exclude an intron. A targeted region may exclude an alternative exon. A targeted region may include part of an intron sequence. A targeted region may include part of an alternative exon. A targeted region may exclude part of an intron sequence. A targeted region may exclude part of an alternative exon. A targeted region may encompass both a region near an alternative exon and at least part of the alternative exon.

Some embodiments refer to or include an alternatively spliced region of a target RNA, such as an alternatively spliced region of an SCN1A RNA. For example, some embodiments of a system or method include or refer to an antisense nucleic acid sequence that targets an alternatively spliced region of a target RNA such as an SCN1A RNA. An example of an alternatively spliced region is exon 20N of an SCN1A RNA. An example of an SCN1A mRNA transcript including exon 20N may be found at www.ncbi.nlm.nih.gov under reference sequence NR_148667.2, as last updated as of the effective filing date.

The alternatively spliced region may be or include an alternatively spliced exon. The alternatively spliced region may be out of frame with a productive transcript. This may lead to production of a downstream stop codon. In some embodiments, inclusion of the alternatively spliced region results in an mRNA (e.g. a mature SCN1A mRNA) having a premature stop codon. Inclusion of a premature stop codon may result in a non-productive mRNA. Inclusion of a premature stop codon may result in the mRNA being non-productive for functional or full-length SCN1A protein. Inclusion of a premature stop codon may result in nonsense mediated decay. In some embodiments, the alternatively spliced regions out of frame with an exon of a productive transcript. In some embodiments, the alternatively spliced region may include a stop codon. In some embodiments, the alternatively spliced region does not include a stop codon.

In some embodiments, the alternatively spliced region may be an alternatively spliced exon. In some embodiments, the alternatively spliced region may not be an alternatively spliced exon. In some embodiments, the alternatively spliced region is an alternatively spliced intron. In some embodiments, the alternatively spliced exon comprises exon 20N (e.g. of a human SCN1A mRNA). In some embodiments, the alternatively spliced exon comprises exon 21N (e.g. of a mouse SCN1A mRNA).

A targeted region may include SEQ ID NO: 153. A targeted region may include part of SEQ ID NO: 153. A targeted region may include a sequence at least 99% identical to SEQ ID NO: 153 or at least 99% identical to part of SEQ ID NO: 153. A targeted region may include a sequence at least 98% identical to SEQ ID NO: 153 or at least 98% identical to part of SEQ ID NO: 153. A targeted region may include a sequence at least 97% identical to SEQ ID NO: 153 or at least 97% identical to part of SEQ ID NO: 153. A targeted region may include a sequence at least 96% identical to SEQ ID NO: 153 or at least 96% identical to part of SEQ ID NO: 153. A targeted region may include a sequence at least 95% identical to SEQ ID NO: 153 or at least 95% identical to part of SEQ ID NO: 153. A targeted region may include a sequence at least 94% identical to SEQ ID NO: 153 or at least 94% identical to part of SEQ ID NO: 153. A targeted region may include a sequence at least 93% identical to SEQ ID NO: 153 or at least 93% identical to part of SEQ ID NO: 153. A targeted region may include a sequence at least 92% identical to SEQ ID NO: 153 or at least 92% identical to part of SEQ ID NO: 153. A targeted region may include a sequence at least 91% identical to SEQ ID NO: 153 or at least 91% identical to part of SEQ ID NO: 153. A targeted region may include a sequence at least 9′% identical to SEQ ID NO: 153 or at least 9′% identical to part of SEQ ID NO: 153.

A targeted region may include SEQ ID NO: 154. A targeted region may include part of SEQ ID NO: 154. A targeted region may include a sequence at least 99% identical to SEQ ID NO: 154 or at least 99% identical to part of SEQ ID NO: 154. A targeted region may include a sequence at least 98% identical to SEQ ID NO: 154 or at least 98% identical to part of SEQ ID NO: 154. A targeted region may include a sequence at least 97% identical to SEQ ID NO: 154 or at least 97% identical to part of SEQ ID NO: 154. A targeted region may include a sequence at least 96% identical to SEQ ID NO: 154 or at least 96% identical to part of SEQ ID NO: 154. A targeted region may include a sequence at least 95% identical to SEQ ID NO: 154 or at least 95% identical to part of SEQ ID NO: 154. A targeted region may include a sequence at least 94% identical to SEQ ID NO: 154 or at least 94% identical to part of SEQ ID NO: 154. A targeted region may include a sequence at least 93% identical to SEQ ID NO: 154 or at least 93% identical to part of SEQ ID NO: 154. A targeted region may include a sequence at least 92% identical to SEQ ID NO: 154 or at least 92% identical to part of SEQ ID NO: 154. A targeted region may include a sequence at least 91% identical to SEQ ID NO: 154 or at least 91% identical to part of SEQ ID NO: 154. A targeted region may include a sequence at least 9′% identical to SEQ ID NO: 154 or at least 9′% identical to part of SEQ ID NO: 154.

In some embodiments, the exon may include a nucleic acid sequence identical to SEQ ID NO: 1. In some embodiments, the exon may include a nucleic acid sequence at least 99% identical to SEQ ID NO: 1. In some embodiments, the exon may include a nucleic acid sequence at least 98% identical to SEQ ID NO: 1. In some embodiments, the exon may include a nucleic acid sequence at least 97% identical to SEQ ID NO: 1. In some embodiments, the exon may include a nucleic acid sequence at least 96% identical to SEQ ID NO: 1. In some embodiments, the exon may include a nucleic acid sequence at least 95% identical to SEQ ID NO: 1. In some embodiments, the exon may include a nucleic acid sequence at least 94% identical to SEQ ID NO: 1. In some embodiments, the exon may include a nucleic acid sequence at least 93% identical to SEQ ID NO: 1. In some embodiments, the exon may include a nucleic acid sequence at least 92% identical to SEQ ID NO: 1. In some embodiments, the exon may include a nucleic acid sequence at least 91% identical to SEQ ID NO: 1. In some embodiments, the exon may include a nucleic acid sequence at least 90% identical to SEQ ID NO: 1. In some embodiments, the exon may include a nucleic acid sequence at least 85% identical to SEQ ID NO: 1. In some embodiments, the exon may include a nucleic acid sequence at least 80% identical to SEQ ID NO: 1. In some embodiments, the exon may include a nucleic acid sequence at least 75% identical to SEQ ID NO: 1. In some embodiments, the exon may include a nucleic acid sequence of at least 70% identical to SEQ ID NO: 1.

In some embodiments, the exon may include a nucleic acid sequence identical to SEQ ID NO: 2. In some embodiments, the exon may include a nucleic acid sequence at least 99% identical to SEQ ID NO: 2. In some embodiments, the exon may include a nucleic acid sequence at least 98% identical to SEQ ID NO: 2. In some embodiments, the exon may include a nucleic acid sequence at least 97% identical to SEQ ID NO: 2. In some embodiments, the exon may include a nucleic acid sequence at least 96% identical to SEQ ID NO: 2. In some embodiments, the exon may include a nucleic acid sequence at least 95% identical to SEQ ID NO: 2. In some embodiments, the exon may include a nucleic acid sequence at least 94% identical to SEQ ID NO: 2. In some embodiments, the exon may include a nucleic acid sequence at least 93% identical to SEQ ID NO: 2. In some embodiments, the exon may include a nucleic acid sequence at least 92% identical to SEQ ID NO: 2. In some embodiments, the exon may include a nucleic acid sequence at least 91% identical to SEQ ID NO: 2. In some embodiments, the exon may include a nucleic acid sequence at least 90% identical to SEQ ID NO: 2. In some embodiments, the exon may include a nucleic acid sequence at least 85% identical to SEQ ID NO: 2. In some embodiments, the exon may include a nucleic acid sequence at least 80% identical to SEQ ID NO: 2. In some embodiments, the exon may include a nucleic acid sequence at least 75% identical to SEQ ID NO: 2. In some embodiments, the exon may include a nucleic acid sequence of at least 70% identical to SEQ ID NO: 2. An antisense nucleic acid sequence may target any of the aforementioned exon sequences. An antisense nucleic acid sequence may target a region of any of the aforementioned exon sequences.

A targeted region may be or comprise a length of nucleotides. For example, a targeted region may be about 10, about 15, about 20, about 25, about 30, about 35, about 40, about 45, about 50, about 60, about 70, about 80, about 90, about 100, about 110, about 120, about 130, about 140, or about 150 nucleotides in length, or a range of lengths defined by any 2 of the aforementioned lengths. In some embodiments, the length is at least 10 nucleotides. In some embodiments, the length is at least 15 nucleotides. In some embodiments, the length is at least 20 nucleotides. In some embodiments, the length is at least 25 nucleotides. In some embodiments, the length is at least 30 nucleotides. In some embodiments, the length is at least 35 nucleotides. In some embodiments, the length is at least 40 nucleotides. In some embodiments, the length is at least 45 nucleotides. In some embodiments, the length is at least 50 nucleotides. In some embodiments, the length is at least 60 nucleotides. In some embodiments, the length is at least 70 nucleotides. In some embodiments, the length is at least 80 nucleotides. In some embodiments, the length is at least 90 nucleotides. In some embodiments, the length is at least 100 nucleotides. In some embodiments, the length is at least 110 nucleotides. In some embodiments, the length is at least 120 nucleotides. In some embodiments, the length is at least 130 nucleotides. In some embodiments, the length is at least 140 nucleotides. In some embodiments, the length is at least 150 nucleotides. In some embodiments, the length is less than 15 nucleotides. In some embodiments, the length is less than 20 nucleotides. In some embodiments, the length is less than 25 nucleotides. In some embodiments, the length is less than 30 nucleotides. In some embodiments, the length is less than 35 nucleotides. In some embodiments, the length is less than 40 nucleotides. In some embodiments, the length is less than 45 nucleotides. In some embodiments, the length is less than 50 nucleotides. In some embodiments, the length is less than 60 nucleotides. In some embodiments, the length is less than 70 nucleotides. In some embodiments, the length is less than 80 nucleotides. In some embodiments, the length is less than 90 nucleotides. In some embodiments, the length is less than 100 nucleotides. In some embodiments, the length is less than 110 nucleotides. In some embodiments, the length is less than 120 nucleotides. In some embodiments, the length is less than 130 nucleotides. In some embodiments, the length is less than 140 nucleotides. In some embodiments, the length is less than 150 nucleotides.

In some embodiments, an antisense nucleic acid sequence targets a targeted region of an SCN1A RNA. In some embodiments, the targeted region is within an exon of the endogenous SCN1A RNA. In some embodiments, the targeted region is within an exon of an endogenous SCN1A mRNA. In some embodiments, an antisense nucleic acid sequence targets an alternatively spliced exon of the endogenous SCN1A RNA. In some embodiments, the alternatively spliced exon includes an exon 20N of an SCN1A RNA.

In some embodiments, the targeted region is within a 5′ half or 5′ end of an intron or exon of the endogenous SCN1A RNA. For example, in some embodiments, the targeted region may be closer to the 5′ end of an intron of the endogenous SCN1A RNA. In some embodiments, the targeted region includes the 5′ end of an intron of the endogenous SCN1A RNA. In some embodiments, the targeted region may be closer to the 5′ end of an exon of the endogenous SCN1A RNA. In some embodiments, the targeted region includes the 5′ end of an exon of the endogenous SCN1A RNA.

In some embodiments, the targeted region is within a 3′ half or 3′ end of an intron or exon of the endogenous SCN1A RNA. For example, in some embodiments, the targeted region may be closer to the 3′ end of an intron of the endogenous SCN1A RNA. In some embodiments, the targeted region includes the 3′ end of an intron of the endogenous SCN1A RNA. In some embodiments, the targeted region may be closer to the 3′ end of an exon of the endogenous SCN1A RNA. In some embodiments, the targeted region includes the 3′ end of an exon of the endogenous SCN1A RNA.

In some embodiments, the targeted region is within 100 nucleotides of an intron/exon junction. In some embodiments, the targeted region is within 10, 20, 30, 40, 50, 60, 70, 80, 90, or 100 nucleotides of an intron/exon junction. In some embodiments, the targeted region is not within 10, 20, 30, 40, 50, 60, 70, 80, 90, or 100 nucleotides of an intron/exon junction.

In some embodiments, the targeted region of the endogenous SCN1A RNA (e.g. SCN1A mRNA or pre-mRNA) includes an intron-exon junction of the endogenous RNA. The intron-exon junction refers to the boundary between an intron and exon and includes the splice site that separates the intron and the exon upon pre-mRNA splicing. In some embodiments, the targeted region of the endogenous SCN1A RNA is within 0 nt and 150 nt of an intron-exon junction. In some embodiments, the targeted region of the endogenous SCN1A RNA is within 10 nt and 150 nt of an intron-exon junction. In some embodiments, the targeted region of the endogenous SCN1A RNA is within 20 nt and 150 nt of an intron-exon junction. In some embodiments, the targeted region of the endogenous SCN1A RNA is within 30 nt and 150 nt of an intron-exon junction. In some embodiments, the targeted region of the endogenous SCN1A RNA is within 40 nt and 150 nt of an intron-exon junction. In some embodiments, the targeted region of the endogenous SCN1A RNA is within 40 nt and 150 nt of an intron-exon junction. In some embodiments, the targeted region of the endogenous SCN1A RNA is within 60 nt and 150 nt of an intron-exon junction. In some embodiments, the targeted region of the endogenous SCN1A RNA is within 70 nt and 150 nt of an intron-exon junction. In some embodiments, the targeted region of the endogenous SCN1A RNA is within 80 nt and 150 nt of an intron-exon junction. In some embodiments, the targeted region of the endogenous SCN1A RNA is within 90 nt and 150 nt of an intron-exon junction. In some embodiments, the targeted region of the endogenous SCN1A RNA is within 100 nt and 150 nt of an intron-exon junction. In some embodiments, the targeted region of the endogenous SCN1A RNA is within 110 nt and 150 nt of an intron-exon junction. In some embodiments, the targeted region of the endogenous SCN1A RNA is within 120 nt and 150 nt of an intron-exon junction. In some embodiments, the targeted region of the endogenous SCN1A RNA is within 130 nt and 150 nt of an intron-exon junction. In some embodiments, the targeted region of the endogenous SCN1A RNA is within 140 nt and 150 nt of an intron-exon junction.

In some embodiments, the targeted region of the endogenous SCN1A RNA is within 0 nt and 140 nt of an intron-exon junction. In some embodiments, the targeted region of the endogenous SCN1A RNA is within 0 nt and 130 nt of an intron-exon junction. In some embodiments, the targeted region of the endogenous SCN1A RNA is within 0 nt and 120 nt of an intron-exon junction. In some embodiments, the targeted region of the endogenous SCN1A RNA is within 0 nt and 110 nt of an intron-exon junction. In some embodiments, the targeted region of the endogenous SCN1A RNA is within 0 nt and 100 nt of an intron-exon junction. In some embodiments, the targeted region of the endogenous SCN1A RNA is within 0 nt and 90 nt of an intron-exon junction. In some embodiments, the targeted region of the endogenous SCN1A RNA is within 0 nt and 80 nt of an intron-exon junction. In some embodiments, the targeted region of the endogenous SCN1A RNA is within 0 nt and 70 nt of an intron-exon junction. In some embodiments, the targeted region of the endogenous SCN1A RNA is within 0 nt and 60 nt of an intron-exon junction. In some embodiments, the targeted region of the endogenous SCN1A RNA is within 0 nt and 50 nt of an intron-exon junction. In some embodiments, the targeted region of the endogenous SCN1A RNA is within 0 nt and 40 nt of an intron-exon junction. In some embodiments, the targeted region of the endogenous SCN1A RNA is within 0 nt and 30 nt of an intron-exon junction. In some embodiments, the targeted region of the endogenous SCN1A RNA is within 0 nt and 20 nt of an intron-exon junction.

In some embodiments, the targeted region of the endogenous SCN1A RNA is within 0 nt, 10 nt, 20 nt, 30 nt, 40 nt, 50 nt, 60 nt, 70 nt, 80 nt, 90 nt, 100 nt, 110 nt, 120 nt, 130 nt, 140 nt, or 150 of an intron-exon junction. In some embodiments, the targeted region of the endogenous SCN1A RNA includes an intro-exon junction.

A target RNA or SCN1A RNA may be or include an SCN1A mRNA. An SCN1A mRNA may be or include an SCN1A pre-mRNA. For example, a target SCN1A RNA may include an SCN1A pre-mRNA. A pre-mRNA may include an mRNA before splicing, or before splicing is completed. Whan an mRNA has fully undergone splicing, it may be referred to as a mature mRNA.

In some embodiments, the target RNA or SCN1A RNA comprises a mammalian SCN1A RNA. In some embodiments, the SCN1A RNA comprises a primate SCN1A RNA. In some embodiments, the SCN1A RNA comprises a human SCN1A RNA. In some embodiments, the SCN1A RNA comprises a rodent or mouse SCN1A RNA.

### SCN1A

Some embodiments relate to sodium channel protein type 1 subunit alpha (SCN1A) protein. For example, a method may be directed at reducing or preventing inclusion of a non-productive exon in a mature SCN1A mRNA, where inclusion of the non-productive exon results in nonsense-mediated decay (NMD). Some examples of SCN1A protein sequences are included at UniProt.org under accession numbers P35498 (human) and A2APX8 (mouse), as last updated as of the effective filing date. The SCN1A protein may include the amino acid sequence of SEQ ID NO: 155, which includes the sequence found at UniProt P35498-1 (human SCN1A protein). An SCN1A protein may include an amino acid sequence at least 85%, at least 90%, at least 91%, at least 92%, at least 93%, at least 94%, at least 95%, at least 96%, at least 97%, at least 98%, or at least 99% identical to SEQ ID NO: 155.

Modified U7 snRNAs

Disclosed herein, in some embodiments, are recombinant small nuclear RNAs (snRNAs) or snRNA sequences. Disclosed herein, in some embodiments, are modified or recombinant U7 small nuclear RNAs (snRNAs) or modified or recombinant U7 snRNA sequences. A modified or recombinant U7 snRNA, or a modified or recombinant U7 snRNA sequence may be included as part of a system, or may be used in a method herein. Disclosed herein, in some embodiments, are systems that include a modified or recombinant U7 snRNA or that include a modified or recombinant U7 snRNA sequence. The modified or recombinant U7 snRNA may be or include an engineered U7 snRNA. Terms such as modified, recombinant, and engineered may be used interchangeably herein. The U7 snRNA sequence may be useful for modifying nucleic acid splicing. In some embodiments, the U7 snRNA sequence may include an exonic splicing silencer (ESS) nucleic acid sequence. Some embodiments do not include an ESS nucleic acid sequence. In some embodiments, the U7 snRNA sequence may include an antisense nucleic acid sequence that targets an alternatively spliced region of a ribonucleic acid (RNA). In some embodiments, the targeted RNA may encode a sodium channel protein type 1 subunit alpha (SCN1A) protein. The U7 snRNA sequence may include a smOPT sequence. The U7 snRNA sequence may include a hairpin.

Described herein, in some embodiments, is a nucleic acid system that may include one or more RNA molecules. In some embodiments, the system may include at least one RNA molecule. In some embodiments, the system may include at least two RNA molecules. In some embodiments, the system may include at least three RNA molecules. In some embodiments, the system may include at least four RNA molecules. In some embodiments, the system may include at least five RNA molecules. In some embodiments, the system may include at most one RNA molecule. In some embodiments, the system may include at most two RNA molecules. In some embodiments, the system may include at most three RNA molecules. In some embodiments, the system may include at most four RNA molecules. In some embodiments, the system may include at most five RNA molecules.

In some embodiments, the RNA may be a modified U7 snRNA. Some U rich small nuclear ribonucleoproteins (snRNPs) include complexes that mediate the splicing of pre-mRNAs. U7 snRNP may be an exception in not being involved in splicing but may be a key factor in 3′ end processing of replication-dependent histone mRNAs. However, by introducing controlled changes in the U7 snRNA histone binding sequence and in the Sm motif, U7 can be used as an effective tool for gene therapy. The modified U7 snRNP may thus be not involved in processing of replication-dependent histone pre-mRNA but instead target splicing by inducing efficient skipping or inclusion of selected exons. Modification of the sequence motif of U7 snRNA can make the snRNP particle hybridize to almost any RNA sequence within the nucleoplasm.

The modified U7 snRNA may include, in the following order from 5′ to 3′: (1) an optional exonic splicing silencer, (2) a targeting sequence (e.g. an antisense sequence) complementary to a region of an SCN1A mRNA, (4) a smOPT sequence, and (5) a hairpin sequence.

In some embodiments, a modified U7 snRNA includes a U7 core sequence, or an aspect of a U7 core sequence. In some embodiments, a recombinant U7 snRNA includes a U7 core sequence, or an aspect of a U7 core sequence. An example of a U7 core sequence is included in Table 1. For the U7 core sequence shown in Table 1, an antisense sequence is in parentheses, a smOPT is in in upper case, and a hairpin is in brackets. The antisense sequence may be or include a targeting sequence herein.

TABLE 1

| Component | Sequence (5′ to 3′) | SEQ ID NO: |
|---|---|---|
| Mouse Exon | GATAATCTTGCTCCAACTTGGATGGGGTGGAGCGGTGGTT | 1 |
| 21N | CCTCCCCTCAGCCCTTTATTATGG | |
| Human Exon | GATAATCTTGCTCCAACTTGGATGGGGTGGAGCGCTGGTT | 2 |
| 20N | CCTCCCCTGAGCCCTTTATTATGG | |
| U7 Core | a(N...N)AATTTTTGGAG[caggttttctgacttcggtcggaaaacccct] | 3 |
| Sequence | | |
| U7 Hairpin | CAGGUUUUCUGACUUCGGUCGGAAAACCCCU | 4 |
| Sequence | | |
| Mm U7 | Aacaacataggagctgtgattggctgttttcagccaatcagcactgactcattt | 5 |
| Promoter | gcatagcctttacaagcggtcacaaactcaagaaacgagcggttttaatagtct | |
| | tttagaatattgtttatcgaaccgaataaggaactgtgctttgtgattcacata | |
| | tcagtggaggggtgtggaaatggcaccttgatctcaccctcatcgaaagtggag | |
| | ttgatgtccttccctggctcgctacagacgcacttccgc | |
| Mm U1a1 | TAAAGACTGTGCATCCGACTCCTACATTTATGAAAGTAAA | 6 |
| Promoter | TGCCTATTGTTAGAACAAAAAAGGCTACAGAACAAAAAA | |
| | CAAAGCGAAATACCATCTGCTTTAGGTTCAGTGGTATTTTC | |
| | CCGCTGACAGGGAGGCGGGTTTTTGGGTACAGGAAACGA | |
| | GTCACTATGGAGGCGGTACTATGTAGATGAGAATTCAGGA | |
| | GCAAACTGGGAAAAGCAACTGCTTCCAAATATTTGTGATT | |
| | TTTACAGTGTAGTTTTGGAAAAACTCTTAGCCTACCAATTC | |
| | TTCTAAGTGTTTTAAAATGTGGGAGCCAGTACACATGAAG | |
| | TTATAGAGTGTTTTAATGAGGCTTAAATATTTACCGTAACT | |
| | ATGAAATGCTACGCATATCATGCTGTTCAGGCTCCGTGGC | |
| | CACGCAACTC | |
| Hs U1-1 | aacaacataggagctgtgattggctgttttcagccaatcagcactgactcAGGGCGACTT | 7 |
| Promoter | CTATGTAGATGAGGCAGCGCAGAGGCTGCTgcctttacaagcggtcacaaactcaagaaa | |
| | cgagcggttttaatagtcttttagaatattgtttatcgaaccgaataaggaactgtgctt | |
| | tgtgattcacatatcagtggaggggtgtggaaatggcaccttgatGGGAGTGCG | |
| | CGAGGCAAGTGACCGTGTGTGgagttgatgtccttccctggctcgctacagac | |
| | gcacttccgc | |
| Modified Mm | aacaacataggagctgtgattggctgttttcagccaatcagcactgactcAGGGCGACTT | 8 |
| U7 Promoter | CTATGTAGATGAGGCAGCGCAGAGGCTGCTgcctttacaagcggtcacaaactcaagaaa | |
| | cgagcggttttaatagtcttttagaatattgtttatcgaaccgaataaggaactgtgctt | |
| | tgtgattcacatatcagtggaggggtgtggaaatggcaccttgatTTACCGTAAC | |
| | TATGAAATGgagttgatgtccttccctggctcgctacagacgcacttccgc | |
| Mm U7 3′ | Cccaatttcactggtctacaatgaaagcaaaacagttctcttccccgctcc | 9 |
| | ccggtgtgtgagagg | |
| Region | ggctttgatccttctctggtttcctaggaaacgcgtat | |
| Hs U1-13′ | ACTTTCTGGAGTTTCAAAAGTAGACTGTACGCTAA | 10 |
| Region | | |

Some embodiments include a modified U7 small nuclear RNA (snRNA). In some embodiments, the modified U7 snRNA includes a U7 core sequence at least 80% identical to a U7 core sequence set forth in Table 1. In some embodiments, the modified U7 snRNA includes a U7 core sequence at least 85% identical to a U7 core sequence set forth in Table 1. In some embodiments, the modified U7 snRNA includes a U7 core sequence at least 90% identical to a U7 core sequence set forth in Table 1. In some embodiments, the modified U7 snRNA includes a U7 core sequence at least 91% identical to a U7 core sequence set forth in Table 1. In some embodiments, the modified U7 snRNA includes a U7 core sequence at least 92% identical to a U7 core sequence set forth in Table 1. In some embodiments, the modified U7 snRNA includes a U7 core sequence at least 93% identical to a U7 core sequence set forth in Table 1. In some embodiments, the modified U7 snRNA includes a U7 core sequence at least 94% identical to a U7 core sequence set forth in Table 1. In some embodiments, the modified U7 snRNA includes a U7 core sequence at least 95% identical to a U7 core sequence set forth in Table 1. In some embodiments, the modified U7 snRNA includes a U7 core sequence at least 96% identical to a U7 core sequence set forth in Table 1. In some embodiments, the modified U7 snRNA includes a U7 core sequence at least 97% identical to a U7 core sequence set forth in Table 1. In some embodiments, the modified U7 snRNA includes a U7 core sequence at least 98% identical to a U7 core sequence set forth in Table 1. In some embodiments, the modified U7 snRNA includes a U7 core sequence at least 99% identical to a U7 core sequence set forth in Table 1. In some embodiments, the modified U7 snRNA includes a U7 core sequence set forth in Table 1. In some embodiments, the modified U7 snRNA is a U7 core sequence set forth in Table 1.

Some embodiments include a modified U7 small nuclear RNA (snRNA). Some examples of sequences of modified U7 snRNAs are in Table 7A. In some embodiments, the modified U7 snRNA is or includes an RNA sequence at least 80% identical to an RNA sequence set forth in Table 7A. In some embodiments, the modified U7 snRNA is or includes an RNA sequence at least 85% identical to an RNA sequence set forth in Table 7A. In some embodiments, the modified U7 snRNA is or includes an RNA sequence at least 90% identical to an RNA sequence set forth in Table 7A. In some embodiments, the modified U7 snRNA is or includes an RNA sequence at least 91% identical to an RNA sequence set forth in Table 7A. In some embodiments, the modified U7 snRNA is or includes an RNA sequence at least 92% identical to an RNA sequence set forth in Table 7A. In some embodiments, the modified U7 snRNA is or includes an RNA sequence at least 93% identical to an RNA sequence set forth in Table 7A. In some embodiments, the modified U7 snRNA is or includes an RNA sequence at least 94% identical to an RNA sequence set forth in Table 7A. In some embodiments, the modified U7 snRNA is or includes an RNA sequence at least 95% identical to an RNA sequence set forth in Table 7A. In some embodiments, the modified U7 snRNA is or includes an RNA sequence at least 96% identical to an RNA sequence set forth in Table 7A. In some embodiments, the modified U7 snRNA is or includes an RNA sequence at least 97% identical to an RNA sequence set forth in Table 7A. In some embodiments, the modified U7 snRNA is or includes an RNA sequence at least 98% identical to an RNA sequence set forth in Table 7A. In some embodiments, the modified U7 snRNA is or includes an RNA sequence at least 99% identical to an RNA sequence set forth in Table 7A. In some embodiments, the modified U7 snRNA is or includes an RNA sequence set forth in Table 7A. In some embodiments, the modified U7 snRNA is an RNA sequence set forth in Table 7A.

Some examples of sequences of modified U7 snRNAs are in Table 7B. In some embodiments, the modified U7 snRNA is or includes an RNA sequence at least 80% identical to an RNA sequence set forth in Table 7B. In some embodiments, the modified U7 snRNA is or includes an RNA sequence at least 85% identical to an RNA sequence set forth in Table 7B. In some embodiments, the modified U7 snRNA is or includes an RNA sequence at least 90% identical to an RNA sequence set forth in Table 7B. In some embodiments, the modified U7 snRNA is or includes an RNA sequence at least 91% identical to an RNA sequence set forth in Table 7B. In some embodiments, the modified U7 snRNA is or includes an RNA sequence at least 92% identical to an RNA sequence set forth in Table 7B. In some embodiments, the modified U7 snRNA is or includes an RNA sequence at least 93% identical to an RNA sequence set forth in Table 7B. In some embodiments, the modified U7 snRNA is or includes an RNA sequence at least 94% identical to an RNA sequence set forth in Table 7B. In some embodiments, the modified U7 snRNA is or includes an RNA sequence at least 95% identical to an RNA sequence set forth in Table 7B. In some embodiments, the modified U7 snRNA is or includes an RNA sequence at least 96% identical to an RNA sequence set forth in Table 7B. In some embodiments, the modified U7 snRNA is or includes an RNA sequence at least 97% identical to an RNA sequence set forth in Table 7B. In some embodiments, the modified U7 snRNA is or includes an RNA sequence at least 98% identical to an RNA sequence set forth in Table 7B. In some embodiments, the modified U7 snRNA is or includes an RNA sequence at least 99% identical to an RNA sequence set forth in Table 7B. In some embodiments, the modified U7 snRNA is or includes an RNA sequence set forth in Table 7B. In some embodiments, the modified U7 snRNA is an RNA sequence set forth in Table 7B.

Some examples of sequences of modified U7 snRNAs are in Table 7C. In some embodiments, the modified U7 snRNA is or includes an RNA sequence at least 80% identical to an RNA sequence set forth in Table 7C. In some embodiments, the modified U7 snRNA is or includes an RNA sequence at least 85% identical to an RNA sequence set forth in Table 7C. In some embodiments, the modified U7 snRNA is or includes an RNA sequence at least 90% identical to an RNA sequence set forth in Table 7C. In some embodiments, the modified U7 snRNA is or includes an RNA sequence at least 91% identical to an RNA sequence set forth in Table 7C. In some embodiments, the modified U7 snRNA is or includes an RNA sequence at least 92% identical to an RNA sequence set forth in Table 7C. In some embodiments, the modified U7 snRNA is or includes an RNA sequence at least 93% identical to an RNA sequence set forth in Table 7C. In some embodiments, the modified U7 snRNA is or includes an RNA sequence at least 94% identical to an RNA sequence set forth in Table 7C. In some embodiments, the modified U7 snRNA is or includes an RNA sequence at least 95% identical to an RNA sequence set forth in Table 7C. In some embodiments, the modified U7 snRNA is or includes an RNA sequence at least 96% identical to an RNA sequence set forth in Table 7C. In some embodiments, the modified U7 snRNA is or includes an RNA sequence at least 97% identical to an RNA sequence set forth in Table 7C. In some embodiments, the modified U7 snRNA is or includes an RNA sequence at least 98% identical to an RNA sequence set forth in Table 7C. In some embodiments, the modified U7 snRNA is or includes an RNA sequence at least 99% identical to an RNA sequence set forth in Table 7C. In some embodiments, the modified U7 snRNA is or includes an RNA sequence set forth in Table 7C. In some embodiments, the modified U7 snRNA is an RNA sequence set forth in Table 7C.

In some embodiments, the system reduces a nonsense-mediated decay (NMD) exon measurement (e.g. an exon 20N measurement) in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, relative to a baseline target measurement. In some embodiments, expression of the system in a cell or a population of cells reduces an exon 20N measurement in a cell or population of cells by at least 10%, at least 20%, at least 30%, at least 40%, at least 50%, at least 60%, at least 70%, at least 80%, or at least 90%, relative to a baseline exon 20N measurement. In some embodiments, the system reduces a nonsense-mediated decay (NMD) exon measurement (e.g. an exon 20N measurement) in a cell or population of cells by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, or less than 95%, relative to a baseline target measurement.

In some embodiments, the system reduces a nonsense-mediated decay (NMD) transcript measurement (e.g. an NMD SCN1A RNA measurement) in a cell or population of cells by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, relative to a baseline target measurement. In some embodiments, expression of the system in a cell or a population of cells increases a productive form of an SCN1A transcript measurement in a cell or population of cells by at least 10%, at least 20%, at least 30%, at least 40%, at least 50%, at least 60%, at least 70%, at least 80%, or at least 90%, relative to a baseline SCN1A transcript measurement. In some embodiments, expression of the system in a cell or a population of cells increases a productive form of an SCN1A transcript measurement in a cell or population of cells by more than 70%, relative to a baseline SCN1A transcript measurement. In some embodiments, the system reduces a nonsense-mediated decay (NMD) transcript measurement (e.g. an NMD SCN1A RNA measurement) in a cell or population of cells by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, or less than 95%, relative to a baseline target measurement.

In some embodiments, expression of the system in a cell or a population of cells an TARGET protein measurement in a cell or population of cells by at least 10%, at least 20%, at least 30%, at least 40%, at least 50%, at least 60%, at least 70%, at least 80%, or at least 90%, relative to a baseline TARGET protein measurement. In some embodiments, expression of the system in a cell or a population of cells an TARGET protein measurement in a cell or population of cells by at least 70%, relative to a baseline TARGET protein measurement. In some embodiments, expression of the system in a cell or a population of cells an SCN1A protein measurement in a cell or population of cells by at least 10%, at least 20%, at least 30%, at least 40%, at least 50%, at least 60%, at least 70%, at least 80%, or at least 90%, relative to a baseline SCN1A protein measurement. In some embodiments, expression of the system in a cell or a population of cells an SCN1A protein measurement in a cell or population of cells by at least 70%, relative to a baseline SCN1A protein measurement. In some embodiments, expression of the system in a cell or a population of cells an target protein measurement in a cell or population of cells by less than 10%, less than 20%, less than 30%, less than 40%, less than 50%, less than 60%, less than 70%, less than 80%, or less than 90%, relative to a baseline target protein measurement. In some embodiments, expression of the system in a cell or a population of cells an target protein measurement in a cell or population of cells by less than 70%, relative to a baseline target protein measurement.

Disclosed herein, in some embodiments, are polynucleotides, comprising: an exonic splicing silencer (ESS) nucleic acid sequence; and an antisense nucleic acid sequence that binds to an alternatively spliced region of a ribonucleic acid (RNA) encoding sodium channel protein type 1 subunit alpha (SCN1A). In some embodiments, the ESS nucleic acid sequence comprises a sequence having at least 80%, at least 85%, at least 90%, at least 95%, at least 98%, at least 99%, or 100% identity to any one of SEQ ID NOs: 11-13, optionally wherein the ESS nucleic acid sequence comprises the nucleic acid sequence of any one of SEQ ID NOs: 11-13. In some embodiments, the antisense nucleic acid sequence comprises a sequence having at least 80%, at least 85%, at least 90%, at least 95%, at least 98%, at least 99%, or 100% identity to any one of SEQ ID NOs: 14 and 18-104, optionally wherein the antisense nucleic acid sequence comprises the nucleic acid sequence of any one of SEQ ID NOs: 14 and 18-104. In some embodiments, the polynucleotide further comprises a Sm binding site. In some embodiments, the Sm binding site comprises a nucleic acid sequence having at least 80%, at least 85%, at least 90%, at least 95%, at least 98%, at least 99%, or 100% identity to the nucleic acid sequence of any one of SEQ ID NOs: 150-151, optionally wherein the Sm binding site comprises the nucleic acid sequence of any one of SEQ ID NOs: 150-151. In some embodiments, the polynucleotide further comprises a hairpin sequence. In some embodiments, the hairpin sequence comprises a nucleic acid sequence having at least 80%, at least 85%, at least 90%, at least 95%, at least 98%, at least 99%, or 100% identity to the nucleic acid sequence of SEQ ID NO: 4, optionally wherein the hairpin sequence comprises the nucleic acid sequence of SEQ ID NO: 4. In some embodiments, the polynucleotide comprising a nucleic acid sequence having at least 80%, at least 85%, at least 90%, at least 95%, at least 98%, at least 99%, or 100% identity to the nucleic acid sequence of any one of SEQ ID NOs: 364-566.

Disclosed herein, in some embodiments, are polynucleotides, comprising: a first sequence comprising a nucleic acid sequence having at least 80%, at least 85%, at least 90%, at least 95%, at least 98%, at least 99%, or 100% identity to any one of SEQ ID NOs: 11-13; and a second sequence comprising a nucleic acid sequence having at least 80%, at least 85%, at least 90%, at least 95%, at least 98%, at least 99%, or 100% identity to any one of SEQ ID NOs: 14 and 18-104. In some embodiments, the first sequence comprises the nucleic acid sequence of any one of SEQ ID NOs: 11-13. In some embodiments, the second sequence comprises the nucleic acid sequence of any one of SEQ ID NOs: 14 and 18-104. In some embodiments, the polynucleotide further a third sequence, wherein the third sequence comprises a nucleic acid sequence having at least 80%, at least 85%, at least 90%, at least 95%, at least 98%, at least 99%, or 100% identity to the nucleic acid sequence of any one of SEQ ID NOs: 150-151, optionally wherein the third sequence comprises the nucleic acid sequence of any one of SEQ ID NOs: 150-151. In some embodiments, the polynucleotide further comprises a fourth sequence, wherein the fourth sequence comprises a nucleic acid sequence having at least 80%, at least 85%, at least 90%, at least 95%, at least 98%, at least 99%, or 100% identity to the nucleic acid sequence of SEQ ID NO: 4, optionally wherein the fourth sequence comprises the nucleic acid sequence of SEQ ID NO: 4. In some embodiments, the polynucleotide comprising a nucleic acid sequence having at least 90% identity to the nucleic acid sequence of any one of SEQ ID NOs: 364-566.

Disclosed herein, in some embodiments, are RNA polynucleotides, comprising a nucleic acid sequence having at least 80%, at least 85%, at least 90%, at least 95%, at least 98%, at least 99%, or 100% identity to the nucleic acid sequence of any one of SEQ ID NOs: 364-566.

### ESS Sequences

Described herein, in some embodiments, are exonic splicing silencer (ESS) sequences. The ESS may be included in a modified or recombinant snRNA or U7 snRNA. The ESS may recruit a protein factor that reduces splicing of the RNA encoding an SCN1A protein. An ESS sequence may refer to an ESS or to a sequence that encodes an ESS. An ESS may include a short region of an exon and is a cis-regulatory element (CREs). CREs are regions of non-coding DNA which regulate transcription of neighboring genes. CREs may include components of genetic regulatory networks that control the timing and the amount that a specific gene is expressed. An ESS may be bound by a negatively acting factor such as a heterogeneous ribonucleoprotein (hnRNP).

ESSs may work by inhibiting the splicing of pre-mRNA transcripts or promoting exon skipping. In some embodiments, the ESS may recruit a protein factor that silences splicing of a target RNA such as an RNA encoding an SCN1A protein. In some embodiments, the ESS may recruit a group of factors that reduce splicing of the target RNA. In some embodiments, the ESS may recruit a group of factors that silence splicing of the RNA encoding an SCN1A protein.

In some embodiments, the ESS may be about 20 nucleotides long. In some embodiments, the ESS may be at least 4 nucleotides long. In some embodiments, the ESS may be at least 5 nucleotides long. In some embodiments, the ESS may be at least 6 nucleotides long. In some embodiments, the ESS may be at least 7 nucleotides long. In some embodiments, the ESS may be at least 8 nucleotides long. In some embodiments, the ESS may be at least 9 nucleotides long. In some embodiments, the ESS may be at least 10 nucleotides long. In some embodiments, the ESS may be at least 11 nucleotides long. In some embodiments, the ESS may be at least 12 nucleotides long. In some embodiments, the ESS may be at least 13 nucleotides long. In some embodiments, the ESS may be at least 14 nucleotides long. In some embodiments, the ESS may be at least 15 nucleotides long. In some embodiments, the ESS may be at least 16 nucleotides long. In some embodiments, the ESS may be at least 17 nucleotides long. In some embodiments, the ESS may be at least 18 nucleotides long. In some embodiments, the ESS may be at least 19 nucleotides long. In some embodiments, the ESS may be at least 20 nucleotides long.

In some embodiments, the ESS nucleic acid sequence may include a nucleic acid sequence identical to an ESS nucleic acid sequence in Tables 2A-2B. In some embodiments, the ESS nucleic acid sequence may include a nucleic acid sequence at least 99% identical to an ESS nucleic acid sequence in Tables 2A-2B. In some embodiments, the ESS nucleic acid sequence may include a nucleic acid sequence at least 98% identical to an ESS nucleic acid sequence in Tables 2A-2B. In some embodiments, the ESS nucleic acid sequence may include a nucleic acid sequence at least 97% identical to an ESS nucleic acid sequence in Tables 2A-2B. In some embodiments, the ESS nucleic acid sequence may include a nucleic acid sequence at least 96% identical to an ESS nucleic acid sequence in Tables 2A-2B. In some embodiments, the ESS nucleic acid sequence may include a nucleic acid sequence at least 95% identical to an ESS nucleic acid sequence in Tables 2A-2B. In some embodiments, the ESS nucleic acid sequence may include a nucleic acid sequence at least 94% identical to an ESS nucleic acid sequence in Tables 2A-2B. In some embodiments, the ESS nucleic acid sequence may include a nucleic acid sequence at least 93% identical to an ESS nucleic acid sequence in Tables 2A-2B. In some embodiments, the ESS nucleic acid sequence may include a nucleic acid sequence at least 92% identical to an ESS nucleic acid sequence in Tables 2A-2B. In some embodiments, the ESS nucleic acid sequence may include a nucleic acid sequence at least 91% identical to an ESS nucleic acid sequence in Tables 2A-2B. In some embodiments, the ESS nucleic acid sequence may include a nucleic acid sequence at least 90% identical to an ESS nucleic acid sequence in Tables 2A-2B. In some embodiments, the ESS nucleic acid sequence may include a nucleic acid sequence at least 85% identical to an ESS nucleic acid sequence in Tables 2A-2B. In some embodiments, the ESS nucleic acid sequence may include a nucleic acid sequence at least 80% identical to an ESS nucleic acid sequence in Tables 2A-2B. In some embodiments, the ESS nucleic acid sequence may include a nucleic acid sequence at least 75% identical to an ESS nucleic acid sequence in Tables 2A-2B. In some embodiments, the ESS nucleic acid sequence may include a nucleic acid sequence at least 70% identical to an ESS nucleic acid sequence in Tables 2A-2B.

In some embodiments, the ESS nucleic acid sequence may be or include ATGATAGGGACTTAGGGTGA (SEQ ID NO: 11). In some embodiments, the ESS nucleic acid sequence may be or include TTTGTTCCGTGGGTGGTTTA (SEQ ID NO: 12). In some embodiments, the ESS nucleic acid sequence may be or include TGGGGGGAGGTAGGTAGGTA (SEQ ID NO: 13).

### U7 Targeting Sequences

Described herein, in some embodiments, are targeting nucleic acid sequences such as snRNA targeting sequences or U7 targeting sequences. A targeting nucleic acid sequence may be or include an antisense nucleic acid sequence. A U7 targeting nucleic acid sequence may be or include a U7 antisense nucleic acid sequence. An snRNA targeting nucleic acid sequence may be or include a snRNA antisense nucleic acid sequence. Described herein, in some embodiments, are antisense nucleic acid sequences such as snRNA antisense sequences or U7 antisense sequences. An antisense sequence may be referred to as a targeting sequence. The antisense nucleic acid sequence may be included in a modified or recombinant snRNA or U7 snRNA. The targeting nucleic acid sequence may target (e.g. bind or be reverse complementary to) a target RNA such as an RNA encoding an SCN1A protein (Nav1.1). The antisense nucleic acid sequence may bind or be reverse complementary to a target RNA such as an RNA encoding an SCN1A protein. The antisense nucleic acid sequence may bind to the target RNA. In some embodiments, an antisense nucleic acid sequence is encoded by a DNA sequence (e.g. a DNA expression construct). Targeting an RNA such as an SCN2A RNA may include binding or being reverse complementary to the target RNA.

Described herein, in some embodiments, is a system wherein the antisense nucleic acid sequence may be 10-60 nucleotides in length. Some U rich small nuclear ribonucleoproteins (snRNPs) are complexes that may mediate the splicing of pre-mRNAs. U7 snRNP may be an exception in being uninvolved in splicing but may be a factor in unique 3′ end processing of replication-dependent histone mRNAs. However, by introducing controlled changes in the U7 snRNA histone binding sequence and in the Sm motif, U7 can be used as an effective tool for gene therapy. In some embodiments, the modified U7 snRNP may not be uninvolved in processing of replication-dependent histone pre-mRNA, but target splicing by inducing efficient skipping or inclusion of selected exons. Modification of the sequence motif of U7 snRNA can make the snRNP particle hybridize to almost any RNA sequence within the nucleoplasm.

An antisense nucleic acid sequence is or include a length of nucleotides. In some embodiments, the antisense nucleic acid sequence is at least 10 nucleotides. In some embodiments, the antisense nucleic acid sequence is at least 11 nucleotides. In some embodiments, the antisense nucleic acid sequence is at least 12 nucleotides. In some embodiments, the antisense nucleic acid sequence is at least 13 nucleotides. In some embodiments, the antisense nucleic acid sequence is at least 14 nucleotides. In some embodiments, the antisense nucleic acid sequence is at least 15 nucleotides. In some embodiments, the antisense nucleic acid sequence is at least 16 nucleotides. In some embodiments, the antisense nucleic acid sequence is at least 17 nucleotides. In some embodiments, the antisense nucleic acid sequence is at least 18 nucleotides. In some embodiments, the antisense nucleic acid sequence is at least 19 nucleotides. In some embodiments, the antisense nucleic acid sequence is at least 20 nucleotides. In some embodiments, the antisense nucleic acid sequence is at least 22 nucleotides. In some embodiments, the antisense nucleic acid sequence is at least 24 nucleotides. In some embodiments, the antisense nucleic acid sequence is at least 26 nucleotides. In some embodiments, the antisense nucleic acid sequence is at least 28 nucleotides. In some embodiments, the antisense nucleic acid sequence is at least 30 nucleotides. In some embodiments, the antisense nucleic acid sequence is at least 32 nucleotides. In some embodiments, the antisense nucleic acid sequence is at least 34 nucleotides. In some embodiments, the antisense nucleic acid sequence is at least 36 nucleotides. In some embodiments, the antisense nucleic acid sequence is at least 38 nucleotides. In some embodiments, the antisense nucleic acid sequence is at least 40 nucleotides. In some embodiments, the antisense nucleic acid sequence is at least 45 nucleotides. In some embodiments, the antisense nucleic acid sequence is at least 50 nucleotides. In some embodiments, the antisense nucleic acid sequence is at least 55 nucleotides. In some embodiments, the antisense nucleic acid sequence is at least 60 nucleotides. In some embodiments, the antisense nucleic acid sequence is at least 65 nucleotides. In some embodiments, the antisense nucleic acid sequence is at least 70 nucleotides. In some embodiments, the antisense nucleic acid sequence is at least 75 nucleotides. In some embodiments, the antisense nucleic acid sequence is at least 80 nucleotides. In some embodiments, the antisense nucleic acid sequence is at least 85 nucleotides. In some embodiments, the antisense nucleic acid sequence is at least 90 nucleotides. In some embodiments, the antisense nucleic acid sequence is at least 95 nucleotides. In some embodiments, the antisense nucleic acid sequence is at least 100 nucleotides. In some embodiments, the antisense nucleic acid sequence is at least 125 nucleotides. In some embodiments, the antisense nucleic acid sequence is at least 150 nucleotides. In some embodiments, the antisense nucleic acid sequence is at least 175 nucleotides. In some embodiments, the antisense nucleic acid sequence is at least 200 nucleotides. In some embodiments, the antisense nucleic acid sequence is at least 225 nucleotides. In some embodiments, the antisense nucleic acid sequence is at least 250 nucleotides. In some embodiments, the antisense nucleic acid sequence is at most 10 nucleotides. In some embodiments, the antisense nucleic acid sequence is at most 11 nucleotides. In some embodiments, the antisense nucleic acid sequence is at most 12 nucleotides. In some embodiments, the antisense nucleic acid sequence is at most 13 nucleotides. In some embodiments, the antisense nucleic acid sequence is at most 14 nucleotides. In some embodiments, the antisense nucleic acid sequence is at most 15 nucleotides. In some embodiments, the antisense nucleic acid sequence is at most 16 nucleotides. In some embodiments, the antisense nucleic acid sequence is at most 17 nucleotides. In some embodiments, the antisense nucleic acid sequence is at most 18 nucleotides. In some embodiments, the antisense nucleic acid sequence is at most 19 nucleotides. In some embodiments, the antisense nucleic acid sequence is at most 20 nucleotides. In some embodiments, the antisense nucleic acid sequence is at most 22 nucleotides. In some embodiments, the antisense nucleic acid sequence is at most 24 nucleotides. In some embodiments, the antisense nucleic acid sequence is at most 26 nucleotides. In some embodiments, the antisense nucleic acid sequence is at most 28 nucleotides. In some embodiments, the antisense nucleic acid sequence is at most 30 nucleotides. In some embodiments, the antisense nucleic acid sequence is at most 32 nucleotides. In some embodiments, the antisense nucleic acid sequence is at most 34 nucleotides. In some embodiments, the antisense nucleic acid sequence is at most 36 nucleotides. In some embodiments, the antisense nucleic acid sequence is at most 38 nucleotides. In some embodiments, the antisense nucleic acid sequence is at most 40 nucleotides. In some embodiments, the antisense nucleic acid sequence is at most 45 nucleotides. In some embodiments, the antisense nucleic acid sequence is at most 50 nucleotides. In some embodiments, the antisense nucleic acid sequence is at most 55 nucleotides. In some embodiments, the antisense nucleic acid sequence is at most 60 nucleotides. In some embodiments, the antisense nucleic acid sequence is at most 65 nucleotides. In some embodiments, the antisense nucleic acid sequence is at most 70 nucleotides. In some embodiments, the antisense nucleic acid sequence is at most 75 nucleotides. In some embodiments, the antisense nucleic acid sequence is at most 80 nucleotides. In some embodiments, the antisense nucleic acid sequence is at most 85 nucleotides. In some embodiments, the antisense nucleic acid sequence is at most 90 nucleotides. In some embodiments, the antisense nucleic acid sequence is at most 95 nucleotides. In some embodiments, the antisense nucleic acid sequence is at most 100 nucleotides. In some embodiments, the antisense nucleic acid sequence is at most 125 nucleotides. In some embodiments, the antisense nucleic acid sequence is at most 150 nucleotides. In some embodiments, the antisense nucleic acid sequence is at most 175 nucleotides. In some embodiments, the antisense nucleic acid sequence is at most 200 nucleotides. In some embodiments, the antisense nucleic acid sequence is at most 225 nucleotides. In some embodiments, the antisense nucleic acid sequence is at most 250 nucleotides. The antisense nucleic acid sequence may, in some embodiments, include a length defined by a range of any two of the aforementioned numbers of nucleotides.

In some embodiments, the antisense nucleic acid sequence binds an alternatively spliced region. The alternatively spliced region may be or include exon 20N, which is an example of a target RNA sequence. In some embodiments, the antisense nucleic acid sequence may bind to the alternatively spliced region at the 5′ end of the nucleic acid. In some embodiments, the antisense nucleic acid sequence binds to the alternatively spliced region at the 3′ end of the nucleic acid. In some embodiments, the antisense nucleic acid sequence bind to the alternatively spliced region in between the 5′ and 3′ ends of the target RNA.

An antisense nucleic acid sequence may hybridize or bind to a target RNA. In some embodiments, the antisense nucleic acid sequence is fully reverse complementary to a portion of the target RNA. In some embodiments, the antisense nucleic acid sequence is partially reverse complementary (e.g. at least 80% reverse complementary or at least 90% reverse complementary) to a portion of the target RNA. In some embodiments, the targeting nucleic acid is at least 99% reverse complementary to a portion of the target RNA. In some embodiments, the targeting nucleic acid is at least 98% reverse complementary to a portion of the target RNA. In some embodiments, the targeting nucleic acid is at least 97% reverse complementary to a portion of the target RNA. In some embodiments, the targeting nucleic acid is at least 96% reverse complementary to a portion of the target RNA. In some embodiments, the targeting nucleic acid is at least 95% reverse complementary to a portion of the target RNA. In some embodiments, the targeting nucleic acid is at least 94% reverse complementary to a portion of the target RNA. In some embodiments, the targeting nucleic acid is at least 93% reverse complementary to a portion of the target RNA. In some embodiments, the targeting nucleic acid is at least 92% reverse complementary to a portion of the target RNA. In some embodiments, the targeting nucleic acid is at least 91% reverse complementary to a portion of the target RNA. In some embodiments, the targeting nucleic acid is at least 90% reverse complementary to a portion of the target RNA. In some embodiments, the targeting nucleic acid is at least 85% reverse complementary to a portion of the target RNA. In some embodiments, the targeting nucleic acid is at least 80% reverse complementary to a portion of the target RNA. In some embodiments, the targeting nucleic acid is at least 75% reverse complementary to a portion of the target RNA. In some embodiments, the targeting nucleic acid is at least 70% reverse complementary to a portion of the target RNA. In some embodiments, the antisense nucleic acid sequence is fully reverse complementary or partially reverse complementary (e.g. at least 90% reverse complementary) to a portion of the target RNA. The portion of the target RNA (e.g. targeted or bound by the antisense nucleic acid sequence) may be or include an alternatively spliced exon such as exon 20N. The portion of the target RNA (e.g. targeted or bound by the antisense nucleic acid sequence) may be or include a region of a target RNA near or adjacent to an alternatively spliced exon such as exon 20N.

An antisense nucleic acid sequence may hybridize or bind to an alternatively spliced region (e.g. of a target RNA). In some embodiments, the antisense nucleic acid sequence is fully reverse complementary to a portion of the alternatively spliced region. In some embodiments, the antisense nucleic acid sequence is partially reverse complementary (e.g. at least 80% reverse complementary or at least 90% reverse complementary) to a portion of the alternatively spliced region. In some embodiments, the targeting nucleic acid is at least 99% reverse complementary to a portion of the alternatively spliced region. In some embodiments, the targeting nucleic acid is at least 98% reverse complementary to a portion of the alternatively spliced region. In some embodiments, the targeting nucleic acid is at least 97% reverse complementary to a portion of the alternatively spliced region. In some embodiments, the targeting nucleic acid is at least 96% reverse complementary to a portion of the alternatively spliced region. In some embodiments, the targeting nucleic acid is at least 95% reverse complementary to a portion of the alternatively spliced region. In some embodiments, the targeting nucleic acid is at least 94% reverse complementary to a portion of the alternatively spliced region. In some embodiments, the targeting nucleic acid is at least 93% reverse complementary to a portion of the alternatively spliced region. In some embodiments, the targeting nucleic acid is at least 92% reverse complementary to a portion of the alternatively spliced region. In some embodiments, the targeting nucleic acid is at least 91% reverse complementary to a portion of the alternatively spliced region. In some embodiments, the targeting nucleic acid is at least 90% reverse complementary to a portion of the alternatively spliced region. In some embodiments, the targeting nucleic acid is at least 85% reverse complementary to a portion of the alternatively spliced region. In some embodiments, the targeting nucleic acid is at least 80% reverse complementary to a portion of the alternatively spliced region. In some embodiments, the targeting nucleic acid is at least 75% reverse complementary to a portion of the alternatively spliced region. In some embodiments, the targeting nucleic acid is at least 70% reverse complementary to a portion of the alternatively spliced region. In some embodiments, the antisense nucleic acid sequence is fully reverse complementary or partially reverse complementary (e.g. at least 90% reverse complementary) to a portion of the alternatively spliced region.

In some embodiments, the portion of the alternatively spliced region comprises nucleotide positions within 50 base pairs (bp) of a 5′ or 3′ end of the alternatively spliced region. In some embodiments, the portion of the alternatively spliced region comprises nucleotide positions within 45 bp of a 5′ or 3′ end of the alternatively spliced region. In some embodiments, the portion of the alternatively spliced region comprises nucleotide positions within 40 bp of a 5′ or 3′ end of the alternatively spliced region. In some embodiments, the portion of the alternatively spliced region comprises nucleotide positions within 35 bp of a 5′ or 3′ end of the alternatively spliced region. In some embodiments, the portion of the alternatively spliced region comprises nucleotide positions within 30 bp of a 5′ or 3′ end of the alternatively spliced region. In some embodiments, the portion of the alternatively spliced region comprises nucleotide positions within 25 bp of a 5′ or 3′ end of the alternatively spliced region. In some embodiments, the portion of the alternatively spliced region comprises nucleotide positions within 20 bp of a 5′ or 3′ end of the alternatively spliced region. In some embodiments, the portion of the alternatively spliced region comprises nucleotide positions within 15 bp of a 5′ or 3′ end of the alternatively spliced region. In some embodiments, the portion of the alternatively spliced region comprises nucleotide positions within 10 bp of a 5′ or 3′ end of the alternatively spliced region. In some embodiments, the portion of the alternatively spliced region excludes nucleotide positions within 45 bp of a 5′ or 3′ end of the alternatively spliced region. In some embodiments, the portion of the alternatively spliced region excludes nucleotide positions within 40 bp of a 5′ or 3′ end of the alternatively spliced region. In some embodiments, the portion of the alternatively spliced region excludes nucleotide positions within 35 bp of a 5′ or 3′ end of the alternatively spliced region. In some embodiments, the portion of the alternatively spliced region excludes nucleotide positions within 30 bp of a 5′ or 3′ end of the alternatively spliced region. In some embodiments, the portion of the alternatively spliced region excludes nucleotide positions within 25 bp of a 5′ or 3′ end of the alternatively spliced region. In some embodiments, the portion of the alternatively spliced region excludes nucleotide positions within 20 bp of a 5′ or 3′ end of the alternatively spliced region. In some embodiments, the portion of the alternatively spliced region excludes nucleotide positions within 15 bp of a 5′ or 3′ end of the alternatively spliced region. In some embodiments, the portion of the alternatively spliced region excludes nucleotide positions within 10 bp of a 5′ or 3′ end of the alternatively spliced region. In some embodiments, the 5′ or 3′ end is a 5′ end. In some embodiments, the 5′ or 3′ end is a 3′ end.

In some embodiments, the portion of the alternatively spliced region is within a 5′ half of the alternatively spliced region. In some embodiments, the portion of the alternatively spliced region is within a 5′ end of the alternatively spliced region. In some embodiments, the portion of the alternatively spliced region is within a 5′ half or 5′ end of the alternatively spliced region. In some embodiments, the portion of the alternatively spliced region is within 25 nucleotides, within 50 nucleotides, within 75 nucleotides, within 100 nucleotides, within 125 nucleotides, within 150 nucleotides, within 175 nucleotides, within 200 nucleotides, within 225 nucleotides, or within 250 nucleotides of a 5′ end of an alternatively spliced region.

In some embodiments, the portion of the alternatively spliced region is within a 3′ half of the alternatively spliced region. In some embodiments, the portion of the alternatively spliced region is within a 3′ end of the alternatively spliced region. In some embodiments, the portion of the alternatively spliced region is within a 3′ half or 3′ end of the alternatively spliced region. In some embodiments, the portion of the alternatively spliced region is within 25 nucleotides, within 50 nucleotides, within 75 nucleotides, within 100 nucleotides, within 125 nucleotides, within 150 nucleotides, within 175 nucleotides, within 200 nucleotides, within 225 nucleotides, or within 250 nucleotides of a 3′ end of an alternatively spliced region.

In some embodiments, the antisense nucleic acid sequence may include a nucleic acid sequence identical to an antisense nucleic acid sequence in Tables 2A-2B. In some embodiments, the antisense nucleic acid sequence may include a nucleic acid sequence at least 99% identical to an antisense nucleic acid sequence in Tables 2A-2B. In some embodiments, the antisense nucleic acid sequence may include a nucleic acid sequence at least 98% identical to an antisense nucleic acid sequence in Tables 2A-2B. In some embodiments, the antisense nucleic acid sequence may include a nucleic acid sequence at least 97% identical to an antisense nucleic acid sequence in Tables 2A-2B. In some embodiments, the antisense nucleic acid sequence may include a nucleic acid sequence at least 96% identical to an antisense nucleic acid sequence in Tables 2A-2B. In some embodiments, the antisense nucleic acid sequence may include a nucleic acid sequence at least 95% identical to an antisense nucleic acid sequence in Tables 2A-2B. In some embodiments, the antisense nucleic acid sequence may include a nucleic acid sequence at least 94% identical to an antisense nucleic acid sequence in Tables 2A-2B. In some embodiments, the antisense nucleic acid sequence may include a nucleic acid sequence at least 93% identical to an antisense nucleic acid sequence in Tables 2A-2B. In some embodiments, the antisense nucleic acid sequence may include a nucleic acid sequence at least 92% identical to an antisense nucleic acid sequence in Tables 2A-2B. In some embodiments, the antisense nucleic acid sequence may include a nucleic acid sequence at least 91% identical to an antisense nucleic acid sequence in Tables 2A-2B. In some embodiments, the antisense nucleic acid sequence may include a nucleic acid sequence at least 90% identical to an antisense nucleic acid sequence in Tables 2A-2B. In some embodiments, the antisense nucleic acid sequence may include a nucleic acid sequence at least 85% identical to an antisense nucleic acid sequence in Tables 2A-2B. In some embodiments, the antisense nucleic acid sequence may include a nucleic acid sequence at least 80% identical to an antisense nucleic acid sequence in Tables 2A-2B. In some embodiments, the antisense nucleic acid sequence may include a nucleic acid sequence at least 75% identical to an antisense nucleic acid sequence in Tables 2A-2B. In some embodiments, the antisense nucleic acid sequence may include a nucleic acid sequence at least 70% identical to an antisense nucleic acid sequence in Tables 2A-2B. In some embodiments, the targeting nucleic acid targets or binds with an RNA that includes reverse complement of any of the aforementioned sequences. In some embodiments, the antisense nucleic acid sequence is encoded by a reverse complement of any of the aforementioned sequences (such as when the antisense nucleic acid sequence is encoded in an expression construct described herein. A reverse complement may include Us in place of Ts, or vice versa.

In some embodiments, use of the target nucleic acid sequence results in a percentage of productive isoform of SCN1A, for example as determined in Table 2C. An example of a productive isoform may include an isoform excluding exon 21N of a mouse SCN1A transcript, or an isoform excluding exon 20N of a human SCN1A transcript. In some embodiments, use of the targeting nucleic acid results in at least 50% of the productive isoform. In some embodiments, use of an engineered snRNA comprising the targeting nucleic acid results in at least 55% of the productive isoform. In some embodiments, use of an engineered snRNA comprising the targeting nucleic acid results in at least 60% of the productive isoform. In some embodiments, use of an engineered snRNA comprising the targeting nucleic acid results in at least 65% of the productive isoform. In some embodiments, use of an engineered snRNA comprising the targeting nucleic acid results in at least 70% of the productive isoform. In some embodiments, use of an engineered snRNA comprising the targeting nucleic acid results in at least 75% of the productive isoform. In some embodiments, use of an engineered snRNA comprising the targeting nucleic acid results in at least 80% of the productive isoform. In some embodiments, use of an engineered snRNA comprising the targeting nucleic acid results in at least 85% of the productive isoform. In some embodiments, use of an engineered snRNA comprising the targeting nucleic acid results in at least 90% of the productive isoform. In some embodiments, use of an engineered snRNA comprising the targeting nucleic acid results in at least 91% of the productive isoform. In some embodiments, use of an engineered snRNA comprising the targeting nucleic acid results in at least 92% of the productive isoform. In some embodiments, use of an engineered snRNA comprising the targeting nucleic acid results in no more than 50% of the productive isoform. In some embodiments, use of an engineered snRNA comprising the targeting nucleic acid results in no more than 55% of the productive isoform. In some embodiments, use of an engineered snRNA comprising the targeting nucleic acid results in no more than 60% of the productive isoform. In some embodiments, use of an engineered snRNA comprising the targeting nucleic acid results in no more than 65% of the productive isoform. In some embodiments, use of an engineered snRNA comprising the targeting nucleic acid results in no more than 70% of the productive isoform. In some embodiments, use of an engineered snRNA comprising the targeting nucleic acid results in no more than 75% of the productive isoform. In some embodiments, use of an engineered snRNA comprising the targeting nucleic acid results in no more than 80% of the productive isoform. In some embodiments, use of an engineered snRNA comprising the targeting nucleic acid results in no more than 85% of the productive isoform. In some embodiments, use of an engineered snRNA comprising the targeting nucleic acid results in no more than 90% of the productive isoform. In some embodiments, use of an engineered snRNA comprising the targeting nucleic acid results in no more than 91% of the productive isoform. In some embodiments, use of an engineered snRNA comprising the targeting nucleic acid results in no more than 92% of the productive isoform.

In some embodiments, the antisense nucleic acid sequence may include the nucleic acid sequence of SEQ ID NO: 23, or a reverse complement thereof. In some embodiments, the antisense nucleic acid sequence is 85%, 86%, 87%, 88%, 89%, 90%, 91%, 92%, 93%, 94%, 95%, 96%, 97%, 98%, 99%, or 100% identical to SEQ ID NO: 23 or a reverse complement thereof, or a range of % identities defined by any two of the aforementioned percentages.

In some embodiments, the antisense nucleic acid sequence may include the nucleic acid sequence of SEQ ID NO: 53, or a reverse complement thereof. In some embodiments, the antisense nucleic acid sequence is 85%, 86%, 87%, 88%, 89%, 90%, 91%, 92%, 93%, 94%, 95%, 96%, 97%, 98%, 99%, or 100% identical to SEQ ID NO: 53 or a reverse complement thereof, or a range of % identities defined by any two of the aforementioned percentages.

In some embodiments, the antisense nucleic acid sequence may include the nucleic acid sequence of SEQ ID NO: 98, or a reverse complement thereof. In some embodiments, the antisense nucleic acid sequence is 85%, 86%, 87%, 88%, 89%, 90%, 91%, 92%, 93%, 94%, 95%, 96%, 97%, 98%, 99%, or 100% identical to SEQ ID NO: 98 or a reverse complement thereof, or a range of % identities defined by any two of the aforementioned percentages.

The antisense nucleic acid sequence may bind to or be reverse complementary to a targeted region. In some embodiments, the antisense nucleic acid sequence binds to an SCN1A mRNA at a region 5′ or 3′ relative to exon 20N. The antisense nucleic acid sequence may bind to an SCN1A mRNA comprising the sequence of SEQ ID NO: 153. The antisense nucleic acid sequence may bind to an SCN1A mRNA comprising the sequence of SEQ ID NO: 154.

In some embodiments, the antisense nucleic acid sequence binds to an SCN1A pre-mRNA at a region 5′ relative to exon 20N. The region may be up to 25 nucleotides, up to 50 nucleotides, up to 75 nucleotides, up to 100 nucleotides, or more, upstream from or 5′ to exon 20N in an SCN1A pre-mRNA.

In some embodiments, the antisense nucleic acid sequence binds to an SCN1A pre-mRNA at a region 3′ relative to exon 20N. The region may be up to 25 nucleotides, up to 50 nucleotides, up to 75 nucleotides, up to 100 nucleotides, or more, downstream from or 3′ to exon 20N in an SCN1A pre-mRNA.

In some embodiments, the engineered U7 snRNA includes an antisense nucleic acid sequence 3′ to the ESS nucleic acid sequence. In some embodiments, the engineered U7 snRNA includes an antisense nucleic acid sequence downstream relative to the ESS nucleic acid sequence. In some embodiments, the engineered U7 snRNA includes an antisense nucleic acid sequence 3′ or downstream relative to the ESS nucleic acid sequence.

### Sm Binding Sites

Described herein, in some embodiments, are systems that include a Sm binding site. The Sm binding site may be included in a modified or recombinant snRNA or U7 snRNA. Sm proteins may bind to a U7 snRNA via the Sm binding site. Once the Sm proteins bind the U7 snRNA in the cytoplasm they may bind to a pre-mRNA and regulate splicing. In some embodiments, the system contains a Sm-like binding site. In some embodiments, the Sm binding site comprises AAUUUGUCUAG (SEQ ID NO: 150). In some embodiments, the Sm binding site comprises AAUUUUUGGAG (SEQ ID NO: 151; smOPT). A modified or recombinant U7 snRNA sequence may include a Sm binding site. A system may encode a modified or recombinant U7 snRNA sequence that includes a Sm binding site.

In some embodiments, the Sm binding site may be 3′ relative to the ESS nucleic acid sequence. In some embodiments, the Sm binding site may be downstream relative to the ESS nucleic acid sequence. In some embodiments, the Sm binding site may be 3′ relative to the antisense nucleic acid sequence. In some embodiments, the Sm binding site may be downstream relative to the antisense nucleic acid sequence. In some embodiments, the Sm binding site is 3′ or downstream relative to the ESS nucleic acid sequence or antisense nucleic acid sequence.

### U7 3′ Hairpins

Described herein, in some embodiments, are systems that include a hairpin sequence. The hairpin sequence may be included in a modified or recombinant snRNA or U7 snRNA. The hairpin sequence may include a U7 hairpin sequence. The U7 hairpin sequence may be a 3′ U7 hairpin sequence. A modified or recombinant U7 snRNA sequence may include the hairpin sequence. A system may encode a modified or recombinant U7 snRNA sequence that includes a hairpin sequence.

Described herein, in some embodiments, is a nucleic acid system that may contain a hairpin sequence comprising a U7 small nuclear RNA (snRNA) hairpin sequence. A hairpin may include an unpaired loop of RNA that is created when a RNA strand folds and forms complementary base pairs with another section of the same strand. The resulting structure may look like a loop or a U-shape. Hairpins may include a common type of secondary structure in RNA molecules and can be formed when two complementary sequences in a single RNA molecule meet and bind each other.

In some embodiments, the U7 snRNA hairpin sequence comprises the nucleic acid sequence of SEQ ID NO: 4. In some embodiments, the U7 snRNA hairpin sequence comprises a nucleic acid sequence at least 90%, at least 91%, at least 92%, at least 93%, at least 94%, at least 95%, at least 96%, at least 97%, at least 98%, or at least 99% identical to SEQ ID NO: 4.

In some embodiments, the hairpin sequence may include a 3′ hairpin sequence. In some embodiments, the hairpin sequence may be 3′ or downstream relative to the ESS nucleic acid sequence. In some embodiments, the hairpin sequence may be 3′ or downstream relative to the antisense nucleic acid sequence. In some embodiments, the hairpin sequence may be 3′ or downstream relative to or the Sm binding site. In some embodiments, the hairpin sequence is 3′ or downstream relative to the ESS nucleic acid sequence, the antisense nucleic acid sequence, or the Sm binding site.

### Expression Constructs

Described herein, in some embodiments, are expression constructs. An expression construct may include an expression cassette. The expression construct may be DNA. The expression construct may encode an RNA system described herein. The expression construct may encode an RNA such as a modified U7 snRNA. The expression construct may encode an ESS sequence, a U7 targeting sequence, a Sm binding site, or a U7 3′ hairpin, or a combination thereof. For example, an expression construct may encode an ESS sequence, a U7 targeting sequence, a Sm binding site, and a U7 3′ hairpin, which may be operably connected to a promoter within the expression construct. The expression construct may be or include a viral vector. The expression construct may be included in a composition herein. The expression construct may be included in a virus or viral delivery agent.

In some embodiments, the system may include an expression cassette. In some embodiments, the expression cassette may include a promoter. In some embodiments, the expression cassette may encode an exonic splicing sequence. In some embodiments, the expression cassette may encode a U7 targeting sequence. In some embodiments, the expression cassette may encode a smOPT sequence. In some embodiments, the expression cassette may encode a U7 3′ hairpin structure. In some embodiments, the expression cassette may include a 3′ terminator sequence. In some embodiments, the expression cassette may include or encode a combination of two or more of the following: a promoter, an exonic splicing sequence, a U7 targeting sequence, a smOPT, a U7 3′ hairpin structure, and a 3′ terminator sequence.

Some embodiments include an arrayed series of modified U7 snRNAs. In some embodiments, the array includes multiple U7 modules. Each module may encode a targeting sequence. The targeting sequences of multiple modules may be the same as each other. The targeting sequences of some modules may be different from each other.

An expression construct may encode multiple engineered or modified snRNAs. For example, an expression construct may encode 1, 2, 3, 4, 5, 6, 7, 8, 9, or 10 copies of a recombinant U7 snRNA. In some embodiments, the expression construct encodes 1 copy of a recombinant U7 snRNA. In some embodiments, the expression construct encodes 2 copies of the recombinant U7 snRNA. In some embodiments, the expression construct encodes 3 copies of the recombinant U7 snRNA. In some embodiments, the expression construct encodes 4 copies of the recombinant U7 snRNA. In some embodiments, the expression construct encodes 5 copies of the recombinant U7 snRNA. In some embodiments, the expression construct encodes 6 copies of the recombinant U7 snRNA. In some embodiments, the expression construct encodes 7 copies of the recombinant U7 snRNA. In some embodiments, the expression construct encodes 8 copies of the recombinant U7 snRNA. In some embodiments, the expression construct encodes 9 copies of the recombinant U7 snRNA. In some embodiments, the expression construct encodes 10 copies of the recombinant U7 snRNA. In some embodiments, all 1, 2, 3, 4, 5, 6, 7, 8, 9, or 10 copies of a recombinant U7 snRNA sequence are operably linked to one or more promoters. For example, multiple copies of the recombinant U7 snRNA sequence may be operably linked to a single promoter. In some embodiments, all 1, 2, 3, 4, 5, 6, 7, 8, 9, or 10 copies of a recombinant U7 snRNA sequence are operably linked to one or more 3′ terminator sequences.

The modified U7 snRNA may be expressed from a U7 cassette. A functional U7 cassette may include, in the following order from 5′ to 3′: (1) a promoter, (2) an optional exonic splicing silencer, (3) a targeting sequence complementary to a region of a target mRNA (e.g. SCN1A mRNA), (4) an smOPT sequence, (5) a hairpin sequence, and (6) a 3′ termination signal. The expressed functional U7 snRNA consists of, in the following order from 5′ to 3′: (1) an optional exonic splicing silencer, (2) a targeting sequence complementary to a region of target mRNA (e.g. SCN1A mRNA), (3) an smOPT sequence, and (4) a hairpin sequence.

Some examples of expression constructs include a DNA sequence encoding an snRNA of Tables 7A-7C, or a sequence at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical thereto.

Disclosed herein, in some embodiments, are polynucleotides, comprising a promoter, a coding sequence, and a terminator sequence, wherein the coding sequence comprises a nucleic acid sequence having at least 80%, at least 85%, at least 90%, at least 95%, at least 98%, at least 99%, or 100% identity to the nucleic acid sequence of any one of SEQ ID NOs: 364-566.

### Promoters

Described herein, in some embodiments, is a nucleic acid system that may contain a promoter sequence. The promoter may be a part of an expression construct. A promoter may include a sequence of DNA to which proteins bind to initiate transcription of a single RNA transcript from the DNA downstream of the promoter. The RNA transcript may encode a protein (mRNA), or can have a function in and of itself, such as tRNA or snRNA. Promoters may be located near the transcription start sites of genes. The promoter may be operably connected with an ESS sequence, a U7 targeting sequence, a Sm binding site, or a U7 3′ hairpin, or a combination thereof. For example, The promoter may be operably connected with an ESS sequence, a U7 targeting sequence, a Sm binding site, and a U7 3′ hairpin within an expression construct.

In some embodiments, the promoter is directly upstream of a transcriptional start site for the engineered snRNA. In some embodiments, the promoter is 5 bps upstream of the transcriptional start site. In some embodiments, the promoter is 10 bps upstream of the transcriptional start site. In some embodiments, the promoter is 15 bps upstream of the transcriptional start site. In some embodiments, the promoter is 20 bps upstream of the transcriptional start site. In some embodiments, the promoter is 25 bps upstream of the transcriptional start site. In some embodiments, the promoter is 30 bps upstream of the transcriptional start site. In some embodiments, the promoter is 35 bps upstream of the transcriptional start site. In some embodiments, the promoter is 40 bps upstream of the transcriptional start site. In some embodiments, the promoter is 45 bps upstream of the transcriptional start site. In some embodiments, the promoter is 50 bps upstream of the transcriptional start site. In some embodiments, the promoter is 55 bps upstream of the transcriptional start site. In some embodiments, the promoter is 60 bps upstream of the transcriptional start site. In some embodiments, the promoter is 65 bps upstream of the transcriptional start site. In some embodiments, the promoter is 70 bps upstream of the transcriptional start site. In some embodiments, the promoter is 75 bps upstream of the transcriptional start site. In some embodiments, the promoter is 80 bps upstream of the transcriptional start site. In some embodiments, the promoter is 85 bps upstream of the transcriptional start site. In some embodiments, the promoter is 90 bps upstream of the transcriptional start site. In some embodiments, the promoter is 95 bps upstream of the transcriptional start site. In some embodiments, the promoter is 100 bps upstream of the transcriptional start site. In some embodiments, the promoter is 200 bps upstream of the transcriptional start site. In some embodiments, the promoter is 300 bps upstream of the transcriptional start site. In some embodiments, the promoter is 400 bps upstream of the transcriptional start site. In some embodiments, the promoter is 500 bps upstream of the transcriptional start site. In some embodiments, the promoter is 600 bps upstream of the transcriptional start site. In some embodiments, the promoter is 700 bps upstream of the transcriptional start site. In some embodiments, the promoter is 800 bps upstream of the transcriptional start site. In some embodiments, the promoter is 900 bps upstream of the transcriptional start site. In some embodiments, the promoter is 1000 bps upstream of the transcriptional start site. Promoters can be about 100-1000 base pairs long, the sequence of which is highly dependent on the gene and product of transcription, type or class of RNA polymerase recruited to the site, and species of organism. In some embodiments, the promoter is 100 base pairs long. In some embodiments, the promoter is 200 base pairs long. In some embodiments, the promoter is 300 base pairs long. In some embodiments, the promoter is 400 base pairs long. In some embodiments, the promoter is 500 base pairs long. In some embodiments, the promoter is 600 base pairs long. In some embodiments, the promoter is 700 base pairs long. In some embodiments, the promoter is 800 base pairs long. In some embodiments, the promoter is 900 base pairs long. In some embodiments, the promoter is 1000 base pairs long.

In some embodiments, the promoter sequence may include a mouse or human promoter sequence. In some embodiments, the promoter sequence may include a mouse promoter sequence. In some embodiments, the promoter sequence may include a human promoter sequence.

In some embodiments, the promoter sequence may include an snRNA promoter sequence. In some embodiments, the promoter sequence may include a U7 snRNA promoter sequence. In some embodiments, the promoter sequence may include a U1 promoter sequence. In some embodiments, the promoter sequence may include a mouse U7 snRNA (“Mm U7”) promoter sequence. In some embodiments, the promoter sequence may include a human U7 snRNA (“Hs U7”) promoter sequence. In some embodiments, the promoter sequence may include a mouse U1a1 (“mu1a1” or “Mm U1a1”) promoter sequence. In some embodiments, the promoter sequence may include a human U1-1 (“HU1” or “Hs U1-1”) promoter sequence. In some embodiments, the promoter sequence may include a fragment or combination of fragments of the promotors listed above. In some embodiments, the promoter sequence comprises a mouse U7 snRNA (“Mm U7”) promoter sequence, a human U7 snRNA (“Hs U7”) promoter sequence, a mouse U1a1 (“mu1a1” or “Mm U1a1”) promoter sequence, or a human U1-1 (“HU1” or “Hs U1-1”) promoter sequence, or a fragment or combination of fragments thereof.

In some embodiments, the promoter sequence may include a U7 snRNA promoter sequence having a distal sequence element (DSE) replaced with a DSE of a U1-1 promoter sequence. In some embodiments, the promoter sequence may include a U7 snRNA promoter sequence having a DSE replaced with a U1a1 promoter sequence. In some embodiments, the promoter sequence comprises a U7 snRNA promoter sequence having a distal sequence element (DSE) replaced with a DSE of a U1-1 or U1a1 promoter sequence. The promoter DSE is a region of the DNA that is normally found upstream of a transcriptional start site. The DSE is usually found upstream, from −250 to −170 bp. In some embodiments, the DSE is at least 10 base pairs long. In some embodiments, the DSE is at least 20 base pairs long. In some embodiments, the DSE is at least 30 base pairs long. In some embodiments, the DSE is at least 40 base pairs long. In some embodiments, the DSE is at least 50 base pairs long. In some embodiments, the DSE is at least 60 base pairs long. In some embodiments, the DSE is at least 70 base pairs long. In some embodiments, the DSE is at least 80 base pairs long. In some embodiments, the DSE is at least 90 base pairs long. In some embodiments, the DSE is at least 100 base pairs long. In some embodiments, the DSE is at least 110 base pairs long. In some embodiments, the DSE is at least 120 base pairs long. In some embodiments, the DSE is at least 130 base pairs long. In some embodiments, the DSE is at least 140 base pairs long. In some embodiments, the DSE is at least 150 base pairs long. In some embodiments, the DSE is at least 160 base pairs long.

In some embodiments, the promoter sequence may include a mouse U7 promoter sequence having a proximal sequence element (PSE) replaced with a PSE of a U1-1 promoter sequence. In some embodiments, the promoter sequence may include a mouse U7 promoter sequence having a PSE replaced with a U1a1 promoter sequence. In some embodiments, the promoter sequence comprises a mouse U7 promoter sequence having a proximal sequence element (PSE) replaced with a PSE of a U1-1 or U1a1 promoter sequence. The promoter PSE may be an essential promoter element located 40-70 bp upstream of the ORF. The promoter PSE may be recognized by a specific transcription factor, snRNA activating protein complex (SNAPc). In some embodiments, the promoter PSE is 5 base pairs long. In some embodiments, the promoter PSE is 10 base pairs long. In some embodiments, the promoter PSE is 15 base pairs long. In some embodiments, the promoter PSE is 20 base pairs long. In some embodiments, the promoter PSE is 25 base pairs long. In some embodiments, the promoter PSE is 30 base pairs long. In some embodiments, the promoter PSE is 35 base pairs long. In some embodiments, the promoter PSE is 40 base pairs long. In some embodiments, the promoter PSE is 45 base pairs long. In some embodiments, the promoter PSE is 50 base pairs long. In some embodiments, the promoter PSE is 55 base pairs long. In some embodiments, the promoter PSE is 60 base pairs long.

Some embodiments include a recombinant promoter sequence. An example is the U1a1 promoter in Table 1. For the modified Mm U7 promoter in Table 1 (SEQ ID NO: 8), a U7 distal sequence element has been replaced with that of human U1-1 and a U7 proximal sequence element has been replaced with that of mouse U1a1. Other examples of recombinant promoter sequence are also provided in Table 1 and Table 5.

In some embodiments, the promoter sequence may include a nucleic acid sequence identical to a promoter sequence in Table 1. In some embodiments, the promoter sequence may include a nucleic acid sequence at least 99% identical to a promoter sequence in Table 1. In some embodiments, the promoter sequence may include a nucleic acid sequence at least 98% identical to a promoter sequence in Table 1. In some embodiments, the promoter sequence may include a nucleic acid sequence at least 97% identical to a promoter sequence in Table 1. In some embodiments, the promoter sequence may include a nucleic acid sequence at least 96% identical to a promoter sequence in Table 1. In some embodiments, the promoter sequence may include a nucleic acid sequence at least 95% identical to a promoter sequence in Table 1. In some embodiments, the promoter sequence may include a nucleic acid sequence at least 94% identical to a promoter sequence in Table 1. In some embodiments, the promoter sequence may include a nucleic acid sequence at least 93% identical to a promoter sequence in Table 1. In some embodiments, the promoter sequence may include a nucleic acid sequence at least 92% identical to a promoter sequence in Table 1. In some embodiments, the promoter sequence may include a nucleic acid sequence at least 91% identical to a promoter sequence in Table 1. In some embodiments, the promoter sequence may include a nucleic acid sequence at least 90% identical to a promoter sequence in Table 1. In some embodiments, the promoter sequence may include a nucleic acid sequence at least 85% identical to a promoter sequence in Table 1. In some embodiments, the promoter sequence may include a nucleic acid sequence at least 80% identical to a promoter sequence in Table 1. In some embodiments, the promoter sequence may include a nucleic acid sequence at least 75% identical to a promoter sequence in Table 1. In some embodiments, the promoter sequence may include a nucleic acid sequence at least 70% identical to a promoter sequence in Table 1.

In some embodiments, the promoter sequence may include a nucleic acid sequence identical to a promoter sequence in Table 5. In some embodiments, the promoter sequence may include a nucleic acid sequence at least 99% identical to a promoter sequence in Table 5. In some embodiments, the promoter sequence may include a nucleic acid sequence at least 98% identical to a promoter sequence in Table 5. In some embodiments, the promoter sequence may include a nucleic acid sequence at least 97% identical to a promoter sequence in Table 5. In some embodiments, the promoter sequence may include a nucleic acid sequence at least 96% identical to a promoter sequence in Table 5. In some embodiments, the promoter sequence may include a nucleic acid sequence at least 95% identical to a promoter sequence in Table 5. In some embodiments, the promoter sequence may include a nucleic acid sequence at least 94% identical to a promoter sequence in Table 5. In some embodiments, the promoter sequence may include a nucleic acid sequence at least 93% identical to a promoter sequence in Table 5. In some embodiments, the promoter sequence may include a nucleic acid sequence at least 92% identical to a promoter sequence in Table 5. In some embodiments, the promoter sequence may include a nucleic acid sequence at least 91% identical to a promoter sequence in Table 5. In some embodiments, the promoter sequence may include a nucleic acid sequence at least 90% identical to a promoter sequence in Table 5. In some embodiments, the promoter sequence may include a nucleic acid sequence at least 85% identical to a promoter sequence in Table 5. In some embodiments, the promoter sequence may include a nucleic acid sequence at least 80% identical to a promoter sequence in Table 5. In some embodiments, the promoter sequence may include a nucleic acid sequence at least 75% identical to a promoter sequence in Table 5. In some embodiments, the promoter sequence may include a nucleic acid sequence at least 70% identical to a promoter sequence in Table 5.

In some embodiments, the promoter sequence may be 5′ or upstream relative to the ESS nucleic acid sequence. In some embodiments, the promoter sequence may be 5′ or upstream relative to the antisense nucleic acid sequence. In some embodiments, the promoter sequence may be 5′ or upstream relative to the Sm binding site. In some embodiments, the promoter sequence may be 5′ or upstream relative to the hairpin sequence. In some embodiments, the promoter sequence is 5′ or upstream relative to the ESS nucleic acid sequence, the antisense nucleic acid sequence, the Sm binding site, or the hairpin sequence.

### 3′ Terminator Sequences

Described herein, in some embodiments, is a nucleic acid system that may contain a terminator sequence. The terminator sequence may be a part of an expression construct. Described herein, in some embodiments, the nucleic acid system may include a terminator sequence. The terminator sequence may include a region of a nucleic acid sequence that marks the end of a gene or operon during transcription. The terminator sequence may mediate transcriptional termination by providing signals in the newly synthesized transcript RNA that triggers processes which release the transcript RNA from the transcriptional complex. The terminator sequence may be operably connected with an ESS sequence, a U7 targeting sequence, a Sm binding site, or a U7 3′ hairpin, or a combination thereof. For example, the terminator sequence may be operably connected with an ESS sequence, a U7 targeting sequence, a Sm binding site, and a U7 3′ hairpin within an expression construct.

In some embodiments, the terminator sequence may include a mouse or human terminator sequence. In some embodiments, the terminator sequence may include an snRNA terminator sequence. In some embodiments, the terminator sequence may include a U7 snRNA terminator sequence. In some embodiments, the terminator sequence may include a U1 terminator sequence. In some embodiments, the terminator sequence may include a Mm U7 terminator sequence. In some embodiments, the terminator sequence may include a Hs U7 terminator sequence. In some embodiments, the terminator sequence may include a mu1a1 terminator sequence. In some embodiments, the terminator sequence may include a HU1 terminator sequence. In some embodiments, the terminator sequence may include a fragment or a combination of fragments of the terminators listed above. In some embodiments, the terminator sequence comprises a Mm U7 terminator sequence, a Hs U7 terminator sequence, a mu1a1 terminator sequence, or a HU1 terminator sequence, or a fragment or combination of fragments thereof.

In some embodiments, the terminator sequence comprises a U7 snRNA terminator sequence having a distal sequence element (DSE) replaced with a DSE of a U1-1 terminator sequence. In some embodiments, the terminator sequence comprises a U7 snRNA terminator sequence having a DSE replaced with a DSE of a U1a1 terminator sequence. In some embodiments, the terminator sequence comprises a U7 snRNA terminator sequence having a distal sequence element (DSE) replaced with a DSE of a U1-1 or U1a1 terminator sequence. The terminator DSE is a region of the DNA that is normally found downstream of the snRNA at the position from +250 to +170 bp. In some embodiments, the terminator DSE is at least 10 base pairs long. In some embodiments, the terminator DSE is at least 20 base pairs long. In some embodiments, the terminator DSE is at least 30 base pairs long. In some embodiments, the terminator DSE is at least 40 base pairs long. In some embodiments, the terminator DSE is at least 50 base pairs long. In some embodiments, the terminator DSE is at least 60 base pairs long. In some embodiments, the terminator DSE is at least 70 base pairs long. In some embodiments, the terminator DSE is at least 80 base pairs long. In some embodiments, the terminator DSE is at least 90 base pairs long. In some embodiments, the terminator DSE is at least 100 base pairs long. In some embodiments, the terminator DSE is at least 110 base pairs long. In some embodiments, the terminator DSE is at least 120 base pairs long. In some embodiments, the terminator DSE is at least 130 base pairs long. In some embodiments, the terminator DSE is at least 140 base pairs long. In some embodiments, the terminator DSE is at least 150 base pairs long. In some embodiments, the terminator DSE is at least 160 base pairs long.

In some embodiments, the terminator sequence may include a mouse U7 snRNA terminator sequence having a proximal sequence element (PSE) replaced with a PSE of a U1-1 terminator sequence. In some embodiments, the terminator sequence may include a mouse U7 snRNA terminator sequence having a PSE replaced with a PSE of a U1a1 terminator sequence. In some embodiments, the terminator sequence comprises a mouse U7 snRNA terminator sequence having a proximal sequence element (PSE) replaced with a PSE of a U1-1 or U1a1 terminator sequence. The terminator PSE may be an essential terminator element located 40-70 bp downstream of the terminator start site. In some embodiments, the terminator PSE is 5 base pairs long. In some embodiments, the terminator PSE is 10 base pairs long. In some embodiments, the terminator PSE is 15 base pairs long. In some embodiments, the terminator PSE is 20 base pairs long. In some embodiments, the terminator PSE is 25 base pairs long. In some embodiments, the terminator PSE is 30 base pairs long. In some embodiments, the terminator PSE is 35 base pairs long. In some embodiments, the terminator PSE is 40 base pairs long. In some embodiments, the terminator PSE is 45 base pairs long. In some embodiments, the terminator PSE is 50 base pairs long. In some embodiments, the terminator PSE is 55 base pairs long. In some embodiments, the terminator PSE is 60 base pairs long.

In some embodiments, the terminator sequence includes a nucleic acid sequence. In some embodiments, the terminator sequence includes a nucleic acid sequence identical to a terminator sequence in Table 1. In some embodiments, the terminator sequence includes a nucleic acid sequence at least 99% identical to a terminator sequence in Table 1. In some embodiments, the terminator sequence includes a nucleic acid sequence at least 98% identical to a terminator sequence in Table 1. In some embodiments, the terminator sequence includes a nucleic acid sequence at least 97% identical to a terminator sequence in Table 1. In some embodiments, the terminator sequence includes a nucleic acid sequence at least 96% identical to a terminator sequence in Table 1. In some embodiments, the terminator sequence includes a nucleic acid sequence at least 95% identical to a terminator sequence in Table 1. In some embodiments, the terminator sequence includes a nucleic acid sequence at least 94% identical to a terminator sequence in Table 1. In some embodiments, the terminator sequence includes a nucleic acid sequence at least 93% identical to a terminator sequence in Table 1. In some embodiments, the terminator sequence includes a nucleic acid sequence at least 92% identical to a terminator sequence in Table 1. In some embodiments, the terminator sequence includes a nucleic acid sequence at least 91% identical to a terminator sequence in Table 1. In some embodiments, the terminator sequence includes a nucleic acid sequence at least 90% identical to a terminator sequence in Table 1. In some embodiments, the terminator sequence includes a nucleic acid sequence at least 85% identical to a terminator sequence in Table 1. In some embodiments, the terminator sequence includes a nucleic acid sequence at least 80% identical to a terminator sequence in Table 1. In some embodiments, the terminator sequence includes a nucleic acid sequence at least 75% identical to a terminator sequence in Table 1. In some embodiments, the terminator sequence includes a nucleic acid sequence at least 70% identical to a terminator sequence in Table 1.

In some embodiments, the terminator sequence includes a nucleic acid sequence identical to a terminator sequence in Table 6. In some embodiments, the terminator sequence includes a nucleic acid sequence at least 99% identical to a terminator sequence in Table 6. In some embodiments, the terminator sequence includes a nucleic acid sequence at least 98% identical to a terminator sequence in Table 6. In some embodiments, the terminator sequence includes a nucleic acid sequence at least 97% identical to a terminator sequence in Table 6. In some embodiments, the terminator sequence includes a nucleic acid sequence at least 96% identical to a terminator sequence in Table 6. In some embodiments, the terminator sequence includes a nucleic acid sequence at least 95% identical to a terminator sequence in Table 6. In some embodiments, the terminator sequence includes a nucleic acid sequence at least 94% identical to a terminator sequence in Table 6. In some embodiments, the terminator sequence includes a nucleic acid sequence at least 93% identical to a terminator sequence in Table 6. In some embodiments, the terminator sequence includes a nucleic acid sequence at least 92% identical to a terminator sequence in Table 6. In some embodiments, the terminator sequence includes a nucleic acid sequence at least 91% identical to a terminator sequence in Table 6. In some embodiments, the terminator sequence includes a nucleic acid sequence at least 90% identical to a terminator sequence in Table 6. In some embodiments, the terminator sequence includes a nucleic acid sequence at least 85% identical to a terminator sequence in Table 6. In some embodiments, the terminator sequence includes a nucleic acid sequence at least 80% identical to a terminator sequence in Table 6. In some embodiments, the terminator sequence includes a nucleic acid sequence at least 75% identical to a terminator sequence in Table 6. In some embodiments, the terminator sequence includes a nucleic acid sequence at least 70% identical to a terminator sequence in Table 6.

In some embodiments, the terminator sequence may include a 3′ terminator sequence. In some embodiments, the terminator sequence is 3′ or downstream relative to the promoter sequence. In some embodiments, the terminator sequence is 3′ or downstream relative to the ESS nucleic acid sequence. In some embodiments, the terminator sequence is 3′ or downstream relative to the antisense nucleic acid sequence. In some embodiments, the terminator sequence is 3′ or downstream relative to the Sm binding site. In some embodiments, the terminator sequence is 3′ or downstream relative to the hairpin sequence. In some embodiments, the terminator sequence is 3′ or downstream relative to the promoter sequence, the ESS nucleic acid sequence, the antisense nucleic acid sequence, the Sm binding site, or the hairpin sequence.

### Compositions

Disclosed herein, in some embodiments, are compositions. The composition may be a pharmaceutical composition. The composition may include RNAs such as recombinant or modified U7 snRNA sequences described herein, or an expression construct. The composition may include an expression construct encoding a recombinant or modified U7 snRNA. The composition may include a viral vector.

Described herein, in some embodiments, the components of the system may be combined together within a single nucleic acid. In some embodiments, the components are separated among multiple nucleic acids.

In some embodiments, a pharmaceutical composition comprising the system and a pharmaceutically acceptable carrier is described. A composition may include a carrier such as a pharmaceutically acceptable carrier. Examples of carriers may include a solution such as water, a buffer, or saline, or a lipid composition.

The composition may include a delivery agent such as a virus, liposome, or nanoparticle. In some embodiments, the composition includes a delivery agent. In some embodiments, the delivery agent includes a virus. In some embodiments, the delivery agent includes a liposome. In some embodiments, the delivery agent includes a nanoparticle.

A virus may include the nucleic acid system. In some embodiments, the virus is a parvovirus. An example of a parvovirus may include a dependoparvovirus. An example of a dependoparvovirus may include an adeno-associated virus (AAV). In some embodiments, the virus may be an adeno-associated virus (AAV). In some embodiments, the AAV is a self-complementary AAV. In some embodiments, the AAV is a single-strand AAV. In some embodiments, the AAV may be serotype AAV1. In some embodiments, the AAV may be serotype AAV2. In some embodiments, the AAV may be serotype AAV4. In some embodiments, the AAV may be serotype AAV5. In some embodiments, the AAV may be serotype AAV6. In some embodiments, the AAV may be serotype AAV7. In some embodiments, the AAV may be serotype AAV8. In some embodiments, the AAV may be serotype AAV9.

In some embodiments, the, AAV may express one U7 cassette. Gene cassettes may include small mobile elements, consisting of a single gene and a recombination site, which may be integrated into larger elements called integrons. Several gene cassettes can be inserted into the same integrin forming a tandem array where cassettes can be expressed together. In some embodiments, the AAV may express an array of two U7 cassettes. In some embodiments, the AAV may express an array of three U7 cassettes. In some embodiments, the AAV may express an array of four U7 cassettes. In some embodiments, the AAV may express an array of five U7 cassettes. In some embodiments, the AAV may express at least one U7 cassette. In some embodiments, the AAV may express an array of at least two U7 cassettes. In some embodiments, the AAV may express an array of at least three U7 cassettes. In some embodiments, the AAV may express an array of at least four U7 cassettes. In some embodiments, the AAV may express an array of at least five U7 cassettes. In some embodiments, the AAV may express at most one U7 cassette. In some embodiments, the AAV may express an array of at most two U7 cassettes. In some embodiments, the AAV may express an array of at most three U7 cassettes. In some embodiments, the AAV may express an array of at most four U7 cassettes. In some embodiments, the AAV may express an array of at most five U7 cassettes.

Disclosed herein, in some embodiments, are viral particles, comprising a polynucleotide comprising one or more copies of a cassette comprising a promoter, a coding sequence, and a terminator sequence, wherein the coding sequence comprises a nucleic acid sequence having at least 80%, at least 85%, at least 90%, at least 95%, at least 98%, at least 99%, or 100% identity to the nucleic acid sequence of any one of SEQ ID NOs: 364-566. In some embodiments, the viral particle is an AAV viral particle. In some embodiments, the polynucleotide comprises 2, 3, or 4 copies of the cassette.

In some embodiments, a cell comprises the system (e.g. modified U7 snRNA). For example, the system may be delivered to a cell. The system may be delivered to the cell in vivo. The system may be delivered to the cell in vitro. The system may be administered to a subject, and thereby enter a cell of the subject. In some embodiments, the cell is a brain cell. In some embodiments, the cell is a neural cell. In some embodiments, the cell is a neuron.

In some embodiments, a tissue or biofluid of a subject comprises the system (e.g. modified U7 snRNA). For example, the system may be delivered to a tissue or biofluid. The system may be delivered to the tissue or biofluid in vivo. The system may be delivered to the tissue or biofluid in vitro. The system may be administered to a subject, and thereby enter a tissue or biofluid of the subject. In some embodiments, the tissue includes neural tissue. In some embodiments, the tissue includes brain tissue. In some embodiments, the tissue includes nerve tissue. In some embodiments, the biofluid includes blood. In some embodiments, the biofluid includes serum. In some embodiments, the biofluid includes serum.

### Methods of Use

Described herein, in some embodiments, are methods. The method may include administering a composition or system described herein. The method may include delivering a composition or system described herein in a cell. The method may include expressing a composition or system described herein in a cell. The cell may be in vivo (e.g. in a living body). The cell may be in vitro. The method may include a method of treatment. The method may include modifying splicing of a target nucleic acid such as an SCN1A RNA. The method may be performed on a subject, or on a cell such as a cell of a subject.

Described herein, in some embodiments, is a method comprising administering a pharmaceutical composition. In some embodiments, the pharmaceutical composition comprises a virus. The composition or virus may be modified. The composition or virus may be recombinant. In some embodiments, the pharmaceutical composition comprises a virus that is an adeno-associated virus (AAV). In some embodiments, the virus comprises a promoter, an exonic splicing silencer sequence, a U7 targeting sequence, a smOPT, a U7 3′ hairpin structure, and a 3′ terminal sequence. Described herein, in some embodiments, is a method comprised of administering a pharmaceutical composition or virus comprised of the nucleic acid system.

The method may be used to treat or modify splicing in a subject. The method may include administering a composition to a subject. The method may be used to treat a cell. The method may include administering a composition to a cell. The cell may be a brain cell. The cell may be a neural cell. The cell may be a neuron.

In some embodiments, the cell is in a subject. In some embodiments, the subject is an animal. In some embodiments, the subject is a vertebrate. In some embodiments, the subject is a mammal. In some embodiments, the subject is a human. In some embodiments, the subject may have one mutated copy of SCN1A DNA per cell. In some embodiments, the subject may have two mutated copies of SCN1A DNA per cell. In some embodiments, the subject may have no mutated copies of SCN1A DNA per cell. A mutated copy of SCN1A DNA may lead to splicing that includes an alternatively spliced exon of the SCN1A. A mutated copy of SCN1A DNA may result in nonsense mediated decay (NMD) of an SCN1A mRNA.

### Modifying Splicing

Described herein, in some embodiments, is a method for modifying splicing. The method may include modifying splicing in a subject. The method may include modifying splicing in a cell. In some embodiments, the method may include contacting a pre-mRNA with a system or composition herein. In some embodiments, the method may include contacting a pre-mRNA. In some embodiments, the pre-mRNA may encode an SCN1A protein. In some embodiments, the method may include contacting a pre-mRNA encoding an SCN1A protein with a recombinant nucleic acid sequence that induces exclusion of exon 20N from a mature mRNA generated by the pre-mRNA. Described herein, in some embodiments, is a method of modifying splicing comprised of contacting a pre-mRNA encoding an SCN1A protein with a recombinant nucleic acid sequence that induces exclusion of exon 20N from a mature mRNA generated by the pre-mRNA.

In some embodiments, the pre-mRNA is in a cell. In some embodiments, the pre-mRNA is in a neural cell. The cell may be a brain cell. The cell may be a neuron.

In some embodiments, the subject has a disorder. The disorder may include a neurodevelopmental disorder. For example, some embodiments include modifying splicing in cells of a subject that has a neurodevelopmental disorder. In some embodiments, the subject is at risk of having the neurodevelopmental disorder.

In some embodiments, the subject has epilepsy. In some embodiments, the subject is at risk of having epilepsy. Epilepsy is a chronic noncommunicable disease of the brain that affects around 50 million people worldwide. Epilepsy can be characterized by recurrent seizures, which are brief episodes of involuntary movement that may involve a part of the body (partial) or the entire body (generalized) and are sometimes accompanied by loss of consciousness and control of bowel or bladder function. In some embodiments, the subject may suffer from tonic-clonic seizures. In some embodiments, the subject may suffer from myoclonic seizures. In some embodiments, the subject may suffer from atypical absence seizures. In some embodiments, the subject may suffer from atonic seizures. In some embodiments, the subject may suffer from focal aware or impaired awareness seizures. In some embodiments, the subject may suffer from tonic seizures. In some embodiments, the subject may suffer from non-convulsive status epilepticus. In some embodiments, the subject may be at risk of having tonic-clonic seizures. In some embodiments, the subject is at risk of having myoclonic seizures. In some embodiments, the subject may be at risk of having atypical absence seizures. In some embodiments, the subject may be at risk of having atonic seizures. In some embodiments, the subject may be at risk of having focal aware or impaired awareness seizures. In some embodiments, the subject may be at risk of having tonic seizures. In some embodiments, the subject may be at risk of having non-convulsive status epilepticus seizures.

In some embodiments, the subject has Dravet syndrome. In some embodiments, the subject is at risk of having Dravet syndrome. In some embodiments, the subject has some level of developmental disability. In some embodiments, the subject has a crouched gait while walking. In some embodiments, the subject is at risk of having some level of developmental disability. In some embodiments, the subject is at risk of having a crouched gait while walking. In some embodiments, the subject has or is at risk of having Dravet syndrome.

Described herein, in some embodiments, is a method of preventing, reducing or inhibiting nonsense-mediated decay (NMD). The NMD may be prevented. The NMD may be reduced. The NMD may be inhibited. The NMD may be prevented, reduced, or inhibited in a subject. The NMD may be prevented, reduced, or inhibited in a cell of a subject. The NMD may be prevented, reduced, or inhibited with regard to an SCN1A transcript. Some embodiments include preventing, reducing or inhibiting nonsense-mediated decay (NMD) by contacting an SCN1A pre-mRNA with a composition or system described herein, such as a modified U7 snRNA. Some embodiments include preventing, reducing or inhibiting nonsense-mediated decay (NMD) by administering composition or system described herein to a subject. The prevention, reduction, or inhibition may be relative to a baseline or control.

Some embodiments include reducing or inhibiting NMD in cells (e.g. cells of a subject) by at least 10%. Some embodiments include reducing or inhibiting NMD in cells by at least 20%. Some embodiments include reducing or inhibiting NMD in cells by at least 30%. Some embodiments include reducing or inhibiting NMD in cells by at least 40%. Some embodiments include reducing or inhibiting NMD in cells by at least 50%. Some embodiments include reducing or inhibiting NMD in cells by at least 60%. Some embodiments include reducing or inhibiting NMD in cells by at least 70%. Some embodiments include reducing or inhibiting NMD in cells by at least 80%. Some embodiments include reducing or inhibiting NMD in cells by at least 90%. Some embodiments include reducing or inhibiting NMD in cells by less than 10%. Some embodiments include reducing or inhibiting NMD in cells by less than 20%. Some embodiments include reducing or inhibiting NMD in cells by less than 30%. Some embodiments include reducing or inhibiting NMD in cells by less than 40%. Some embodiments include reducing or inhibiting NMD in cells by less than 50%. Some embodiments include reducing or inhibiting NMD in cells by less than 60%. Some embodiments include reducing or inhibiting NMD in cells by less than 70%. Some embodiments include reducing or inhibiting NMD in cells by less than 80%. Some embodiments include reducing or inhibiting NMD in cells by less than 90%. Some embodiments include reducing or inhibiting NMD in cells by less than 95%. Some embodiments include reducing or inhibiting NMD in cells by less than 100%. Some embodiments include a range of any of the above percentages.

Described herein, in some embodiments, is a method comprised of administering to the subject a composition that silences or reduces said splicing, the composition comprising the system, virus, or composition described above. In some embodiments, the method comprises administering to the subject a composition that silences the splicing. In some embodiments, the method comprises administering to the subject a composition that reduces the splicing.

In some embodiments, the method may increase an amount of a productive isoform of SCN1A (e.g. SCN1A protein or RNA). In some embodiments, the increase in the amount of a productive isoform of SCN1A is relative to a control. In some embodiments, the increase in the amount of a productive isoform of SCN1A is relative to a baseline amount of said productive isoform. In some embodiments, the method increases an amount of a productive isoform of SCN1A, relative to a control or baseline amount of said productive isoform.

In some embodiments, the method may increase an amount of a productive isoform of SCN1A mRNA. In some embodiments, the increase in the amount of a productive isoform of SCN1A mRNA is relative to a control. In some embodiments, the increase in the amount of a productive isoform of SCN1A mRNA is relative to a baseline amount of said productive isoform. In some embodiments, the method increases an amount of a productive isoform of SCN1A mRNA, relative to a control or baseline amount of said productive isoform.

In some embodiments, the method may increase an amount of a NaV1.1 channel or protein (e.g. SCN1A protein). In some embodiments, the method may increase an amount of a NaV1.1 channel or protein (e.g. SCN1A protein), relative to a control. In some embodiments, the method may increase an amount of a NaV1.1 channel or protein (e.g. SCN1A protein), relative to a baseline amount of the NaV1.1 channel or protein. In some embodiments, the method increases an amount of a NaV1.1 channel or protein (e.g. SCN1A protein), relative to a control or baseline amount of the NaV1.1 channel or protein. The improvement may be by at least 10%.

In some embodiments, the method improves sodium transport. In some embodiments, the method improves sodium transport relative to a control. In some embodiments, the method improves sodium transport relative to a baseline amount. In some embodiments, the method improves sodium transport, relative to a control or baseline amount. The improvement may be by at least 10%.

Some embodiments relate to or include a method of reducing an amount of a non-productive SCN1A transcript. The non-productive SCN1A transcript may include a mature SCN1A mRNA that includes exon 20N. The amount of non-productive SCN1A transcript may be reduced in a cell or subject. The amount of non-productive SCN1A transcript may be reduced relative to a baseline measurement. The amount of non-productive SCN1A transcript may be reduced relative to a control measurement. The amount of non-productive SCN1A transcript may be reduced by at least 10%, by at least 20%, by at least 30%, by at least 40%, by at least 50%, by at least 60%, by at least 70%, by at least 80%, or by at least 90%. In some embodiments, the amount of non-productive SCN1A transcript is reduced by less than 10%, by less than 20%, by less than 30%, by less than 40%, by less than 50%, by less than 60%, by less than 70%, by less than 80%, by less than 90%, or by less than 100%. The amount of non-productive SCN1A transcript may be reduced by a range of percentages herein.

The amount of non-productive SCN1A transcript may be measured in a sample of a subject. The sample may include a biofluid such as blood, serum, plasma, or cerebrospinal fluid, or may include a tissue sample such as neural or brain tissue. The baseline amount of non-productive SCN1A transcript may be measured in a baseline sample obtained before treatment of the subject (e.g. before administration of a composition herein to the subject). The amount of productive SCN1A transcript may be measured with an assay method such as a PCR assay. Examples of PCR assays may include quantitative PCR (qPCR) or reverse transcription quantitative PCR (RT-qPCR). The amount of non-productive SCN1A transcript may be normalized to a control, such as a measurement of a housekeeping mRNA.

Some embodiments relate to or include a method of increasing an amount of a productive SCN1A transcript. The productive SCN1A transcript may include a mature SCN1A mRNA that does not include exon 20N. The amount of productive SCN1A transcript may be increased in a cell or subject. The amount of productive SCN1A transcript may be increased relative to a baseline measurement. The amount of productive SCN1A transcript may be increased relative to a control measurement. The amount of productive SCN1A transcript may be increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 225%, or by at least 250%. In some embodiments, the amount of productive SCN1A transcript is increased by less than 10%, by less than 20%, by less than 30%, by less than 40%, by less than 50%, by less than 60%, by less than 70%, by less than 80%, by less than 90%, by less than 100%, by less than 125%, by less than 150%, by less than 175%, by less than 200%, by less than 225%, by less than 250%, by less than 275%, by less than 300%, by less than 350%, or by less than 400%. The amount of productive SCN1A transcript may be increased by a range of percentages herein.

The amount of productive SCN1A transcript may be measured in a sample of a subject. The sample may include a biofluid such as blood, serum, plasma, or cerebrospinal fluid, or may include a tissue sample such as neural or brain tissue. The baseline amount of productive SCN1A transcript may be measured in a baseline sample obtained before treatment of the subject. The amount of productive SCN1A transcript may be measured with an assay method such as a PCR assay. The amount of productive SCN1A transcript may be normalized to a control, such as a measurement of a housekeeping mRNA.

Some embodiments relate to or include a method of increasing an amount of NaV1.1 protein. The amount of NaV1.1 protein may be increased in a cell or subject. The amount of NaV1.1 protein may be increased relative to a baseline measurement. The amount of NaV1.1 protein may be increased relative to a control measurement. The amount of NaV1.1 protein may be increased by at least 10%, by at least 20%, by at least 30%, by at least 40%, by at least 50%, by at least 60%, by at least 70%, by at least 80%, by at least 90%, by at least 100%, by at least 125%, by at least 150%, or by at least 160%. In some embodiments, the amount of NaV1.1 protein is increased by less than 10%, by less than 20%, by less than 30%, by less than 40%, by less than 50%, by less than 60%, by less than 70%, by less than 80%, by less than 90%, by less than 100%, by less than 125%, by less than 150%, by less than 160%, by less than 175%, or by less than 200%. The amount of NaV1.1 protein may be increased by a range of percentages herein.

The amount of NaV1.1 protein may be measured in a sample of a subject. The sample may include a biofluid such as blood, serum, plasma, or cerebrospinal fluid, or may include a tissue sample such as neural or brain tissue. The baseline amount of NaV1.1 protein may be measured in a baseline sample obtained before treatment of the subject. The amount of NaV1.1 protein may be measured with an assay method such as an immunoblot. The amount of NaV1.1 protein may be normalized to a control, such as a measurement of total protein or of a housekeeping protein.

### Treating a Disorder

Described herein, in some embodiments, is a method for treating a disorder in a subject in need thereof. In some embodiments, the subject has the disorder. In some embodiments, the subject is identified as having the disorder. In some embodiments, the subject is at risk of having the disorder. In some embodiments, the subject is identified as at risk of having the disorder. The treatment may have a prophylactic effect. The method may include any aspect of another method described herein, such as modifying splicing, reducing an amount of a non-productive SCN1A transcript relative to a baseline or control measurement, increasing an amount of a non-productive SCN1A transcript relative to a baseline or control measurement, or increasing an amount of NaV1.1 protein relative to a baseline or control measurement.

The disorder may be or include a genetic disorder. In some embodiments, the subject has or is at risk of having a genetic disorder. The disorder may be or include a neurodevelopmental disorder. In some embodiments, the subject has or is at risk of having a neurodevelopmental disorder. In some embodiments, the neurodevelopmental disorder comprises an intellectual disability or epilepsy. The disorder may include an intellectual disability. The disorder may include epilepsy. The disorder may include Dravet syndrome.

Described herein, in some embodiments, is a method for preventing a disorder in a subject in need thereof. Described herein, in some embodiments, is a method for treating a disorder in a subject in need thereof. In some embodiments, the method comprises administering a therapeutically effective amount of a synthetic composition that may silence splicing of an alternatively spliced region of a ribonucleic acid (RNA). In some embodiments, the method comprises administering a therapeutically effective amount of a synthetic composition that may reduce splicing of an alternatively spliced region of RNA. In some embodiments, the method comprises administering a therapeutically effective amount of a synthetic composition that may silence splicing of an alternatively spliced region of an RNA encoding SCN1A. In some embodiments, the method comprises administering a therapeutically effective amount of a synthetic composition that may reduce splicing of an alternatively spliced region of an SCN1A RNA. In some embodiments, the alternatively spliced region may include an exon. In some embodiments, the alternatively spliced region may include exon 20N of the RNA.

Described herein, in some embodiments, is a method of treating or preventing a neurodevelopmental disorder in a subject in need thereof, comprising administering a therapeutically effective amount of a recombinant U7 small nuclear RNA (snRNA) composition that silences or reduces splicing of an alternatively spliced region of an RNA encoding SCN1A. In some embodiments, the neurodevelopmental disorder comprises an intellectual disability or epilepsy.

In some embodiments, the administration may increase the amount of a productive isoform of SCN1A RNA in the subject. In some embodiments, the administration may increase the amount of a productive isoform of SCN1A RNA in the subject relative to a control. In some embodiments, the administration may increase the amount of a productive isoform of SCN1A RNA in the subject relative to a baseline amount of said productive isoform. In some embodiments, the method increases an amount of a productive isoform of SCN1A RNA in the subject, relative to a control or baseline amount of said productive isoform.

In some embodiments, the administration may increase the amount of a NaV1.1 channel or protein (e.g. SCN1A protein) in the subject. In some embodiments, the administration may increase the amount of a NaV1.1 channel or protein (e.g. SCN1A protein) in the subject relative to a control. In some embodiments, the administration may increase the amount of a NaV1.1 channel or protein (e.g. SCN1A protein) in the subject relative to a baseline amount of the NaV1.1 channel or protein. In some embodiments, the administration increases an amount of a NaV1.1 channel or protein (e.g. SCN1A protein) in the subject, relative to a control or baseline amount of the NaV1.1 channel or protein.

In some embodiments, the administration may improve sodium transport in the subject. In some embodiments, the administration may improve sodium transport in the subject relative to a control. In some embodiments, the administration may improve sodium transport in the subject relative to a baseline amount of sodium transport. In some embodiments, the administration improves sodium transport in the subject, relative to a control or baseline amount.

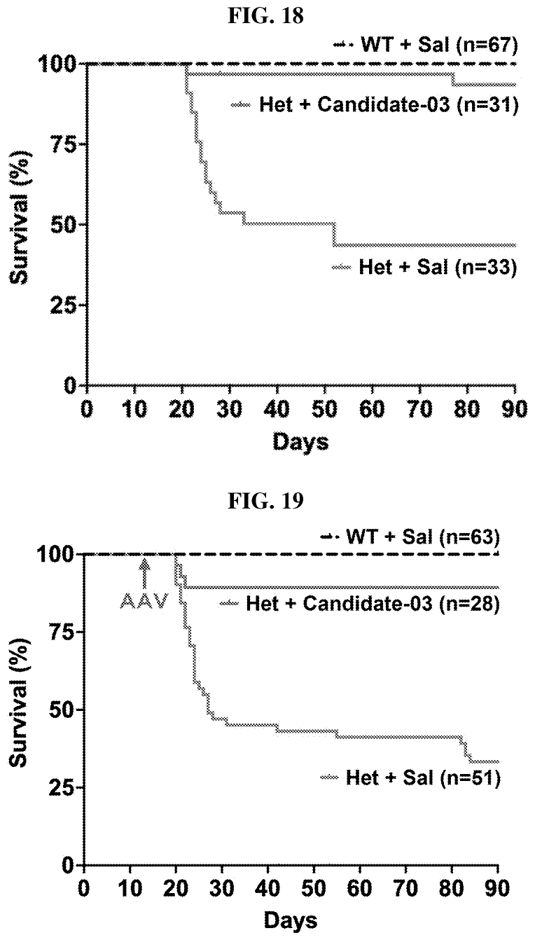

The method or administration may improve a survival chance of a subject, for example as shown in FIG. 18 or FIG. 19. The method may reduce a number of seizures, seizure frequency, or seizure duration as provided herein.

The disorder may include epilepsy. The epilepsy of a subject having a disorder may be treated or improved. The disorder may include seizures. The seizures of a subject having a disorder may be treated or improved. For example, a seizure number, frequency, or duration may be reduced in a subject having a disorder upon being treated as provided herein.

### Treating Epilepsy

The disorder may include epilepsy. The disorder may include a seizure. In some embodiments, the subject has epilepsy. In some embodiments, the subject is at risk of having epilepsy. Epilepsy is a chronic noncommunicable disease of the brain that affects around 50 million people worldwide. Epilepsy can be characterized by recurrent seizures, which are brief episodes of involuntary movement that may involve a part of the body (partial) or the entire body (generalized) and are sometimes accompanied by loss of consciousness and control of bowel or bladder function. In some embodiments, the subject may suffer from tonic-clonic seizures. In some embodiments, the subject may suffer from myoclonic seizures. In some embodiments, the subject may suffer from atypical absence seizures. In some embodiments, the subject may suffer from atonic seizures. In some embodiments, the subject may suffer from focal aware or impaired awareness seizures. In some embodiments, the subject may suffer from tonic seizures. In some embodiments, the subject may suffer from non-convulsive status epilepticus. In some embodiments, the subject may be at risk of having tonic-clonic seizures. In some embodiments, the subject is at risk of having myoclonic seizures. In some embodiments, the subject may be at risk of having atypical absence seizures. In some embodiments, the subject may be at risk of having atonic seizures. In some embodiments, the subject may be at risk of having focal aware or impaired awareness seizures. In some embodiments, the subject may be at risk of having tonic seizures. In some embodiments, the subject may be at risk of having non-convulsive status epilepticus seizures.

Described herein, in some embodiments, is a method for treating epilepsy in a subject in need thereof. Described herein, in some embodiments, is a method for preventing epilepsy in a subject in need thereof. In some embodiments, the method comprises administering a therapeutically effective amount of a synthetic composition that may silence splicing of an alternatively spliced region of a ribonucleic acid (RNA). In some embodiments, the method comprises administering a therapeutically effective amount of a synthetic composition that may reduce splicing of an alternatively spliced region of RNA. In some embodiments, the method comprises administering a therapeutically effective amount of a synthetic composition that may silence splicing of an alternatively spliced region of RNA encoding an SCN1A protein. In some embodiments, the method comprises administering a therapeutically effective amount of a synthetic composition that may reduce splicing of an alternatively spliced region of RNA encoding a sodium channel protein type 1 subunit alpha (SCN1A) protein. Described herein, in some embodiments, is a method of treating or preventing epilepsy in a subject in need thereof, comprised of administering a therapeutically effective amount of a synthetic composition that silences or reduces splicing of an alternatively spliced region of a ribonucleic acid (RNA) encoding a sodium channel protein type 1 subunit alpha (SCN1A) protein. In some embodiments, the alternatively spliced region may include an exon. In some embodiments, the alternatively spliced region may include exon 20N of the RNA.

In some embodiments, the subject is in need of treatment for an epileptic disorder. In some embodiments, the subject is in need of treatment for Dravet syndrome. The method may improve a subject's survival chances as provided herein.

In some embodiments, the administration may increase the amount of a productive isoform of SCN1A in the subject. In some embodiments, the administration may increase the amount of a productive isoform of SCN1A in the subject relative to a control. In some embodiments, the administration may increase the amount of a productive isoform of SCN1A in the subject relative to a baseline amount of said productive isoform. In some embodiments, the method increases an amount of a productive isoform of SCN1A in the subject, relative to a control or baseline amount of said productive isoform.

In some embodiments, the administration may increase the amount of a NaV1.1 channel or protein (e.g. SCN1A protein) in the subject. In some embodiments, the administration may increase the amount of a NaV1.1 channel or protein (e.g. SCN1A protein) in the subject relative to a control. In some embodiments, the administration may increase the amount of a NaV1.1 channel or protein (e.g. SCN1A protein) in the subject relative to a baseline amount of the NaV1.1 channel or protein. In some embodiments, the administration increases an amount of a NaV1.1 channel or protein (e.g. SCN1A protein) in the subject, relative to a control or baseline amount of the NaV1.1 channel or protein.

In some embodiments, the administration may improve sodium transport in the subject. In some embodiments, the administration may improve sodium transport in the subject relative to a control. In some embodiments, the administration may improve sodium transport in the subject relative to a baseline amount of sodium transport. In some embodiments, the administration improves sodium transport in the subject, relative to a control or baseline amount.

In some embodiments, the administration prevents the subject from having seizures. In some embodiments, the administration reduces the amount of the seizures of the subject relative to a baseline amount. In some embodiments, the administration reduces the severity of the seizures of the subject relative to a baseline severity. In some embodiments, the administration prevents the subject from having seizures, or reduces an amount or severity of the seizures of the subject relative to a baseline amount or severity.

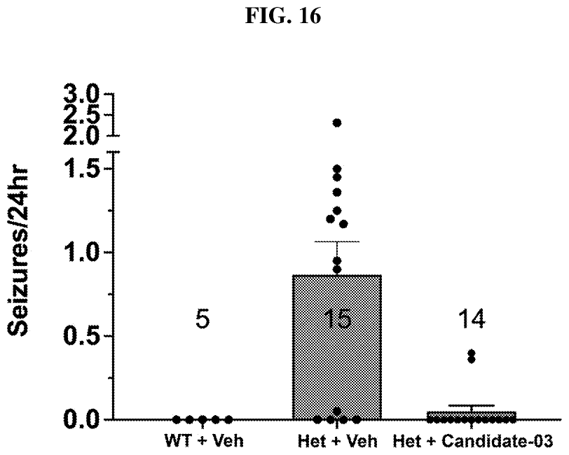

The method or administration may reduce a number of seizures or a seizure rate in a subject, for example as shown in FIG. 16. The method or administration may reduce a number of seizures in a subject. In some embodiments, the administration reduces a number of seizures in the subject by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or by a range of percentages defined by any 2 of the aforementioned percentages. In some embodiments, the administration reduces a number of seizures in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100%, relative to a baseline a number of seizures prior to the administration. In some embodiments, the administration reduces a number of seizures in a subject by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 96%, less than 97%, less than 98%, less than 99%, or less than 100%, relative to a baseline a number of seizures prior to the administration. In some embodiments, the administration reduces a number of seizures in a subject by 5-100%, relative to a baseline a number of seizures prior to the administration. In some embodiments, the administration reduces a number of seizures in a subject by 10-95%, relative to a baseline a number of seizures prior to the administration. In some embodiments, the administration reduces a number of seizures in a subject by 25-95%, relative to a baseline a number of seizures prior to the administration. In some embodiments, the administration reduces a number of seizures in a subject by 50-95%, relative to a baseline a number of seizures prior to the administration. In some embodiments, the administration reduces a number of seizures in a subject by 75-95%, relative to a baseline a number of seizures prior to the administration.

The number of seizures reduced may be over a period of time. The period of time may be 1 hour, 2 hours, 3 hours, 4 hours, 5 hours, 6 hours, 9 hours, 12 hours, 18 hours, 24 hours, 36 hours, 2 days, 3 days, 4 days, 5 days 6 days 7 days, or 1 month, or a range defined by any 2 of the aforementioned periods of time. The period of time may be about 1 hour, about 2 hours, about 3 hours, about 4 hours, about 5 hours, about 6 hours, about 9 hours, about 12 hours, about 18 hours, about 24 hours, about 36 hours, about 2 days, about 3 days, about 4 days, about 5 days 6 days 7 days, or about 1 month, or a range defined by any 2 of the aforementioned periods of time. The period of time may be at least 1 hour, at least 2 hours, at least 3 hours, at least 4 hours, at least 5 hours, at least 6 hours, at least 9 hours, at least 12 hours, at least 18 hours, at least 24 hours, at least 36 hours, at least 2 days, at least 3 days, at least 4 days, at least 5 days 6 days 7 days, or at least 1 month, or a range defined by any 2 of the aforementioned periods of time.

The method or administration may reduce a seizure rate in a subject. In some embodiments, the administration reduces a seizure rate in the subject by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or by a range of percentages defined by any 2 of the aforementioned percentages. In some embodiments, the administration reduces a seizure rate in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 100%, relative to a baseline seizure rate prior to the administration. In some embodiments, the administration reduces a seizure rate in a subject by less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 96%, less than 97%, less than 98%, less than 99%, or less than 100%, relative to a baseline seizure rate prior to the administration. In some embodiments, the administration reduces a seizure rate in a subject by 5-100%, relative to a baseline seizure rate prior to the administration. In some embodiments, the administration reduces a seizure rate in a subject by 10-95%, relative to a baseline seizure rate prior to the administration. In some embodiments, the administration reduces a seizure rate in a subject by 25-95%, relative to a baseline seizure rate prior to the administration. In some embodiments, the administration reduces a seizure rate in a subject by 50-95%, relative to a baseline seizure rate prior to the administration. In some embodiments, the administration reduces a seizure rate in a subject by 75-95%, relative to a baseline seizure rate prior to the administration.

A reduced seizure rate may last for a period of time. The period of time may be 1 hour, 2 hours, 3 hours, 4 hours, 5 hours, 6 hours, 9 hours, 12 hours, 18 hours, 24 hours, 36 hours, 2 days, 3 days, 4 days, 5 days 6 days 7 days, or 1 month, or a range defined by any 2 of the aforementioned periods of time. The period of time may be about 1 hour, about 2 hours, about 3 hours, about 4 hours, about 5 hours, about 6 hours, about 9 hours, about 12 hours, about 18 hours, about 24 hours, about 36 hours, about 2 days, about 3 days, about 4 days, about 5 days 6 days 7 days, or about 1 month, or a range defined by any 2 of the aforementioned periods of time. The period of time may be at least 1 hour, at least 2 hours, at least 3 hours, at least 4 hours, at least 5 hours, at least 6 hours, at least 9 hours, at least 12 hours, at least 18 hours, at least 24 hours, at least 36 hours, at least 2 days, at least 3 days, at least 4 days, at least 5 days 6 days 7 days, or at least 1 month, or a range defined by any 2 of the aforementioned periods of time.

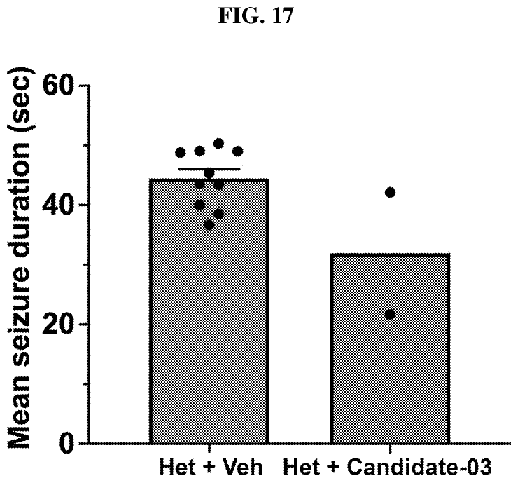

The method or administration may reduce a seizure duration in a subject, for example as shown in FIG. 17. In some embodiments, the administration reduces a seizure duration in the subject by about 5%, about 10%, about 15%, about 20%, about 25%, about 30%, about 35%, about 40%, about 45%, or about 50%, or by a range of percentages defined by any 2 of the aforementioned percentages. In some embodiments, the administration reduces a seizure duration in a subject by at least about 5%, at least about 10%, at least about 15%, at least about 20%, at least about 25%, at least about 30%, at least about 35%, at least about 40%, at least about 45%, or at least about 50%, relative to a baseline seizure duration prior to the administration. In some embodiments, the administration reduces a seizure duration in a subject by less than about 5%, less than about 10%, less than about 15%, less than about 20%, less than about 25%, less than about 30%, less than about 35%, less than about 40%, less than about 45%, or less than about 50%, relative to a baseline seizure duration prior to the administration. In some embodiments, the administration reduces a seizure duration in a subject by 5-50%, relative to a baseline seizure duration prior to the administration. In some embodiments, the administration reduces a seizure duration in a subject by 10-40%, relative to a baseline seizure duration prior to the administration.

A reduced duration of seizures may last for a period of time. The period of time may be 1 hour, 2 hours, 3 hours, 4 hours, 5 hours, 6 hours, 9 hours, 12 hours, 18 hours, 24 hours, 36 hours, 2 days, 3 days, 4 days, 5 days 6 days 7 days, or 1 month, or a range defined by any 2 of the aforementioned periods of time. The period of time may be about 1 hour, about 2 hours, about 3 hours, about 4 hours, about 5 hours, about 6 hours, about 9 hours, about 12 hours, about 18 hours, about 24 hours, about 36 hours, about 2 days, about 3 days, about 4 days, about 5 days 6 days 7 days, or about 1 month, or a range defined by any 2 of the aforementioned periods of time. The period of time may be at least 1 hour, at least 2 hours, at least 3 hours, at least 4 hours, at least 5 hours, at least 6 hours, at least 9 hours, at least 12 hours, at least 18 hours, at least 24 hours, at least 36 hours, at least 2 days, at least 3 days, at least 4 days, at least 5 days 6 days 7 days, or at least 1 month, or a range defined by any 2 of the aforementioned periods of time.

### Treating Dravet Syndrome

The disorder may include Dravet syndrome. Described herein, in some embodiments, is a method for treating Dravet syndrome in a subject in need thereof. In some embodiments, the method may prevent Dravet syndrome in a subject in need thereof. In some embodiments, the method comprises administering a therapeutically effective amount of a synthetic composition that may silence splicing of an alternatively spliced region of a ribonucleic acid (RNA) encoding a sodium channel protein type 1 subunit alpha (SCN1A) protein. In some embodiments, the method comprises administering a therapeutically effective amount of a synthetic composition that may reduce splicing of an alternatively spliced region of a ribonucleic acid (RNA) encoding a sodium channel protein type 1 subunit alpha (SCN1A) protein. Described herein, in some embodiments, is a method of treating Dravet syndrome in a subject in need thereof, comprised of administering a therapeutically effective amount of a synthetic composition that silences or reduces splicing of an alternatively spliced region of a ribonucleic acid (RNA) encoding a sodium channel protein type 1 subunit alpha (SCN1A) protein. In some embodiments, the alternatively spliced region comprises exon 20N of the RNA, or an equivalent region depending on the subject's species.

In some embodiments, the administration may increase an amount of a productive isoform of SCN1A in the subject. In some embodiments, the administration may increase an amount of a productive isoform of SCN1A in the subject relative to a control. In some embodiments, the administration may increase an amount of a productive isoform of SCN1A in the subject relative to a baseline amount of said productive isoform. In some embodiments, the administration increases an amount of a productive isoform of SCN1A in the subject, relative to a control or baseline amount of said productive isoform.

In some embodiments, the administration may increase an amount of a NaV1.1 channel or protein (e.g. SCN1A protein) in the subject. In some embodiments, the administration may increase an amount of a NaV1.1 channel or protein (e.g. SCN1A protein) in the subject relative to a control amount of the NaV1.1 channel or protein. In some embodiments, the administration may increase an amount of a NaV1.1 channel or protein (e.g. SCN1A protein) in the subject relative to a baseline amount of the NaV1.1 channel or protein. In some embodiments, the administration increases an amount of a NaV1.1 channel or protein (e.g. SCN1A protein) in the subject, relative to a control or baseline amount of the NaV1.1 channel or protein.

In some embodiments, the administration may improve sodium transport in the subject. In some embodiments, the administration may improve sodium transport in the subject relative to a control. In some embodiments, the administration may improve sodium transport in the subject relative to a baseline amount. In some embodiments, the administration improves sodium transport in the subject, relative to a control or baseline amount.

In some embodiments, the administration may prevent the subject from having seizures. Characteristics of seizures vary and depend on where in the brain the disturbance first starts, and how far it spreads. Temporary symptoms occur, such as loss of awareness or consciousness, and disturbances of movement, sensation (including vision, hearing and taste), mood, or other cognitive functions. Subjects with epilepsy tend to have more physical problems (such as fractures and bruising from injuries related to seizures) as well as higher rates of psychological conditions, including anxiety and depression. The risk of premature death in people with epilepsy is up to three times higher than in the general population. In some embodiments, the administration may reduce the amount of the seizures of the subject relative to a baseline amount. In some embodiments, the administration may reduce the severity of the seizures of the subject relative to a baseline severity. In some embodiments, the administration prevents the subject from having seizures, or reduces an amount or severity of the seizures of the subject relative to a baseline amount or severity. The method may reduce a number of seizures, seizure frequency, or seizure duration as provided herein, or may improve a subject's chance for survival also as provided herein.

The Dravet syndrome may include epilepsy. The epilepsy of a subject having Dravet syndrome may be treated or improved. The Dravet syndrome may include seizures. The seizures of a subject having Dravet syndrome may be treated or improved. For example, a seizure number, frequency, or duration may be reduced in a subject with Dravet syndrome upon treatment.

### Administration

Disclosed herein, in some embodiments, are methods that include administering a composition. The administration may be to a subject. The administration may be to a human subject. The administered composition may include an engineered snRNA. The administered composition may include an expression vector. The administered composition may include an expression vector encoding an engineered snRNA. The administered composition may include a pharmaceutical composition. The administered composition may include a virus. The administered composition may include a virus comprising an expression vector. The administered composition may include a liposome. The administered composition may include a nanoparticle.

The administration may be by a route of administration. The administration be systemic. The administration be intravenous. The administration may include an injection.

The administration may be at a site of administration. The administration may be intracerebroventricular (ICV). The administration may be retroorbital.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 15% of that number. The term “about” a range refers to that range minus 15% of its lowest value and plus 15% of its greatest value.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

Some embodiments relate to a small nuclear RNA (snRNA). An snRNA may include a class of small RNA molecules found within splicing speckles or Cajal bodies of a eukaryotic cell nucleus. A length of an unmodified snRNA may average about 150 nucleotides. An snRNA may be transcribed by RNA polymerase II or RNA polymerase III. An snRNA may function in the processing of pre-messenger RNA (hnRNA) in the nucleus. Any of these aspects may be modified or missing in a modified or engineered snRNA. An snRNA may associate with a protein or set of proteins to form a complex. The complex may be referred to as a small nuclear ribonucleoprotein (snRNP). Some examples of human snRNA components of such complexes may include: U1 spliceosomal RNA, U2 spliceosomal RNA, U4 spliceosomal RNA, U5 spliceosomal RNA, or U6 spliceosomal RNA. An snRNA may have a high uridine content.

Some embodiments relate to a U7 snRNA. A U7 snRNA may include an RNA molecule and a component of a small nuclear ribonucleoprotein complex (U7 snRNP). The U7 snRNA may affect histone pre-mRNA processing. The U7 snRNA may be modified or engineered. In some embodiments, the modified or engineered U7 snRNA does not affect histone pre-mRNA processing, or has little effect on such. In some embodiments, a U7 snRNA has a 5′ end that binds an HDE (histone downstream element), a conserved purine-rich region, located 15 nucleotides downstream a histone mRNA cleavage site. Any of these aspects may be modified or missing in a modified or engineered U7 snRNA. Binding of an HDE region by a U7 snRNA, through complementary base-pairing, may affect recruitment of cleavage factors during histone pre-mRNA processing.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

A percent sequence identity may be determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the % sequence identity. A sequence identity may include a sequence identity to a reverse complement. In determining a sequence identity, thymine (T) and uracil (U) may be interchangeable. T and U may be interchangeable when describing an oligonucleotide. In some embodiments, Ts and Us are interchangeable depending on whether the oligonucleotide is an RNA or DNA, where RNA includes U and DNA includes T.

A discrepancy between the written description and a sequence listing submitted herein may be resolved in favor of the written description.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Numbered Embodiments

This disclosure includes the following embodiments:

1. A system for modifying nucleic acid splicing, comprising:
an exonic splicing silencer (ESS) nucleic acid sequence; and
an antisense nucleic acid sequence that targets an alternatively spliced region of a ribonucleic acid (RNA) encoding a sodium channel protein type 1 subunit alpha (SCN1A) protein.

2. The system of embodiment 1, wherein the system comprises an engineered U7 snRNA comprising the ESS nucleic acid sequence and the antisense nucleic acid sequence.

3. The system of embodiment 1 or 2, wherein the alternatively spliced region comprises an alternative exon of the RNA encoding SCN1A, wherein inclusion of the alternative exon in a mature SCN1A mRNA results in nonsense mediated decay (NMD) of a transcription product of the mature SCN1A mRNA.

4. The system of any one of embodiments 1-3, wherein the alternatively spliced region comprises exon 20N of the RNA encoding SCN1A.

5. The system of embodiment 4, wherein the exon 20N comprises a nucleic acid sequence at least 80%, at least 85%, at least 90%, at least 95%, at least 98%, at least 99%, or 100% identical to SEQ ID NO: 2.

6. The system of any one of embodiments 1-5, wherein the ESS recruits a protein factor or group of factors that reduce or silence splicing of the RNA encoding SCN1A.

7. The system of any one of embodiments 1-6, wherein the antisense nucleic acid sequence binds to at least a portion of the alternatively spliced region.

8 The system of embodiment 7, wherein the portion of the alternatively spliced region is within a 5′ half or 5′ end of the alternatively spliced region.

9. The system of embodiment 7, wherein the portion of the alternatively spliced region comprises nucleotide positions within 50 bp of a 5′ or 3′ end of the alternatively spliced region.

10. The system of any one of embodiments 1-9, wherein the antisense nucleic acid sequence is 3′ or downstream relative to the ESS nucleic acid sequence.

11. The system of any one of embodiments 1-10, wherein the engineered U7 snRNA further comprises a Sm binding site.

12. The system of embodiment 11, wherein the Sm binding site is 3′ or downstream relative to the ESS nucleic acid sequence or antisense nucleic acid sequence.

13. The system of any one of embodiments 1-12, wherein the engineered U7 snRNA further comprises a 3′ U7 snRNA hairpin sequence.

14. The system of embodiment 13, wherein the hairpin sequence is 3′ or downstream relative to the ESS nucleic acid sequence, the antisense nucleic acid sequence, or the Sm binding site.

15. The system of any one of embodiments 1-14, wherein the system comprises an expression cassette encoding an engineered U7 snRNA, the engineered U7 snRNA comprising the ESS nucleic acid sequence and the antisense nucleic acid sequence.

16. The system of embodiment 15, wherein the expression cassette comprises a promoter operably linked to a sequence encoding the engineered U7 snRNA.

17. The system of embodiment 16, wherein the expression cassette comprises a second sequence encoding a second copy of the engineered U7 snRNA, and the promoter is further operably linked to the second sequence.

18. The system of embodiment 17, wherein the expression cassette comprises a third sequence encoding a third copy of the engineered U7 snRNA, and the promoter is further operably linked to the third sequence.

19. The system of any one of embodiments 16-18, wherein the promoter comprises a mouse U1 snRNA promoter sequence, a human U1 snRNA promoter sequence, a mouse U7 snRNA promoter sequence, a human U7 snRNA promoter sequence, or a combination thereof.

20. The system of any one of embodiments 15-19, wherein the expression cassette comprises a 3′ terminator sequence operably linked to a sequence encoding the engineered U7 snRNA.

21. The system of embodiment 20, wherein the 3′ terminator sequence comprises a mouse U1 snRNA terminator sequence, a human U1 snRNA terminator sequence, a mouse U7 snRNA terminator sequence, a human U7 snRNA terminator sequence, or a combination thereof.

22. The system of embodiment 1, wherein expression of the system in a cell or a population of cells reduces an exon 20N measurement in a cell or population of cells by at least 10%, relative to a baseline exon 20N measurement.

23. The system of any one of embodiments 1-22, wherein expression of the system in a cell or a population of cells reduces an SCN1A NMD transcript measurement in a cell or population of cells by at least 10%, relative to a baseline SCN1A NMD transcript measurement.

24. The system of any one of embodiments 1-23, wherein expression of the system in a cell or a population of cells increases a productive form of an SCN1A transcript measurement in a cell or population of cells by at least 10%, relative to a baseline SCN1A transcript measurement.

25. The system of any one of embodiments 1-24, wherein expression of the system in a cell or a population of cells increases a productive form of an SCN1A transcript measurement in a cell or population of cells by more than 70%, relative to a baseline SCN1A transcript measurement.

26. The system any one of embodiments 1-25, wherein expression of the system in a cell or a population of cells an SCN1A protein measurement in a cell or population of cells by at least 10%, relative to a baseline SCN1A protein measurement.

27. The system of any one of embodiments 1-26, wherein expression of the system in a cell or a population of cells an SCN1A protein measurement in a cell or population of cells by at least 70%, relative to a baseline SCN1A protein measurement.

28. A system for modifying nucleic acid splicing, comprising:
an engineered U7 small nuclear RNA (snRNA) comprising an antisense nucleic acid sequence that binds to an alternatively spliced region of a ribonucleic acid (RNA) encoding a sodium channel protein type 1 subunit alpha (SCN1A) protein.

29. The system of embodiment 28, wherein the engineered U7 snRNA further comprises an exonic splicing silencer (ESS) nucleic acid sequence.

30. The system of embodiment 28, wherein the engineered U7 snRNA does not comprise an ESS nucleic acid sequence.

31. A pharmaceutical composition comprising the system of any one of embodiments 1-30 and a pharmaceutically acceptable carrier.

32. An engineered virus comprising the system of any one of embodiments 1-30.

33. The engineered virus of embodiment 32, comprising an adeno-associated virus (AAV).

34. A method, comprising: administering the engineered virus of embodiment 32 or 33 to a subject.

35. A method of modifying splicing, comprising: contacting a pre-mRNA encoding SCN1A with an engineered U7 snRNA or with a vector encoding the engineered U7 snRNA, wherein the engineered U7 snRNA induces exclusion of exon 20N from a mature mRNA generated by the pre-mRNA.

36. The method of embodiment 35, wherein the method increases an amount of a productive isoform of SCN1A, relative to a control or baseline amount of said productive isoform.

37. The method of embodiment 35 or 36, wherein the contact or administration decreases an amount of a non-productive isoform of SCN1A in the subject, relative to a control or baseline amount of said non-productive isoform.

38. The method of any one of embodiments 35-37, wherein the method increases an amount of a NaV1.1, relative to a control or baseline amount of the NaV1.1.

39. A method of treating or preventing epilepsy in a subject in need thereof, comprising: administering a therapeutically effective amount of a recombinant U7 snRNA composition that silences or reduces splicing of an alternatively spliced region of an RNA encoding SCN1A.

40. The method of embodiment 39, further comprising identifying the subject as having epilepsy or as being at risk of having epilepsy, and selecting the treatment based on said identifying.

41. The method of embodiment 39 or 40, wherein the administration prevents the subject from having seizures, or reduces an amount or severity of the seizures of the subject relative to a baseline amount or severity.

42. The method of any one of embodiments 39-41, wherein the administration reduce a seizure rate in the subject, relative to a baseline seizure rate before the administration.

43. The method of embodiment 42, wherein the seizure rate is reduced in the subject by at least 10%, relative to the baseline seizure rate.

44. The method of embodiment 42 or 43, wherein the seizure rate is reduced in the subject by at least 90%, relative to the baseline seizure rate.

45. The method of any one of embodiments 39-44, wherein the administration reduce a seizure duration in the subject, relative to a baseline seizure duration before the administration.

46. The method of embodiment 45, wherein the seizure duration is reduced in the subject by at least 10%, relative to the baseline seizure duration.

47. A method of treating or Dravet Syndrome in a subject in need thereof, comprising: administering a therapeutically effective amount of a synthetic composition comprising a recombinant U7 snRNA that silences or reduces splicing of an alternatively spliced region of a RNA encoding SCN1A.

48. The method of embodiment 47, further comprising identifying the subject as having Dravet syndrome, and selecting the treatment based on said identifying.

49. The method of embodiment 47 or 48, wherein the administration reduces or improves a symptom of the Drave syndrome in the subject.

50. The method of any one of embodiments 47-49, wherein the administration prevents the subject from having seizures, or reduces an amount or severity of the seizures of the subject relative to a baseline amount or severity.

### EXAMPLES

The following examples are included for illustrative purposes only and are not intended to limit the scope of the invention. The data in these examples are indicative of improvements in the compositions and methods described herein over existing compositions and methods.

### Example 1: Screen to Identify U7-Based Splice Modulation Candidates of SCN1A

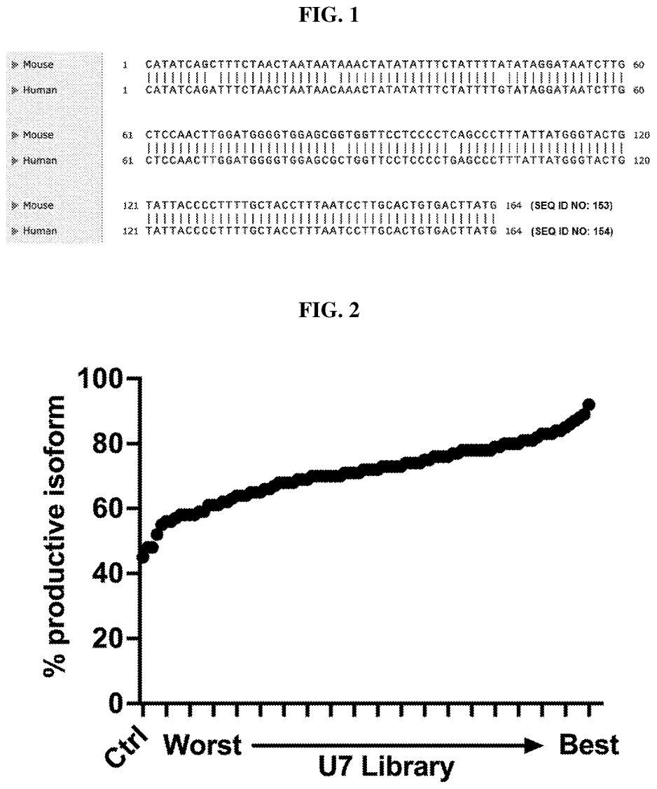

The alternatively spliced NMD exon of SEQ ID NO: 2 present in humans (exon 20N) is 96% conserved in mice (62/64 identity) (mouse exon 21N, SEQ ID NO: 1). The region encompassing the NMD exon, excluding 50 bp upstream and downstream, has similar identity (159/164 identity) (FIG. 1) A screen was performed in mouse neuroblast cells (Neuro-2a cell line) with a plasmid library encoding U7 expression cassettes (which included a promoter, ESS, antisense sequence, smOPT, hairpin, and 3′ terminator) designed to target the entire NMD exon and flanking regions. The library consisted of 32 unique U7 targeting sequences of 20, 24, 26, or 30 nucleotides in length. Each targeting sequence was paired with each of three unique exonic splicing silencer (ESS) sequences (Tables 2A-2B). Neuro-2a cells were then transfected with plasmid DNA and suppression of NMD exon inclusion was analyzed via end point RT-PCR. This method identified 19 U7 targeting sequence/ESS combinations capable of increasing SCN1A isoform expression by 50% or greater (FIG. 2, Table 2C).

TABLE 2A

| Construct | ESS Sequence | ESS SEQ | Mouse U7 Antisense | SEQ ID |
|---|---|---|---|---|
| Name | (5′ to 3′) | ID NO: | Sequence (5′ to 3′) | NO: |
| Scramble- | ATGATAGGGACTTAGGGT | 11 | GCACCCCATCCCCCACTACA | 14 |
| 01 | GA | | | |
| N5-01 | ATGATAGGGACTTAGGGT | 11 | AACCACCGCTCCACCCCATC | 18 |
| | GA | | | |
| N5-02 | ATGATAGGGACTTAGGGT | 11 | CAAGTTGGAGCAAGATTATC | 19 |
| | GA | | | |
| N5-03 | ATGATAGGGACTTAGGGT | 11 | CTATATAAAATAGAAATATA | 20 |
| | GA | | | |
| N5-04 | ATGATAGGGACTTAGGGT | 11 | TAGTTTATTATTAGTTAGAA | 21 |
| | GA | | | |
| N5-05 | ATGATAGGGACTTAGGGT | 11 | GAGGAACCACCGCTCCACCCC | 22 |
| | GA | | ATC | |
| N5-06 | ATGATAGGGACTTAGGGT | 11 | CATCCAAGTTGGAGCAAGATT | 23 |
| | GA | | ATC | |
| N5-07 | ATGATAGGGACTTAGGGT | 11 | TATCCTATATAAAATAGAAAT | 24 |
| | GA | | ATA | |
| N5-08 | ATGATAGGGACTTAGGGT | 11 | TATATAGTTTATTATTAGTTAG | 25 |
| | GA | | AA | |
| N5-09 | ATGATAGGGACTTAGGGT | 11 | GGGAGGAACCACCGCTCCACC | 26 |
| | GA | | CCATC | |
| N5-10 | ATGATAGGGACTTAGGGT | 11 | CCCATCCAAGTTGGAGCAAGA | 27 |
| | GA | | TTATC | |
| N5-11 | ATGATAGGGACTTAGGGT | 11 | ATTATCCTATATAAAATAGAA | 28 |
| | GA | | ATATA | |
| N5-12 | ATGATAGGGACTTAGGGT | 11 | AATATATAGTTTATTATTAGTT | 29 |
| | GA | | AGAA | |
| N5-13 | ATGATAGGGACTTAGGGT | 11 | TGAGGGGAGGAACCACCGCTC | 30 |
| | GA | | CACCCCATC | |
| N5-14 | ATGATAGGGACTTAGGGT | 11 | CCACCCCATCCAAGTTGGAGC | 31 |
| | GA | | AAGATTATC | |
| N5-15 | ATGATAGGGACTTAGGGT | 11 | CAAGATTATCCTATATAAAAT | 32 |
| | GA | | AGAAATATA | |
| N5-16 | ATGATAGGGACTTAGGGT | 11 | TAGAAATATATAGTTTATTATT | 33 |
| | GA | | AGTTAGAA | |
| N3-01 | ATGATAGGGACTTAGGGT | 11 | AGTGCAAGGATTAAAGGTAG | 34 |
| | GA | | | |
| N3-02 | ATGATAGGGACTTAGGGT | 11 | CAAAAGGGGTAATACAGTAC | 35 |
| | GA | | | |
| N3-03 | ATGATAGGGACTTAGGGT | 11 | CCATAATAAAGGGCTGAGGG | 36 |
| | GA | | | |
| N3-04 | ATGATAGGGACTTAGGGT | 11 | GAGGAACCACCGCTCCACCC | 37 |
| | GA | | | |
| N3-05 | ATGATAGGGACTTAGGGT | 11 | TCACAGTGCAAGGATTAAAGG | 38 |
| | GA | | TAG | |
| N3-06 | ATGATAGGGACTTAGGGT | 11 | GTAGCAAAAGGGGTAATACA | 39 |
| | GA | | GTAC | |
| N3-07 | ATGATAGGGACTTAGGGT | 11 | GTACCCATAATAAAGGGCTGA | 40 |
| | GA | | GGG | |
| N3-08 | ATGATAGGGACTTAGGGT | 11 | AGGGGAGGAACCACCGCTCCA | 41 |
| | GA | | CCC | |
| N3-09 | ATGATAGGGACTTAGGGT | 11 | AGTCACAGTGCAAGGATTAAA | 42 |
| | GA | | GGTAG | |
| N3-10 | ATGATAGGGACTTAGGGT | 11 | AGGTAGCAAAAGGGGTAATA | 43 |
| | GA | | CAGTAC | |
| N3-11 | ATGATAGGGACTTAGGGT | 11 | CAGTACCCATAATAAAGGGCT | 44 |
| | GA | | GAGGG | |
| N3-12 | ATGATAGGGACTTAGGGT | 11 | TGAGGGGAGGAACCACCGCTC | 45 |
| | GA | | CACCC | |
| N3-13 | ATGATAGGGACTTAGGGT | 11 | CATAAGTCACAGTGCAAGGAT | 46 |
| | GA | | TAAAGGTAG | |
| N3-14 | ATGATAGGGACTTAGGGT | 11 | TTAAAGGTAGCAAAAGGGGTA | 47 |
| | GA | | ATACAGTAC | |
| N3-15 | ATGATAGGGACTTAGGGT | 11 | AATACAGTACCCATAATAAAG | 48 |
| | GA | | GGCTGAGGG | |
| N3-16 | ATGATAGGGACTTAGGGT | 11 | GGGCTGAGGGGAGGAACCAC | 49 |
| | GA | | CGCTCCACCC | |
| N5- | TTTGTTCCGTGGGTGGTTT | 12 | AACCACCGCTCCACCCCATC | 18 |
| ESSalt1-01 | A | | | |
| N5- | TTTGTTCCGTGGGTGGTTT | 12 | CAAGTTGGAGCAAGATTATC | 19 |
| ESSalt1-02 | A | | | |
| N5- | TTTGTTCCGTGGGTGGTTT | 12 | CTATATAAAATAGAAATATA | 20 |
| ESSalt1-03 | A | | | |
| N5- | TTTGTTCCGTGGGTGGTTT | 12 | TAGTTTATTATTAGTTAGAA | 21 |
| ESSalt1-04 | A | | | |
| N5- | TTTGTTCCGTGGGTGGTTT | 12 | GAGGAACCACCGCTCCACCCC | 22 |
| ESSalt1-05 | A | | ATC | |
| N5- | TTTGTTCCGTGGGTGGTTT | 12 | CATCCAAGTTGGAGCAAGATT | 23 |
| ESSalt1-06 | A | | ATC | |
| N5- | TTTGTTCCGTGGGTGGTTT | 12 | TATCCTATATAAAATAGAAAT | 24 |
| ESSalt1-07 | A | | ATA | |
| N5- | TTTGTTCCGTGGGTGGTTT | 12 | TATATAGTTTATTATTAGTTAG | 25 |
| ESSalt1-08 | A | | AA | |
| N5- | TTTGTTCCGTGGGTGGTTT | 12 | GGGAGGAACCACCGCTCCACC | 26 |
| ESSalt1-09 | A | | CCATC | |
| Scramble- | TTTGTTCCGTGGGTGGTTT | 12 | GGAGAATCACCTTATAGGAT | 15 |
| 02 | A | | | |
| N5- | TTTGTTCCGTGGGTGGTTT | 12 | CCCATCCAAGTTGGAGCAAGA | 27 |
| ESSalt1-10 | A | | TTATC | |
| N5- | TTTGTTCCGTGGGTGGTTT | 12 | ATTATCCTATATAAAATAGAA | 28 |
| ESSalt1-11 | A | | ATATA | |
| N5- | TTTGTTCCGTGGGTGGTTT | 12 | AATATATAGTTTATTATTAGTT | 29 |
| ESSalt1-12 | A | | AGAA | |
| N5- | TTTGTTCCGTGGGTGGTTT | 12 | TGAGGGGAGGAACCACCGCTC | 30 |
| ESSalt1-13 | A | | CACCCCATC | |
| N5- | TTTGTTCCGTGGGTGGTTT | 12 | CCACCCCATCCAAGTTGGAGC | 31 |
| ESSalt1-14 | A | | AAGATTATC | |
| N5- | TTTGTTCCGTGGGTGGTTT | 12 | CAAGATTATCCTATATAAAAT | 32 |
| ESSalt1-15 | A | | AGAAATATA | |
| N5- | TTTGTTCCGTGGGTGGTTT | 12 | TAGAAATATATAGTTTATTATT | 33 |
| ESSalt1-16 | A | | AGTTAGAA | |
| N3- | TTTGTTCCGTGGGTGGTTT | 12 | AGTGCAAGGATTAAAGGTAG | 34 |
| ESSalt1-01 | A | | | |
| N3- | TTTGTTCCGTGGGTGGTTT | 12 | CAAAAGGGGTAATACAGTAC | 35 |
| ESSalt1-02 | A | | | |
| N3- | TTTGTTCCGTGGGTGGTTT | 12 | CCATAATAAAGGGCTGAGGG | 36 |
| ESSalt1-03 | A | | | |
| N3- | TTTGTTCCGTGGGTGGTTT | 12 | GAGGAACCACCGCTCCACCC | 37 |
| ESSalt1-04 | A | | | |
| Scramble- | TTTGTTCCGTGGGTGGTTT | 12 | GAATAATAACATAATAATAT | 16 |
| 03 | A | | | |

ESSalt1-05

| | | | | |
|---|---|---|---|---|
| N3- | TTTGTTCCGTGGGTGGTTT | 12 | TCACAGTGCAAGGATTAAAGG | 38 |
| ESSalt1-05 | A | | TAG | |
| N3- | TTTGTTCCGTGGGTGGTTT | 12 | GTAGCAAAAGGGGTAATACA | 39 |
| ESSalt1-06 | A | | GTAC | |
| N3- | TTTGTTCCGTGGGTGGTTT | 12 | GTACCCATAATAAAGGGCTGA | 40 |
| ESSalt1-07 | A | | GGG | |
| N3- | TTTGTTCCGTGGGTGGTTT | 12 | AGGGGAGGAACCACCGCTCCA | 41 |
| ESSalt1-08 | A | | CCC | |
| N3- | TTTGTTCCGTGGGTGGTTT | 12 | AGTCACAGTGCAAGGATTAAA | 42 |
| ESSalt1-09 | A | | GGTAG | |
| N3- | TTTGTTCCGTGGGTGGTTT | 12 | AGGTAGCAAAAGGGGTAATA | 43 |
| ESSalt1-10 | A | | CAGTAC | |
| N3- | TTTGTTCCGTGGGTGGTTT | 12 | CAGTACCCATAATAAAGGGCT | 44 |
| ESSalt1-11 | A | | GAGGG | |
| N3- | TTTGTTCCGTGGGTGGTTT | 12 | TGAGGGGAGGAACCACCGCTC | 45 |
| ESSalt1-12 | A | | CACCC | |
| N3- | TTTGTTCCGTGGGTGGTTT | 12 | CATAAGTCACAGTGCAAGGAT | 46 |
| ESSalt1-13 | A | | TAAAGGTAG | |
| N3- | TTTGTTCCGTGGGTGGTTT | 12 | TTAAAGGTAGCAAAAGGGGTA | 47 |
| ESSalt1-14 | A | | ATACAGTAC | |
| N3- | TTTGTTCCGTGGGTGGTTT | 12 | AATACAGTACCCATAATAAAG | 48 |
| ESSalt1-15 | A | | GGCTGAGGG | |
| N3- | TTTGTTCCGTGGGTGGTTT | 12 | GGGCTGAGGGGAGGAACCAC | 49 |
| ESSalt1-16 | A | | CGCTCCACCC | |
| N5- | TGGGGGGAGGTAGGTAGG | 13 | AACCACCGCTCCACCCCATC | 18 |
| ESSalt2-01 | TA | | | |
| N5- | TGGGGGGAGGTAGGTAGG | 13 | CAAGTTGGAGCAAGATTATC | 19 |
| ESSalt2-02 | TA | | | |
| N5- | TGGGGGGAGGTAGGTAGG | 13 | CTATATAAAATAGAAATATA | 20 |
| ESSalt2-03 | TA | | | |
| N5- | TGGGGGGAGGTAGGTAGG | 13 | TAGTTTATTATTAGTTAGAA | 21 |
| ESSalt2-04 | TA | | | |
| N5- | TGGGGGGAGGTAGGTAGG | 13 | GAGGAACCACCGCTCCACCCC | 22 |
| ESSalt2-05 | TA | | ATC | |
| N5- | TGGGGGGAGGTAGGTAGG | 13 | CATCCAAGTTGGAGCAAGATT | 23 |
| ESSalt2-06 | TA | | ATC | |
| N5- | TGGGGGGAGGTAGGTAGG | 13 | TATCCTATATAAAATAGAAAT | 24 |
| ESSalt2-07 | TA | | ATA | |
| N5- | TGGGGGGAGGTAGGTAGG | 13 | TATATAGTTTATTATTAGTTAG | 25 |
| ESSalt2-08 | TA | | AA | |
| N5- | TGGGGGGAGGTAGGTAGG | 13 | GGGAGGAACCACCGCTCCACC | 26 |
| ESSalt2-09 | TA | | CCATC | |
| N5- | TGGGGGGAGGTAGGTAGG | 13 | CCCATCCAAGTTGGAGCAAGA | 27 |
| ESSalt2-10 | TA | | TTATC | |
| N5- | TGGGGGGAGGTAGGTAGG | 13 | ATTATCCTATATAAAATAGAA | 28 |
| ESSalt2-11 | TA | | ATATA | |
| N5- | TGGGGGGAGGTAGGTAGG | 13 | TGAGGGGAGGAACCACCGCTC | 30 |
| ESSalt2-13 | TA | | CACCCCATC | |
| N5- | TGGGGGGAGGTAGGTAGG | 13 | CCACCCCATCCAAGTTGGAGC | 31 |
| ESSalt2-14 | TA | | AAGATTATC | |
| N5- | TGGGGGGAGGTAGGTAGG | 13 | CAAGATTATCCTATATAAAAT | 32 |
| ESSalt2-15 | TA | | AGAAATATA | |
| N3- | TGGGGGGAGGTAGGTAGG | 13 | AGTGCAAGGATTAAAGGTAG | 34 |
| ESSalt2-01 | TA | | | |
| N3- | TGGGGGGAGGTAGGTAGG | 13 | CAAAAGGGGTAATACAGTAC | 35 |
| ESSalt2-02 | TA | | | |
| N3- | TGGGGGGAGGTAGGTAGG | 13 | CCATAATAAAGGGCTGAGGG | 36 |
| ESSalt2-03 | TA | | | |
| N3- | TGGGGGGAGGTAGGTAGG | 13 | GAGGAACCACCGCTCCACCC | 37 |
| ESSalt2-04 | TA | | | |
| N3- | TGGGGGGAGGTAGGTAGG | 13 | TCACAGTGCAAGGATTAAAGG | 38 |
| ESSalt2-05 | TA | | TAG | |
| N3- | TGGGGGGAGGTAGGTAGG | 13 | GTAGCAAAAGGGGTAATACA | 39 |
| ESSalt2-06 | TA | | GTAC | |
| N3- | TGGGGGGAGGTAGGTAGG | 13 | GTACCCATAATAAAGGGCTGA | 40 |
| ESSalt2-07 | TA | | GGG | |
| N3- | TGGGGGGAGGTAGGTAGG | 13 | AGGGGAGGAACCACCGCTCCA | 41 |
| ESSalt2-08 | TA | | CCC | |
| N3- | TGGGGGGAGGTAGGTAGG | 13 | AGTCACAGTGCAAGGATTAAA | 42 |
| ESSalt2-09 | TA | | GGTAG | |
| N3- | TGGGGGGAGGTAGGTAGG | 13 | AGGTAGCAAAAGGGGTAATA | 43 |
| ESSalt2-10 | TA | | CAGTAC | |
| N3- | TGGGGGGAGGTAGGTAGG | 13 | CAGTACCCATAATAAAGGGCT | 44 |
| ESSalt2-11 | TA | | GAGGG | |
| N3- | TGGGGGGAGGTAGGTAGG | 13 | CATAAGTCACAGTGCAAGGAT | 46 |
| ESSalt2-13 | TA | | TAAAGGTAG | |
| N3- | TGGGGGGAGGTAGGTAGG | 13 | TTAAAGGTAGCAAAAGGGGTA | 47 |
| ESSalt2-14 | TA | | ATACAGTAC | |
| N3- | TGGGGGGAGGTAGGTAGG | 13 | AATACAGTACCCATAATAAAG | 48 |
| ESSalt2-15 | TA | | GGCTGAGGG | |
| Scramble- | TGGGGGGAGGTAGGTAGG | 13 | GAAATTTATGTATTGATTAT | 17 |
| 04 | TA | | | |

TABLE 2B

| Construct | Humanized U7 Antisense | SEQ ID | % Identity to |
|---|---|---|---|
| Name | Sequence (5′ to 3′) | NO: | Mouse Sequence |
| N5-01 | AACCAGCGCTCCACCCCATC | 152 | 95.0 |
| N5-02 | CAAGTTGGAGCAAGATTATC | 19 | 100.0 |
| N5-03 | CTATACAAAATAGAAATATA | 50 | 95.0 |
| N5-04 | TAGTTTGTTATTAGTTAGAA | 51 | 95.0 |
| N5-05 | GAGGAACCAGCGCTCCACCCCATC | 52 | 95.8 |
| N5-06 | CATCCAAGTTGGAGCAAGATTATC | 23 | 100.0 |
| N5-07 | TATCCTATACAAAATAGAAATATA | 53 | 95.8 |
| N5-08 | TATATAGTTTGTTATTAGTTAGAA | 54 | 95.8 |
| N5-09 | GGGAGGAACCAGCGCTCCACCCCATC | 55 | 96.2 |
| N5-10 | CCCATCCAAGTTGGAGCAAGATTATC | 27 | 100.0 |
| N5-11 | ATTATCCTATACAAAATAGAAATATA | 56 | 96.2 |
| N5-12 | AATATATAGTTTGTTATTAGTTAGAA | 57 | 96.2 |
| N5-13 | TCAGGGGAGGAACCAGCGCTCCACCCCATC | 58 | 93.3 |
| N5-14 | CCACCCCATCCAAGTTGGAGCAAGATTATC | 31 | 100.0 |
| N5-15 | CAAGATTATCCTATACAAAATAGAAATATA | 59 | 96.7 |
| N5-16 | TAGAAATATATAGTTTGTTATTAGTTAGAA | 60 | 96.7 |
| N3-01 | AGTGCAAGGATTAAAGGTAG | 34 | 100.0 |
| N3-02 | CAAAAGGGGTAATACAGTAC | 35 | 100.0 |
| N3-03 | CCATAATAAAGGGCTCAGGG | 61 | 95.0 |
| N3-04 | GAGGAACCAGCGCTCCACCC | 62 | 95.0 |
| N3-05 | TCACAGTGCAAGGATTAAAGGTAG | 38 | 100.0 |
| N3-06 | GTAGCAAAAGGGGTAATACAGTAC | 39 | 100.0 |
| N3-07 | GTACCCATAATAAAGGGCTCAGGG | 63 | 95.8 |
| N3-08 | AGGGGAGGAACCAGCGCTCCACCC | 64 | 95.8 |
| N3-09 | AGTCACAGTGCAAGGATTAAAGGTAG | 42 | 100.0 |
| N3-10 | AGGTAGCAAAAGGGGTAATACAGTAC | 43 | 100.0 |
| N3-11 | CAGTACCCATAATAAAGGGCTCAGGG | 65 | 96.2 |
| N3-12 | TCAGGGGAGGAACCAGCGCTCCACCC | 66 | 92.3 |
| N3-13 | CATAAGTCACAGTGCAAGGATTAAAGGTAG | 46 | 100.0 |
| N3-14 | TTAAAGGTAGCAAAAGGGGTAATACAGTAC | 47 | 100.0 |
| N3-15 | AATACAGTACCCATAATAAAGGGCTCAGGG | 67 | 96.7 |
| N3-16 | GGGCTCAGGGGAGGAACCAGCGCTCCACCC | 68 | 93.3 |
| N5-ESSalt1-01 | AACCAGCGCTCCACCCCATC | 69 | 95.0 |
| N5-ESSalt1-02 | CAAGTTGGAGCAAGATTATC | 19 | 100.0 |
| N5-ESSalt1-03 | CTATACAAAATAGAAATATA | 70 | 95.0 |
| N5-ESSalt1-04 | TAGTTTGTTATTAGTTAGAA | 71 | 95.0 |
| N5-ESSalt1-05 | GAGGAACCAGCGCTCCACCCCATC | 72 | 95.8 |
| N5-ESSalt1-06 | CATCCAAGTTGGAGCAAGATTATC | 23 | 100.0 |
| N5-ESSalt1-07 | TATCCTATACAAAATAGAAATATA | 73 | 95.8 |
| N5-ESSalt1-08 | TATATAGTTTGTTATTAGTTAGAA | 74 | 95.8 |
| N5-ESSalt1-09 | GGGAGGAACCAGCGCTCCACCCCATC | 75 | 96.2 |
| N5-ESSalt1-10 | CCCATCCAAGTTGGAGCAAGATTATC | 27 | 100.0 |
| N5-ESSalt1-11 | ATTATCCTATACAAAATAGAAATATA | 76 | 96.2 |
| N5-ESSalt1-12 | AATATATAGTTTGTTATTAGTTAGAA | 77 | 96.2 |
| N5-ESSalt1-13 | TCAGGGGAGGAACCAGCGCTCCACCCCATC | 78 | 93.3 |
| N5-ESSalt1-14 | CCACCCCATCCAAGTTGGAGCAAGATTATC | 31 | 100.0 |
| N5-ESSalt1-15 | CAAGATTATCCTATACAAAATAGAAATATA | 79 | 96.7 |
| N5-ESSalt1-16 | TAGAAATATATAGTTTGTTATTAGTTAGAA | 80 | 96.7 |
| N3-ESSalt1-01 | AGTGCAAGGATTAAAGGTAG | 34 | 100.0 |
| N3-ESSalt1-02 | CAAAAGGGGTAATACAGTAC | 35 | 100.0 |
| N3-ESSalt1-03 | CCATAATAAAGGGCTCAGGG | 81 | 95.0 |
| N3-ESSalt1-04 | GAGGAACCAGCGCTCCACCC | 82 | 95.0 |
| N3-ESSalt1-05 | TCACAGTGCAAGGATTAAAGGTAG | 38 | 100.0 |
| N3-ESSalt1-06 | GTAGCAAAAGGGGTAATACAGTAC | 39 | 100.0 |
| N3-ESSalt1-07 | GTACCCATAATAAAGGGCTCAGGG | 83 | 95.8 |
| N3-ESSalt1-08 | AGGGGAGGAACCAGCGCTCCACCC | 84 | 95.8 |
| N3-ESSalt1-09 | AGTCACAGTGCAAGGATTAAAGGTAG | 42 | 100.0 |
| N3-ESSalt1-10 | AGGTAGCAAAAGGGGTAATACAGTAC | 43 | 100.0 |
| N3-ESSalt1-11 | CAGTACCCATAATAAAGGGCTCAGGG | 85 | 96.2 |
| N3-ESSalt1-12 | TCAGGGGAGGAACCAGCGCTCCACCC | 86 | 92.3 |
| N3-ESSalt1-13 | CATAAGTCACAGTGCAAGGATTAAAGGTAG | 46 | 100.0 |
| N3-ESSalt1-14 | TTAAAGGTAGCAAAAGGGGTAATACAGTAC | 47 | 100.0 |
| N3-ESSalt1-15 | AATACAGTACCCATAATAAAGGGCTCAGGG | 87 | 96.7 |
| N3-ESSalt1-16 | GGGCTCAGGGGAGGAACCAGCGCTCCACCC | 88 | 93.3 |
| N5-ESSalt2-01 | AACCAGCGCTCCACCCCATC | 89 | 95.0 |
| N5-ESSalt2-02 | CAAGTTGGAGCAAGATTATC | 19 | 100.0 |
| N5-ESSalt2-03 | CTATACAAAATAGAAATATA | 90 | 95.0 |
| N5-ESSalt2-04 | TAGTTTGTTATTAGTTAGAA | 91 | 95.0 |
| N5-ESSalt2-05 | GAGGAACCAGCGCTCCACCCCATC | 92 | 95.8 |
| N5-ESSalt2-06 | CATCCAAGTTGGAGCAAGATTATC | 23 | 100.0 |
| N5-ESSalt2-07 | TATCCTATACAAAATAGAAATATA | 93 | 95.8 |
| N5-ESSalt2-08 | TATATAGTTTGTTATTAGTTAGAA | 94 | 95.8 |
| N5-ESSalt2-09 | GGGAGGAACCAGCGCTCCACCCCATC | 95 | 96.2 |
| N5-ESSalt2-10 | CCCATCCAAGTTGGAGCAAGATTATC | 27 | 100.0 |
| N5-ESSalt2-11 | ATTATCCTATACAAAATAGAAATATA | 96 | 96.2 |
| N5-ESSalt2-13 | TCAGGGGAGGAACCAGCGCTCCACCCCATC | 97 | 93.3 |
| N5-ESSalt2-14 | CCACCCCATCCAAGTTGGAGCAAGATTATC | 31 | 100.0 |
| N5-ESSalt2-15 | CAAGATTATCCTATACAAAATAGAAATATA | 98 | 96.7 |
| N3-ESSalt2-01 | AGTGCAAGGATTAAAGGTAG | 34 | 100.0 |
| N3-ESSalt2-02 | CAAAAGGGGTAATACAGTAC | 35 | 100.0 |
| N3-ESSalt2-03 | CCATAATAAAGGGCTCAGGG | 99 | 95.0 |
| N3-ESSalt2-04 | GAGGAACCAGCGCTCCACCC | 100 | 95.0 |
| N3-ESSalt2-05 | TCACAGTGCAAGGATTAAAGGTAG | 38 | 100.0 |
| N3-ESSalt2-06 | GTAGCAAAAGGGGTAATACAGTAC | 39 | 100.0 |
| N3-ESSalt2-07 | GTACCCATAATAAAGGGCTCAGGG | 101 | 95.8 |
| N3-ESSalt2-08 | AGGGGAGGAACCAGCGCTCCACCC | 102 | 95.8 |
| N3-ESSalt2-09 | AGTCACAGTGCAAGGATTAAAGGTAG | 42 | 100.0 |
| N3-ESSalt2-10 | AGGTAGCAAAAGGGGTAATACAGTAC | 43 | 100.0 |
| N3-ESSalt2-11 | CAGTACCCATAATAAAGGGCTCAGGG | 103 | 96.2 |
| N3-ESSalt2-13 | CATAAGTCACAGTGCAAGGATTAAAGGTAG | 46 | 100.0 |
| N3-ESSalt2-14 | TTAAAGGTAGCAAAAGGGGTAATACAGTAC | 47 | 100.0 |
| N3-ESSalt2-15 | AATACAGTACCCATAATAAAGGGCTCAGGG | 104 | 96.7 |

TABLE 2C

| Construct Name | Relative Productive Transcript (Mouse, N2a cells) |
|---|---|
| N5-ESSalt2-15 | 1.73 |
| N5-ESSalt2-02 | 1.67 |
| N5-02 | 1.65 |
| N3-13 | 1.63 |
| N3-16 | 1.62 |
| N5-10 | 1.60 |
| N3-07 | 1.58 |
| N5-06 | 1.58 |
| N5-01 | 1.56 |
| N5-04 | 1.56 |
| N5-ESSalt2-14 | 1.56 |
| N3-09 | 1.54 |
| N3-ESSalt2-01 | 1.52 |
| N5-08 | 1.52 |
| N5-ESSalt2-04 | 1.52 |
| N3-10 | 1.50 |
| N5-09 | 1.50 |
| N5-11 | 1.50 |
| N5-15 | 1.50 |
| N5-07 | 1.48 |
| N5-ESSalt1-12 | 1.48 |
| N3-05 | 1.46 |
| N3-ESSalt1-13 | 1.46 |
| N3-ESSalt2-13 | 1.46 |
| N5-03 | 1.46 |
| N5-05 | 1.46 |
| N5-12 | 1.46 |
| N5-14 | 1.46 |
| N3-01 | 1.45 |
| N5-ESSalt1-15 | 1.45 |
| N3-14 | 1.43 |
| N3-ESSalt2-05 | 1.43 |
| N5-13 | 1.43 |
| N5-ESSalt1-13 | 1.43 |
| N3-02 | 1.41 |
| N3-ESSalt1-01 | 1.41 |
| N3-ESSalt1-06 | 1.39 |
| N5-16 | 1.39 |
| N5-ESSalt1-01 | 1.39 |
| N5-ESSalt1-05 | 1.39 |
| N3-04 | 1.37 |
| N3-12 | 1.37 |
| N3-ESSalt1-02 | 1.37 |
| N3-ESSalt1-09 | 1.37 |
| N3-ESSalt2-09 | 1.37 |
| N3-06 | 1.35 |
| N3-ESSalt1-10 | 1.35 |
| N5-ESSalt1-02 | 1.35 |
| N5-ESSalt2-11 | 1.35 |
| N3-08 | 1.33 |
| N3-11 | 1.33 |
| N3-ESSalt2-14 | 1.33 |
| N5-ESSalt1-04 | 1.33 |
| N3-03 | 1.31 |
| N3-ESSalt1-05 | 1.31 |
| N3-ESSalt1-14 | 1.31 |
| N3-ESSalt2-02 | 1.31 |
| N5-ESSalt1-14 | 1.31 |
| N5-ESSalt2-06 | 1.31 |
| N5-ESSalt2-10 | 1.31 |
| N5-ESSalt1-08 | 1.30 |
| N5-ESSalt1-09 | 1.30 |
| N5-ESSalt2-07 | 1.30 |
| N3-ESSalt1-16 | 1.28 |
| N3-ESSalt2-06 | 1.28 |
| N3-ESSalt2-15 | 1.28 |
| N5-ESSalt1-11 | 1.28 |
| N3-15 | 1.26 |
| N3-ESSalt1-11 | 1.24 |
| N5-ESSalt2-03 | 1.24 |
| N3-ESSalt1-15 | 1.22 |
| N3-ESSalt2-04 | 1.22 |
| N5-ESSalt1-10 | 1.22 |
| N5-ESSalt1-07 | 1.20 |
| N5-ESSalt1-16 | 1.20 |
| N5-ESSalt2-13 | 1.20 |
| N3-ESSalt2-03 | 1.18 |
| N3-ESSalt2-10 | 1.16 |
| N5-ESSalt1-06 | 1.16 |
| N3-ESSalt1-08 | 1.15 |
| N3-ESSalt2-11 | 1.15 |
| Scramble-01 | 1.15 |
| N3-ESSalt1-07 | 1.11 |
| N5-ESSalt2-08 | 1.11 |
| N3-ESSalt1-03 | 1.09 |
| N3-ESSalt1-04 | 1.09 |
| N3-ESSalt1-12 | 1.09 |
| N5-ESSalt2-05 | 1.09 |
| N5-ESSalt2-01 | 1.07 |
| N3-ESSalt2-07 | 1.05 |
| N5-ESSalt1-03 | 1.05 |
| Scramble-04 | 1.03 |
| Scramble-02 | 0.98 |
| N3-ESSalt2-08 | 0.90 |
| N5-ESSalt2-09 | 0.90 |
| Scramble-03 | 0.85 |

### Example 2: U7 Constructs with 5′ Targeting Sequence Directly Fused to 3′ Targeting Sequence

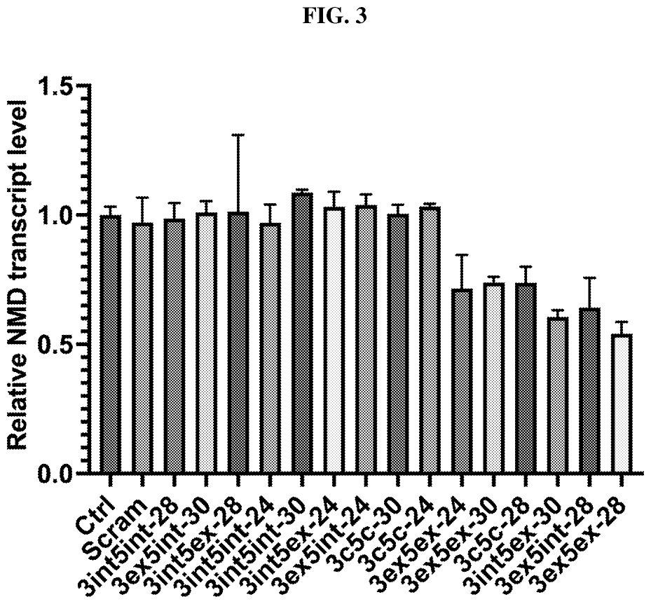

A second U7-based method for suppression of Scn1A NMD transcript was tested. A previous study reported decreased inclusion of an alternative exon using U7 vectors with targeting sequences composed of a 5′-targeting sequence directly fused to a 3′ targeting sequence. 15 different U7 constructs were engineered with different combinations of fused 5′/3′ targeting sequences of varied lengths where targeted sequences were either exonic, intronic, or centered upon the intron/exon junction (Tables 3A-3B). The constructs were tested for their abilities to suppress NMD transcript inclusion in Neuro-2a cells as in Example 1. NMD transcript suppression was observed in 5 out of 15 constructs tested, but none of the sequences suppressed NMD transcript levels by 50% (FIG. 3, Table 3C). Thus although, the different combinations of fused 5′/3′ targeting sequences did suppress NMD transcript levels, they all showed inferior efficacy to the hits identified in the original screen described in Example 1.

TABLE 3A

| Construct | 5′ Antisense Sequence | SEQ ID | 3′ Targeting Antisense | SEQ ID |
|---|---|---|---|---|
| Name | (5′ to 3′) | NO: | (5′ to 3′) | NO: |
| Control | N/A | | N/A | |
| Scramble | GCACCCCATCCCCCACTA | 14 | N/A | |
| | CA | | | |
| 3c5c-24 | GTAATACAGTACCCATAA | 105 | AGCAAGATTATCCTATATAAA | 120 |
| | TAAAGG | | ATA | |
| 3c5c-28 | GGGTAATACAGTACCCAT | 106 | GGAGCAAGATTATCCTATATA | 121 |
| | AATAAAGGGC | | AAATAGA | |
| 3c5c-30 | GGGGTAATACAGTACCCA | 107 | TGGAGCAAGATTATCCTATAT | 122 |
| | TAATAAAGGGCT | | AAAATAGAA | |
| 3ex5ex-24 | AGGGGTAATACAGTACCC | 108 | AGATTATCCTATATAAAATAG | 123 |
| | ATAATA | | AAA | |
| 3ex5ex-28 | AAAAGGGGTAATACAGTA | 109 | AAGATTATCCTATATAAAATA | 124 |
| | CCCATAATAA | | GAAATAT | |
| 3ex5ex-30 | CAAAAGGGGTAATACAGT | 110 | CAAGATTATCCTATATAAAAT | 125 |
| | ACCCATAATAAA | | AGAAATATA | |
| 3ex5int-24 | TACAGTACCCATAATAAA | 111 | TTGGAGCAAGATTATCCTATA | 126 |
| | GGGCTG | | TAA | |
| 3ex5int-28 | ATACAGTACCCATAATAA | 112 | AAGTTGGAGCAAGATTATCCT | 127 |
| | AGGGCTGAGG | | ATATAAA | |
| 3ex5int30 | AATACAGTACCCATAATA | 113 | CAAGTTGGAGCAAGATTATCC | 128 |
| | AAGGGCTGAGGG | | TATATAAAA | |
| 3int5ex-24 | AGGGGTAATACAGTACCC | 114 | TTGGAGCAAGATTATCCTATA | 129 |
| | ATAATA | | TAA | |
| 3int5ex-28 | AAAAGGGGTAATACAGTA | 115 | AAGTTGGAGCAAGATTATCCT | 130 |
| | CCCATAATAA | | ATATAAA | |
| 3int5ex-30 | CAAAAGGGGTAATACAGT | 116 | CAAGTTGGAGCAAGATTATCC | 131 |
| | ACCCATAATAAA | | TATATAAAA | |
| 3int5int-24 | TACAGTACCCATAATAAA | 117 | AGATTATCCTATATAAAATAG | 132 |
| | GGGCTG | | AAA | |
| 3int5int-28 | ATACAGTACCCATAATAA | 118 | AAGATTATCCTATATAAAATA | 133 |
| | AGGGCTGAGG | | GAAATAT | |
| 3int5int-30 | AATACAGTACCCATAATA | 119 | CAAGATTATCCTATATAAAAT | 134 |
| | AAGGGCTGAGGG | | AGAAATATA | |

TABLE 3B

| Construct | | SEQ ID |
|---|---|---|
| Name | Full Antisense Sequence (5′ to 3′) | NO: |
| Control | N/A | |
| Scramble | GCACCCCATCCCCCACTACA | 14 |
| 3c5c-24 | GTAATACAGTACCCATAATAAAGGAGCAAGATTATCCTATATAAAATA | 135 |
| 3c5c-28 | GGGTAATACAGTACCCATAATAAAGGGCGGAGCAAGATTATCCTATATA | 136 |
| | AAATAGA | |
| 3c5c-30 | GGGGTAATACAGTACCCATAATAAAGGGCTTGGAGCAAGATTATCCTAT | 137 |
| | ATAAAATAGAA | |
| 3ex5ex-24 | AGGGGTAATACAGTACCCATAATAAGATTATCCTATATAAAATAGAAA | 138 |
| 3ex5ex-28 | AAAAGGGGTAATACAGTACCCATAATAAAAGATTATCCTATATAAAATA | 139 |
| | GAAATAT | |
| 3ex5ex-30 | CAAAAGGGGTAATACAGTACCCATAATAAACAAGATTATCCTATATAAA | 140 |
| | ATAGAAATATA | |
| 3ex5int-24 | TACAGTACCCATAATAAAGGGCTGTTGGAGCAAGATTATCCTATATAA | 141 |
| 3ex5int-28 | ATACAGTACCCATAATAAAGGGCTGAGGAAGTTGGAGCAAGATTATCCT | 142 |
| | ATATAAA | |
| 3ex5int30 | AATACAGTACCCATAATAAAGGGCTGAGGGCAAGTTGGAGCAAGATTAT | 143 |
| | CCTATATAAAA | |
| 3int5ex-24 | AGGGGTAATACAGTACCCATAATATTGGAGCAAGATTATCCTATATAA | 144 |
| 3int5ex-28 | AAAAGGGGTAATACAGTACCCATAATAAAAGTTGGAGCAAGATTATCCT | 145 |
| | ATATAAA | |
| 3int5ex-30 | CAAAAGGGGTAATACAGTACCCATAATAAACAAGTTGGAGCAAGATTAT | 146 |
| | CCTATATAAAA | |
| 3int5int-24 | TACAGTACCCATAATAAAGGGCTGAGATTATCCTATATAAAATAGAAA | 147 |
| 3int5int-28 | ATACAGTACCCATAATAAAGGGCTGAGGAAGATTATCCTATATAAAATA | 148 |
| | GAAATAT | |
| 3int5int-30 | AATACAGTACCCATAATAAAGGGCTGAGGGCAAGATTATCCTATATAAA | 149 |
| | ATAGAAATATA | |

TABLE 3C

| Construct | Relative NMD |
|---|---|
| Name | Transcript Level |
| Control | 1 |
| Scramble | 0.9708 |
| 3c5c-24 | 1.0047 |
| 3c5c-28 | 0.7382 |
| 3c5c-30 | 1.0385 |
| 3ex5ex-24 | 1.0328 |
| 3ex5ex-28 | 0.5406 |
| 3ex5ex-30 | 0.751 |
| 3ex5int-24 | 1.0319 |
| 3ex5int-28 | 0.6416 |
| 3ex5int30 | 1.0099 |
| 3int5ex-24 | 1.0866 |
| 3int5ex-28 | 0.967 |
| 3int5ex-30 | 0.6057 |
| 3int5int-24 | 1.0128 |
| 3int5int-28 | 0.967 |
| 3int5int-30 | 0.9856 |

### Example 3 Characterization of 5′ and 3′ U7 Construct Pairs

Previous work has also shown improved modulation of splicing using pairs of U7 constructs targeting the 5′ and 3′ end of a target exon. The U7 screen described in example 1 identified a cluster of high-efficacy target sequences at the 5′ end of mouse exon 21N. The top five identified 5′ targeting sequences were paired with top 3′ targeting sequence (Tables 4A-4B). The paired constructs were then compared against single-U7 constructs for each targeting sequence. Evaluation of NMD transcript levels in Neuro-2a cells, as performed in Example 1, showed that the combination of two U7 constructs targeting the 5′ and 3′ ends of the target exon partially inhibited NMD suppression when compared to the 5′- and 3′-targeting sequences alone (FIG. 4, Table 4C). This example shows that the pairing of constructs targeting the 5′ and 3′ end of a target exon is worse at NMD transcript suppression than a single construct.

TABLE 4A

| | 5′ Targeted Sequence | | | |
|---|---|---|---|---|
| Construct | | SEQ ID | Antisense Sequence | SEQ ID |
| Name | ESS (5′ to 3′) | NO: | (5′ to 3′) | NO: |
| N5-ESSalt2-15 | TGGGGGGAGGTAGGTAGGTA | 13 | CAAGATTATCCTATATAAA | 32 |
| | | | ATAGAAATATA | |
| N5-ESSalt2-02 | TGGGGGGAGGTAGGTAGGTA | 13 | CAAGTTGGAGCAAGATTAT | 19 |
| | | | C | |
| N5-02 | ATGATAGGGACTTAGGGTGA | 11 | CAAGTTGGAGCAAGATTAT | 19 |
| | | | C | |
| N5-10 | ATGATAGGGACTTAGGGTGA | 11 | CCCATCCAAGTTGGAGCAA | 27 |
| | | | GATTATC | |
| N5-06 | ATGATAGGGACTTAGGGTGA | 11 | CATCCAAGTTGGAGCAAGA | 23 |
| | | | TTATC | |
| N3-13 + N5- | TGGGGGGAGGTAGGTAGGTA | 13 | CAAGATTATCCTATATAAA | 32 |
| ESSal2-15 | | | ATAGAAATATA | |
| N3-13 + N5- | TGGGGGGAGGTAGGTAGGTA | 13 | CAAGTTGGAGCAAGATTAT | 19 |
| ESSalt2-02 | | | C | |
| N3-13 + N5-02 | ATGATAGGGACTTAGGGTGA | 11 | CAAGTTGGAGCAAGATTAT | 19 |
| | | | C | |
| N3-13 + N5-10 | ATGATAGGGACTTAGGGTGA | 11 | CCCATCCAAGTTGGAGCAA | 27 |
| | | | GATTATC | |
| N3-13 + N5-06 | ATGATAGGGACTTAGGGTGA | 11 | CATCCAAGTTGGAGCAAGA | 23 |
| | | | TTATC | |

TABLE 4B

| | | 3′ Targeted Sequence | | |
|---|---|---|---|---|
| Construct | | SEQ ID | Antisense Sequence | SEQ ID |
| Name | ESS (5′ to 3′) | NO: | (5′ to 3′) | NO: |
| N5-ESSalt2-15 | N/A | N/A | N/A | N/A |
| N5-ESSalt2-02 | N/A | N/A | N/A | N/A |
| N5-02 | N/A | N/A | N/A | N/A |
| N5-10 | N/A | N/A | N/A | N/A |
| N5-06 | N/A | N/A | N/A | N/A |
| N3-13 + N5- | ATGATAGGGACTTAGGGTGA | 11 | CATAAGTCACAGTGCAAGG | 46 |
| ESSal2-15 | | | ATTAAAGGTAG | |
| N3-13 + N5- | ATGATAGGGACTTAGGGTGA | 11 | CATAAGTCACAGTGCAAGG | 46 |
| ESSalt2-02 | | | ATTAAAGGTAG | |
| N3-13 + N5-02 | ATGATAGGGACTTAGGGTGA | 11 | CATAAGTCACAGTGCAAGG | 46 |
| | | | ATTAAAGGTAG | |
| N3-13 + N5-10 | ATGATAGGGACTTAGGGTGA | 11 | CATAAGTCACAGTGCAAGG | 46 |
| | | | ATTAAAGGTAG | |
| N3-13 + N5-06 | ATGATAGGGACTTAGGGTGA | 11 | CATAAGTCACAGTGCAAGG | 46 |
| | | | ATTAAAGGTAG | |

TABLE 4C

| Construct | Relative NMD |
|---|---|
| Name | Transcript Level |
| N5-ESSalt2-15 | 0.4261 |
| N5-ESSalt2-02 | 0.2451 |
| N5-02 | 0.2341 |
| N5-10 | 0.2836 |
| N5-06 | 0.2631 |
| N3-13 + N5-ESSal2-15 | 0.6895 |
| N3-13 + N5-ESSalt2-02 | 0.7586 |
| N3-13 + N5-02 | 0.6593 |
| N3-13 + N5-10 | 0.6043 |
| N3-13 + N5-06 | 0.7126 |

### Example 4: Validation of U7 Candidates Identified in Screen in Post-Mitotic Cells

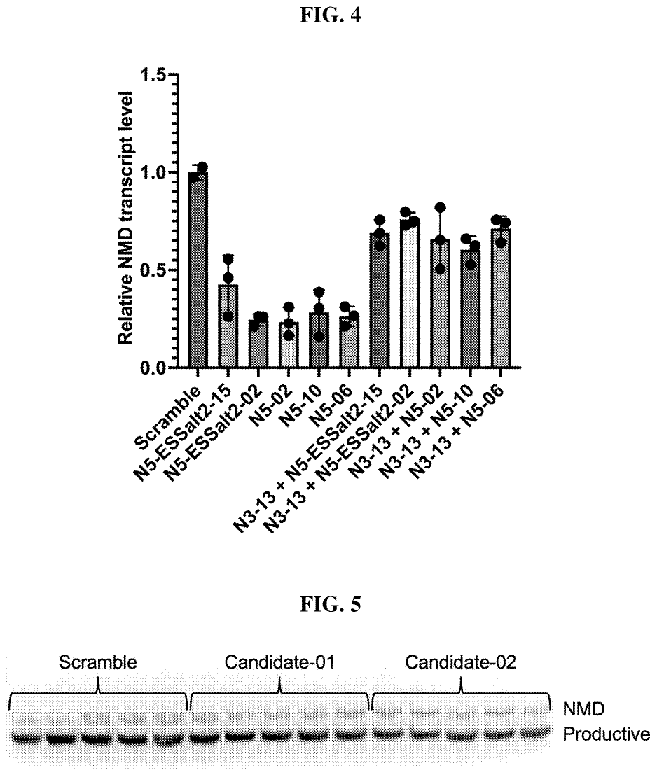

Two candidates identified in Example 1 (Candidate 1: N5-ESSalt2-15 and Candidate 2: N5-05) were further tested for their ability to increase production of the productive transcript. First, AAVs encoding these two constructs were produced and systemic injection of wildtype mice at P28 was performed. Brain tissue was collected at 4-weeks post-injection and levels of productive and NMD transcripts were assessed. Although widespread AAV transduction was observed in the brain, there was no significant reduction of NMD transcript levels or increased productive Sen1a transcript levels (FIG. 5). Endogenous U7 expression is regulated by the cell cycle, suggesting that expression may be weak in post-mitotic cells of the central nervous system. Thus, additional approaches were explored to assess whether increasing U7 expression could overcome this barrier, resulting in efficient splice modulation in the central nervous system.

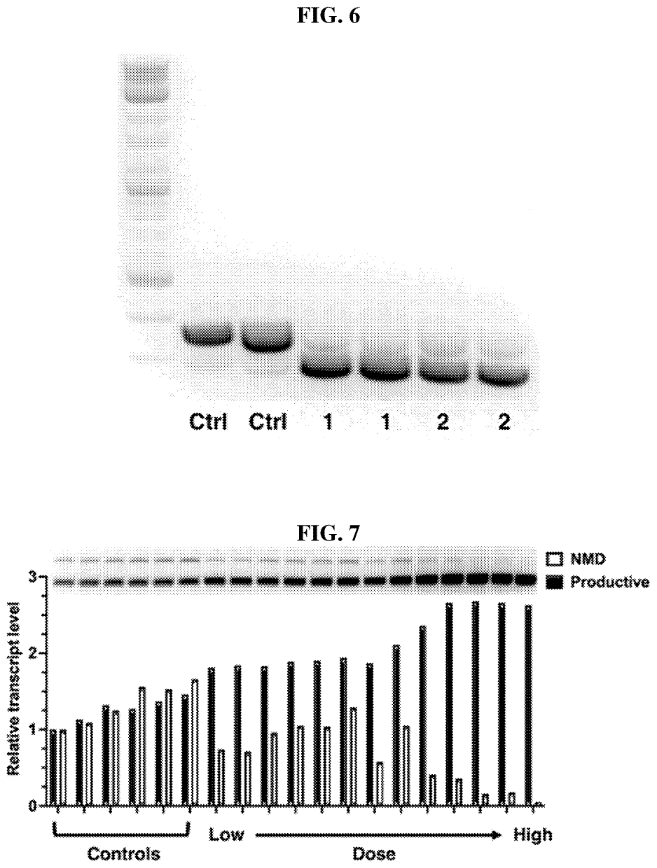

To increase U7 expression, AAV constructs encoding an array of four U7 expression cassettes of either candidates 1 or 2 were generated. Each construct contained 4 U7 cassettes, each consisting of the U7 promoter, ESS, antisense sequence, smOPT, hairpin, and 3′ terminator sequence separated by a spacer consisting of random nucleotides. Primary mouse hippocampal neurons were treated with the AAVs expressing the U7 cassettes of candidates 1 or 2 and then cultured for 1 week. After 1 week of culture, RNA was collected and analyzed for the amount of NMD transcript and the productive transcript via end-point RT-PCR. FIG. 6 shows control cells treated with the scrambled U7 sequence express low amounts of the productive transcript but high levels of NMD transcript, as evidenced by the weak low molecular weight band (productive transcript) and the strong high molecular weight transcript (NMD transcript). All samples treated with either U7 candidate 1 or 2 show near-complete suppression of the NMD transcript and high levels of the productive transcript. This example demonstrates that increasing the expression levels of the candidate U7 constructs through increasing U7 cassette copy number can suppress inclusion of the NMD transcript and increase expression of the productive transcript in post-mitotic neuronal cells.

### Example 5: In Vivo Validation of U7 Candidates

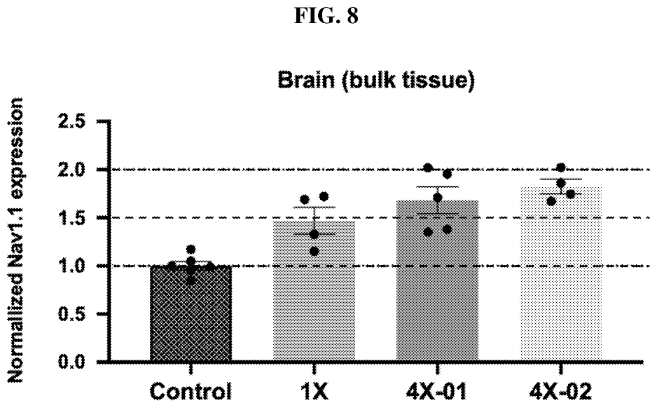

Wild type C57B1/6J mice were injected at P28 with varying doses of AAVs encoding 4 copies of either scramble, candidate 1 or candidate 2 U7 cassettes (described in Example 4). At 3-weeks post injection, RNA and protein were isolated from brain tissue. Analysis of NMD transcript levels showed that the 4× U7 constructs can suppress NMD transcript formation in a dose-dependent manner. High-dose delivery of AAV can almost completely suppress NMD transcript production and nearly triple productive transcript levels (FIG. 7). Western blot analysis of protein confirmed NaV1.1 levels were double those of control samples in animals injected with 4× AAVs (FIG. 8). This example shows that both U7 candidates expressed via AAV are able to suppress NMD transcripts and increase productive transcripts at both the mRNA and protein level in vivo when expressed at sufficient levels.

### Example 6: Additional Vector Modifications

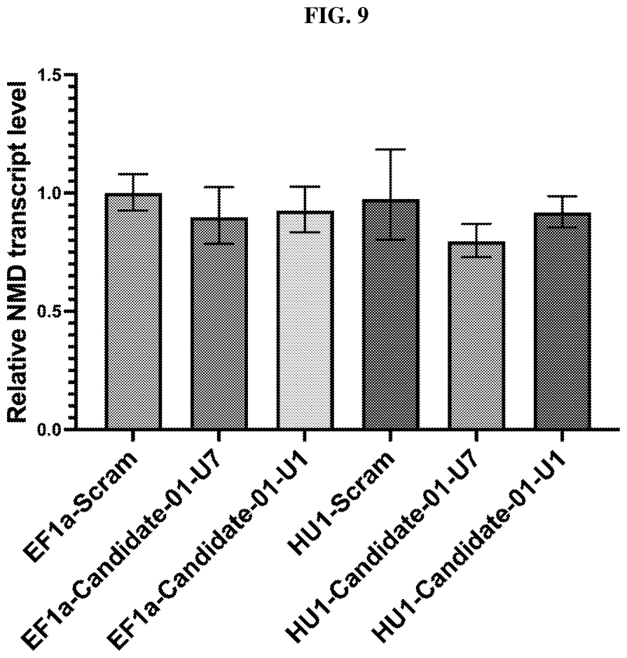

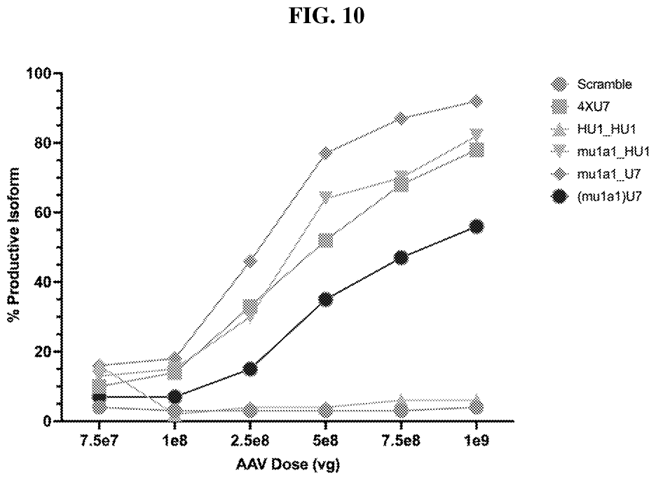

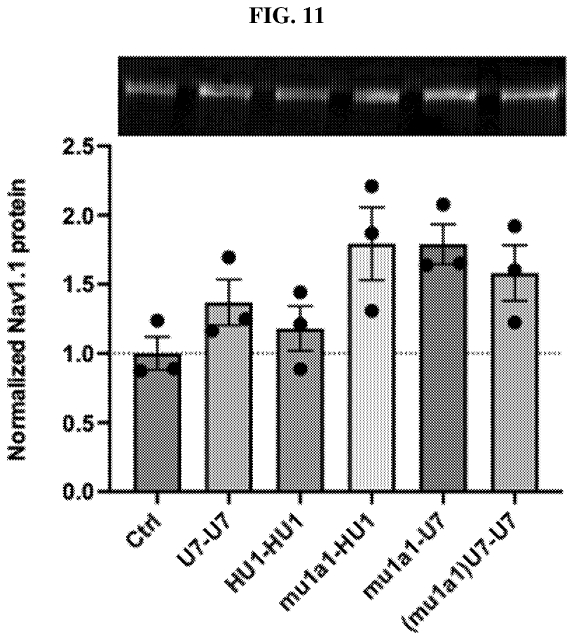

Although administration of high doses of AAVs expressing the U7 candidates resulted in near-complete elimination of NMD transcripts in vivo, delivery of AAV-based gene therapies to the human brain suffer from low efficiency, both in terms of cell numbers and genome copies transduced per cell. Based on this observation, designing gene therapy vectors with high molecular efficacy even at low genome copies per cell is imperative for clinical success. To increase U7 expression from each construct the U7 promoter and the 3′ termination signal sequence were modified. Like U7, expression of the U1 family of small RNAs is regulated by RNA polymerase II. However, U1 RNAs are ubiquitously expressed at high levels compared to U7. Based on this observation, the 5′ and 3′ regulatory elements of U1 were tested for their ability to drive stronger expression of the U7 backbone compared to the endogenous U7 sequences. Additionally, the RNA polymerase II dependent element was switched out with the standard PolII-dependent promoter (ex. EF1alpha) to test the ability of the standard PolII-dependent promoter to increase U7 expression. These two concepts were tested by engineering constructs comprising the human HU1-1 promoter or the EF1alpha core promoter. The HU1-1 and U7 3′ regulatory elements may be important for proper folding and functional U7 expression, so the different promoters were paired with either the standard U7 3′ signal or with the U7 3′ signal immediately followed by the HU1-1 3′ signal (Table 1). Initial testing of the ability of these constructs to suppress Scn1A NMD transcript inclusion in Neuro-2a cells was done with a set of U7 expression plasmids with either the scramble targeting sequence or the Candidate 1 targeting sequence. However, analysis of NMD transcript levels showed that these vectors lacked functional U7 activity (FIG. 9). Functional expression of snRNAs relies upon proper RNA folding after transcription, which is orchestrated by protein factors recruited by the distal and proximal sequence elements (DSE and PSE) encoded by snRNA promoters, as well as regulatory sequences located within the transcriptional terminator sequences. Thus, two potential reasons why this initial set of vectors with modified regulatory elements failed are 1) improper recruitment of accessory factors by the EF1a promoter and 2) incorrect secondary structure formation due to combined effects from the promoter and 3′ signals. To test this, U7 constructs with ESS/antisense candidate 1 were engineered by combining the core U7 structure (ESS-antisense sequence-smOPT-hairpin) with varying combinations of the mouse U7 promoter, mouse U1a1 promoter, human U1-1 promoter, mouse U7 promoter with its distal sequence element replaced with that of the human U1-1 and its proximal sequence element replaced with that of mouse U1a1, and either the mouse U7 3′ terminator or the human U1-1 3′ terminator. The different combinations were then tested for their ability to suppress SCN1A NMD transcript inclusion in cultured primary cortical neurons. The mu1a1 promoter paired with either the HU1 terminator or U7 terminator was as efficient or greater than the 4× standard U7 array, but the U7 promoter with modified PSE and DSE elements worked less efficiently (FIG. 10). Pairing the HU1 promoter and terminator did not result in any functional U7 expression (FIG. 10). AAVs encoding these U7 constructs were then injected into wildtype mice at a low dose and their ability to increase NaV1.1 levels through their suppression of SCN1A NMD transcript formation was assessed. Based on NaV1.1 protein levels, pairing the mu1a1 promoter with the HU1 terminator or U7 terminator once again outperformed the standard 4× U7 array. In contrast to experiments in human neurons, replacing the U7 promoter DSE and PSE with that of mu1a1 was also capable of increasing functional U7 expression (FIG. 11). Once again, the combination of the HU1 promoter and terminator did not result in any functional U7 expression. This example shows that multiple combinations of promoters and 3′ terminal sequences are sufficient to increase functional U7 expression and ultimately the production of NaV1.1 in vivo when compared to wild-type U7. However, not all promoter/terminator combinations are functional and there can be species-specific differences in the efficiency of those that are function, as demonstrated by the (mu1a1) U7 promoter.

### Example 7: Therapeutic Validation of U7 Candidates

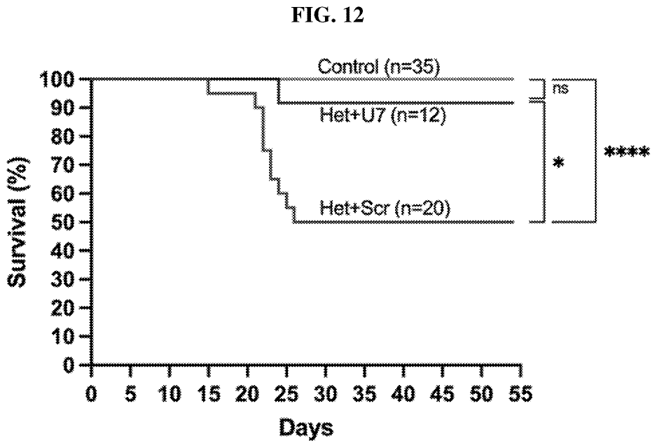

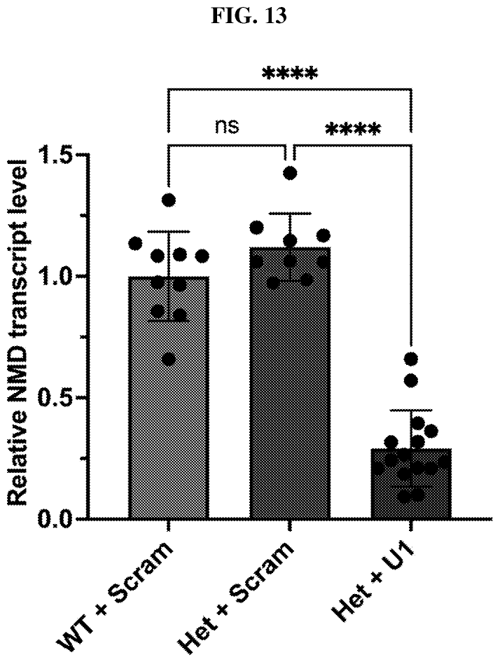

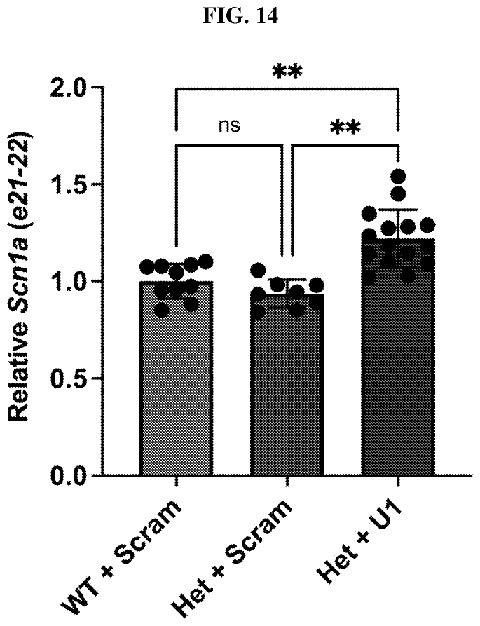

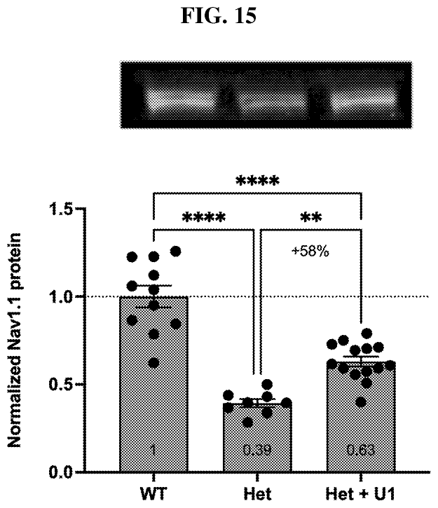

To test the therapeutic potential of a modified U7 construct identified in Example 6 (mu1a1-Candidate 1-HU1), AAV encoding a 3× array of this candidate was produced. The AAV was then systemically injected into Scna1 heterozygous mice via retroorbital injection at postnatal day 14. Beginning at approximately postnatal day 17, untreated Scn1A heterozygous mutants begin to spontaneously die from sudden epileptic death. To test the efficacy of the U7 construct, the survival in a cohort of animals injected with either the modified U7 vector or a scramble control was evaluated, along with Scn1a transcript and NaV1.1 levels. Significant rescue of the spontaneous death phenotype was observed, with 11/12 U7-injected animals surviving until P55 compared to 10/20 scramble-injected animals (FIG. 12). At ~P55, whole brain tissue of surviving animals was harvested and the levels of Scn1A NMD transcript, the total Scn1A mRNA, and the NaV1.1 protein were assessed. A significant reduction of NMD transcript levels (FIG. 13), an increase in total Scn1A transcript levels (FIG. 14), and a 58% increase in NaV1.1 levels compared to scramble-injected heterozygous mutants (FIG. 15) was observed. This example demonstrated a strong molecular and functional rescue of Dravet-associated phenotypes in a disease-relevant Scn1A mutant mouse model.

### Example 8: Therapeutic Validation of U7 Candidates

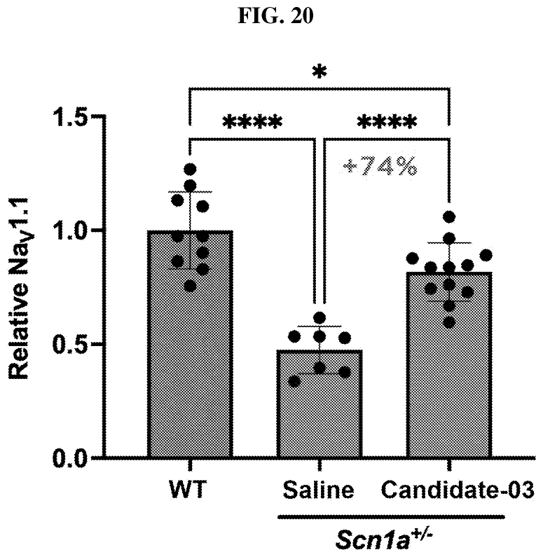

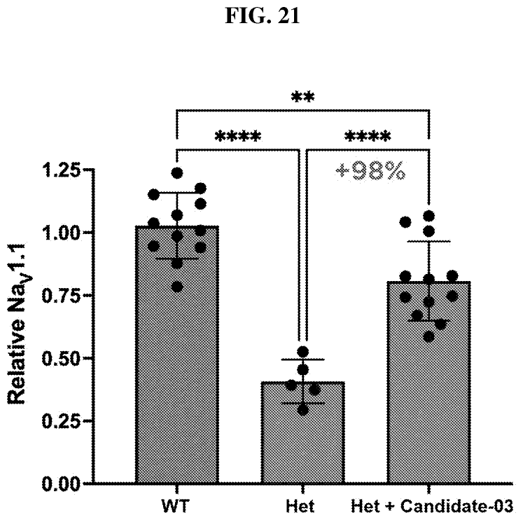

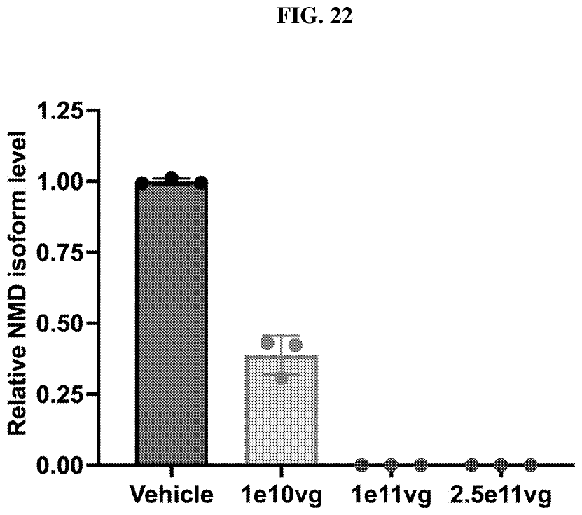

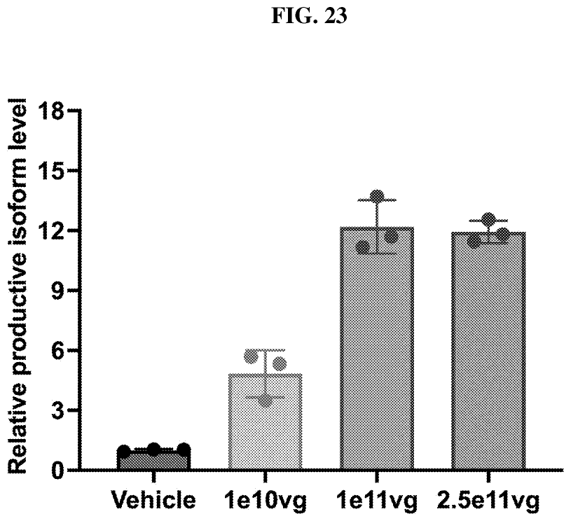

This example continues the experiments of Example 7. The therapeutic potential of an optimized U7 vector, referred to here as Candidate 3 (scAAV-(3X)mu1a1-N5-06-HU1), was tested via EEG-based analysis of seizure suppression. Candidate 3 is a different configuration of Candidate 2, and includes a self-complementary AAV (scAAV) driving a 3× array of mu1a1-Candidate 2-HU1. Senla^{+/-} mice were administered 5e10 vector genomes (vg) of Candidate 3 packaged with AAV9 capsid via ICV injection at postnatal day 2. EEG electrodes were implanted at ~P20 and EEG recording was subsequently performed between P24 and P45. Analysis of saline-injected controls showed significant seizure activity, with 10/15 animals experiencing spontaneous seizures at an average rate of >1 seizure per 24-hour period (FIG. 16). In contrast, only 2/14 animals injected with AAV encoding Candidate 3 exhibited spontaneous seizure activity at an average rate of <0.5 seizures per 24-hour period (FIG. 16). AAV-injected animals exhibiting spontaneous seizures also showed a trend toward shorter seizure durations (FIG. 17). To assess potential differences in efficacy related to delivery method and timing, additional survival studies were performed with AAV9-packaged virus injected via ICV at P2 (5e10vg) and PHP.eB-packaged virus injected at P14 (1e14vg/kg). Injection at both timepoints showed near-complete suppression of SUDEP (FIG. 18-19) and analysis of brain tissue collected from surviving animals at P90 revealed robust rescue of NaV1.1 protein levels (FIG. 20-21). Last, to assess target engagement in human neurons, differentiated ReNcell CX cultures were treated with either AAV formulation buffer or 1e10vg, 1e11vg, or 2.5e11vg of AAV encoding Candidate 3 and cultured for 7 days. Purified RNA was used for end-point RT-PCR to assess suppression of NMD isoform production, as well as production of productive isoform. RT-PCR showed dose-dependent suppression of NMD isoform splicing (FIG. 22) with concomitant up-regulation of productive SCN1A isoform production (FIG. 23), validating the target engagement with Candidate 3 across species. These data support the use of Candidate 3 for therapeutic restoration of NaV1.1 levels in the treatment of SCN1A haploinsufficiency disorders.

### Example 9: Example Regulatory Element Sequences

Table 5 and Table 6 include some examples of recombinant regulatory sequences, including recombinant promoters (Table 5) and recombinant terminator sequences (Table 6) that may be included in an expression construct herein.

TABLE 5
Example promoter sequences

| Sequence | | SEQ ID |
|---|---|---|
| Name | Sequence (5′ to 3′) | NO: |
| hRNU1-1- | TTTCTTTTGTAATCCGAAACATTCTAGTCTGCGAATTAAAAGCCATTATTTGAA | 318 |
| Promoter | GAAGGATGCCCCGGCTCCATCTGGCCACCGAAAGATTGCTCCTTAACACAGG | |
| | CTAAGGACCAGCTTCTTTGGGAGAGAACAGACGCAGGGGGGGGAGGGAAAA | |
| | AGGGAGAGGCAGACGTCACTTCCTCTTGGCGACTCTGGCAGCAGATTGGTCG | |
| | GTTGAGTGGCAGAAAGGCAGACGGGGACTGGGCAAGGCACTGTCGGTGACAT | |
| | CACGGACAGGGCGACTTCTATGTAGATGAGGCAGCGCAGAGGCTGCTGCTTC | |
| | GCCACTTGCTGCTTCGCCACGAAGGGAGTTCCCGTGCCCTGGGAGCGGGTTCA | |
| | GGACCGCTGATCGGAAGTGAGAATCCCAGCTGTGTGTCAGGGCTGGAAAGGG | |
| | CTCGGGAGTGCGCGGGGCAAGTGACCGTGTGTGTAAAGAGTGAGGCGTATGA | |
| | GGCTGTGTCGGGGCAGAGGCACAACGTTTC | |
| hRNU1-2- | GTTTCTTCTGTAATCCGAAACATTCTAGTCTGCGAATTAAAAGCCATTATTTGA | 319 |
| Promoter | AGAAGGATGCCCCGGCTCCATCTGGCCACCGAAAGGTTGCTCCTTAACACAG | |
| | GCTAAGGACCAGCTTTTTTGGGAGAGAACAGACGCAGGGGGGGGAGGGAAA | |
| | AAGGGAGAGGCAGACGTCACTTCCTCTTGGCGGCTCTGGCAGCAGATTGGTC | |
| | AGTTGAGTGGCAGAAAAGCAGACGGGGACTGGGCAAGGCACTGTCGGTGAC | |
| | ATCACGGACAGGGCGACTTCTATGTAGATGAGGCAGCGCAGAGGCTGCTGCT | |
| | TCGCCACTTGCTGCTTCGCCACGAAGGAGTTCCCCTGCCCTGGGAGCGGGTTC | |
| | AGGACCGCGGATCGGAAGAGAGAATCCCAGCTGTGTGTCAGGGCTGGAAAGG | |
| | GCTCGGGAGTGCGCGGGGCAAGTGACCGTGTGTGTAAAGAGTGAGGCGTATG | |
| | AGGCTGTGTCGGGGCAGAGCCCGAAGATCTC | |
| hRNU1-3- | GTTTCTTTTGTAATCCGAAACATTCTAGTCTGCGAATTAAAAGCCATTATTTGA | 320 |
| Promoter | AGAAGGATGCCCCGGCTCCATCTGGCCACCGAAAGGTTGCTCCTTAACACAG | |
| | GCTAAGGACCAGCTTCTTTGGGAGAGAACAGACGCAGGGGGGGGAGGGAAA | |
| | AAGGGAGAGGCAGACGTCACTTCCCCTTGGCGGCTCTGGCAGCAGATTGGTC | |
| | GGTTGAGTGGCAGAAAGGCAGACGGGGACTGGGCAAGGCACTGTCGGTGAC | |
| | ATCACGGACAGGGCGACTTCTATGTAGATGAGGCAGCGCAGAGGCTGCTGCT | |
| | TCGCCACTTGCTGCTTCGCCACGAAGGAGTTCCCGTGCCCTGGGAGCGGGTTC | |
| | AGGACCGCGGATCGGAAGTGAGAATCCCAGCTGTGTGTCAGGGCTGGAAAGG | |
| | GCTCGGGAGTGCGCGGGGCAAGTGACCGTGTGTGTAAAGAGTGAGGCGTATG | |
| | AGGCTGTGTCGGGGCAGAGCCCGAAGATCTC | |
| hRNU2-1- | TCTCCCCGCCCGCGGGCGGCGAAGTAAAGGCCCAGCGCAGCCCGCGCTCCTG | 321 |
| Promoter | CCCTGGAGCCTCGTCTTTCTCCAGGAAAACGTGGACCGCTCTCCGCCGACAGG | |
| | TCTCTTCCACAGACCCCTGTCGCCTTCGCCCCCGGTCTCTTCCGGTTCTGTCTT | |
| | TTCGCTGGCTCGATACGAACAAGGAAGTCGCCCCCAGCGGAGCCCCGGCTCC | |
| | CCCAGGCAGAGGCGGCCCCGGGGGCGGAGTCAACGGCGGAGGCCACGCCCTC | |
| | TGTGAAAGGGCGGGGCATGCAAATTCGAAATGAAAGCCCGGGAACGCCGGA | |
| | AGAAGCACGGGTGTAAGATTTCCCTTTTCAAAGGCAGAGAATAAGAAATCAG | |
| | CCCGAGAGTGTAAGGGCGTCAATAGCGCTGTGGACGAGACAGAGGGAATGG | |
| | GGCAAGGAGCGAGGCTGGGGCTCTCACCGCGACTTGAATGTGGATGAGAGTG | |
| | GGACGGTGACGGCGGGCGCGAAGGCGAGCGC | |
| hU3-Promoter | CCAACAAGAGAGACCCTCATCTCTACAAAATATTTTTTAAAATAACTGGGCCT | 322 |
| | CATGGTGCATTCCTGTAATCTGAGCTAGTTGGGAGGATAGCTAGAGCCCAGG | |
| | AGATCGAGGCTGCAGAGAACCGTGATCACACGACTGCACTCCAGCCTGGGTG | |
| | ACAGAGCAAGACCCTGTCTCAATAGATAGATAGATAGATAGATAGATAGATA | |
| | GATAGATAGATAGATAGATAAAAACAGAGAGGTGAAAAATATGGTTTTAAGG | |
| | GTTCTGTTTAAGAATACTCTGTTCCAACATGGAGTTTGTCAACTCTAAAGTGG | |
| | TAAAAAACAAGACAGATTGTAGACATTTTCATAAAAGGCAGGATTATTTGGT | |
| | GTCACCAAATTAAATATGTTAATGGTCACAGCGGAGGCCGACTCAATCAGCCT | |
| | TTTATTCTGCCAGGCTATTTGATACTGCTGAGGGCTTACCTATTTCCTGAGGAG | |
| | AAAAATAGTCATTAAAATGACCTTATA | |
| hRNU4-1- | GGGCATGAAAGATAAAGTTCTGGAGGTAAGTAGTGATGATGGTTACACAACA | 323 |
| Promoter | ATGTGAACATATTCTATGCCAGTGAATTTACACTTAAGGTTAAAATGGTAAAT | |
| | TTTATGTATACTTTGGCATAATTTAAAAAAAAGTTTGAAAAAGAAAAAAGAC | |
| | AAAACAATAATCGTCTTTGGAGAAGTCTGTTACACTGAATCTGGAGCCAAGTG | |
| | AACAGAACCCTGATTTTGATCCCTTTTCTCTCAAAAGCCCTTCGCAGTCTCTGA | |
| | ATTAAGTCTATTAGCATGTTCCTCCCATAGTGCTTTGCTTCATATCAACAAAAA | |
| | CCTAGCTAAGTGAAATCAGCAACGATATGCAGAAACCACCTACGCAGGTCAC | |
| | AAACATCTTTCTATGATTGTATAATTTTCAAGCAAGCAATAAGTGAAGATTTT | |
| | TCCATAGGCCCTAAACTCACCTTTGCGAAATAGGAAGCTGGTTTATTGGGAGT | |
| | GATGAGCAGGGGGCGTAACAAATT | |
| hRNU5A-1- | GAGACAGGGCTTAAAAGTCCTGGGCAATTAGCTGGGCACAGTGTCGTGCGCC | 324 |
| Promoter | TGTAATCCCACCTAGTTGGGAGGCTGAGGCAGAAGAATCTCTTGAACCCGAG | |
| | GTAGAGGTTGCAGTGAGCCGAGATCGCGCCACTGCACTCCAGCCTGGATGAC | |
| | AGAGTGAGACCCTGTCTCAAAAAAAAAAAAAGTCCTGGGCAGCAAGGCCTCC | |
| | ACTTCACCCCCTAAAGGTTGCCCCAAGAGCACCGTGTGACTGCTAAGGTATTT | |
| | CCGGAGTCTAAAGACGATTATTCAGGTCTCATTTGCATACCCATAATACACTG | |
| | CAAACAGTATTTTTTTCGGAAAAACATTTATATATTGCTTGACATTTTTAAGTA | |
| | TGAGAATTTTGCATGCAGAATTTTTTTGTATAAACTTTCTCAGGTAGTAACCCT | |
| | TGGGATTAGTAGACACCATCAGTGTACTAGGAATTGCAGTTACCCGAAAATTG | |
| | AGTTACAGAAGTAACTGGT | |
| hRNU6-1- | CGACTCGCAACCTTTTCGGGGTCCCGAGTCCAACACCCGTGGGAATCCCATGG | 325 |
| Promoter | GCACCATGGCCCCTCGCTCCAAAAATGCTTTCGCGTCGCGCAGACACTGCTCG | |
| | GTAGTTTCGGGGATCAGCGTTTGAGTAAGAGCCCGCGTCTGAACCCTCCGCGC | |
| | CGCCCCGGCCCCAGTGGAAAGACGCGCAGGCAAAACGCACCACGTGACGGA | |
| | GCGTGACCGCGCGCCGAGCGCGCGCCAAGGTCGGGCAGGAAGAGGGCCTATT | |
| | TCCCATGATTCCTTCATATTTGCATATACGATACAAGGCTGTTAGAGAGATAA | |
| | TTAGAATTAATTTGACTGTAAACACAAAGATATTAGTACAAAATACGTGACGT | |
| | AGAAAGTAATAATTTCTTGGGTAGTTTGCAGTTTTAAAATTATGTTTTAAAAT | |
| | GGACTATCATATGCTTACCGTAACTTGAAAGTATTTCGATTTCTTGGCTTTATA | |
| | TATCTTGTGGAAAGGACGAAACACC | |
| RNU7-1- | CAAAGCCGGTTATGAAGCGGGGGTGGGGTGGGCTAGTTTTAATAGGTCCAGG | 326 |
| Promoter | CGATTAGTACCTGGCATCCTTAACCACCTACAGTTTGAGAAGGGAGTGGGTGA | |
| | TAAAAGCCTGGAAGGGAAGGGAAATCGGGCCGGGCATTAGGCTCCATCGCTC | |
| | ATCAATAGACAAGGCCTTTAGGAAACTGCGACAACGGCTTTTGCTCTGGGCCT | |
| | TTACTGCCGAATCCAGGTCTCCGGGCTTAACAACAACGAAGGGGCTGTGACT | |
| | GGCTGCTTTCTCAACCAATCAGCACCGAACTCATTTGCATGGGCTGAGAACAA | |
| | ATGTTCGCGAACTCTAGAAATGAATGACTTAAGTAAGTTCCTTAGAATATTAT | |
| | TTTTCCTACTGAAAGTTACCACATGCGTCGTTGTTTATACAGTAATAGGAACA | |
| | AGAAAAAAGTCACCTAAGCTCACCCTCATCAATTGTGGAGTTCCTTTATATCC | |
| | CATCTTCTCTCCAAACACATACGCAG | |
| hRNU11- | CACGCCTGCCTGGGCTGCTCCAAGGTGGTGGCTAGGGGTGGCACAAGTTACA | 327 |
| Promoter | CATTTTCAATTTGGATAGGTTGTGATTCTTGTCTTCCCCAGTCAGATACCTACA | |
| | GCCGCTGAGAGCCGGTATTCCAACGCCATGAGACGCCTTCTGTCGTGAGCAGT | |
| | TGACAAGAAAGACCCAGCACTTACTGCAACCGGAAGGAGCGGTTCCTCCCTC | |
| | GCTCGCGCAGCCGCTCTTCCCCGCCACTCCCTCGGTGCCCGCCAGCACATTCC | |
| | CAGCAAGCCCTGAGTATATTTGCATATCAACTCACTACATTTTTTTCTTCTAAC | |
| | TAAAAAATCGAAAGGACAAATTCCAGATTCTCCTTGTGAAGTCTTCCTTTCAG | |
| | TTCAGAAGAAATGGAATTCGCTCTTCAACTTCAGGAAGTTGAAATAAAGAGTT | |
| | GCTTGGATTTTGTGTTCACCTTTACCAAAAAATGGATTTGGTAACACTGCCAC | |
| | CCTGCTTTGGTGACAGAGAAAGC | |
| hRNU12- | TCGAGCTCCACTAGGCCGAGGACCCTAAACCCCGAAGAAAGGAAAGGCCTCT | 328 |
| Promoter | CACCGTCCTTTCGCCGCCGCCCCGCGCTTCCTGATGAGTTCGTCCAGGGAGAT | |
| | GTCCGCCATCTTGCTCCGCCGAGCAAGCCGAAAGCAGTCGAGACCCCGCGAG | |
| | CCCGCCCCTTCGGCCGCTAGGGAGCGACGAACTACTTCCGGCGTCCGCGGACC | |
| | AACTCTCGCGACAGCCAGCTCAAAGCAGGCAAGAACCGGAAGGGGGGGGA | |
| | CGTTCCCCGTGAGCCTTCGCGGTGCTGGCTGCTCATCTGCATACGGAAGTTCG | |
| | GCACATTATGAATTATTTATTTTCCTCGAGGGAAAAAATTAAATGAAAAGCAA | |
| | CAAAATACATTATTAACAAGTGAGACAAACTTCAATGGAACTGGATCATGAC | |
| | CTCAACAGTCAACTACGATAGTCATCATACGCCTAATGAGAATAGAATTCATT | |
| | ACCTAGGAAATAAACTAAAAACGTCCTT | |
| hRN7SK- | GACAAGAGAAGGACTGAACTGGGGGAGTGAGGGAAGAATGCGAGAGTGAGA | 329 |
| Promoter | CTCAGAGAGTAAAGGGGAGGGAAAAAAAAGAAGAGAGAAAGGGCGAGGAA | |
| | GGGGGCGACCGCAGGAGGGAGACAGACCTGGAGTCCACTCGGAGGCCTGGA | |
| | GCCGCACAAAGCGCCAGGTCAGCGGTCCCGGCTGGGTGAGACCAGCAGGCGG | |
| | CTCTAGCGCGCGGGAGCTGGGCGAGGCTCCGGGACGACCTCACCAATGGAGA | |
| | CTGCAGTATTTAGCATGCCCCACCCATCTGCAAGGCATTCTGGATAGTGTCAA | |
| | AACAGCCGGAAATCAAGTCCGTTTATCTCAAACTTTAGCATTTTGGGAATAAA | |
| | TGATATTTGCTATGCTGGTTAAATTAGATTTTAGTTAAATTTCCTGCTGAAGCT | |
| | CTAGTACGATAAGCAACTTGACCTAAGTGTAAAGTTGAGACTTCCTTCAGGTT | |
| | TATATAGCTTGTGCGCCGCTTGGGTACCTCG | |
| mRnu1a1- | CACAAGTAATGTACTTTTAAAATTAACCTTTCTATACCGGTTTTCACAGTGCG | 330 |
| Promoter | GCTGTTTTTGTTGTTCCTCTTAGTGTTAATTCACACTAAAGACTGTGCATCCGA | |
| | CTCCTACATTTATGAAAGTAAATGCCTATTGTTAGAACAAAAAAGGCTACAGA | |
| | ACAAAAAACAAAGCGAAATACCATCTGCTTTAGGTTCAGTGGTATTTTCCCGC | |
| | TGACAGGGAGGCGGGTTTTTGGGTACAGGAAACGAGTCACTATGGAGGCGGT | |
| | ACTATGTAGATGAGAATTCAGGAGCAAACTGGGAAAAGCAACTGCTTCCAAA | |
| | TATTTGTGATTTTTACAGTGTAGTTTTGGAAAAACTCTTAGCCTACCAATTCTT | |
| | CTAAGTGTTTTAAAATGTGGGAGCCAGTACACATGAAGTTATAGAGTGTTTTA | |
| | ATGAGGCTTAAATATTTACCGTAACTATGAAATGCTACGCATATCATGCTGTT | |
| | CAGGCTCCGTGGCCACGCAACTC | |
| mRnu1b1- | AGTCCTTGCTCTTAACTACTGAACCAACTCTCCCGTCCTATAAGTAAATTTTCA | 331 |
| Promoter | GAGCACTAATCTATTCTACTTCAAGTTTCTCCTGCATCAGATCCAGGCCAGCT | |
| | GGAGCGAGCTACACGGTTACCCCCCCTCCTCCCAAAGGGGAAAATAAGGATC | |
| | AGGTGAAAATGTTTGGAAATTACAATAGTAGAATATAAAGAAAGAATATGAA | |
| | GGCGGCCATGAAGGGGTAGAAAGGTAGATGACCAGGCGTCATCCCTGATGTC | |
| | ACGGGTATGGGCGTGGTGTATGCAGATAAGCGCGCAGGGTCTGCCGGGTCCG | |
| | CGCTCCGTGCAGAAATGTGAGGGGTTTCTCCCGCGGCGAGGGAGGGAGCGCG | |
| | GATACGCGGGTGTCAGCAGAGCGCAAGAATGTCGTGTGCGACGAGGGAGACC | |
| | CGAGATCGGCCAGGGCTAAGTGACCGTGTGTTAAGAGTGTAGAGGCGACGGG | |
| | TGTGAGCCGGGGCCGGGCAGTGGGAAAGC | |
| mRnu1b6- | CGGTGGGGCTGGCTCCTGTGCTGCCCGACTCCTGTGCTCCGACTTGGAGAAGA | 332 |
| Promoter | CGGGCGTCCGTTCGTCCATACCCAAAAGAGTCGGCCACAAGGATGTTGGCAC | |
| | CTGGGGGGGGCCACGAGCGGCCAGTTAGGGGTAAGAGACGGTGCAAGGTG | |
| | ACTTGTCCCTGGTCCTTCACCGCTCCAGCACTGCGCCAGTGAGAAAGTCCAAC | |
| | AGGCCCCGGGATCGGGCGGCGAAAGGAAGGGGCGTGATGTCACTCCTGGGTG | |
| | GCTGACGGGGGGGGGTGTATGCAGATGAGGGTCGCGCAGGGGCTGCGGCGTC | |
| | CGCAGCGGCCGGGGAAACGCGAGAGATTTCTCGCGCGGCGTGGGAGAGAAC | |
| | GCGGGTCGCCAGATGTCAGCGGAGCGCCGAGACGGCGTGTGCGACGAGGGA | |
| | GACCCGAGATCGGGCGGGGCTAAGTGACCGTGTGTTAAGAGTGCAGGGGCGG | |
| | CGGGTGTGAGCCGGGGCCGGGCGCTGGGAGAGC | |
| mRnu2-10- | GCTCCTCCTAGGAGCTAACACTGGAGCCAGGGGGATCAAAGCTGTGTAGTGA | 333 |
| Promoter | GTGTCACAAAACATCCTAGCACTAAGGAGACTGAGACGGGAACATTGTAGTA | |
| | AGTTCTAGCCTGGGCTACAGTAAGAGACGTAGTCTCAAACAAGGAAATGCGC | |
| | TATAAATAAATAAGATGGAGCTTCAATGGTCTGTGCATAACTGGGGGGTTGA | |
| | AGGAGCCTTCCAAAGGCTGGGTTCCGGCTCCGTTCAGTCGTTGACTTGAGCTG | |
| | GGCGGGGCATGCAAATAAGGACGCCGCCTGGTCTCCGCGGAACTCTGGGAAC | |
| | GAAAAGCGCAACAAAATCCTCTCGGCCCCCTTGGATTCCTCGGGCTTTCCCTG | |
| | GCGTAAAGGTGGTGGACAGGATAGAAGAGGAGTTTCGGCGTCGGGCGCGTGT | |
| | TTGCGGTCGCGCGAGTGTTGGCGACCGTGAGTTGAAGGTGTGTGTGGTGTTGA | |
| | GTGTATGGGGCGTGTGGGCGCAGTTCGGT | |
| mRnu3b1- | ACAGAATTTACTAAATAGAGAGTGCAGTCATGGAATTGCCATTTTCCCCACGT | 334 |
| Promoter | GGTTTTTCGTTTGTTCCTCACCGGAGGCCTCCGGTTTGAGCCCGAAGTCTAAA | |
| | CCGGTGCGCGTCTGCGAGCTCGCGTGCGCCTCCGCGGCCGGAGCACAGTCGC | |
| | GGGCGGGCATCAGGGCGGTGCGTGGCCGTCAGTGACGCATGACCCTTCGCAG | |
| | GCAGAAGTGGGACGGGTCAAGAGAGGACTGTGATCCGATTGGCTGTTCGGCG | |
| | ACCGTATGCTAATCGCGGTGACAGTCTGCGTGGTGTAGGGACACGGTAGCGA | |
| | CTCAAAATGCCCCGTGGACGGGGTCTGGAGAGGTGTCGGTCTGTCAGAGAAG | |
| | GTGTCCCCGCGAGGGCAGACGCGGTGTGAGATAGAGGCGCGGACAGGGATGT | |
| | CACGGGTCGTCTGTGCGTCAGTCGCCGTGACTAAGATGTGGGGGCGGCCGGT | |
| | GCAGTGCGGCGCGGTGTGGAGCGTGAGGTTT | |
| mRnu5g- | TTATCTACGTTGGTTTTACTACAGAAACTACTGTTCTCAGTAAAAGCAGAGAG | 335 |
| Promoter | AGTACAATGCCAACAGCTTTGAAATCCTCTGTGCTCTTGTGCCAATCCCACCC | |
| | TCAGAACAGGGAGTCTGTCGGTTATGTTTTCGTCCTTTACAGTTCTTCGCCGTC | |
| | TAATCCATTGGAAACCTCCTTTATTTAAAGGAAACCGTGTCCAAATTCAGTGT | |
| | ACAGAGGTGCAAACCTCCGCCCCGCTCCTTGTAGGAAGCCCACGGGAACTGG | |
| | GCCAATCGGACGGCGCTTACTCGGCTCATTTACATACCCATAACACACCGCGG | |
| | CTAATGCAAATATTTTCGTGAAAAGATCTTCCATTACTCGAAGTTTTAAAACT | |
| | AAAAACAAAATATTATTTCAAAAGATCATGAAGAAATTTCTGTCGGTAACAG | |
| | CAGTTTCAATTGATAAATCACCATCACTTATCTAGGAGGTTCTGTTACTCTAG | |
| | AAGTGAATTAAGCAGGACAGCTGT | |
| mRnu6- | GGCATGTGATTTTGTTTTGTTTAGGTTTTTTTTTAACTTTATTTGATCAACATGA | 336 |
| Promoter | AATAATACAATAAGCTATGTCTTCTAACCAGCAGGCAGCCAGCCAGGAGCCA | |
| | TGTGTAGCAGTGTTAGAAGCTGCAGCAGGCATGTGGGTAATTGTGGGGAGCT | |
| | TTTAGGAACAGCTAGGGAGCTGTGTGGAGCTGCAGAGATGCAGAGGAGCCTG | |
| | GAGGAACAGACCAGTGCTGTGGGCTGGGAGGAGCCCCCCTCACAGGTACTTG | |
| | CTCAACGGCTCCACGACCATAAGCCTTGAATAAGTTACATGGACATTTTACAA | |
| | ATGGCACAGGATTGATTATGTCTGCTGCCAGAAGCCTCATGTCGATAAGGAG | |
| | AGAGCTGGCCAAAAGGTAAAGTATGCTAGCTCCATACAGTGCAATAGCCACG | |
| | TAGTCTCACAGAGAAAGAGGCAGGCCTGCATCATATCCAAATATGGCCAAGA | |
| | ATTTTATTAAATTAAAATGCAAACAGCA | |
| mRnu7- | TATTTTTAATGAAGCAGGGCCATGGAGCCCAAGCTGGCCTTGAACTTCTCAGC | 337 |
| Promoter | TTTTGTGCTGGTATCACAGAAATGTGGCGCTATGCCCGGTCGGGCCTGATGTT | |
| | CCAGAGTCTTTGCAGTGTGGGGAACTTGTAAGGATGAGACTACAAGACATCG | |
| | GGCCACATCGCCTGCCACTACTTAAGTCCGATTCACTTCGGCTTTAGCTCCAA | |
| | GCCTTTAATCTCGCGAAGCTCTTTTTTTTTTTTTAACAACATAGGAGCTGTGAT | |
| | TGGCTGTTTTCAGCCAATCAGCACTGACTCATTTGCATAGCCTTTACAAGCGG | |
| | TCACAAACTCAAGAAACGAGCGGTTTTAATAGTCTTTTAGAATATTGTTTATC | |
| | GAACCGAATAAGGAACTGTGCTTTGTGATTCACATATCAGTGGAGGGGTGTG | |
| | GAAATGGCACCTTGATCTCACCCTCATCGAAAGTGGAGTTGATGTCCTTCCCT | |
| | GGCTCGCTACAGACGCACTTCCGC | |
| mRnu11- | GTTCTTTGCTTTTACAGACCGTCTCCTGTGTGTCCCTGTCAGAGACCTGAATTC | 338 |
| Promoter | GGTTCCTAGCACCCATATGTTAGAACATAACGTCCAAGTTGGAGGCTACAAGT | |
| | GGCACTAAATTACACATTATCAGTTTTGACAGGTTGGGGCACCGTGTAACCCT | |
| | AGTAACTTAGCGAAAGGCTCCTCGAACCCAACATCCCACCTCTAGCTGGGAG | |
| | CAGCTGTTTTGCACCAGCGCCGCCTACCTCTCCCTCCGCGTCGGCCAGAGCAT | |
| | TCCCAGCAAGCACTGCTCTGATTTGCATATTGACTCACGGACTTTCCTTTACTG | |
| | CTATACAAAAAACGACACAACAGTTCTCTTTGTGAAGAGCTTTCCCTCAGCCC | |
| | GAGAAGAATCGATAGTTTGCCTTTTAGCTTCGACAATATACAGTTGAAAGTTG | |
| | GTTAGACTTTATTTTCACCTTTACCGTAAGATGGGTGGACCGACAATGCCACT | |
| | GTGCTTCCGAACGCGCTCGAGC | |
| mRnu12- | GAAGCAGCGAGGCCCCTACGAGCCTCGAGGAAAAGGGAAAGGGCCGCTCAC | 339 |
| Promoter | CGTCCCTTGGCCGCCGTCCCGCGCTTCCGGATGAGCTCGTCCAGCGAGAGATC | |
| | GGCCATCTTGCGGCGCGCAGCTAGCGGAGAACGGAGGAGAAGCCGACTGCGG | |
| | CGAGCCCGCCCCGCGGCCGGAGTCAAGAGCGGCTTCCGGCAACCCGGACGGC | |
| | CAACTCTCGCGACAGCTCAAACGAAAAGCTCGAGACGGAAGGGGTGGAGCCT | |
| | CTCTCCCCGTGAGCCTTTGCGGTGCTCGCTTCTAATTTGCATGGAGGAGTCCG | |
| | GAGCCTTATGGGCTCTCTCTCCCCCCTCCTCCTTTTGCTAATGAGGAATTAGAA | |
| | TTATATCGATCATAAACCATTAAAAAGCATTATCAAGAGGACAAGTCTTTTAG | |
| | TCATACAGCCGATCGGACACTCACCATACTTGTAATGAGAATATAATTTCTTA | |
| | CCCCTGAGGAACGTAGACACGTTCCTC | |
| mRn7sk- | CGCTTCGGTGTGTCACTTCGGCCACAGAGGGAGACAGACGATTACGGCCCAC | 340 |
| Promoter | GGAAGGCCGCGTGAGTCTCGTGAAGCTGGAGATGCGCTGGGTCTACCTTTCGC | |
| | CGTCCGCTCCGCCTTGGCAGAGGCGCCGACACGCACCGAAGGCAGAGAACCC | |
| | ATCTCGTATGGGACCGCACCGGACACGGGCGCCTGGGCCAGGAGCAGAGCCG | |
| | GGCCGTAGAATAGACATGGCCGTCGGGGGCGGGGCTTCGGAAGGTTTAACCA | |
| | ATCCAAACTGTTGTATTTTGCATAGCCCCAAAGCATTTTGGTTAACAGTAAAA | |
| | ACATCCTAAATTTAAGTATTTTAATTTAAACTTAGAACGAAGCGAGTATAAAA | |
| | AGGATTATTTAACCCTAAAACGGATTCAGGATTTGTTATAATATCAAGTACAG | |
| | TCGGCTACATAAGGTCACCACATGTGTAAAGTTACAAAATTCTATGGCCTTAT | |
| | ATACCTACCAAGAGCCTGCTTACTCTC | |

TABLE 6
Example terminator sequences

| Sequence | | SEQ ID |
|---|---|---|
| Name | Sequence (5′ to 3′) | NO: |
| hRNU1-1- | ACTTTCTGGAGTTTCAAAAACAGACTGTACGCCAAGGGTCATATCTTTTTTTGT | 341 |
| Terminator | ATTGGTTTGTGTCTTGGTTGGTGTCTTAGGTGTTAATCCTACAGTGGAGGGTTT | |
| | ATGGGAATAGGAAGTAACATGTCGCCTGCATGTCATAGGAGAAAAAGCGAGC | |
| | ATCAGCCGTATCGGCTTTGTAACACAAATTAGCTATCGTGAAGTCCGCTCAGC | |
| | TCTTCCCTTTCTACCCTGGCTGCTTTTTGCAGGGATTGGTCCGTGGTCTCCAGT | |
| | CTCTTGGGTTCTCACCCTGTGTGAAAATCTTCGTGTTTTTCCCTACCCCCCAGT | |
| | CACCTCTTACACAGCCTCTGCTTCCAAGCGCAGCCCCCACAGGAGTTTGTAGG | |
| | ATTTCTGTGCTAGCGGGGAATGTGTTCTCACCTCATAGAGCCAGGTAGAAATT | |
| | ATGCAGATGGGCAGTTCTCTGGGAAGAAAGCAGGGCCTTTGGGGCTCTCAGT | |
| | GTCCCCGTTGGGTTGTAGACA | |
| hRNU1-2- | ACTTTCTGGAGTTTCTAAAAGTAGACTGTACGCTAAGGGTCATATCTTTTTTTG | 342 |
| Terminator | TTTTGGTTTGTGTCTTGGTTGGCGTCTTAAATGTTAATCCTACAGTGGAGGGCT | |
| | GGCGAATAGGAAGTAACATGTCGCCTGCACGCCATAGGAGAAAAAGCGAGC | |
| | ATCAGCCGTATCGGCTTTGTAACACAAATTAGCTATCGTGAAGTCCGCTCAGC | |
| | TCTTCCCTTTCTACCCTGGCTGCTTTTTGCAGGGATTGGTCCGTGGTCTCCAGT | |
| | CTCTTGGGTTCTCACCCTGTGTGAAAATCTTCCTGTTTTTCCCTACCCCCCAAG | |
| | TCACCTCTTACACAGCCTCTGCTTCCAAGCGCAGCCCCCACAGGAGTTTGTAG | |
| | GATTTCTGTGCTAGCGGGGAGTGTGTTCTCACCTCATAGAGCCAGGTAGAAAC | |
| | TACGCAGATGGGTGCTGTTCTCCAGGAAGAAAGCAGGGCCTTTGGGGCTCTC | |
| | AGTGTCCCCGTTGGGTTGTAGA | |
| hRNU1-3- | ACTTTCTGGAGTTTCAAAAACAGACCGTACGCCAAGGGTCATGTCTTTTTTCG | 343 |
| Terminator | TATTGGTTTGTGTCTTAGTTGTTAATCCTACAGTGGAGGCCTGGGGAATAAGA | |
| | AGTAACATGTGGCCTGCACGCCATAGGAGAAAAAGCGAGCATCAGCCGTATC | |
| | GGCTTTGTAACACAAATTAGCTATCGTGAAGTCCGCTCAGCTCTTCCATTTCTA | |
| | CCCTGGCTGCTTTTTGCAGGGATTGGTCCGTGGTCTCCAGTCTCTTGGGTTCTC | |
| | ACCCTGTGTGAAAATCTTCGTGTTTTTCCCTACCCCCCAAGTCACCTCTTACAC | |
| | AGCCTCTGCTTCCAAGCGCAGCCCCCACAGGAGTTTGTAGGATTTCTGTGCTA | |
| | GCGGGGAGTGTGTTCTCACCTCATAGAGCCAGGTAGAAATTATGCAGATGGG | |
| | CGCTGTTCTCTGGGAAGAAAGCAGGGCCTTTGGGGCTCTCAGTGTCCCCGTTG | |
| | GGTTGTAGACATAACACTCTTA | |
| hRNU2-1- | CCTCCGGGGATACAACGTGTTTCCTAAAAGTAGAGGGAGGTAAGAGACGGTA | 344 |
| Terminator | GCACCTGCGGGGCGGCTTGCACGCCGAGTGCCTGTGACGCGCCGGCTTAACTT | |
| | AACTGCTTCCCTGAAGTACCTTGAGGTTCCTGATGTGCGGGCGGTAGACGGTA | |
| | GGCTTATGCGGCACGCTGTCGTTTCCACCGTGGCTACTGCGCTTTGGGAAGGC | |
| | CACGACCTCCTCCTTTGGGGAGGTCCTTAGGATCTCAGCTTGGCAGTCGAGTG | |
| | GGTGGCGACCTTTTAAAGGAATGGGACCCACCCGGAGTTC | |
| hU3- | TTCCCTTCTTGTAATACAAAATAATAACAACCACAGAGGACATCGCAAGTAA | 345 |
| Terminator | AATGTGTATAACAACCCCATCTAAAGAACCCTATCATCTTTCTACTATCTGTCT | |
| | ACCTAAATATGTATAGTATAAATAACCCTATATAGCACTCTAAGCAACTTAAG | |
| | TTAGGTAACTTAGAAAACTCTTTTTTTTTTTTGAGACTGAGTCTCACTCTGTCG | |
| | CACCCAGGCTGGAGTGCAGTGGCGTGATCTCGGCTCACTGCAACCTCCACCTT | |
| | CTGGGTTCAAGTGATTCTCCCATCTCAGTCTCCCAAGTAGCTGGGATTACAGG | |
| | CACGCCACCATGCCCTGCTACATTTTTTTTTTTTTAGTAGAGATGAGGTTTCAC | |
| | CACATTGGCCAGGCTGTTCTCGAACTCCTGACCTCAGGTGATCCGCCCACCTT | |
| | GGCCTCATGAAGTGCTGGGATTACAGGTGTGAGCCACTGTACCCAGCCAGAA | |
| | AACTCTTAAAAAAC | |
| hRNU4-1- | CTGAATTTTCTTGCAGTTGAACAACAGAGGCTTTTTTTGTGTGTGTGGGGGTG | 346 |
| Terminator | CTTGGTTTTGGGAGGTTGAAGAGTACTTGTTCGCAAACTCTCTAAATGAGAGA | |
| | TGGGGAAGTGTCTTACCAGCTGATTCGCGAGAGCACCTGCAAGTTTACCTGTT | |
| | TTAGTGATTTGCGTTTATGAAAGTTGGAGTTGCTGATTTTGTGATCTCTACTTT | |
| | TAAGGTAAAAGCAGGGCGGTGTTGACTTTTCTTGCCAGTTGGCTGTCCTGTTT | |
| | TCTTTCTCGCACTGTTTTCTTTTTGAGACGAAGTCTGTCGCTCAGGCTGGAGTG | |
| | CACTGGCGTGATCTCGGCTCGCTGCAAACTTTGCCGAACCCCTGTTTAGCGAT | |
| | TTCCGTTTG | |
| hRNU5A-1- | ATATGTGGTAATCCAACAATAGAAATTATTTTTAAGTTTGTGTGTTCCTTTTTC | 347 |
| Terminator | TGTTCAATGGTGCTTTTGATATTGTTGTAAAGCAGTGACTAGCAGATTCTGTGT | |
| | GGTAAAAGCACTGAACCCTGTTTAGTTCTTTTCTCCCTACCTTATTGGTAATTA | |
| | TTTCTGTGTATATATGTTCCAAAGAAATGCGGCTTGTATATTCATGCTTTTCAA | |
| | CTTTTTGTGATAGACAGTTGATACACAAGTAGTACCTCCTCCGGGATCTTCTTA | |
| | GTCAAGACTGTTTGGTAGATGCATGTTTTTTGTTACTTGTAATGTACAGTATTC | |
| | AGTTCATTTTCTTTTTGCAGCACTGTCCGGTTGAAACACAACGCGAGGATCGG | |
| | GCGTGGTGGCTCACGCCTGTAATCCCAGCACTTTGGGAGGCCGAGGCAGGAC | |
| | AATCAAGACCAATCCTGGTCATCAAGGCAGGAGAATCAAGACCATCGAGGTC | |
| | AGGAGATCAAGACCATCCT | |
| hRNU6-1- | ACATCAGGTTGTTTTTCTGTTTTTACATCAGGTTGTTTTTCTGTTTGGTTTTTTT | 348 |
| Terminator | TTTACACCACGTTTATACGCCGGTGCACGGTTTACCACTGAAAACACCTTTCA | |
| | TCTACAGGTGATATCTTTTAACACAAATAAAATGTAGTAGTCCTAGGAGACGG | |
| | AATAGAAGGAGGTGGGGCCTAGGCAGATTCATCTCTGCGGTGCATTTTGCCTC | |
| | TGGCCCTCGGGCCCTAGTTTGCGATCTTTCCCATCTTGCCTCTGCTTCCCTCTG | |
| | CACAATCCTGAGTATTGCAGTACCTTACACTTAAGGAAATTTCGTCTTGCCAA | |
| | AGTTGATCCATTTCAGCTCCGAAACATAACAGGAACTAGTGCAACGATAAAC | |
| | GTATGAGGAACTTGCAATAAGAGTGACAGAATCCTTTGAATCAAAAAAAAAA | |
| | AAAGCCACCACTTAATGAGTATCTACCATATGTCAAGGGCTTCACCAATATTT | |
| | CATTGAAATATTCCAGACCA | |
| RNU7-1- | CTTATGATGTTTGTTGCCAATGATAGATTGTTTTCACTGTGCAAAAATTATGGG | 349 |
| Terminator | TAGTTTTGGTGGTCTTGATGCAGTTGTAAGCTTGGGGTATGAAGGTTTGGGCC | |
| | ACGCCTGGGCGCTTCCGGCTGCGCCGGATGCTGTTTCCTTTCCGCTCCCAGGG | |
| | GCGTTGGGAACGGTTGTAGGACGTGGCTCTTTATTCGTGAGTTTTCCATTTACC | |
| | TCCGCTGAACCTAGAGCTTCAGACGCCCTATGGCGTCCGCCTCGACCCAACCG | |
| | GCGGCCTTGAGCGCTGAGCAAGCAAAGGGTGAGAATCGTCCTAGTCAAGGCA | |
| | TAGGCTGCTGGCCTGGGGTAGTCAAGGCATGGGCTGCTGGTCTGGGGAGGAT | |
| | GCGGGCGGAGGATGTGGGGCGACAAACCTGGCTACGTCCGCCGGGAAAATGG | |
| | GGTAGGGGACGCCGGGTACCGTCCTTCTAAGTGGGGCGCTTGCCCCCAAGAC | |
| | TTGGGATCTTATTGGGTTACCTACA | |
| hRNU11- | TCTTTACTGTTATATGTTAGGCGAAATATTACGCGTTTGGAGTAAGTGGTGCTT | 350 |
| Terminator | TTTGTAACTGAAAAGAGATTCTGTGTGTGTTTTTTTTTTTTTAGAGGCTGCATA | |
| | GTTATTTGTATGAACCGCAGGTGACCTAATTAGCAAGGTAATTCAGACCTGTA | |
| | TCCTAGCACTTAAGAAGTGGAAGTGGGATAACCCTGCGTGTTCTCGTGTGGGA | |
| | GGCTAGCTTAAGCAACAAAGCAGGAAGGCCCCACCAACAAAAGAATTTAAAA | |
| | ATTATACGTGCGTGGTGGCGCGCGTATGAGGAAATTATTTGGCAACTGTGTTA | |
| | CAGTATAGCAGCAAATGGAATAGACCTAGTCCCTGACTTTAGGAGACCTGCCT | |
| | CAGCCCCCCGAATGGCTGGGACTACAGGCGTGCGCCTCCGCGCCCAGCTAATT | |
| | TTTGCATTTTTGGTAGAGACGGGTTCACCATGTTGGCCAGGATGGTCTCCATC | |
| | TCTTCATCTGGTGATCCGCC | |
| hRNU12- | ACCTTATTCACGCCTAAAAAGTAGACTGACTGTGGGGTGGTCGTGTTTTTTGT | 351 |
| Terminator | TTCTTGTTGGTAGGTGGTGAATGCGTTTTTTTCGTTGTTTTCTCCGTTACTCAG | |
| | GCTGCCAGTTGCTTGGCAGTCTTGTCGCTGGCTGTGGACGCTCTGCACTTCATC | |
| | ACCGTCCCCAGCTGCGCTTTGAGCCGGTCTAAGGTTGCCCACAGTATTGACGT | |
| | TGGGACCCACGAAACTGGCAATTTCCATGTGTGCGGCCTTACCCAGTTCTCCC | |
| | AAACTTCTGCGTATAACCGCTTCTCTGTGCTTCAGAAAGATAAGTCCGGCCCC | |
| | GGCCTCACACAGCAGCTCAGTTCGTGACAGCGTTTAATATGTAAAATGGAGC | |
| | AAATAATTTATAGGGCGTTGGGAGAGTTAAAGAGACTAATTCATTTACGTGTT | |
| | AAATACAGGGAATGGCTTTTGCATTGATATAACCCCAGCCTCCAGCATGCGCC | |
| | AGACCTGACGTAGGTGTTCGGC | |
| hRN7SK- | TTTGACCCATTACCCATCTAAGTTAGATGCTTTTTTAAATGTTTTTTAATTTTTA | 352 |
| Terminator | AATTTTTAATTTTTTTCATTATTTATTTTTTATTTTTGAGACGGATCTCGGCTCA | |
| | CTGTAACCTCCACCTCCCGGGTTCAAGCGATTCTCCTGACTCAGCCTCCCGAG | |
| | TAGCTGGGATTACAGGCGCGCGCCACCATGCCCGGCTAATTTTTCTATTTTTA | |
| | GTAAAGACGGGGTTTCGCCATGTTGACCAGGGTGGTCTTGAACTCCTGACGTC | |
| | AGGTGATCCTCCTGCCTCGTGCTGGGATTGCAGGCGTGAGCCACCGCTCCCGG | |
| | CCCTTAGATGCTTTTTAATCAGCAAGTACGCAATGCTTTCCTGACAGGAATGT | |
| | CAAGTCCGTCAGGAAATCCACATAAGGGTGTTAAGCAGGCAAAGTACCACAT | |
| | ACAGATTAAAATCAAAAGCACTGTCAACGCTTATTTAATTTATACAGTAGACC | |
| | CTAACTTTTCTAATTTTGTG | |
| mRnu1a1- | GTTTACTTGGTTTTAAAAATAGCTTGCACTAGCGATACCGCGAATATGGTTAT | 353 |
| Terminator | TAGGTTTGTTAGGCACAGTCGTGTCTTACTATAGAAAAATAACGTAGTGTTCA | |
| | TTTTGCTGCCTGTATGTGTTAATTTGTCCTTATTGCGCCATTGTTCTTGTTAAGT | |
| | CTTCTGTAAGGAGTTGCGGGTTTCAAACTGTCAGTCTGAGAGCATAAGCTCAC | |
| | TTTTCAGTCAGTTGAATTGAATGTTACATTCGATTGGTTCTGGATATTGATATT | |
| | ACCCAAGGCTTGTCCTGTTTGTAAGTCTTTTCCTACACCTGACAATATGATTAT | |
| | GTTTACAATGTCCCTAGAACTCTGGGTGTATCGTTCCCACTGAATAAAATTTG | |
| | ATCTCTAAGTTGACCTATAAATTTAAGTACCAAAAAACTGGTTTGTAAGATTT | |
| | TGGAAGGCAGAGGCAGGGAAGAGACCAACGTGGTCTACATATAAAGTACCA | |
| | GGTACATATAAAGTACCAGGT | |
| mRnu1b1- | ATTTTTGTGGTGCTAAAAGTTAGATTGCATTCCGCCCTTCTCACGTCTCTTTAC | 354 |
| Terminator | GTGTTGTTTGCGAGGCGTGGCCTCGGAGGTAAATGGTCTCCTTTTACCTCAGA | |
| | TAAGCTTACCTTTGCTCTGTGAGTAAGAATGTAGCTACACTTCCAAATTTAGC | |
| | TATTGTCCTTTGCGGTCCAAATGAAAATATTACCTTCCGATCTAACAATGTTA | |
| | AACTAGTCCCAGGCCCTTCTATCGATTGCCAGAATAGACTAGACTACTACACA | |
| | GACACACCCTCTCAAATCAGGCCAGCTCCTGCAACCTGCACCTACAGTTTGTC | |
| | CCCTGGGACAAATGAAAGGACAGGCCCTCTTTCCCCCCCCCCCCCAAATCAGA | |
| | GCACACACAGATCTTGGGGTCTGGGGCAGAAATTTCGTTCATGGAAAAAACA | |
| | AACAAACAAACAAACACGATCAAAGGAAGCTCGTTCCCTTTGGCCTCAGGGT | |
| | TTCTTAGACGCCGTGTTCCC | |
| mRnu1b6- | ATAACTGGTAACCAAAAGCAGATTACTTTCCCCCTTGTGGTCTTCGCGTTTGC | 355 |
| Terminator | GGACGGGAGAGAACTTTCGGCCATTTTCCTTTTAGAAATTTAACTTCTTGAAG | |
| | GCCACCAGGTGACCCTTACTTCCCTCTCACCTTTCCTTCTACCTGTACTTTCTCT | |
| | ATTAAAAAGCCTGCAGAAGTACCCTTGACAAATTGCCAAAAATGACACCTAA | |
| | AAAAAAAAAAAGGAAAAACGACACCTTAGGTAATTATAGGTTGTAAGAAAA | |
| | AGATTTGACCTCTTGGAGTTTTTATTTCTTAATACCAAAGGACACCCAAGGAA | |
| | CGTATCTCTCAAAATTGGGTTTCAAACCTCAGCCGTTTCCTTCTTAGTTCCAGT | |
| | GCTTAACACGCAACAGACCTGCACACCACTACACTGCATACGAGTTTTAAAGT | |
| | TAATAAATCAGTCACCCCTCCATAGTCTTCTACCTACTTGAAGACAGAAACGG | |
| | CTGCTAGGAAGCAATTCTTC | |
| mRnu2-10- | CTGGGGAATAAGAGTGGTTTAAAGAAAGAACAATTTGAGAGATCTGCTTAGA | 356 |
| Terminator | TCTAATAAAACCCAAGTATAGTTTCTTTAGACGTCTTATATCTCACGTAGGAC | |
| | CTCTTATTTCAGACTCACTGCAAGAACAAGCTAAAGAACTATATACAGTCAGG | |
| | AACAAGGTGAGCCATCGCGGTTCTCAGTCCCATGATCTTAAGAGTTTTTCCTC | |
| | TTCCCCTCTTCCCTGGCTGTCCTGGAGCTCACTCTGTAGACCAGGCTGGTCTCA | |
| | AACTCAGAAATCCACTTACCTCTGCCTCCCAAGGGCTGGGATTAAAGGCATGC | |
| | ACCACCATTGCCCGGCAGGGTATGTGGACTATGCTGTCCTTCAACTCAGAGAT | |
| | CGTGGTACTTTAAATCTCATTTTCCGAAAGAAAAGAAAAGAAAGGAAGGGGG | |
| | GAAATAAAGCCAGTATATTTTGCACCCACAATCTGAAGTTCTACAAATATTGT | |
| | TAGAAAATGCAAAGGACCCAAGTC | |
| mRnu3b1- | CTCGGTTTATCTTAAAGGGTTTTAAATATAGAAAGCTTCCTTAGTCTTATGGGT | 357 |
| Terminator | GAGAGTGCTGTTTAAGCCTTACCTAGGTATGTCTTTGAACATTTATTACAGATT | |
| | GTTATTTGTGACTATGACCACTCAGGTGTTCTGTTCTTGTGAATTTAGACACAC | |
| | TCAGAAAGGCTACACACTCAAAGACCACACCAAAGTGAAGTACTATCTGGGC | |
| | CGTGAAACTGGTCACATATCAGTTATTTTGCAGTAAAACAAGGAAGTCAATCA | |
| | TACTGCTTCAGTGGGTCAGGGAGTCAAGAATGCACAGTACAAGAGCTGGTGA | |
| | GATTCCTCAGCAGGTAAGAGCACTGAATGCTCTTCCAAAGATTCTGAGTTCAA | |
| | ATCCCAGCAACCACGTGGTGGATTTGAACAAAGGATGAAAACAACCATCCAT | |
| | AACCAGATCTGATGCCCTCTTCTGGAGTGTCAAAAGACAGCTATGGTGTACTT | |
| | ACATAAATAAATAAGTAAAAACA | |
| mRnu5g- | CCACATTTTGTGTTTAAAAATAGAAATATTTAAGTGAGATCAGTTTAAATCTG | 358 |
| Terminator | CTTTATCTAGGGTGTCTAACTGCTTGCATCTTTTTAACTTTTCCTTATTTGTGAG | |
| | AAGGTCTGTAAAACTTATTATATGTTAGTACACTGTAGCTGTCTTCAGACACT | |
| | CCAGAAGAGGGATTCAGATCACCTTACAGATGGTTTTGAGCCACCATGTGGTT | |
| | GCTGGGATTTGAACTCAGGACCTTTAGAAAAGTAGTTGGGTGCTCTTACCCGC | |
| | TGAGCCATCTCACCAGCCCTTTTTTTTTCTTTTTTCCAGTTCGGGTTTCTCTGTG | |
| | TGTAGCCCTGGCTGTCCTGGAACTCATTCTGTAGACCGGGCTGGCCTCGAACT | |
| | CAGAAATCTGCCTGCCTCTGCCTCCCAAGTGCTGGGATTAAAGGAATGTGCCT | |
| | TCATTGCCCGGCTCTAGGGCTTTTTGTTGTTGTTTGTTTGTTTGTTTGTTTGTTT | |
| | GCTTTTGTGGATGGCCT | |
| mRnu6- | AAAACCAGTAAGGGAGAAAGGTCAAGTAACATATAAAGGAAGGCCTATCAG | 359 |
| Terminator | AATTACACCAGACTTTTCACCAGAGACTATGAAAGCCAGAAGAGCCTGGACA | |
| | GATGTTATACAGACACTAAGAGAACACAAATGCCAGCCCAGGCTACTATACC | |
| | TGGCCAAACTCTCAATTACCATAGATGGAGAAACCAAAGTATTCCACGACAA | |
| | AAACAAATTCACACAATATCTTTCCACGAATCCAGCCCTTCAAAGGATAATGA | |
| | CAGAAAAGAAGCAATACAAGGACGGAAATCACGCCCTAGAACAACCAAGAA | |
| | ACTAATCATTCAACAAACCAAAAAGAAGACAGCCACAAGAACAGAATGCCA | |
| | ACTCTAACAACAAAAATAAAAGGAAGCAACAATTACTTTTCCTTAATATCTCT | |
| | TAATATCAATGGACTCAATTCCCCAATAAAAAGACATAGACTAACAGACTGG | |
| | CTACACAAACAGGACCCAACATTCTGCTGCTTA | |
| mRnu7- | CCCAATTTCACTGGTCTACAATGAAAGCAAAACAGTTCTCTTCCCCGCTCCCC | 360 |
| Terminator | GGTGTGTGAGAGGGGCTTTGATCCTTCTCTGGTTTCCTAGGAAACGCGTATGT | |
| | GCTAGAGCCACGCTCTGAGACTTCCGCCTCGTGCGGTCCCGCTTCCTTTCTGCC | |
| | TCCTCTGGCCTGCATCCGTGGGGGAGGTGGCTGGCTGCAGGGTTCTCCGGATC | |
| | TCTTCTGTGTTCCCACTACTCAAGCACCGAGTGGCGTTCTATGGCGTCCGCCTC | |
| | GGCTCAGCCCGCGGCCCTGAGCGCGGAGCAGGCCAAGGGTGAGGAAAGAGC | |
| | CTAGTCTGGGTGTGGGCCGTCTTCTGGGGGGAGGGCGCGATACTATTCTTTGC | |
| | CTCTCCAAGTGGGGCCCTTGCCCATTAGGGCGAAGGGTTGGGAGGACAAGCT | |
| | TATCGGGTTGCTTGCAGGCCTCGGCCCACTAATTCCCTGCCTCTTCCCTCCTCT | |
| | GGGCTCGCCCGCCGTCCTCCGCG | |
| mRnu11- | GAATTTGTTAAATTACAGGAGGAAGCTTTTTTAGTTCATTTTACAAATACTTTC | 361 |
| Terminator | TTGTTAGGTAGATAATACCTTCTAAGGTTATTATCTGGATCTTCTAAGTCTTTT | |
| | TATCGCTAGCAAGTCTAAGTTTCTGGACTTTGGAATTAGAAGTTAATGTCGTA | |
| | GGCGGGCATGGTGGTCCACGCCTTTAATCCCAGAGTTTGGGAGGCAGACGCA | |
| | GGTGGTTTTCTGAGTTCGAGGCCATCCTGCTCTTCAATGTTAGTTCCAGGACA | |
| | GCCAGGGTTATACAAAGAAACCCTGTCTCGAGAAACAAAACAAACAAAAAA | |
| | AGAGTAGCTGAGTGCTCTGAGTTGTCTCTCCAGCCCCTTAGCTATTTATTTTCA | |
| | TTTAAATTATTTTTGTTTTATTCGCTTGTGTGCCTGAGTTTGAATTTCCCGGGAT | |
| | TAACAGTTGAAGACTAGGGCTGGTGAGATGGCTCAGCGGTTAAGATTGCCGA | |
| | CTGCTCTTCCAAAGGTCCTGAG | |
| mRnu12- | ACCCCCTAATTCTACAAGAATAGAAAGCTTAGCAGCTCCTCTGAATGGTTGCT | 362 |
| Terminator | CTCAGGTGCTAGTGGTTGCAAGTCGTATGTTGATGCCTGCCTTTGCCCTACCTG | |
| | GTGTGTGAGCGTGAGCGCGCCCCACCTCTCCAGCCTCTCTCTTACTGTTTTTCA | |
| | GACAAGATGGGACTGTTGTTCTCCTACTGCTATGTACCTCAATTTCAATTGATC | |
| | ATGCCTGGACATAACTGCCCATGTTGTAAGGTCGTCTACGTTGTAAGGTCGTG | |
| | CTCAGTTTCTGATGCCTAGACTAGTCACCTTTCCGTTTGCCTCCTGACTGTGTA | |
| | AATGAGGCAAATGAAGGTGGTCTCTTAACTGACATGTTAGTGTTTGACCTATG | |
| | GGATAGTTTCTCATTGTTGAGACAGAATTTCTAGAAGCTGGTCCGCTGAGCGG | |
| | CTAAGAGCACTTGCTGATATTGCAAAGGATCTACCTGGGTTTGGTTCTCAGCA | |
| | CTCACATGGAAGTTCACAT | |
| mRn7sk- | TGTAGTTCCTGCAATTTAATTTTCGTTTAAAACTTTATTTTATTCTTTAACTTAA | 363 |
| Terminator | AAAAAATATATA | |

### Example 10: Example snRNA Sequences and Example Expression Construct Sequences

Tables 7A-7C include some example snRNA sequences. Some such RNA sequences may be useful in a composition or method herein. A DNA sequence encoding an snRNA sequence of any of Tables 7A-7C may have a similar sequence but have Ts instead of Us. Some such DNA sequences may be useful in a composition or method herein, such as in an expression construct or vector, or in a method of treatment that includes administering a composition comprising the expression construct or vector.

TABLE 7A
Example U7 snRNA sequences targeting SCN1A mRNA-corresponding with constructs
in Table 2A

| Sequence | | |
|---|---|---|
| Name | snRNA Sequences (5′ to 3′) | SEQ ID NO: |
| Scramble-01 | AUGAUAGGGACUUAGGGUGAGCACCCCAUCCCCCACUACAAAUUUUUG | 364 |
| | GAGcaggUUUUcUgacUUcggUcggaaaaccccU | |
| N5-01 | AUGAUAGGGACUUAGGGUGAAACCACCGCUCCACCCCAUCAAUUUUUG | 365 |
| | GAGcaggUUUUcUgacUUcggUcggaaaaccccU | |
| N5-02 | AUGAUAGGGACUUAGGGUGACAAGUUGGAGCAAGAUUAUCAAUUUUUG | 366 |
| | GAGcaggUUUUcUgacUUcggUcggaaaaccccU | |
| N5-03 | AUGAUAGGGACUUAGGGUGACUAUAUAAAAUAGAAAUAUAAAUUUUU | 367 |
| | GGAGcaggUUUUcUgacUUcggUcggaaaaccccU | |
| N5-04 | AUGAUAGGGACUUAGGGUGAUAGUUUAUUAUUAGUUAGAAAAUUUUU | 368 |
| | GGAGcaggUUUUcUgacUUcggUcggaaaaccccU | |
| N5-05 | AUGAUAGGGACUUAGGGUGAGAGGAACCACCGCUCCACCCCAUCAAUU | 369 |
| | UUUGGAGcaggUUUUcUgacUUcggUcggaaaaccccU | |
| N5-06 | AUGAUAGGGACUUAGGGUGACAUCCAAGUUGGAGCAAGAUUAUCAAUU | 370 |
| | UUUGGAGcaggUUUUcUgacUUcggUcggaaaaccccU | |
| N5-07 | AUGAUAGGGACUUAGGGUGAUAUCCUAUAUAAAAUAGAAAUAUAAAU | 371 |
| | UUUUGGAGcaggUUUUcUgacUUcggUcggaaaaccccU | |
| N5-08 | AUGAUAGGGACUUAGGGUGAUAUAUAGUUUAUUAUUAGUUAGAAAAU | 372 |
| | UUUUGGAGcaggUUUUcUgacUUcggUcggaaaaccccU | |
| N5-09 | AUGAUAGGGACUUAGGGUGAGGGAGGAACCACCGCUCCACCCCAUCAA | 373 |
| | UUUUUGGAGcaggUUUUcUgacUUcggUcggaaaaccccU | |
| N5-10 | AUGAUAGGGACUUAGGGUGACCCAUCCAAGUUGGAGCAAGAUUAUCAA | 374 |
| | UUUUUGGAGcaggUUUUcUgacUUcggUcggaaaaccccU | |
| N5-11 | AUGAUAGGGACUUAGGGUGAAUUAUCCUAUAUAAAAUAGAAAUAUAA | 375 |
| | AUUUUUGGAGcaggUUUUcUgacUUcggUcggaaaaccccU | |
| N5-12 | AUGAUAGGGACUUAGGGUGAAAUAUAUAGUUUAUUAUUAGUUAGAAA | 376 |
| | AUUUUUGGAGcaggUUUUcUgacUUcggUcggaaaaccccU | |
| N5-13 | AUGAUAGGGACUUAGGGUGAUGAGGGGAGGAACCACCGCUCCACCCCA | 377 |
| | UCAAUUUUUGGAGcaggUUUUcUgacUUcggUcggaaaaccccU | |
| N5-14 | AUGAUAGGGACUUAGGGUGACCACCCCAUCCAAGUUGGAGCAAGAUUA | 378 |
| | UCAAUUUUUGGAGcaggUUUUcUgacUUcggUcggaaaaccccU | |
| N5-15 | AUGAUAGGGACUUAGGGUGACAAGAUUAUCCUAUAUAAAAUAGAAAUA | 379 |
| | UAAAUUUUUGGAGcaggUUUUcUgacUUcggUcggaaaaccccU | |
| N5-16 | AUGAUAGGGACUUAGGGUGAUAGAAAUAUAUAGUUUAUUAUUAGUUA | 380 |
| | GAAAAUUUUUGGAGcaggUUUUcUgacUUcggUcggaaaaccccU | |
| N3-01 | AUGAUAGGGACUUAGGGUGAAGUGCAAGGAUUAAAGGUAGAAUUUUU | 381 |
| | GGAGcaggUUUUcUgacUUcggUcggaaaaccccU | |
| N3-02 | AUGAUAGGGACUUAGGGUGACAAAAGGGGUAAUACAGUACAAUUUUUG | 382 |
| | GAGcaggUUUUcUgacUUcggUcggaaaaccccU | |
| N3-03 | AUGAUAGGGACUUAGGGUGACCAUAAUAAAGGGCUGAGGGAAUUUUUG | 383 |
| | GAGcaggUUUUcUgacUUcggUcggaaaaccccU | |
| N3-04 | AUGAUAGGGACUUAGGGUGAGAGGAACCACCGCUCCACCCAAUUUUUG | 384 |
| | GAGcaggUUUUcUgacUUcggUcggaaaaccccU | |
| N3-05 | AUGAUAGGGACUUAGGGUGAUCACAGUGCAAGGAUUAAAGGUAGAAUU | 385 |
| | UUUGGAGcaggUUUUcUgacUUcggUcggaaaaccccU | |
| N3-06 | AUGAUAGGGACUUAGGGUGAGUAGCAAAAGGGGUAAUACAGUACAAUU | 386 |
| | UUUGGAGcaggUUUUcUgacUUcggUcggaaaaccccU | |
| N3-07 | AUGAUAGGGACUUAGGGUGAGUACCCAUAAUAAAGGGCUGAGGGAAUU | 387 |
| | UUUGGAGcaggUUUUcUgacUUcggUcggaaaaccccU | |
| N3-08 | AUGAUAGGGACUUAGGGUGAAGGGGAGGAACCACCGCUCCACCCAAUU | 388 |
| | UUUGGAGcaggUUUUcUgacUUcggUcggaaaaccccU | |
| N3-09 | AUGAUAGGGACUUAGGGUGAAGUCACAGUGCAAGGAUUAAAGGUAGAA | 389 |
| | UUUUUGGAGcaggUUUUcUgacUUcggUcggaaaaccccU | |
| N3-10 | AUGAUAGGGACUUAGGGUGAAGGUAGCAAAAGGGGUAAUACAGUACAA | 390 |
| | UUUUUGGAGcaggUUUUcUgacUUcggUcggaaaaccccU | |
| N3-11 | AUGAUAGGGACUUAGGGUGACAGUACCCAUAAUAAAGGGCUGAGGGAA | 391 |
| | UUUUUGGAGcaggUUUUcUgacUUcggUcggaaaaccccU | |
| N3-12 | AUGAUAGGGACUUAGGGUGAUGAGGGGAGGAACCACCGCUCCACCCAA | 392 |
| | UUUUUGGAGcaggUUUUcUgacUUcggUcggaaaaccccU | |
| N3-13 | AUGAUAGGGACUUAGGGUGACAUAAGUCACAGUGCAAGGAUUAAAGGU | 393 |
| | AGAAUUUUUGGAGcaggUUUUcUgacUUcggUcggaaaaccccU | |
| N3-14 | AUGAUAGGGACUUAGGGUGAUUAAAGGUAGCAAAAGGGGUAAUACAG | 394 |
| | UACAAUUUUUGGAGcaggUUUUcUgacUUcggUcggaaaaccccU | |
| N3-15 | AUGAUAGGGACUUAGGGUGAAAUACAGUACCCAUAAUAAAGGGCUGAG | 395 |
| | GGAAUUUUUGGAGcaggUUUUcUgacUUcggUcggaaaaccccU | |
| N3-16 | AUGAUAGGGACUUAGGGUGAGGGCUGAGGGGAGGAACCACCGCUCCAC | 396 |
| | CCAAUUUUUGGAGcaggUUUUcUgacUUcggUcggaaaaccccU | |
| N5-ESSalt1- | UUUGUUCCGUGGGUGGUUUAAACCACCGCUCCACCCCAUCAAUUUUUG | 397 |
| 01 | GAGcaggUUUUcUgacUUcggUcggaaaaccccU | |
| N5-ESSalt1- | UUUGUUCCGUGGGUGGUUUACAAGUUGGAGCAAGAUUAUCAAUUUUUG | 398 |
| 02 | GAGcaggUUUUcUgacUUcggUcggaaaaccccU | |
| N5-ESSalt1- | UUUGUUCCGUGGGUGGUUUACUAUAUAAAAUAGAAAUAUAAAUUUUU | 399 |
| 03 | GGAGcaggUUUUcUgacUUcggUcggaaaaccccU | |
| N5-ESSalt1- | UUUGUUCCGUGGGUGGUUUAUAGUUUAUUAUUAGUUAGAAAAUUUUU | 400 |
| 04 | GGAGcaggUUUUcUgacUUcggUcggaaaaccccU | |
| N5-ESSalt1- | UUUGUUCCGUGGGUGGUUUAGAGGAACCACCGCUCCACCCCAUCAAUU | 401 |
| 05 | UUUGGAGcaggUUUUcUgacUUcggUcggaaaaccccU | |
| N5-ESSalt1- | UUUGUUCCGUGGGUGGUUUACAUCCAAGUUGGAGCAAGAUUAUCAAUU | 402 |
| 06 | UUUGGAGcaggUUUUcUgacUUcggUcggaaaaccccU | |
| N5-ESSalt1- | UUUGUUCCGUGGGUGGUUUAUAUCCUAUAUAAAAUAGAAAUAUAAAUU | 403 |
| 07 | UUUGGAGcaggUUUUcUgacUUcggUcggaaaaccccU | |
| N5-ESSalt1- | UUUGUUCCGUGGGUGGUUUAUAUAUAGUUUAUUAUUAGUUAGAAAAU | 404 |
| 08 | UUUUGGAGcaggUUUUcUgacUUcggUcggaaaaccccU | |
| N5-ESSalt1- | UUUGUUCCGUGGGUGGUUUAGGGAGGAACCACCGCUCCACCCCAUCAA | 405 |
| 09 | UUUUUGGAGcaggUUUUcUgacUUcggUcggaaaaccccU | |
| Scramble-02 | UUUGUUCCGUGGGUGGUUUAGGAGAAUCACCUUAUAGGAUAAUUUUUG | 406 |
| | GAGcaggUUUUcUgacUUcggUcggaaaaccccU | |
| N5-ESSalt1- | UUUGUUCCGUGGGUGGUUUACCCAUCCAAGUUGGAGCAAGAUUAUCAA | 407 |
| 10 | UUUUUGGAGcaggUUUUcUgacUUcggUcggaaaaccccU | |
| N5-ESSalt1- | UUUGUUCCGUGGGUGGUUUAAUUAUCCUAUAUAAAAUAGAAAUAUAAA | 408 |
| 11 | UUUUUGGAGcaggUUUUcUgacUUcggUcggaaaaccccU | |
| N5-ESSalt1- | UUUGUUCCGUGGGUGGUUUAAAUAUAUAGUUUAUUAUUAGUUAGAAA | 409 |
| 12 | AUUUUUGGAGcaggUUUUcUgacUUcggUcggaaaaccccU | |
| N5-ESSalt1- | UUUGUUCCGUGGGUGGUUUAUGAGGGGAGGAACCACCGCUCCACCCCA | 410 |
| 13 | UCAAUUUUUGGAGcaggUUUUcUgacUUcggUcggaaaaccccU | |
| N5-ESSalt1- | UUUGUUCCGUGGGUGGUUUACCACCCCAUCCAAGUUGGAGCAAGAUUA | 411 |
| 14 | UCAAUUUUUGGAGcaggUUUUcUgacUUcggUcggaaaaccccU | |
| N5-ESSalt1- | UUUGUUCCGUGGGUGGUUUACAAGAUUAUCCUAUAUAAAAUAGAAAUA | 412 |
| 15 | UAAAUUUUUGGAGcaggUUUUcUgacUUcggUcggaaaaccccU | |
| N5-ESSalt1- | UUUGUUCCGUGGGUGGUUUAUAGAAAUAUAUAGUUUAUUAUUAGUUA | 413 |
| 16 | GAAAAUUUUUGGAGcaggUUUUcUgacUUcggUcggaaaaccccU | |
| N3-ESSalt1- | UUUGUUCCGUGGGUGGUUUAAGUGCAAGGAUUAAAGGUAGAAUUUUU | 414 |
| 01 | GGAGcaggUUUUcUgacUUcggUcggaaaaccccU | |
| N3-ESSalt1- | UUUGUUCCGUGGGUGGUUUACAAAAGGGGUAAUACAGUACAAUUUUUG | 415 |
| 02 | GAGcaggUUUUcUgacUUcggUcggaaaaccccU | |
| N3-ESSalt1- | UUUGUUCCGUGGGUGGUUUACCAUAAUAAAGGGCUGAGGGAAUUUUUG | 416 |
| 03 | GAGcaggUUUUcUgacUUcggUcggaaaaccccU | |
| N3-ESSalt1- | UUUGUUCCGUGGGUGGUUUAGAGGAACCACCGCUCCACCCAAUUUUUG | 417 |
| 04 | GAGcaggUUUUcUgacUUcggUcggaaaaccccU | |
| Scramble-03 | UUUGUUCCGUGGGUGGUUUAGAAUAAUAACAUAAUAAUAUAAUUUUU | 418 |
| | GGAGcaggUUUUcUgacUUcggUcggaaaaccccU | |
| N3-ESSalt1- | UUUGUUCCGUGGGUGGUUUAUCACAGUGCAAGGAUUAAAGGUAGAAUU | 419 |
| 05 | UUUGGAGcaggUUUUcUgacUUcggUcggaaaaccccU | |
| N3-ESSalt1- | UUUGUUCCGUGGGUGGUUUAGUAGCAAAAGGGGUAAUACAGUACAAUU | 420 |
| 06 | UUUGGAGcaggUUUUcUgacUUcggUcggaaaaccccU | |
| N3-ESSalt1- | UUUGUUCCGUGGGUGGUUUAGUACCCAUAAUAAAGGGCUGAGGGAAUU | 421 |
| 07 | UUUGGAGcaggUUUUcUgacUUcggUcggaaaaccccU | |
| N3-ESSalt1- | UUUGUUCCGUGGGUGGUUUAAGGGGAGGAACCACCGCUCCACCCAAUU | 422 |
| 08 | UUUGGAGcaggUUUUcUgacUUcggUcggaaaaccccU | |
| N3-ESSalt1- | UUUGUUCCGUGGGUGGUUUAAGUCACAGUGCAAGGAUUAAAGGUAGAA | 423 |
| 09 | UUUUUGGAGcaggUUUUcUgacUUcggUcggaaaaccccU | |
| N3-ESSalt1- | UUUGUUCCGUGGGUGGUUUAAGGUAGCAAAAGGGGUAAUACAGUACAA | 424 |
| 10 | UUUUUGGAGcaggUUUUcUgacUUcggUcggaaaaccccU | |
| N3-ESSalt1- | UUUGUUCCGUGGGUGGUUUACAGUACCCAUAAUAAAGGGCUGAGGGAA | 425 |
| 11 | UUUUUGGAGcaggUUUUcUgacUUcggUcggaaaaccccU | |
| N3-ESSalt1- | UUUGUUCCGUGGGUGGUUUAUGAGGGGAGGAACCACCGCUCCACCCAA | 426 |
| 12 | UUUUUGGAGcaggUUUUcUgacUUcggUcggaaaaccccU | |
| N3-ESSalt1- | UUUGUUCCGUGGGUGGUUUACAUAAGUCACAGUGCAAGGAUUAAAGGU | 427 |
| 13 | AGAAUUUUUGGAGcaggUUUUcUgacUUcggUcggaaaaccccU | |
| N3-ESSalt1- | UUUGUUCCGUGGGUGGUUUAUUAAAGGUAGCAAAAGGGGUAAUACAGU | 428 |
| 14 | ACAAUUUUUGGAGcaggUUUUcUgacUUcggUcggaaaaccccU | |
| N3-ESSalt1- | UUUGUUCCGUGGGUGGUUUAAAUACAGUACCCAUAAUAAAGGGCUGAG | 429 |
| 15 | GGAAUUUUUGGAGcaggUUUUcUgacUUcggUcggaaaaccccU | |
| N3-ESSalt1- | UUUGUUCCGUGGGUGGUUUAGGGCUGAGGGGAGGAACCACCGCUCCAC | 430 |
| 16 | CCAAUUUUUGGAGcaggUUUUcUgacUUcggUcggaaaaccccU | |
| N5-ESSalt2- | UGGGGGGAGGUAGGUAGGUAAACCACCGCUCCACCCCAUCAAUUUUUG | 431 |
| 01 | GAGcaggUUUUcUgacUUcggUcggaaaaccccU | |
| N5-ESSalt2- | UGGGGGGAGGUAGGUAGGUACAAGUUGGAGCAAGAUUAUCAAUUUUU | 432 |
| 02 | GGAGcaggUUUUcUgacUUcggUcggaaaaccccU | |
| N5-ESSalt2- | UGGGGGGAGGUAGGUAGGUACUAUAUAAAAUAGAAAUAUAAAUUUUU | 433 |
| 03 | GGAGcaggUUUUcUgacUUcggUcggaaaaccccU | |
| N5-ESSalt2- | UGGGGGGAGGUAGGUAGGUAUAGUUUAUUAUUAGUUAGAAAAUUUUU | 434 |
| 04 | GGAGcaggUUUUcUgacUUcggUcggaaaaccccU | |
| N5-ESSalt2- | UGGGGGGAGGUAGGUAGGUAGAGGAACCACCGCUCCACCCCAUCAAUU | 435 |
| 05 | UUUGGAGcaggUUUUcUgacUUcggUcggaaaaccccU | |
| N5-ESSalt2- | UGGGGGGAGGUAGGUAGGUACAUCCAAGUUGGAGCAAGAUUAUCAAUU | 436 |
| 06 | UUUGGAGcaggUUUUcUgacUUcggUcggaaaaccccU | |
| N5-ESSalt2- | UGGGGGGAGGUAGGUAGGUAUAUCCUAUAUAAAAUAGAAAUAUAAAU | 437 |
| 07 | UUUUGGAGcaggUUUUcUgacUUcggUcggaaaaccccU | |
| N5-ESSalt2- | UGGGGGGAGGUAGGUAGGUAUAUAUAGUUUAUUAUUAGUUAGAAAAU | 438 |
| 08 | UUUUGGAGcaggUUUUcUgacUUcggUcggaaaaccccU | |
| N5-ESSalt2- | UGGGGGGAGGUAGGUAGGUAGGGAGGAACCACCGCUCCACCCCAUCAA | 439 |
| 09 | UUUUUGGAGcaggUUUUcUgacUUcggUcggaaaaccccU | |
| N5-ESSalt2- | UGGGGGGAGGUAGGUAGGUACCCAUCCAAGUUGGAGCAAGAUUAUCAA | 440 |
| 10 | UUUUUGGAGcaggUUUUcUgacUUcggUcggaaaaccccU | |
| N5-ESSalt2- | UGGGGGGAGGUAGGUAGGUAAUUAUCCUAUAUAAAAUAGAAAUAUAA | 441 |
| 11 | AUUUUUGGAGcaggUUUUcUgacUUcggUcggaaaaccccU | |
| N5-ESSalt2- | UGGGGGGAGGUAGGUAGGUAUGAGGGGAGGAACCACCGCUCCACCCCA | 442 |
| 13 | UCAAUUUUUGGAGcaggUUUUcUgacUUcggUcggaaaaccccU | |
| N5-ESSalt2- | UGGGGGGAGGUAGGUAGGUACCACCCCAUCCAAGUUGGAGCAAGAUUA | 443 |
| 14 | UCAAUUUUUGGAGcaggUUUUcUgacUUcggUcggaaaaccccU | |
| N5-ESSalt2- | UGGGGGGAGGUAGGUAGGUACAAGAUUAUCCUAUAUAAAAUAGAAAU | 444 |
| 15 | AUAAAUUUUUGGAGcaggUUUUcUgacUUcggUcggaaaaccccU | |
| N3-ESSalt2- | UGGGGGGAGGUAGGUAGGUAAGUGCAAGGAUUAAAGGUAGAAUUUUU | 445 |
| 01 | GGAGcaggUUUUcUgacUUcggUcggaaaaccccU | |
| N3-ESSalt2- | UGGGGGGAGGUAGGUAGGUACAAAAGGGGUAAUACAGUACAAUUUUU | 446 |
| 02 | GGAGcaggUUUUcUgacUUcggUcggaaaaccccU | |
| N3-ESSalt2- | UGGGGGGAGGUAGGUAGGUACCAUAAUAAAGGGCUGAGGGAAUUUUU | 447 |
| 03 | GGAGcaggUUUUcUgacUUcggUcggaaaaccccU | |
| N3-ESSalt2- | UGGGGGGAGGUAGGUAGGUAGAGGAACCACCGCUCCACCCAAUUUUUG | 448 |
| 04 | GAGcaggUUUUcUgacUUcggUcggaaaaccccU | |
| N3-ESSalt2- | UGGGGGGAGGUAGGUAGGUAUCACAGUGCAAGGAUUAAAGGUAGAAU | 449 |
| 05 | UUUUGGAGcaggUUUUcUgacUUcggUcggaaaaccccU | |
| N3-ESSalt2- | UGGGGGGAGGUAGGUAGGUAGUAGCAAAAGGGGUAAUACAGUACAAU | 450 |
| 06 | UUUUGGAGcaggUUUUcUgacUUcggUcggaaaaccccU | |
| N3-ESSalt2- | UGGGGGGAGGUAGGUAGGUAGUACCCAUAAUAAAGGGCUGAGGGAAUU | 451 |
| 07 | UUUGGAGcaggUUUUcUgacUUcggUcggaaaaccccU | |
| N3-ESSalt2- | UGGGGGGAGGUAGGUAGGUAAGGGGAGGAACCACCGCUCCACCCAAUU | 452 |
| 08 | UUUGGAGcaggUUUUcUgacUUcggUcggaaaaccccU | |
| N3-ESSalt2- | UGGGGGGAGGUAGGUAGGUAAGUCACAGUGCAAGGAUUAAAGGUAGA | 453 |
| 09 | AUUUUUGGAGcaggUUUUcUgacUUcggUcggaaaaccccU | |
| N3-ESSalt2- | UGGGGGGAGGUAGGUAGGUAAGGUAGCAAAAGGGGUAAUACAGUACA | 454 |
| 10 | AUUUUUGGAGcaggUUUUcUgacUUcggUcggaaaaccccU | |
| N3-ESSalt2- | UGGGGGGAGGUAGGUAGGUACAGUACCCAUAAUAAAGGGCUGAGGGAA | 455 |
| 11 | UUUUUGGAGcaggUUUUcUgacUUcggUcggaaaaccccU | |
| N3-ESSalt2- | UGGGGGGAGGUAGGUAGGUACAUAAGUCACAGUGCAAGGAUUAAAGGU | 456 |
| 13 | AGAAUUUUUGGAGcaggUUUUcUgacUUcggUcggaaaaccccU | |
| N3-ESSalt2- | UGGGGGGAGGUAGGUAGGUAUUAAAGGUAGCAAAAGGGGUAAUACAG | 457 |
| 14 | UACAAUUUUUGGAGcaggUUUUcUgacUUcggUcggaaaaccccU | |
| N3-ESSalt2- | UGGGGGGAGGUAGGUAGGUAAAUACAGUACCCAUAAUAAAGGGCUGAG | 458 |
| 15 | GGAAUUUUUGGAGcaggUUUUcUgacUUcggUcggaaaaccccU | |
| Scramble-04 | UGGGGGGAGGUAGGUAGGUAGAAAUUUAUGUAUUGAUUAUAAUUUUU | 459 |
| | GGAGcaggUUUUcUgacUUcggUcggaaaaccccU | |

TABLE 7B
Example U7 snRNA sequences targeting SCN1A mRNA-corresponding with constructs
in Table 2B

| Sequence | | |
|---|---|---|
| Name | snRNA Sequences (5′ to 3′) | SEQ ID NO: |
| N5-01 | AUGAUAGGGACUUAGGGUGAAACCAGCGCUCCACCCCAUCAAUUUUUG | 460 |
| | GAGcaggUUUUcUgacUUcggUcggaaaaccccU | |
| N5-02 | AUGAUAGGGACUUAGGGUGACAAGUUGGAGCAAGAUUAUCAAUUUUUG | 461 |
| | GAGcaggUUUUcUgacUUcggUcggaaaaccccU | |
| N5-03 | AUGAUAGGGACUUAGGGUGACUAUACAAAAUAGAAAUAUAAAUUUUU | 462 |
| | GGAGcaggUUUUcUgacUUcggUcggaaaaccccU | |
| N5-04 | AUGAUAGGGACUUAGGGUGAUAGUUUGUUAUUAGUUAGAAAAUUUUU | 463 |
| | GGAGcaggUUUUcUgacUUcggUcggaaaaccccU | |
| N5-05 | AUGAUAGGGACUUAGGGUGAGAGGAACCAGCGCUCCACCCCAUCAAUU | 464 |
| | UUUGGAGcaggUUUUcUgacUUcggUcggaaaaccccU | |
| N5-06 | AUGAUAGGGACUUAGGGUGACAUCCAAGUUGGAGCAAGAUUAUCAAUU | 465 |
| | UUUGGAGcaggUUUUcUgacUUcggUcggaaaaccccU | |
| N5-07 | AUGAUAGGGACUUAGGGUGAUAUCCUAUACAAAAUAGAAAUAUAAAUU | 466 |
| | UUUGGAGcaggUUUUcUgacUUcggUcggaaaaccccU | |
| N5-08 | AUGAUAGGGACUUAGGGUGAUAUAUAGUUUGUUAUUAGUUAGAAAAU | 467 |
| | UUUUGGAGcaggUUUUcUgacUUcggUcggaaaaccccU | |
| N5-09 | AUGAUAGGGACUUAGGGUGAGGGAGGAACCAGCGCUCCACCCCAUCAA | 468 |
| | UUUUUGGAGcaggUUUUcUgacUUcggUcggaaaaccccU | |
| N5-10 | AUGAUAGGGACUUAGGGUGACCCAUCCAAGUUGGAGCAAGAUUAUCAA | 469 |
| | UUUUUGGAGcaggUUUUcUgacUUcggUcggaaaaccccU | |
| N5-11 | AUGAUAGGGACUUAGGGUGAAUUAUCCUAUACAAAAUAGAAAUAUAAA | 470 |
| | UUUUUGGAGcaggUUUUcUgacUUcggUcggaaaaccccU | |
| N5-12 | AUGAUAGGGACUUAGGGUGAAAUAUAUAGUUUGUUAUUAGUUAGAAA | 471 |
| | AUUUUUGGAGcaggUUUUcUgacUUcggUcggaaaaccccU | |
| N5-13 | AUGAUAGGGACUUAGGGUGAUCAGGGGAGGAACCAGCGCUCCACCCCA | 472 |
| | UCAAUUUUUGGAGcaggUUUUcUgacUUcggUcggaaaaccccU | |
| N5-14 | AUGAUAGGGACUUAGGGUGACCACCCCAUCCAAGUUGGAGCAAGAUUA | 473 |
| | UCAAUUUUUGGAGcaggUUUUcUgacUUcggUcggaaaaccccU | |
| N5-15 | AUGAUAGGGACUUAGGGUGACAAGAUUAUCCUAUACAAAAUAGAAAUA | 474 |
| | UAAAUUUUUGGAGcaggUUUUcUgacUUcggUcggaaaaccccU | |
| N5-16 | AUGAUAGGGACUUAGGGUGAUAGAAAUAUAUAGUUUGUUAUUAGUUA | 475 |
| | GAAAAUUUUUGGAGcaggUUUUcUgacUUcggUcggaaaaccccU | |
| N3-01 | AUGAUAGGGACUUAGGGUGAAGUGCAAGGAUUAAAGGUAGAAUUUUU | 476 |
| | GGAGcaggUUUUcUgacUUcggUcggaaaaccccU | |
| N3-02 | AUGAUAGGGACUUAGGGUGACAAAAGGGGUAAUACAGUACAAUUUUUG | 477 |
| | GAGcaggUUUUcUgacUUcggUcggaaaaccccU | |
| N3-03 | AUGAUAGGGACUUAGGGUGACCAUAAUAAAGGGCUCAGGGAAUUUUUG | 478 |
| | GAGcaggUUUUcUgacUUcggUcggaaaaccccU | |
| N3-04 | AUGAUAGGGACUUAGGGUGAGAGGAACCAGCGCUCCACCCAAUUUUUG | 479 |
| | GAGcaggUUUUcUgacUUcggUcggaaaaccccU | |
| N3-05 | AUGAUAGGGACUUAGGGUGAUCACAGUGCAAGGAUUAAAGGUAGAAUU | 480 |
| | UUUGGAGcaggUUUUcUgacUUcggUcggaaaaccccU | |
| N3-06 | AUGAUAGGGACUUAGGGUGAGUAGCAAAAGGGGUAAUACAGUACAAUU | 481 |
| | UUUGGAGcaggUUUUcUgacUUcggUcggaaaaccccU | |
| N3-07 | AUGAUAGGGACUUAGGGUGAGUACCCAUAAUAAAGGGCUCAGGGAAUU | 482 |
| | UUUGGAGcaggUUUUcUgacUUcggUcggaaaaccccU | |
| N3-08 | AUGAUAGGGACUUAGGGUGAAGGGGAGGAACCAGCGCUCCACCCAAUU | 483 |
| | UUUGGAGcaggUUUUcUgacUUcggUcggaaaaccccU | |
| N3-09 | AUGAUAGGGACUUAGGGUGAAGUCACAGUGCAAGGAUUAAAGGUAGAA | 484 |
| | UUUUUGGAGcaggUUUUcUgacUUcggUcggaaaaccccU | |
| N3-10 | AUGAUAGGGACUUAGGGUGAAGGUAGCAAAAGGGGUAAUACAGUACAA | 485 |
| | UUUUUGGAGcaggUUUUcUgacUUcggUcggaaaaccccU | |
| N3-11 | AUGAUAGGGACUUAGGGUGACAGUACCCAUAAUAAAGGGCUCAGGGAA | 486 |
| | UUUUUGGAGcaggUUUUcUgacUUcggUcggaaaaccccU | |
| N3-12 | AUGAUAGGGACUUAGGGUGAUCAGGGGAGGAACCAGCGCUCCACCCAA | 487 |
| | UUUUUGGAGcaggUUUUcUgacUUcggUcggaaaaccccU | |
| N3-13 | AUGAUAGGGACUUAGGGUGACAUAAGUCACAGUGCAAGGAUUAAAGGU | 488 |
| | AGAAUUUUUGGAGcaggUUUUcUgacUUcggUcggaaaaccccU | |
| N3-14 | AUGAUAGGGACUUAGGGUGAUUAAAGGUAGCAAAAGGGGUAAUACAG | 489 |
| | UACAAUUUUUGGAGcaggUUUUcUgacUUcggUcggaaaaccccU | |
| N3-15 | AUGAUAGGGACUUAGGGUGAAAUACAGUACCCAUAAUAAAGGGCUCAG | 490 |
| | GGAAUUUUUGGAGcaggUUUUcUgacUUcggUcggaaaaccccU | |
| N3-16 | AUGAUAGGGACUUAGGGUGAGGGCUCAGGGGAGGAACCAGCGCUCCAC | 491 |
| | CCAAUUUUUGGAGcaggUUUUcUgacUUcggUcggaaaaccccU | |
| N5-ESSalt1- | AUGAUAGGGACUUAGGGUGAAACCAGCGCUCCACCCCAUCAAUUUUUG | 492 |
| 01 | GAGcaggUUUUcUgacUUcggUcggaaaaccccU | |
| N5-ESSalt1- | UUUGUUCCGUGGGUGGUUUACAAGUUGGAGCAAGAUUAUCAAUUUUUG | 493 |
| 02 | GAGcaggUUUUcUgacUUcggUcggaaaaccccU | |
| N5-ESSalt1- | UUUGUUCCGUGGGUGGUUUACUAUACAAAAUAGAAAUAUAAAUUUUUG | 494 |
| 03 | GAGcaggUUUUcUgacUUcggUcggaaaaccccU | |
| N5-ESSalt1- | UUUGUUCCGUGGGUGGUUUAUAGUUUGUUAUUAGUUAGAAAAUUUUU | 495 |
| 04 | GGAGcaggUUUUcUgacUUcggUcggaaaaccccU | |
| N5-ESSalt1- | UUUGUUCCGUGGGUGGUUUAGAGGAACCAGCGCUCCACCCCAUCAAUU | 496 |
| 05 | UUUGGAGcaggUUUUcUgacUUcggUcggaaaaccccU | |
| N5-ESSalt1- | UUUGUUCCGUGGGUGGUUUACAUCCAAGUUGGAGCAAGAUUAUCAAUU | 497 |
| 06 | UUUGGAGcaggUUUUcUgacUUcggUcggaaaaccccU | |
| N5-ESSalt1- | UUUGUUCCGUGGGUGGUUUAUAUCCUAUACAAAAUAGAAAUAUAAAUU | 498 |
| 07 | UUUGGAGcaggUUUUcUgacUUcggUcggaaaaccccU | |
| N5-ESSalt1- | UUUGUUCCGUGGGUGGUUUAUAUAUAGUUUGUUAUUAGUUAGAAAAU | 499 |
| 08 | UUUUGGAGcaggUUUUcUgacUUcggUcggaaaaccccU | |
| N5-ESSalt1- | UUUGUUCCGUGGGUGGUUUAGGGAGGAACCAGCGCUCCACCCCAUCAA | 500 |
| 09 | UUUUUGGAGcaggUUUUcUgacUUcggUcggaaaaccccU | |
| N5-ESSalt1- | UUUGUUCCGUGGGUGGUUUACCCAUCCAAGUUGGAGCAAGAUUAUCAA | 501 |
| 10 | UUUUUGGAGcaggUUUUcUgacUUcggUcggaaaaccccU | |
| N5-ESSalt1- | UUUGUUCCGUGGGUGGUUUAAUUAUCCUAUACAAAAUAGAAAUAUAAA | 502 |
| 11 | UUUUUGGAGcaggUUUUcUgacUUcggUcggaaaaccccU | |
| N5-ESSalt1- | UUUGUUCCGUGGGUGGUUUAAAUAUAUAGUUUGUUAUUAGUUAGAAA | 503 |
| 12 | AUUUUUGGAGcaggUUUUcUgacUUcggUcggaaaaccccU | |
| N5-ESSalt1- | UUUGUUCCGUGGGUGGUUUAUCAGGGGAGGAACCAGCGCUCCACCCCA | 504 |
| 13 | UCAAUUUUUGGAGcaggUUUUcUgacUUcggUcggaaaaccccU | |
| N5-ESSalt1- | UUUGUUCCGUGGGUGGUUUACCACCCCAUCCAAGUUGGAGCAAGAUUA | 505 |
| 14 | UCAAUUUUUGGAGcaggUUUUcUgacUUcggUcggaaaaccccU | |
| N5-ESSalt1- | UUUGUUCCGUGGGUGGUUUACAAGAUUAUCCUAUACAAAAUAGAAAUA | 506 |
| 15 | UAAAUUUUUGGAGcaggUUUUcUgacUUcggUcggaaaaccccU | |
| N5-ESSalt1- | UUUGUUCCGUGGGUGGUUUAUAGAAAUAUAUAGUUUGUUAUUAGUUA | 507 |
| 16 | GAAAAUUUUUGGAGcaggUUUUcUgacUUcggUcggaaaaccccU | |
| N3-ESSalt1- | UUUGUUCCGUGGGUGGUUUAAGUGCAAGGAUUAAAGGUAGAAUUUUU | 508 |
| 01 | GGAGcaggUUUUcUgacUUcggUcggaaaaccccU | |
| N3-ESSalt1- | UUUGUUCCGUGGGUGGUUUACAAAAGGGGUAAUACAGUACAAUUUUUG | 509 |
| 02 | GAGcaggUUUUcUgacUUcggUcggaaaaccccU | |
| N3-ESSalt1- | UUUGUUCCGUGGGUGGUUUACCAUAAUAAAGGGCUCAGGGAAUUUUUG | 510 |
| 03 | GAGcaggUUUUcUgacUUcggUcggaaaaccccU | |
| N3-ESSalt1- | UUUGUUCCGUGGGUGGUUUAGAGGAACCAGCGCUCCACCCAAUUUUUG | 511 |
| 04 | GAGcaggUUUUcUgacUUcggUcggaaaaccccU | |
| N3-ESSalt1- | UUUGUUCCGUGGGUGGUUUAUCACAGUGCAAGGAUUAAAGGUAGAAUU | 512 |
| 05 | UUUGGAGcaggUUUUcUgacUUcggUcggaaaaccccU | |
| N3-ESSalt1- | UUUGUUCCGUGGGUGGUUUAGUAGCAAAAGGGGUAAUACAGUACAAUU | 513 |
| 06 | UUUGGAGcaggUUUUcUgacUUcggUcggaaaaccccU | |
| N3-ESSalt1- | UUUGUUCCGUGGGUGGUUUAGUACCCAUAAUAAAGGGCUCAGGGAAUU | 514 |
| 07 | UUUGGAGcaggUUUUcUgacUUcggUcggaaaaccccU | |
| N3-ESSalt1- | UUUGUUCCGUGGGUGGUUUAAGGGGAGGAACCAGCGCUCCACCCAAUU | 515 |
| 08 | UUUGGAGcaggUUUUcUgacUUcggUcggaaaaccccU | |
| N3-ESSalt1- | UUUGUUCCGUGGGUGGUUUAAGUCACAGUGCAAGGAUUAAAGGUAGAA | 516 |
| 09 | UUUUUGGAGcaggUUUUcUgacUUcggUcggaaaaccccU | |
| N3-ESSalt1- | UUUGUUCCGUGGGUGGUUUAAGGUAGCAAAAGGGGUAAUACAGUACAA | 517 |
| 10 | UUUUUGGAGcaggUUUUcUgacUUcggUcggaaaaccccU | |
| N3-ESSalt1- | UUUGUUCCGUGGGUGGUUUACAGUACCCAUAAUAAAGGGCUCAGGGAA | 518 |
| 11 | UUUUUGGAGcaggUUUUcUgacUUcggUcggaaaaccccU | |
| N3-ESSalt1- | UUUGUUCCGUGGGUGGUUUAUCAGGGGAGGAACCAGCGCUCCACCCAA | 519 |
| 12 | UUUUUGGAGcaggUUUUcUgacUUcggUcggaaaaccccU | |
| N3-ESSalt1- | UUUGUUCCGUGGGUGGUUUACAUAAGUCACAGUGCAAGGAUUAAAGGU | 520 |
| 13 | AGAAUUUUUGGAGcaggUUUUcUgacUUcggUcggaaaaccccU | |
| N3-ESSalt1- | UUUGUUCCGUGGGUGGUUUAUUAAAGGUAGCAAAAGGGGUAAUACAGU | 521 |
| 14 | ACAAUUUUUGGAGcaggUUUUcUgacUUcggUcggaaaaccccU | |
| N3-ESSalt1- | UUUGUUCCGUGGGUGGUUUAAAUACAGUACCCAUAAUAAAGGGCUCAG | 522 |
| 15 | GGAAUUUUUGGAGcaggUUUUcUgacUUcggUcggaaaaccccU | |
| N3-ESSalt1- | UUUGUUCCGUGGGUGGUUUAGGGCUCAGGGGAGGAACCAGCGCUCCAC | 523 |
| 16 | CCAAUUUUUGGAGcaggUUUUcUgacUUcggUcggaaaaccccU | |
| N5-ESSalt2- | UUUGUUCCGUGGGUGGUUUAAACCAGCGCUCCACCCCAUCAAUUUUUG | 524 |
| 01 | GAGcaggUUUUcUgacUUcggUcggaaaaccccU | |
| N5-ESSalt2- | UUUGUUCCGUGGGUGGUUUACAAGUUGGAGCAAGAUUAUCAAUUUUUG | 525 |
| 02 | GAGcaggUUUUcUgacUUcggUcggaaaaccccU | |
| N5-ESSalt2- | UUUGUUCCGUGGGUGGUUUACUAUACAAAAUAGAAAUAUAAAUUUUUG | 526 |
| 03 | GAGcaggUUUUcUgacUUcggUcggaaaaccccU | |
| N5-ESSalt2- | UGGGGGGAGGUAGGUAGGUAUAGUUUGUUAUUAGUUAGAAAAUUUUU | 527 |
| 04 | GGAGcaggUUUUcUgacUUcggUcggaaaaccccU | |
| N5-ESSalt2- | UGGGGGGAGGUAGGUAGGUAGAGGAACCAGCGCUCCACCCCAUCAAUU | 528 |
| 05 | UUUGGAGcaggUUUUcUgacUUcggUcggaaaaccccU | |
| N5-ESSalt2- | UGGGGGGAGGUAGGUAGGUACAUCCAAGUUGGAGCAAGAUUAUCAAUU | 529 |
| 06 | UUUGGAGcaggUUUUcUgacUUcggUcggaaaaccccU | |
| N5-ESSalt2- | UGGGGGGAGGUAGGUAGGUAUAUCCUAUACAAAAUAGAAAUAUAAAU | 530 |
| 07 | UUUUGGAGcaggUUUUcUgacUUcggUcggaaaaccccU | |
| N5-ESSalt2- | UGGGGGGAGGUAGGUAGGUAUAUAUAGUUUGUUAUUAGUUAGAAAAU | 531 |
| 08 | UUUUGGAGcaggUUUUcUgacUUcggUcggaaaaccccU | |
| N5-ESSalt2- | UGGGGGGAGGUAGGUAGGUAGGGAGGAACCAGCGCUCCACCCCAUCAA | 532 |
| 09 | UUUUUGGAGcaggUUUUcUgacUUcggUcggaaaaccccU | |
| N5-ESSalt2- | UGGGGGGAGGUAGGUAGGUACCCAUCCAAGUUGGAGCAAGAUUAUCAA | 533 |
| 10 | UUUUUGGAGcaggUUUUcUgacUUcggUcggaaaaccccU | |
| N5-ESSalt2. | UGGGGGGAGGUAGGUAGGUAAUUAUCCUAUACAAAAUAGAAAUAUAA | 534 |
| 11 | AUUUUUGGAGcaggUUUUcUgacUUcggUcggaaaaccccU | |
| N5-ESSalt2- | UGGGGGGAGGUAGGUAGGUAUCAGGGGAGGAACCAGCGCUCCACCCCA | 535 |
| 13 | UCAAUUUUUGGAGcaggUUUUcUgacUUcggUcggaaaaccccU | |
| N5-ESSalt2- | UGGGGGGAGGUAGGUAGGUACCACCCCAUCCAAGUUGGAGCAAGAUUA | 536 |
| 14 | UCAAUUUUUGGAGcaggUUUUcUgacUUcggUcggaaaaccccU | |
| N5-ESSalt2- | UGGGGGGAGGUAGGUAGGUACAAGAUUAUCCUAUACAAAAUAGAAAUA | 537 |
| 15 | UAAAUUUUUGGAGcaggUUUUcUgacUUcggUcggaaaaccccU | |
| N3-ESSalt2- | UGGGGGGAGGUAGGUAGGUAAGUGCAAGGAUUAAAGGUAGAAUUUUU | 538 |
| 01 | GGAGcaggUUUUcUgacUUcggUcggaaaaccccU | |
| N3-ESSalt2- | UGGGGGGAGGUAGGUAGGUACAAAAGGGGUAAUACAGUACAAUUUUU | 539 |
| 02 | GGAGcaggUUUUcUgacUUcggUcggaaaaccccU | |
| N3-ESSalt2- | UGGGGGGAGGUAGGUAGGUACCAUAAUAAAGGGCUCAGGGAAUUUUUG | 540 |
| 03 | GAGcaggUUUUcUgacUUcggUcggaaaaccccU | |
| N3-ESSalt2- | UGGGGGGAGGUAGGUAGGUAGAGGAACCAGCGCUCCACCCAAUUUUUG | 541 |
| 04 | GAGcaggUUUUcUgacUUcggUcggaaaaccccU | |
| N3-ESSalt2- | UGGGGGGAGGUAGGUAGGUAUCACAGUGCAAGGAUUAAAGGUAGAAU | 542 |
| 05 | UUUUGGAGcaggUUUUcUgacUUcggUcggaaaaccccU | |
| N3-ESSalt2- | UGGGGGGAGGUAGGUAGGUAGUAGCAAAAGGGGUAAUACAGUACAAU | 543 |
| 06 | UUUUGGAGcaggUUUUcUgacUUcggUcggaaaaccccU | |
| N3-ESSalt2- | UGGGGGGAGGUAGGUAGGUAGUACCCAUAAUAAAGGGCUCAGGGAAUU | 544 |
| 07 | UUUGGAGcaggUUUUcUgacUUcggUcggaaaaccccU | |
| N3-ESSalt2- | UGGGGGGAGGUAGGUAGGUAAGGGGAGGAACCAGCGCUCCACCCAAUU | 545 |
| 08 | UUUGGAGcaggUUUUcUgacUUcggUcggaaaaccccU | |
| N3-ESSalt2- | UGGGGGGAGGUAGGUAGGUAAGUCACAGUGCAAGGAUUAAAGGUAGA | 546 |
| 09 | AUUUUUGGAGcaggUUUUcUgacUUcggUcggaaaaccccU | |
| N3-ESSalt2- | UGGGGGGAGGUAGGUAGGUAAGGUAGCAAAAGGGGUAAUACAGUACA | 547 |
| 10 | AUUUUUGGAGcaggUUUUcUgacUUcggUcggaaaaccccU | |
| N3-ESSalt2- | UGGGGGGAGGUAGGUAGGUACAGUACCCAUAAUAAAGGGCUCAGGGAA | 548 |
| 11 | UUUUUGGAGcaggUUUUcUgacUUcggUcggaaaaccccU | |
| N3-ESSalt2- | UGGGGGGAGGUAGGUAGGUACAUAAGUCACAGUGCAAGGAUUAAAGGU | 549 |
| 13 | AGAAUUUUUGGAGcaggUUUUcUgacUUcggUcggaaaaccccU | |
| N3-ESSalt2- | UGGGGGGAGGUAGGUAGGUAUUAAAGGUAGCAAAAGGGGUAAUACAG | 550 |
| 14 | UACAAUUUUUGGAGcaggUUUUcUgacUUcggUcggaaaaccccU | |
| N3-ESSalt2- | UGGGGGGAGGUAGGUAGGUAAAUACAGUACCCAUAAUAAAGGGCUCAG | 551 |
| 15 | GGAAUUUUUGGAGcaggUUUUcUgacUUcggUcggaaaaccccU | |

TABLE 7C
Example U7 snRNA sequences targeting SCN1A mRNA-corresponding with constructs
in Table 3B

| Sequence | | SEQ ID |
|---|---|---|
| Name | snRNA Sequences (5′ to 3′) | NO: |
| 3c5c-24 | AUGAUAGGGACUUAGGGUGAGCACCCCAUCCCCCACUACAAAUUUU | 552 |
| | UGGAGcaggUUUUcUgacUUcggUcggaaaaccccU | |
| 3c5c-28 | AUGAUAGGGACUUAGGGUGAGUAAUACAGUACCCAUAAUAAAGGAG | 553 |
| | CAAGAUUAUCCUAUAUAAAAUAAAUUUUUGGAGcaggUUUUcUgacUUc | |
| | ggUcggaaaaccccU | |
| 3c5c-30 | AUGAUAGGGACUUAGGGUGAGGGUAAUACAGUACCCAUAAUAAAGG | 554 |
| | GCGGAGCAAGAUUAUCCUAUAUAAAAUAGAAAUUUUUGGAGcaggUU | |
| | UUcUgacUUcggUcggaaaaccccU | |
| 3ex5ex-24 | AUGAUAGGGACUUAGGGUGAGGGGUAAUACAGUACCCAUAAUAAAG | 555 |
| | GGCUUGGAGCAAGAUUAUCCUAUAUAAAAUAGAAAAUUUUUGGAGc | |
| | aggUUUUcUgacUUcggUcggaaaaccccU | |
| 3ex5ex-28 | AUGAUAGGGACUUAGGGUGAAGGGGUAAUACAGUACCCAUAAUAAG | 556 |
| | AUUAUCCUAUAUAAAAUAGAAAAAUUUUUGGAGcaggUUUUcUgacUUc | |
| | ggUcggaaaaccccU | |
| 3ex5ex-30 | AUGAUAGGGACUUAGGGUGAAAAAGGGGUAAUACAGUACCCAUAAU | 557 |
| | AAAAGAUUAUCCUAUAUAAAAUAGAAAUAUAAUUUUUGGAGcaggUU | |
| | UUcUgacUUcggUcggaaaaccccU | |
| 3ex5int-24 | AUGAUAGGGACUUAGGGUGACAAAAGGGGUAAUACAGUACCCAUAA | 558 |
| | UAAACAAGAUUAUCCUAUAUAAAAUAGAAAUAUAAAUUUUUGGAGC | |
| | aggUUUUcUgacUUcggUcggaaaaccccU | |
| 3ex5int-28 | AUGAUAGGGACUUAGGGUGAUACAGUACCCAUAAUAAAGGGCUGUU | 559 |
| | GGAGCAAGAUUAUCCUAUAUAAAAUUUUUGGAGcaggUUUUcUgacUUc | |
| | ggUcggaaaaccccU | |
| 3ex5int30 | AUGAUAGGGACUUAGGGUGAAUACAGUACCCAUAAUAAAGGGCUGA | 560 |
| | GGAAGUUGGAGCAAGAUUAUCCUAUAUAAAAAUUUUUGGAGcaggUU | |
| | UUcUgacUUcggUcggaaaaccccU | |
| 3int5ex-24 | AUGAUAGGGACUUAGGGUGAAAUACAGUACCCAUAAUAAAGGGCUG | 561 |
| | AGGGCAAGUUGGAGCAAGAUUAUCCUAUAUAAAAAAUUUUUGGAGc | |
| | aggUUUUcUgacUUcggUcggaaaaccccU | |
| 3int5ex-28 | AUGAUAGGGACUUAGGGUGAAGGGGUAAUACAGUACCCAUAAUAUU | 562 |
| | GGAGCAAGAUUAUCCUAUAUAAAAUUUUUGGAGcaggUUUUcUgacUUc | |
| | ggUcggaaaaccccU | |
| 3int5ex-30 | AUGAUAGGGACUUAGGGUGAAAAAGGGGUAAUACAGUACCCAUAAU | 563 |
| | AAAAGUUGGAGCAAGAUUAUCCUAUAUAAAAAUUUUUGGAGcaggUU | |
| | UUcUgacUUcggUcggaaaaccccU | |
| 3int5int-24 | AUGAUAGGGACUUAGGGUGACAAAAGGGGUAAUACAGUACCCAUAA | 564 |
| | UAAACAAGUUGGAGCAAGAUUAUCCUAUAUAAAAAAUUUUUGGAGc | |
| | aggUUUUcUgacUUcggUcggaaaaccccU | |
| 3int5int-28 | AUGAUAGGGACUUAGGGUGAUACAGUACCCAUAAUAAAGGGCUGAG | 565 |
| | AUUAUCCUAUAUAAAAUAGAAAAAUUUUUGGAGcaggUUUUcUgacUUc | |
| | ggUcggaaaaccccU | |
| 3int5int-30 | AUGAUAGGGACUUAGGGUGAAUACAGUACCCAUAAUAAAGGGCUGA | 566 |
| | GGAAGAUUAUCCUAUAUAAAAUAGAAAUAUAAUUUUUGGAGcaggUU | |
| | UUcUgacUUcggUcggaaaaccccU | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for modifying nucleic acid splicing, comprising:
an exonic splicing silencer (ESS) nucleic acid sequence; and
an antisense nucleic acid sequence that targets an alternatively spliced region of a ribonucleic acid (RNA) encoding a sodium channel protein type 1 subunit alpha (SCN1A) protein.

2. The system of claim 1, wherein the system comprises an engineered U7 snRNA comprising the ESS nucleic acid sequence and the antisense nucleic acid sequence.

3. The system of claim 1, wherein the alternatively spliced region comprises an alternative exon of the RNA encoding SCN1A, wherein inclusion of the alternative exon in a mature SCN1A mRNA results in nonsense mediated decay (NMD) of a transcription product of the mature SCN1A mRNA.

4. The system of claim 1, wherein the alternatively spliced region comprises exon 20N of the RNA encoding SCN1A.

5. The system of claim 4, wherein the exon 20N comprises a nucleic acid sequence at least 80%, at least 85%, at least 90%, at least 95%, at least 98%, at least 99%, or 100% identical to SEQ ID NO: 2.

6. The system of claim 1, wherein the ESS recruits a protein factor or group of factors that reduce or silence splicing of the RNA encoding SCN1A.

7. The system of claim 1, wherein the antisense nucleic acid sequence binds to at least a portion of the alternatively spliced region.

8. The system of claim 7, wherein the portion of the alternatively spliced region is within a 5′ half or 5′ end of the alternatively spliced region.

9. The system of claim 7, wherein the portion of the alternatively spliced region comprises nucleotide positions within 50 bp of a 5′ or 3′ end of the alternatively spliced region.

10. The system of claim 1, wherein the antisense nucleic acid sequence is 3′ or downstream relative to the ESS nucleic acid sequence.

11. The system of claim 1, wherein the engineered U7 snRNA further comprises a Sm binding site.

12. The system of claim 11, wherein the Sm binding site is 3′ or downstream relative to the ESS nucleic acid sequence or antisense nucleic acid sequence.

13. The system of claim 1, wherein the engineered U7 snRNA further comprises a 3′ U7 snRNA hairpin sequence.

14. The system of claim 13, wherein the hairpin sequence is 3′ or downstream relative to the ESS nucleic acid sequence, the antisense nucleic acid sequence, or the Sm binding site.

15. The system of claim 1, wherein the system comprises an expression cassette encoding an engineered U7 snRNA, the engineered U7 snRNA comprising the ESS nucleic acid sequence and the antisense nucleic acid sequence.

16. The system of claim 15, wherein the expression cassette comprises a promoter operably linked to a sequence encoding the engineered U7 snRNA.

17. The system of claim 16, wherein the expression cassette comprises a second sequence encoding a second copy of the engineered U7 snRNA, and the promoter is further operably linked to the second sequence.

18. The system of claim 17, wherein the expression cassette comprises a third sequence encoding a third copy of the engineered U7 snRNA, and the promoter is further operably linked to the third sequence.

19. The system of claim 16, wherein the promoter comprises a mouse U1 snRNA promoter sequence, a human U1 snRNA promoter sequence, a mouse U7 snRNA promoter sequence, a human U7 snRNA promoter sequence, or a combination thereof.

20. The system of claim 15, wherein the expression cassette comprises a 3′ terminator sequence operably linked to a sequence encoding the engineered U7 snRNA.

21. The system of claim 20, wherein the 3′ terminator sequence comprises a mouse U1 snRNA terminator sequence, a human U1 snRNA terminator sequence, a mouse U7 snRNA terminator sequence, a human U7 snRNA terminator sequence, or a combination thereof.

22. The system of claim 1, wherein expression of the system in a cell or a population of cells reduces an exon 20N measurement in a cell or population of cells by at least 10%, relative to a baseline exon 20N measurement.

23. The system of claim 1, wherein expression of the system in a cell or a population of cells reduces an SCN1A NMD transcript measurement in a cell or population of cells by at least 10%, relative to a baseline SCN1A NMD transcript measurement.

24. The system of claim 1, wherein expression of the system in a cell or a population of cells increases a productive form of an SCN1A transcript measurement in a cell or population of cells by at least 10%, relative to a baseline SCN1A transcript measurement.

25. The system of claim 1, wherein expression of the system in a cell or a population of cells increases a productive form of an SCN1A transcript measurement in a cell or population of cells by more than 70%, relative to a baseline SCN1A transcript measurement.

26. The system of claim 1, wherein expression of the system in a cell or a population of cells an SCN1A protein measurement in a cell or population of cells by at least 10%, relative to a baseline SCN1A protein measurement.

27. The system of claim 1, wherein expression of the system in a cell or a population of cells an SCN1A protein measurement in a cell or population of cells by at least 70%, relative to a baseline SCN1A protein measurement.

28. A system for modifying nucleic acid splicing, comprising:
an engineered U7 small nuclear RNA (snRNA) comprising an antisense nucleic acid sequence that binds to an alternatively spliced region of a ribonucleic acid (RNA) encoding a sodium channel protein type 1 subunit alpha (SCN1A) protein.

29. The system of claim 28, wherein the engineered U7 snRNA further comprises an exonic splicing silencer (ESS) nucleic acid sequence.

30. The system of claim 28, wherein the engineered U7 snRNA does not comprise an ESS nucleic acid sequence.

31. A pharmaceutical composition comprising the system of any one of claims 1-30 and a pharmaceutically acceptable carrier.

32. An engineered virus comprising the system of any one of claims 1-30.

33. The engineered virus of claim 32, comprising an adeno-associated virus (AAV).

34. A method, comprising: administering the engineered virus of claim 32 to a subject.

35. A method of modifying splicing, comprising: contacting a pre-mRNA encoding SCN1A with an engineered U7 snRNA or with a vector encoding the engineered U7 snRNA, wherein the engineered U7 snRNA induces exclusion of exon 20N from a mature mRNA generated by the pre-mRNA.

36. The method of claim 35, wherein the method increases an amount of a productive isoform of SCN1A, relative to a control or baseline amount of said productive isoform.

37. The method of claim 35, wherein the contact or administration decreases an amount of a non-productive isoform of SCN1A in the subject, relative to a control or baseline amount of said non-productive isoform.

38. The method of claim 35, wherein the method increases an amount of a NaV1.1, relative to a control or baseline amount of the NaV1.1.

39. A method of treating or preventing epilepsy in a subject in need thereof, comprising: administering a therapeutically effective amount of a recombinant U7 snRNA composition that silences or reduces splicing of an alternatively spliced region of an RNA encoding SCN1A.

40. The method of claim 39, further comprising identifying the subject as having epilepsy or as being at risk of having epilepsy, and selecting the treatment based on said identifying.

41. The method of claim 39, wherein the administration prevents the subject from having seizures, or reduces an amount or severity of the seizures of the subject relative to a baseline amount or severity.

42. The method of claim 39, wherein the administration reduce a seizure rate in the subject, relative to a baseline seizure rate before the administration.

43. The method of claim 42, wherein the seizure rate is reduced in the subject by at least 10%, relative to the baseline seizure rate.

44. The method of claim 42, wherein the seizure rate is reduced in the subject by at least 90%, relative to the baseline seizure rate.

45. The method of claim 39, wherein the administration reduce a seizure duration in the subject, relative to a baseline seizure duration before the administration.

46. The method of claim 45, wherein the seizure duration is reduced in the subject by at least 10%, relative to the baseline seizure duration.

47. A method of treating or Dravet Syndrome in a subject in need thereof, comprising: administering a therapeutically effective amount of a synthetic composition comprising a recombinant U7 snRNA that silences or reduces splicing of an alternatively spliced region of a RNA encoding SCN1A.

48. The method of claim 47, further comprising identifying the subject as having Dravet syndrome, and selecting the treatment based on said identifying.

49. The method of claim 47, wherein the administration reduces or improves a symptom of the Drave syndrome in the subject.

50. The method of claim 47, wherein the administration prevents the subject from having seizures, or reduces an amount or severity of the seizures of the subject relative to a baseline amount or severity.
